(19) United States
(12) Patent Application Publication
(10) Pub. No.: US 2009/0203705 A1
(43) Pub. Date: Aug. 13, 2009

(54) Spiro Compounds As NPY Y5 Receptor Antagonists

(76) Inventors: Matteo Biagetti, Verona (IT); Stefania Anna Contini, c/o Verona (IT); Thorsten Genski, c/o Verona (IT); Sebastien Guery, c/o Verona (IT); Colin Philip Leslie, Verona (IT); Angelica Mazzali, c/o Verona (IT); Domenica Antonia Pizzi, Verona (IT); Fabio Maria Sabbatini, Verona (IT); Catia Seri, c/o Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/360,166

(22) Filed: Jan. 27, 2009

(30) Foreign Application Priority Data
Jan. 29, 2008 (GB) 0801597.6
Oct. 17, 2008 (GB) 0819112.4

(51) Int. Cl. A61K 31/501; C07D 413/14; A61P 3/04

(52) U.S. Cl. 514/252.02; 544/238; 514/252.03

(57) ABSTRACT

The present invention relates to novel compounds of formula (I), or a pharmaceutically acceptable salt thereof,

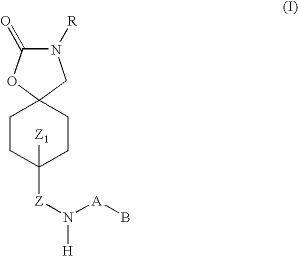

wherein
R is an aryl or heteroaryl, which may be substituted by one or more: halogen, C₁-C₄ alkyl, C1-C4 alkoxy, C₁-C₄ haloalkyl, C₁-C₄ haloalkoxy, cyano;
Z₁ is H, C₁-C₄ alkyl or F;
Z is CH₂, CH(C₁-C₄ alkyl), C(C₁-C₄ alkyl)₂ or a bond;
A is a 6-10 membered aryl or heteroaryl, which may be substituted by one or more: halogen, C₁-C₄ alkyl, C₁-C₄ alkoxy, C₁-C₄ haloalkyl, C₁-C₄ haloalkoxy, cyano; or —C(═O)—X; or —O(CH₂)₀₋₁R₁;
B is hydrogen or is a 5-6 membered heteroaryl, or a 4-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxyl, cyano; A and B being linked via any atom;
R₁ is —(C₁-C₄)alkyl(C₁-C₄)alkoxy; or C₃-C₈ cycloalkyl; or R₁ is an aryl or heteroaryl, which may be substituted by one or more: halogen, C₁-C₄ alkyl, C₁-C₄ alkoxy, C₁-C₄ haloalkyl, C₁-C₄ haloalkoxy, cyano; or R₁ is a 4-6 membered heterocycle, which may be substituted by one or more: halogen, C₁-C₄ alkyl, C₁-C₄ alkoxy, C₁-C₄ haloalkyl, C₁-C₄ haloalkoxy, cyano;
X is OR₂ or NR₃R₄;
R₂ is C₁-C₄ alkyl;
R₃ is hydrogen or together with R₄ and the nitrogen form a 5-6 saturated membered ring;
R₄ is C₃-C₈ cycloalkyl;
   processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

The present application claims priority of GB0801597.6 and GB0819112.4.

The present invention relates to novel compounds, processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

Neuropeptide Y (hereinafter referred to as NPY), a peptide consisting of 36 amino acids, was first isolated from porcine brain by Tatemoto et al. in 1982 [Nature, 296: 659 (1982)]. NPY is widely distributed in central and peripheral nervous systems and plays various roles as one of the most abundant peptides in the nervous system. NPY acts as an orexigenic substance in the central nervous system and markedly promotes fat accumulation via the mediation of the secretion of various hormones or the action of the nervous system. It is known that the continuous intracerebroventricular administration of NPY induces obesity and insulin resistance based on these actions (International Journal of Obesity, vol. 19: 517 (1995); Endocrinology, vol. 133: 1753 (1993)). It is also known that NPY has central effects that are related to diseases such as depression, anxiety, schizophrenia, pain, dementia and the like (Drugs, vol. 52, 371 (1996). Furthermore, in the periphery, NPY coexists with norepinephrine in sympathetic nerve endings and is involved in the tonicity of the sympathetic nervous system. It is known that peripheral administration of NPY causes vasoconstriction and enhances the activities of other vasoconstrictive substances such as norepinephrine (British Journal of Pharmacology, vol. 95: 419 (1988)). It is also reported that NPY could participate in the development of cardiac hypertrophy as a result of the sympathetic stimulation (Proceeding National Academic Science USA, Vol. 97, 1595 (2000)).

Endogenous receptor proteins that bind NPY and related peptides as ligands have been identified and distinguished, and several such proteins have been cloned and expressed. Six different receptor subtypes [Y1, Y2, Y3, Y4(PP), Y5, Y6] are recognised today based upon binding profile, pharmacology and/or composition if identity is known.

The Y5 subtype was isolated, characterized and reported recently in U.S. Pat. No. 5,602,024 (WO 96/16542). The effects mediated by the NPY Y5 receptor include eating stimulation and accumulation of fat (Nature, vol. 382, 168 (1996); American Journal of Physiology, vol. 277, R1428 (1999)). It is reported that the NPY Y5 receptor also mediates some CNS effects, such as seizure and epilepsy, or pain and morphine withdrawal symptoms (Natural Medicine, vol. 3, 761 (1997); Proceeding Academic Science USA, vol. 96, 13518 (1999); The Journal of Pharmacology and Experimental Therapeutics, vol. 284, 633 (1998)). In the periphery, the NPY Y5 receptor is reported to be involved in diuresis and the hypoglycemic effect caused by NPY (British Journal of Pharmacology, vol. 120, 1335 (1998); Endocrinology, vol. 139, 3018 (1998)). NPY is also reported to enhance cardiac hypertrophy as a result of sympathetic accentuation (Proceeding National Academic Science USA, Vol. 97, 1595 (2000)).

The effects of NPY occur by binding to the NPY receptors in the central or peripheral nervous system. Therefore, the action of NPY can be prevented by blocking the binding to NPY receptors. Substances that antagonize NPY binding to NPY receptors may be useful for the prophylaxis or treatment of various diseases related to NPY, such as cardiovascular disorders (for example hypertension, nephropathy, heart disease, vasospasm), central nervous system disorders (for example bulimia, binge eating, depression, anxiety, seizure, epilepsy, dementia, pain, alcoholism, drug withdrawal), metabolic diseases (for example obesity, diabetes, hormone abnormality), sexual and reproductive dysfunction, gastrointestinal motility disorder, respiratory disorder, inflammation or glaucoma and the like (Trends in Pharmacological Sciences, 15, 153 (1994); Life Science, 55, 551 (1994); Drugs, vol. 52, 371 (1996); The Journal of Allergy and Immunology, vol. 101, S345 (1998); Nature, vol. 396, 366 (1998); The Journal of Pharmacology and Experimental Therapeutics, vol. 284, 633 (1998); Trends in Pharmacological Science, vol. 20, 104 (1999); Proceeding National Academic Science USA, vol. 97, 1595 (2000)).

The object of the present invention is to provide a compound of formula (I) or a pharmaceutically acceptable salt thereof:

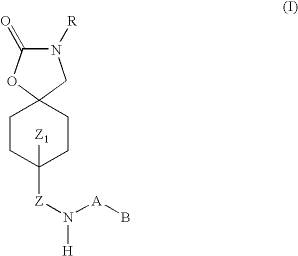

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
Z₁ is H, C₁-C₄ alkyl or F;
Z is CH₂, CH(C₁-C₄ alkyl), C(C₁-C₄ alkyl)₂ or a bond;
A is a 6-10 membered aryl or heteroaryl, which may be substituted by one or more: halogen, C₁-C₄ alkyl, C₁-C₄ alkoxy, C₁-C₄ haloalkyl, C₁-C₄ haloalkoxy, cyano; or C(═O)—X; or —O(CH₂)₀₋₁R₁;
B is hydrogen or is a 5-6 membered heteroaryl, or a 4-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxy, cyano; A and B being linked via any atom;
R₁ is —(C₁-C₄)alkyl(C₁-C₄)alkoxy; or C₃-C₈ cycloalkyl; or R₁ is an aryl or heteroaryl, which may be substituted by one or more: halogen, C₁-C₄ alkyl, C₁-C₄ alkoxy, C₁-C₄ haloalkyl, C₁-C₄ haloalkoxy, cyano; or R₁ is a 4-6 membered heterocycle, which may be substituted by one or more: halogen, C₁-C₄ alkyl, C₁-C₄ alkoxy, C₁-C₄ haloalkyl, C₁-C₄ haloalkoxy, cyano;
X is OR₂ or NR₃R₄
R₂ is C₁-C₄ alkyl;
R₃ is hydrogen or together with R₄ and the nitrogen form a 5-6 saturated membered ring;
R₄ is C₃-C₈ cycloalkyl.

The compounds of the present invention may be in the form of and/or may be administered as a pharmaceutically acceptable salt. For a review on suitable salts see Berge et al, J. Pharm. Sci., 1977, 66, 1-19.

Typically, a pharmaceutically acceptable salt may be readily prepared by using a desired acid or base as appropriate. The salt may precipitate from solution and be collected by filtration or may be recovered by evaporation of the solvent.

Suitable pharmaceutically acceptable addition salts are formed from acids which form non-toxic salts. Examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, pyruvate, oxalate, oxaloacetate, trifluoroacetate, saccharate, benzoate, methanesulphonate, ethanesulphonate, benzenesulphonate, p-toluenesulphonate and isethionate.

In one embodiment, the pharmaceutically acceptable salt is a hydrochloride salt, a dihydrochloride salt or formate salt.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Pharmaceutically acceptable salts may also be prepared from other salts, including other pharmaceutically acceptable salts, of the compound of formula (I) using conventional methods.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

In addition, prodrugs are also included within the context of this invention. As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

The term prodrug also encompasses any covalently bonded carriers that release a compound of structure (I) in vivo when such a prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein amine groups are bonded to any group that, when administered to a patient, cleaves to form the amine groups. Thus, representative examples of prodrugs include (but are not limited to) acetate, formate and benzoate derivatives of amine functional groups of the compounds of structure (I).

Hereinafter, compounds of formula (I) (whether in solvated or unsolvated form) or their pharmaceutically acceptable salt (whether in solvated or unsolvated form) or prodrugs thereof defined in any aspect of the invention (except intermediate compounds in chemical processes) are referred to as “compounds of the invention”.

With regard to stereoisomers, the compounds of formula (I) may have one or more asymmetric carbon atoms and may occur as racemates, racemic mixtures and as individual enantiomers or diastereomers. All such isomeric forms are included within the present invention, including mixtures thereof.

When a specific enantiomer of a compound of formula (I) is required, this may be obtained for example by resolution of a corresponding enantiomeric mixture of a compound of formula (I) using conventional methods, such as H.P.L.C. of the corresponding racemate using a suitable chiral support or by fractional crystallisation of the diastereoisomeric salts formed by reaction of the corresponding racemate with a suitable optically active acid or base, as appropriate. Or a specific enantiomer may also be prepared from a corresponding optically pure intermediate. Separation of diastereoisomers or cis and trans isomers or syn and anti isomers may be achieved by conventional techniques, e.g. by fractional crystallisation, chromatography or H.P.L.C. of a stereoisomeric mixture.

Furthermore, some of the crystalline forms of the compounds of structure (I) may exist as polymorphs, which are included in the present invention.

The term C₁-C₄ alkyl as used herein as a group or a part of the group refers to a linear or branched alkyl group containing from one to four carbon atoms; examples of such groups include methyl, ethyl, propyl, isopropyl, n-butyl, isobutyl and tert butyl.

The term halogen refers to a fluorine, chlorine, bromine or iodine atom.

The term C₁-C₄ haloalkyl means an alkyl group having one to four carbon atoms and wherein at least one hydrogen atom is replaced with halogen such as for example a trifluoromethyl group and the like.

The term C₁-C₄ alkoxy group means be a linear or a branched chain alkoxy group, for example methoxy, ethoxy, propoxy, prop-2-oxy, butoxy, but-2-oxy or methylprop-2-oxy and the like.

The term C₁-C₄ haloalkoxy group means be a C₁-C₄ alkoxy group as defined before substituted with at least one halogen, preferably fluorine, such as OCHF₂, or OCF₃.

The term C₃-C₈ cycloalkyl means a saturated monocyclic hydrocarbon ring of three to eight carbon atoms. Examples of such groups include cyclopropyl, cyclobutyl, cyclopentyl, cyclohexyl, cycloheptyl or cyclooctyl and the like.

The term aryl means an aromatic carbocyclic moiety of 6 to 12 members. Examples of such aryls include (but are not limited to): phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono- and bicyclic ring systems.

Representative heteroaryls include (but are not limited to): furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, benzoisothiazolyl, pyridazinyl, pyrimidinyl, pyrazinyl, triazinyl, cinnolinyl, phthalazinyl, triazolyl, tetrazolyl, quinazolinyl, benzodioxolyl, benzothiadiazolyl, benzooxadiazolyl, imidazo[1,2-a]pyrazinyl, imidazo[1,2-b]pyridazinyl, isothiazolyl, thiadiazolyl, [1,2,4]thiazol[1, 5-9]pyridinyl.

Representative 6-10 membered heteroaryls include (but are not limited to): benzofuranyl, benzothiophenyl, indolyl, isoindolyl, azaindolyl, pyridyl, quinolinyl, isoquinolinyl, benzoxazolyl, benzimidazolyl, benzothiazolyl, benzoisothiazolyl, pyridazinyl, pyrimidinyl, pyrazinyl, triazinyl, cinnolinyl, phthalazinyl, quinazolinyl, benzodioxolyl, benzothiadiazolyl, benzooxadiazolyl, imidazo[1,2-a]pyrazinyl, imidazo[1,2-b]pyridazinyl.

Representative 5-6 membered heteroaryls include (but are not limited to): furyl, thiophenyl, pyrrolyl, indolyl, pyridyl, oxazolyl, isooxazolyl, pyrazolyl, imidazolyl, thiazolyl, isothiazolyl, pyridazinyl, pyrimidinyl, pyrazinyl, triazinyl, triazolyl, tetrazolyl, isothiazolyl, thiadiazolyl.

The term 5-6 heterocyclyl means to a 5-6 membered monocyclic ring which may be saturated or partially unsaturated containing 1 to 4 heteroatoms selected from oxygen, sulphur and NH or N(C₁-C₄ alkyl). Examples of such monocyclic rings include pyrrolidinyl, pyrazolidinyl, oxazolidinyl, piperidinyl, piperazinyl, morpholinyl, thiomorpholinyl, thiazolidinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, 3,6-dihydropyridinyl and the like.

The term 4-6 heterocyclyl means a 4-6 membered monocyclic ring which may be saturated or partially unsaturated containing 1 to 4 heteroatoms selected from oxygen, sulphur and nitrogen. Examples of such monocyclic rings include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl, 3,6-dihydropyridinyl and the like.

With regard to stereoisomers, the compounds of formula (I),

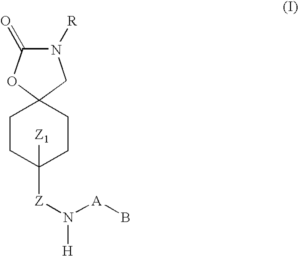

can exist as two stereoisomers represented by formulas (Ia) and (Ib).

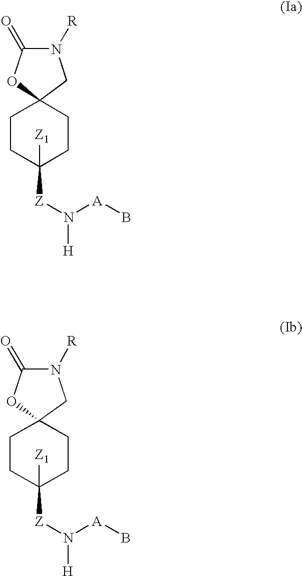

In one embodiment compound of formula (Ia) are provided in which the stereochemistry is “cis”, except when Z₁ is F wherein the stereochemistry is “trans”. In another embodiment of the present invention, compounds of formula (Ib) are provided and in which the stereochemistry is “trans”, except when Z₁ is F wherein the stereochemistry is “cis”.

“Trans” stereochemistry is due to highest priority groups, according to Kahn-Prelog-lngold classification, attached to the cyclohexane ring being on opposite sides of the cyclohexane ring. “Trans” stereochemistry can be designated also as “trans configuration” or “anti”; in the case of formula (Ib) the description (5r, 8r) can also be used to describe the “trans” stereochemistry.

In one aspect, the present invention provides a compound of formula (I), (Ia) and (Ib) in which:
R is phenyl or 5-6 membered heteroaryl; which may be substituted by one or more: halogen, C₁-C₄ alkyl, C₁-C₄ alkoxy, C₁-C₄ haloalkyl, C₁-C₄ haloalkoxy, cyano;
Z₁ is H;
Z is CH₂;
A is a 6-10 membered heteroaryl, which may be substituted by one or more: halogen, C₁-C₄ alkyl, C₁-C₄ alkoxy, C₁-C₄ haloalkyl, C₁-C₄ haloalkoxy, cyano; or C(═O)—X; or —O(CH₂)₀₋₁R₁;
B is hydrogen or is a 5-6 membered heteroaryl, or a 4-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxy, cyano; A and B being linked via any atom;
R₁ is —(C₁-C₄)alkyl(C₁-C₄)alkoxy; or C₃-C₈ cycloalkyl; or R₁ is an aryl or heteroaryl; which may be substituted by one or more: halogen, C₁-C₄ alkyl, C₁-C₄ alkoxy, C₁-C₄ haloalkyl, C₁-C₄ haloalkoxy, cyano; or R₁ is a 4-6 membered heterocycle, which may be substituted by one or more: halogen, C₁-C₄ alkyl, C₁-C₄ alkoxy, C₁-C₄ haloalkyl, C₁-C₄ haloalkoxy, cyano;
X is OR₂ or NR₃R₄
R₂ is C₁-C₄ alkyl;
R₃ is hydrogen or together with R₄ and the nitrogen form a 5-6 saturated membered ring;
R₄ is C₃-C₈ cycloalkyl.

In another embodiment a compound of formula (I)′ or a pharmaceutically acceptable salt thereof is provided, wherein

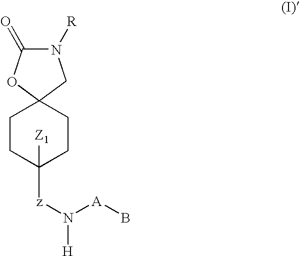

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
Z₁ is H, C₁-C₄ alkyl or F;
Z is CH₂, CH(C₁-C₄ alkyl), C(C₁-C₄ alkyl)₂ or a bond;
A is a 6-10 membered aryl or heteroaryl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxy, cyano; A and B being linked via any atom.

With regard to stereoisomers, the compounds of formula (I)′ can exist as two stereoisomers represented by formulas (Ia)′ and (Ib)′.

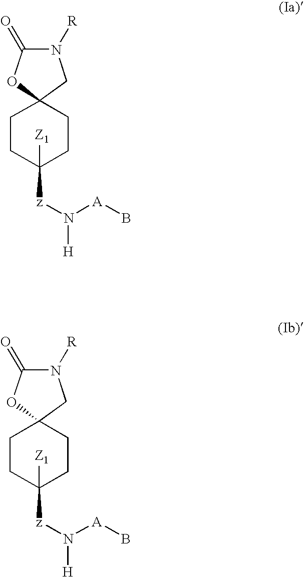

In one aspect, the present invention provides a compound of formula (I)′, (Ia)′ and (Ib)′ in which:
R is phenyl or pyridyl, pyrazolyl, pyridazinyl, pyrazinyl; which may be substituted by one or more: halogen, C₁-C₄ alkyl, C₁-C₄ alkoxy, C₁-C₄ haloalkyl, C₁-C₄ haloalkoxy, cyano;
A is selected from a group consisting of: pyridyl, pyridazinyl, pyrazinyl, pyrimidinyl, benzothiazolyl, benzoisothiazolyl, benzimidazolyl, imidazo[1,2-b]pyridizanyl; which may be substituted by one or more: halogen, C₁-C₄ alkyl, C₁-C₄ alkoxy, C₁-C₄ haloalkyl, C₁-C₄ haloalkoxy, cyano;
B is phenyl or pyridine, pyrimidine, pyrazine, pyridazine, imidazole, pyrazole, thiazole, thiadiazole, isoxazole, pyrrolidine; which may be substituted by one or more: halogen, C₁-C₄ alkyl, C₁-C₄ alkoxy, C₁-C₄ haloalkyl, C₁-C₄ haloalkoxy, cyano.

### EXAMPLE COMPOUNDS OF THE PRESENT INVENTION INCLUDE

(trans)-3-phenyl-8-({[5-(trifluoromethyl)-2-pyridinyl]amino}methyl)-1-oxa-3-azaspiro[4.5]-decan-2-one;
(trans)-3-(2-pyridinyl)-8-({[5-(trifluoromethyl)-2-pyridinyl]amino}methyl)-1-oxa-3-azaspiro[4.5]decan-2-one;
(trans)-3-(2-pyridinyl)-8-({[5-(2-pyrimidinyl)-2-pyridinyl]amino}methyl)-1-oxa-3-azaspiro[4.5]decan-2-one;
(trans)-8-({[5-(1-methyl-1H-imidazol-5-yl)-2-pyridinyl]amino}methyl)-3-(2-pyridinyl)-1-oxa-3-azaspiro[4.5]decan-2-one;
(trans)-3-(2-pyridinyl)-8-({[5-(1,3-thiazol-5-yl)-2-pyridinyl]amino}methyl)-1-oxa-3-azaspiro[4.5]decan-2-one;
(trans)-8-({[5-(5-methyl-1,3,4-thiadiazol-2-yl)-2-pyridinyl]amino}methyl)-3-(2-pyridinyl)-1-oxa-3-azaspiro[4.5]decan-2-one;
(trans)-8-[({5-[(difluoromethyl)oxy]-2-pyridinyl}amino)methyl]-3-(2-pyridinyl)-1-oxa-3-azaspiro[4.5]decan-2-one;
(trans)-8-[({5-[(cyclopropylmethyl)oxy]-2-pyridinyl}amino)methyl]-3-(2-pyridinyl)-1-oxa-3-azaspiro[4.5]decan-2-one;
(trans)-8-({[5-(cyclobutyloxy)-2-pyridinyl]amino}methyl)-3-(2-pyridinyl)-1-oxa-3-azaspiro[4.5]decan-2-one;
(trans)-5-phenyl-2-pyridinyl)amino]methyl}-3-(2-pyrazinyl)-1-oxa-3-azaspiro[4.5]decan-2-one;
(trans)-8-{[(5-phenyl-2-pyridinyl)amino]methyl}-3-(3-pyridazinyl)-1-oxa-3-azaspiro[4.5]decan-2-one;
(trans)-3-(2-fluoro-3-pyridinyl)-8-({[5-(trifluoromethyl)-2-pyridinyl]amino}methyl)-1-oxa-3-azaspiro[4.5]decan-2-one;
(trans)-3-(3-pyridazinyl)-8-({[5-(trifluoromethyl)-2-pyridinyl]amino}methyl)-1-oxa-3-azaspiro[4.5]decan-2-one;
(trans)-3-(1-methyl-1H-pyrazol-3-yl)-8-({[5-(trifluoromethyl)-2-pyridinyl]amino}methyl)-1-oxa-3-azaspiro[4.5]decan-2-one;
(trans)-3-(2-pyridinyl)-8-({[5-(1,3-thiazol-2-yl)-2-pyridinyl]amino}methyl)-1-oxa-3-azaspiro[4.5]decan-2-one;
(trans)-8-({[5-(3,5-dimethyl-4-isoxazolyl)-2-pyridinyl]amino}methyl)-3-(2-pyridinyl)-1-oxa-3-azaspiro[4.5]decan-2-one;
(trans)-8-({[5-(2-methyl-1,3-thiazol-4-yl)-2-pyridinyl]amino}methyl)-3-(2-pyridinyl)-1-oxa-3-azaspiro[4.5]decan-2-one;
(trans)-8-({[5-(4-pyridazinyl)-2-pyridinyl]amino}methyl)-3-(2-pyridinyl)-1-oxa-3-azaspiro[4.5]decan-2-one;
(trans)-3-(2-pyridinyl)-8-({[5-(1,3,5-trimethyl-1H-pyrazol-4-yl)-2-pyridinyl]amino}methyl)-1-oxa-3-azaspiro[4.5]decan-2-one;
(trans)-8-({[5-(3-methyl-1H-pyrazol-4-yl)-2-pyridinyl]amino}methyl)-3-(2-pyridinyl)-1-oxa-3-azaspiro[4.5]decan-2-one;
(trans)-8-({[5-(4-morpholinyl)-2-pyridinyl]amino}methyl)-3-(2-pyridinyl)-1-oxa-3-azaspiro[4.5]decan-2-one;
(trans)-8-({[5-(cyclopentyloxy)-2-pyridinyl]amino}methyl)-3-(2-pyridinyl)-1-oxa-3-azaspiro[4.5]decan-2-one;
(trans)-8-({[5-(cyclopentyloxy)-2-pyridinyl]amino}methyl)-3-(3-pyridazinyl)-1-oxa-3-azaspiro[4.5]decan-2-one;
(trans)-8-({[5-(cyclohexyloxy)-2-pyridinyl]amino}methyl)-3-(3-pyridazinyl)-1-oxa-3-azaspiro[4.5]decan-2-one;
(trans)-3-(3-pyridinyl)-8-({[6-(trifluoromethyl)-2-pyridinyl]amino}methyl)-1-oxa-3-azaspiro[4.5]decan-2-one;
(trans)-3-(3-pyridazinyl)-8-({[5-(2-pyrimidinyl)-2-pyridinyl]amino}methyl)-1-oxa-3-azaspiro[4.5]decan-2-one;
(trans)-8-{[(5-bromo-2-pyridinyl)amino]methyl}-3-(2-pyridinyl)-1-oxa-3-azaspiro[4.5]decan-2-one;
methyl 6-({[2-oxo-3-(3-pyridazinyl)-1-oxa-3-azaspiro[4.5]dec-8-yl]methyl}amino)-3-pyridinecarboxylate;
(trans)-8-({[5-(1H-pyrazol-1-yl)-2-pyridinyl]amino}methyl)-3-(2-pyridinyl)-1-oxa-3-azaspiro[4.5]decan-2-one;
(trans)-3-(2-fluoro-3-pyridinyl)-8-({[5-(2-pyrimidinyl)-2-pyridinyl]amino}methyl)-1-oxa-3-azaspiro[4.5]decan-2-one;
(trans)-3-(1H-pyrazol-4-yl)-8-({[5-(2-pyrimidinyl)-2-pyridinyl]amino}methyl)-1-oxa-3-azaspiro[4.5]decan-2-one;
N-cyclohexyl-6-({[(trans)-2-oxo-3-(2-pyridinyl)-1-oxa-3-azaspiro[4.5]dec-8-yl]methyl}amino)-3-pyridinecarboxamide;
(trans)-3-(2-pyridinyl)-8-({[5-(1-pyrrolidinylcarbonyl)-2-pyridinyl]amino}methyl)-1-oxa-3-azaspiro[4.5]decan-2-one;
(trans)-8-{[(3-fluoro-2,3′-bipyridin-6′-yl)amino]methyl}-3-(2-fluoro-3-pyridinyl)-1-oxa-3-azaspiro[4.5]decan-2-one;
(trans)-8-({[5-(1-piperidinylcarbonyl)-2-pyridinyl]amino}methyl)-3-(2-pyridinyl)-1-oxa-3-azaspiro[4.5]decan-2-one;
(trans)-3-(3-pyridazinyl)-8-({[6-(trifluoromethyl)-2,3′-bipyridin-6′-yl]amino}methyl)-1-oxa-3-azaspiro[4.5]decan-2-one;
(trans)-3-(3-pyridazinyl)-8-[({5-[2-(trifluoromethyl)phenyl]-2-pyridinyl}amino) methyl]-1-oxa-3-azaspiro[4.5]decan-2-one;
(trans)-3-(3-pyridazinyl)-8-({[5-(2-pyridinyloxy)-2-pyridinyl]amino}methyl)-1-oxa-3-azaspiro[4.5]decan-2-one;
(trans)-8-{[(4-phenyl-2-pyridinyl)amino]methyl}-3-(3-pyridazinyl)-1-oxa-3-azaspiro[4.5]decan-2-one;
(trans)-8-[(2,3′-bipyridin-6′-ylamino)methyl]-3-(3-pyridazinyl)-1-oxa-3-azaspiro[4.5]decan-2-one;
(trans)-8-{[(5-methyl-2,3′-bipyridin-6′-yl)amino]methyl}-3-(3-pyridazinyl)-1-oxa-3-azaspiro[4.5]decan-2-one;
(trans)-8-{[(6-fluoro-2,3′-bipyridin-6′-yl)amino]methyl}-3-(3-pyridazinyl)-1-oxa-3-azaspiro[4.5]decan-2-one;
(trans)-8-{[(3-fluoro-6-methyl-2,3′-bipyridin-6′-yl)amino]methyl}-3-(3-pyridazinyl)-1-oxa-3-azaspiro[4.5]decan-2-one;
(trans)-8-{[(5-fluoro-2,3′-bipyridin-6′-yl)amino]methyl}-3-(3-pyridazinyl)-1-oxa-3-azaspiro[4.5]decan-2-one;
(trans)-3-(3-pyridazinyl)-8-{[(5-{3-[(trifluoromethyl)oxy]phenyl}-2-pyridinyl)amino]methyl}-1-oxa-3-azaspiro[4.5]decan-2-one;
(trans)-8-({[5-(2-fluorophenyl)-2-pyridinyl]amino}methyl)-3-(3-pyridazinyl)-1-oxa-3-azaspiro[4.5]decan-2-one;
(trans)-8-[(3,3′-bipyridin-6-ylamino)methyl]-3-(3-pyridazinyl)-1-oxa-3-azaspiro[4.5]decan-2-one;
(trans)-8-{[(6′-fluoro-3,3′-bipyridin-6-yl)amino]methyl}-3-(3-pyridazinyl)-1-oxa-3-azaspiro[4.5]decan-2-one;
(trans)-8-{[(2′-fluoro-3,3′-bipyridin-6-yl)amino]methyl}-3-(3-pyridazinyl)-1-oxa-3-azaspiro[4.5]decan-2-one;
(trans)-8-{[(3-fluoro-2,3′-bipyridin-6′-yl)amino]methyl}-3-(3-pyridazinyl)-1-oxa-3-azaspiro[4.5]decan-2-one;
(trans)-8-{[(6-methyl-2,3′-bipyridin-6′-yl)amino]methyl}-3-(3-pyridazinyl)-1-oxa-3-azaspiro[4.5]decan-2-one;
6′-({[(trans)-2-oxo-3-(3-pyridazinyl)-1-oxa-3-azaspiro[4.5]dec-8-yl]methyl}amino)-2,3′-bipyridine-6-carbonitrile;
(trans)-3-(3-pyridazinyl)-8-({[5-(3-pyridazinyl)-2-pyridinyl]amino}methyl)-1-oxa-3-azaspiro[4.5]decan-2-one;
3-[6-({[(trans)-2-oxo-3-(3-pyridazinyl)-1-oxa-3-azaspiro[4.5]dec-8-yl]methyl}amino)-3-pyridinyl]benzonitrile;
(trans)-3-(3-pyridinyl)-8-({[6-(trifluoromethyl)-3-pyridinyl]amino}methyl)-1-oxa-3-azaspiro[4.5]decan-2-one;
(trans)-3-(3-pyridinyl)-8-({[5-(trifluoromethyl)-3-pyridinyl]amino}methyl)-1-oxa-3-azaspiro[4.5]decan-2-one;
(trans)-8-({[6-(4-morpholinyl)-3-pyridinyl]amino}methyl)-3-(2-pyridinyl)-1-oxa-3-azaspiro[4.5]decan-2-one;
(trans)-8-({[6-(2-fluorophenyl)-3-pyridinyl]amino}methyl)-3-(2-pyridinyl)-1-oxa-3-azaspiro[4.5]decan-2-one;
5-({[(trans)-2-oxo-3-(2-pyridinyl)-1-oxa-3-azaspiro[4.5]dec-8-yl]methyl}amino)-2-pyridinecarbonitrile;
(trans)-8-({[5-(2-fluorophenyl)-2-pyrazinyl]amino}methyl)-3-(2-pyridinyl)-1-oxa-3-azaspiro[4.5]decan-2 one;
(trans)-8-({[5-(2-fluorophenyl)-2-pyrazinyl]amino}methyl)-3-(3-pyridazinyl)-1-oxa-3-azaspiro[4.5]decan-2-one;
(trans)-8-({[5-(2-fluorophenyl)-2-pyrimidinyl]amino}methyl)-3-phenyl-1-oxa-3-azaspiro[4.5]decan-2-one;
(trans)-8-({[5-(2-fluorophenyl)-2-pyrimidinyl]amino}methyl)-3-(2-pyridinyl)-1-oxa-3-azaspiro[4.5]decan-2-one;
(trans)-3-(2-pyridinyl)-8-({[5-(1,3-thiazol-2-yl)-2-pyrimidinyl]amino}methyl)-1-oxa-3-azaspiro[4.5]decan-2-one;
(trans)-3-(2-pyridinyl)-8-({[5-(2-pyridinyl)-2-pyrimidinyl]amino}methyl)-1-oxa-3-azaspiro[4.5]decan-2-one;
(trans)-8-({[5-(2-pyrazinyl)-2-pyrimidinyl]amino}methyl)-3-(2-pyridinyl)-1-oxa-3-azaspiro[4.5]decan-2-one;
(trans)-8-({[5-(1-methyl-1H-imidazol-2-yl)-2-pyrimidinyl]amino}methyl)-3-(2-pyridinyl)-1-oxa-3-azaspiro[4.5]decan-2-one;
(trans)-8-{[(5-bromo-2-pyrimidinyl)amino]methyl}-3-(2-pyridinyl)-1-oxa-3-azaspiro[4.5]decan-2-one;
(trans)-8-[(2,5′-bipyrimidin-2′-ylamino)methyl]-3-(2-pyridinyl)-1-oxa-3-azaspiro[4.5]decan-2-one;
(trans)-8-({[5-(4-pyridazinyl)-2-pyrimidinyl]amino}methyl)-3-(2-pyridinyl)-1-oxa-3-azaspiro[4.5]decan-2-one;
(trans)-8-{[(4-phenyl-2-pyrimidinyl)amino]methyl}-3-(3-pyridazinyl)-1-oxa-3-azaspiro[4.5]decan-2-one;
(trans)-8-({[4-(1-piperidinyl)-2-pyrimidinyl]amino}methyl)-3-(3-pyridazinyl)-1-oxa-3-azaspiro[4.5]decan-2-one;
(trans)-8-[({4-[(phenylmethyl)oxy]-2-pyrimidinyl}amino)methyl]-3-(3-pyridazinyl)-1-oxa-3-azaspiro[4.5]decan-2-one;
(trans)-3-(2-pyridinyl)-8-({[6-(1-pyrrolidinyl)-3-pyridazinyl]amino}methyl)-1-oxa-3-azaspiro[4.5]decan-2-one;
(trans)-8-({[6-(2-fluorophenyl)-3-pyridazinyl]amino}methyl)-3-(2-pyridinyl)-1-oxa-3-azaspiro[4.5]decan-2-one;
(trans)-8-({[6-(3,5-difluorophenyl)-3-pyridazinyl]amino}methyl)-3-(3-pyridazinyl)-1-oxa-3-azaspiro[4.5]decan-2-one;
(trans)-8-({[6-(2-methylphenyl)-3-pyridazinyl]amino}methyl)-3-(3-pyridazinyl)-1-oxa-3-azaspiro[4.5]decan-2-one;
(trans)-8-({[6-(3,5-dimethylphenyl)-3-pyridazinyl]amino}methyl)-3-(3-pyridazinyl)-1-oxa-3-azaspiro[4.5]decan-2-one;
(trans)-8-({[6-(3-fluorophenyl)-3-pyridazinyl]amino}methyl)-3-(2-fluoro-3-pyridinyl)-1-oxa-3-azaspiro[4.5]decan-2-one;
(trans)-8-({[6-(2-fluorophenyl)-3-pyridazinyl]amino}methyl)-3-(2-fluoro-3-pyridinyl)-1-oxa-3-azaspiro[4.5]decan-2-one;
(trans)-3-(3-pyridazinyl)-8-({[6-(2-pyridinyl)-3-pyridazinyl]amino}methyl)-1-oxa-3-azaspiro[4.5]decan-2-one;
(trans)-3-(3-pyridazinyl)-8-({[6-(tetrahydro-2H-pyran-4-yloxy)-3-pyridazinyl]amino}methyl)-1-oxa-3-azaspiro[4.5]decan-2-one;
(trans)-8-({[6-({2-[(1,1-dimethylethyl)oxy]ethyl}oxy)-3-pyridazinyl]amino}methyl)-3-(3-pyridazinyl)-1-oxa-3-azaspiro[4.5]decan-2-one;
(trans)-8-{[(6-{[(3-methyl-3-oxetanyl)methyl]oxy}-3-pyridazinyl)amino]methyl}-3-(3-pyridazinyl)-oxa-3-azaspiro[4.5]decan-2-one;
(trans)-8-{[(6-chloro-3-pyridazinyl)amino]methyl}-3-(2-pyridinyl)-1-oxa-3-azaspiro[4.5]decan-2-one;
(trans)-8-{[(6-iodo-3-pyridazinyl)amino]methyl}-3-(2-pyridinyl)-1-oxa-3-azaspiro[4.5]decan-2-one;
(trans)-3-(2-pyridinyl)-8-({[6-(2-pyridinyl)-3-pyridazinyl]amino}methyl)-1-oxa-3-azaspiro[4.5]decan-2-one;
(trans)-8-({[6-(2-pyrazinyl)-3-pyridazinyl]amino}methyl)-3-(2-pyridinyl)-1-oxa-3-azaspiro[4.5]decan-2-one;
(trans)-3-(2-pyridinyl)-8-({[6-(3-pyridinyl)-3-pyridazinyl]amino}methyl)-1-oxa-3-azaspiro[4.5]decan-2-one;
(trans)-8-({[6-(phenyloxy)-3-pyridazinyl]amino}methyl)-3-(3-pyridazinyl)-1-oxa-3-azaspiro[4.5]decan-2-one;
(trans)-8-({[6-(2-fluorophenyl)-3-pyridazinyl]amino}methyl)-3-(5-methyl-1,3,4-thiadiazol-2-yl)-1-oxa-3-azaspiro[4.5]decan-2-one;
(trans)-8-({[6-(2-fluorophenyl)-3-pyridazinyl]amino}methyl)-3-(4-pyridazinyl)-1-oxa-3-azaspiro[4.5]decan-2-one;
(trans)-8-({[6-(2-fluorophenyl)-3-pyridazinyl]amino}methyl)-3-(2-fluoro-4-pyridinyl)-1-oxa-3-azaspiro[4.5]decan-2-one;
(trans)-8-({[6-(cyclopentyloxy)-3-pyridazinyl]amino}methyl)-3-(3-pyridazinyl)-1-oxa-3-azaspiro[4.5]decan-2-one;
(trans)-8-({[6-(2-fluorophenyl)-3-pyridazinyl]amino}methyl)-3-(6-methyl-3-pyridazinyl)-1-oxa-3-azaspiro[4.5]decan-2-one;
(trans)-3-(6-chloro-3-pyridazinyl)-8-({[6-(2-fluorophenyl)-3-pyridazinyl]amino}methyl)-1-oxa-3-azaspiro[4.5]decan-2-one;
(trans)-8-({[6-(3,5-difluorophenyl)-3-pyridazinyl]amino}methyl)-3-(2-fluoro-3-pyridinyl)-1-oxa-3-azaspiro[4.5]decan-2-one;
(trans)-8-({[6-(3,5-difluorophenyl)-3-pyridazinyl]amino}methyl)-3-(6-fluoro-3-pyridinyl)-1-oxa-3-azaspiro[4.5]decan-2-one;
(trans)-8-({[6-(3,5-difluorophenyl)-3-pyridazinyl]amino}methyl)-3-(2-methyl-4-pyridinyl)-1-oxa-3-azaspiro[4.5]decan-2-one;
(trans)-8-({[6-(3,5-difluorophenyl)-3-pyridazinyl]amino}methyl)-3-[6-(trifluoromethyl)-3-pyridazinyl]-1-oxa-3-azaspiro[4.5]decan-2-one;
(trans)-8-({[6-(cyclopentyloxy)-3-pyridazinyl]amino}methyl)-3-(2-fluoro-3-pyridinyl)-1-oxa-3-azaspiro[4.5]decan-2-one;
(trans)-8-({[6-(2-fluorophenyl)-3-pyridazinyl]amino}methyl)-3-(2-methyl-4-pyridinyl)-1-oxa-3-azaspiro[4.5]decan-2-one;
(trans)-8-({[6-(2-fluorophenyl)-3-pyridazinyl]amino}methyl)-3-(6-methyl-3-pyridinyl)-1-oxa-3-azaspiro[4.5]decan-2-one;
(trans)-8-({[6-(2-fluorophenyl)-3-pyridazinyl]amino}methyl)-3-(3-pyridazinyl)-1-oxa-3-azaspiro[4.5]decan-2-one;
(trans)-8-({[6-(1-piperidinyl)-3-pyridazinyl]amino}methyl)-3-(3-pyridazinyl)-1-oxa-3-azaspiro[4.5]decan-2-one;
(trans)-8-({[6-(2,5-difluorophenyl)-3-pyridazinyl]amino}methyl)-3-(3-pyridazinyl)-1-oxa-3-azaspiro[4.5]decan-2-one;
(trans)-8-({[6-(2,3-difluorophenyl)-3-pyridazinyl]amino}methyl)-3-(3-pyridazinyl)-1-oxa-3-azaspiro[4.5]decan-2-one;
(trans)-8-({[6-(6-fluoro-2-pyridinyl)-3-pyridazinyl]amino}methyl)-3-(3-pyridazinyl)-1-oxa-3-azaspiro[4.5]decan-2-one;
(trans)-8-{[(6-phenyl-3-pyridazinyl)amino]methyl}-3-(3-pyridazinyl)-1-oxa-3-azaspiro[4.5]decan-2-one;
(trans)-8-({[6-(2-methylphenyl)-3-pyridazinyl]amino}methyl)-3-(2-methyl-4-pyridinyl)-1-oxa-3-azaspiro[4.5]decan-2-one;
(trans)-3-(3-pyridazinyl)-8-[({6-[3-(trifluoromethyl)phenyl]-3-pyridazinyl}amino)methyl]-1-oxa-3-azaspiro[4.5]decan-2-one;
(trans)-3-(2-methyl-4-pyridinyl)-8-[({6-[3-(trifluoromethyl)phenyl]-3-pyridazinyl}amino)methyl]-1-oxa-3-azaspiro[4.5]decan-2-one;
(trans)-8-[({6-[3,5-bis(trifluoromethyl)phenyl]-3-pyridazinyl}amino)methyl]-3-(3-pyridazinyl)-1-oxa-3-azaspiro[4.5]decan-2-one;
(trans)-8-({[6-(3,5-difluorophenyl)-4-methyl-3-pyridazinyl]amino}methyl)-3-(3-pyridazinyl)-1-oxa-3-azaspiro[4.5]decan-2-one;
(trans)-8-({[6-(3,5-difluorophenyl)-5-methyl-3-pyridazinyl]amino}methyl)-3-(3-pyridazinyl)-1-oxa-3-azaspiro[4.5]decan-2-one;
(trans)-8-({[6-(3,5-difluorophenyl)-3-pyridazinyl]amino}methyl)-3-(2-pyridinyl)-1-oxa-3-azaspiro[4.5]decan-2-one;
(trans)-8-({[6-(3-fluorophenyl)-3-pyridazinyl]amino}methyl)-3-(3-pyridazinyl)-1-oxa-3-azaspiro[4.5]decan-2-one;
(trans)-8-({[6-(cyclohexyloxy)-3-pyridazinyl]amino}methyl)-3-(3-pyridazinyl)-1-oxa-3-azaspiro[4.5]decan-2-one;
(trans)-8-({[6-(cyclohexyloxy)-3-pyridazinyl]amino}methyl)-3-(2-pyridinyl)-1-oxa-3-azaspiro[4.5]decan-2-one;
(trans)-8-({[6-(cyclopentyloxy)-3-pyridazinyl]amino}methyl)-3-(2-pyridinyl)-1-oxa-3-azaspiro[4.5]decan-2-one;
(trans)-8-({[6-(2-oxo-1(2H)-pyridinyl)-3-pyridazinyl]amino}methyl)-3-(3-pyridazinyl)-1-oxa-3-azaspiro[4.5]decan-2-one;
(trans)-8-[({6-[(2-methylphenyl)oxy]-3-pyridazinyl}amino)methyl]-3-(3-pyridazinyl)-1-oxa-3-azaspiro[4.5]decan-2-one;
(trans)-8-[({6-[(3-fluorophenyl)oxy]-3-pyridazinyl}amino)methyl]-3-(3-pyridazinyl)-1-oxa-3-azaspiro[4.5]decan-2-one;
(trans)-3-(3-pyridazinyl)-8-{[(6-{[2-(trifluoromethyl)phenyl]oxy}-3-pyridazinyl)amino]methyl}-1-oxa-3-azaspiro[4.5]decan-2-one;
(trans)-8-[({6-[(4-fluorophenyl)oxy]-3-pyridazinyl}amino)methyl]-3-(3-pyridazinyl)-1-oxa-3-azaspiro[4.5]decan-2-one;
(trans)-8-[({6-[(2,4-dimethylphenyl)oxy]-3-pyridazinyl}amino)methyl]-3-(3-pyridazinyl)-1-oxa-3-azaspiro[4.5]decan-2-one;
(trans)-3-(3-pyridazinyl)-8-({[6-({2-[(trifluoromethyl)oxy]phenyl}oxy)-3-pyridazinyl]amino}-methyl)-1-oxa-3-azaspiro[4.5]decan-2-one;
(trans)-8-[(1,3-benzothiazol-2-ylamino)methyl]-3-(2-pyridinyl)-1-oxa-3-azaspiro[4.5]decan-2-one;
(trans)-8-{[(5,6-dichloro-1,3-benzothiazol-2-yl)amino]methyl}-3-(2-pyridinyl)-1-oxa-3-azaspiro[4.5]decan-2-one;
(trans)-3-(3-pyridazinyl)-8-[({6-[(trifluoromethyl)oxy]-1,3-benzothiazol-2-yl}amino)methyl]-1-oxa-3-azaspiro[4.5]decan-2-one;
(trans)-3-(3-pyridazinyl)-8-({[6-(trifluoromethyl)-1,3-benzothiazol-2-yl]amino}methyl)-1-oxa-3-azaspiro[4.5]decan-2-one;
(trans)-8-[(1,2-benzisothiazol-3-ylamino)methyl]-3-(3-pyridazinyl)-1-oxa-3-azaspiro[4.5]decan-2-one;
(trans)-8-{[(5,6-dichloro-1H-benzimidazol-2-yl)amino]methyl}-3-(2-pyridinyl)-1-oxa-3-azaspiro[4.5]decan-2-one;
(trans)-3-(3-pyridazinyl)-8-({[2-(trifluoromethyl)imidazo[1,2-b]pyridazin-6-yl]amino}methyl)-1-oxa-3-azaspiro[4.5]decan-2-one;
(trans)-8-{[(2-phenylimidazo[1,2-b]pyridazin-6-yl)amino]methyl}-3-(3-pyridazinyl)-1-oxa-3-azaspiro[4.5]decan-2-one;
(trans)-3-(3-pyridazinyl)-8-({[2-(2-pyridinyl)imidazo[1,2-b]pyridazin-6-yl]amino}methyl)-1-oxa-3-azaspiro[4.5]decan-2-one;
(trans)-8-({[2-(3-fluorophenyl)imidazo[1,2-b]pyridazin-6-yl]amino}methyl)-3-(3-pyridazinyl)-1-oxa-3-azaspiro[4.5]decan-2-one;
(trans)-8-({[2-(2-fluorophenyl)imidazo[1,2-b]pyridazin-6-yl]amino}methyl)-3-(3-pyridazinyl)-1-oxa-3-azaspiro[4.5]decan-2-one;
(trans)-8-({[2-(3,5-difluorophenyl)imidazo[1,2-b]pyridazin-6-yl]amino}methyl)-3-(3-pyridazinyl)-1-oxa-3-azaspiro[4.5]decan-2-one;
   or pharmaceutically acceptable salts thereof.

In another embodiment the present invention provides a compound of formula (IIA), or a pharmaceutically acceptable salt thereof:

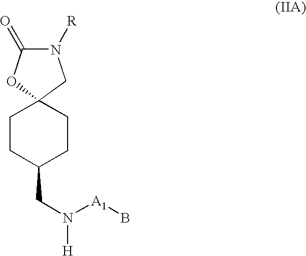

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₁ is pyridinyl, which may be substituted by one or more: halogen, C₁-C₄ alkyl, C₁-C₄ alkoxy, C₁-C₄ haloalkyl, C₁-C₄ haloalkoxy, cyano; or C(═O)—X; or —O(CH₂)₀₋₁R₁;
B is hydrogen or is a 5-6 membered heteroaryl, or a 4-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxy, cyano; A₁ and B being linked via any atom;
R₁ is —(C₁-C₄)alkyl(C₁-C₄)alkoxy; or C₃-C₈ cycloalkyl; or R₁ is an aryl or heteroaryl, which may be substituted by one or more: halogen, C₁-C₄ alkyl, C₁-C₄ alkoxy, C₁-C₄ haloalkyl, C₁-C₄ haloalkoxy, cyano; or R₁ is a 4-6 membered heterocycle, which may be substituted by one or more: halogen, C₁-C₄ alkyl, C₁-C₄ alkoxy, C₁-C₄ haloalkyl, C₁-C₄ haloalkoxy, cyano;
X is OR₂ or NR₃R₄
R₂ is C₁-C₄ alkyl;
R₃ is hydrogen or together with R₄ and the nitrogen form a 5-6 saturated membered ring;
R₄ is C₃-C₈ cycloalkyl.

In a further embodiment the present invention provides a compound of formula (IIA)′, or a pharmaceutically acceptable salt thereof, corresponding to compounds of formula (IIA) in which R is defined as for compounds of formula (IIA) and wherein
A₁ is pyridinyl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, cyano; A and B being linked via any atom.

In a further embodiment the present invention provides a compound of formula (IIA)^{II} and (IIA)^{III}, or a pharmaceutically acceptable salt thereof, corresponding to compounds of formula (IIA) and (IIA)′ in which A₁ is 2-pyridinyl, which may be substituted as above defined.

In a further embodiment the present invention provides a compound of formula (IIA)^{IV} and (IIA)^{V} or a pharmaceutically acceptable salt thereof, corresponding to compounds of formula (IIA) and (IIA)′ in which A₁ is 3-pyridinyl, which may be substituted as above defined.

In a still further embodiment the present invention provides a compound of formula (IIA), (IIA)′, (IIA)^{II}, (IIA)^{III}, (IIA)^{IV} and (IIA)^{V}, in which:
R is phenyl, or a 5-6 membered heteroaryl; which may be substituted by one or more: fluorine, bromine, chlorine, C₁-C₄ alkyl, C₁-C₄ alkoxy, C₁-C₄ haloalkyl, C₁-C₄ haloalkoxy, cyano; and A₁, B, R₁, X, R₂, R₃ are defined as above.

In a still further embodiment the present invention provides a compound of formula (IIA), (IIA)′, (IIA)^{II}, (IIA)^{III}, (IIA)^{IV} and (IIA)_{V}, in which:
R is phenyl, 2-pyridinyl, 3-pyridinyl, 2-pyrazinyl, 3-pyridazinyl, 4-pyrazolyl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₁ is 2-pyridinyl or 3-pyridinyl, which may be substituted by one or more: fluorine, bromine, chlorine, C₁-C₄ alkyl, C₁-C₄ alkoxy, C₁-C₄ haloalkyl, C₁-C₄ haloalkoxy, cyano; and, if present, by —C(═O)—X; or —O(CH₂)₀₋₁R₁;
B is hydrogen, phenyl, pyridinyl, pyrimidinyl, imidazolyl, thiazolyl, isooxazolyl, pyridazinyl, pyrazolyl, morpholinyl; which may be substituted by one or more: fluorine, bromine, chlorine, C₁-C₄ alkyl, C₁-C₄ alkoxy, C₁-C₄ haloalkyl, C₁-C₄ haloalkoxy, cyano; A₁ and B being linked via any atom;
R₁ is cyclopropyl, cyclobutyl, cyclopentyl, cyclohexyl, —CHF₂, —CF₃;
X is —OMe or NR₃R₄;
R₃ together with R₄ form piperidine or pyrrolidine.

In a further embodiment the present invention provides a compound of formula (IIB), or a pharmaceutically acceptable salt thereof:

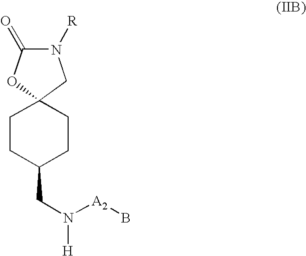

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₂ is pyrazinyl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, cyano; A₂ and B being linked via any atom.

In a further embodiment the present invention provides a compound of formula (IIB), or a pharmaceutically acceptable salt thereof, in which
R is pyridyl or pyridazinyl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₂ is pyrazinyl, which may be substituted by one or more: fluorine, bromine, chlorine, C₁-C₄ alkyl, C₁-C₄ alkoxy, C₁-C₄ haloalkyl, C₁-C₄ haloalkoxy, cyano;
B is hydrogen.

In a further embodiment the present invention provides a compound of formula (IIC), or a pharmaceutically acceptable salt thereof:

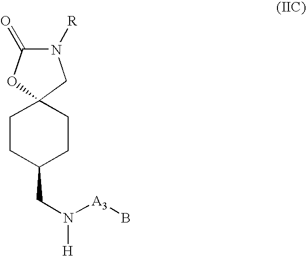

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₃ is pyrimidinyl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxyl, cyano; A₃ and B being linked via any atom.

In a further embodiment the present invention provides a compound of formula (IIC), or a pharmaceutically acceptable salt thereof, in which:
R is phenyl, 2-pyridazinyl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₃ is 2-pyrimidinyl, which may be substituted by one or more: fluorine, bromine, chlorine, C₁-C₄ alkyl, C₁-C₄ alkoxy, C₁-C₄ haloalkyl, C₁-C₄ haloalkoxy, cyano;
B is hydrogen; or phenyl, thiazolyl, pyrazinyl, imidazolyl, pyridazinyl, piperidinyl, which may be substituted by one or more: fluorine, bromine, chlorine, C₁-C₄ alkyl, C₁-C₄ alkoxy, C₁-C₄ haloalkyl, C₁-C₄ haloalkoxy, cyano.

In a further embodiment the present invention provides a compound of formula (IID), or a pharmaceutically acceptable salt thereof and wherein:

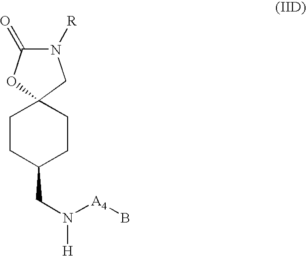

R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₄ is pyridazinyl, which may be substituted by one or more: halogen, C₁-C₄ alkyl, C₁-C₄ alkoxy, C₁-C₄ haloalkyl, C₁-C₄ haloalkoxy, cyano; or C(═O)—X; or —O(CH₂)₀₋₁R₁;
B is hydrogen or is a 5-6 membered heteroaryl, or a 4-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, hydroxy, cyano; A₄ and B being linked via any atom;
R₁ is —(C₁-C₄)alkyl(C₁-C₄)alkoxy; or C₃-C₈ cycloalkyl; or R₁ is an aryl or heteroaryl, which may be substituted by one or more: halogen, C₁-C₄ alkyl, C₁-C₄ alkoxy, C₁-C₄ haloalkyl, C₁-C₄ haloalkoxy, cyano; or R₁ is a 4-6 membered heterocycle, which may be substituted by one or more: halogen, C₁-C₄ alkyl, C₁-C₄ alkoxy, C₁-C₄ haloalkyl, C₁-C₄ haloalkoxy, cyano;
X is OR₂ or NR₃R₄;
R₂ is C₁-C₄ alkyl;
R₃ is hydrogen or together with R₄ and the nitrogen form a 5-6 saturated membered ring;
R₄ is C₃-C₈ cycloalkyl.

In a further embodiment the present invention provides a compound of formula (IID), or a pharmaceutically acceptable salt thereof, in which:
R is 2-pyridinyl, 3-pyridinyl, 4-pyridinyl, 4-pyridazinyl, 3-pyridazinyl or 1,3,4-thiadiazol-2-yl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₄ is 3,6-pyridazinyl, which may be substituted by one or more: halogen, C₁-C₄ alkyl, C₁-C₄ alkoxy, C₁-C₄ haloalkyl, C₁-C₄ haloalkoxy, cyano, —O(CH₂)₀₋₁R₁;
B is hydrogen, phenyl, pyridinyl, pyrazinyl, pyrrolidinyl, piperidinyl, 2-oxo-1(2H)-pyridinyl; which may be substituted by one or more: fluorine, bromine, chlorine, C₁-C₄ alkyl, C₁-C₄ alkoxy, C₁-C₄ haloalkyl, C₁-C₄ haloalkoxy, hydroxyl, cyano; A₄ and B being linked via any atom;
R₁ is pyranyl, phenyl, cyclopentyl, cyclohexyl, or —(CH₂)(3-methyl-3-oxetanyl).

In a still further embodiment the present invention provides a compound of formula (IID), or a pharmaceutically acceptable salt thereof, in which:
R is 2-pyridinyl, 3-pyridinyl, 4-pyridinyl, 4-pyridazinyl, 3-pyridazinyl, or 1,3,4-thiadiazol-2-yl; which may be substituted by one or more: fluorine, C₁-C₄ alkyl;
A₄ is 3,6-pyridazinyl, which may be substituted by one or more —O(CH₂)₀₋₁R₁;
B is hydrogen;
R₁ is phenyl, cyclopentyl, or cyclohexyl, which may be substituted by one or more: fluorine, C₁-C₄ alkyl.

In a further embodiment the present invention provides a compound of formula (IID)′, or a pharmaceutically acceptable salt thereof,

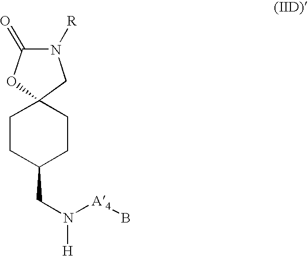

corresponding to compounds of formula (IID) in which R is defined as for compounds of formula (IID) and wherein
A₄′ is pyridazinyl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, cyano; A₄′ and B being linked via any atom.

In a still further embodiment the present invention provides a compound of formula (IID)′, or a pharmaceutically acceptable salt thereof, in which:
R is 2-pyridinyl, 3-pyridinyl, 4-pyridinyl, 4-pyridazinyl, 3-pyridazinyl or 1,3,4-thiadiazol-2-yl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₄′ is 3,6-pyridazinyl, which may be substituted by one or more: halogen, C₁-C₄ alkyl, C₁-C₄ alkoxy, C₁-C₄ haloalkyl, C₁-C₄ haloalkoxy, cyano;
B is hydrogen, phenyl, pyridinyl, pyrazinyl, pyrrolidinyl or piperidinyl, which may be substituted by one or more: fluorine, bromine, chlorine, C₁-C₄ alkyl, C₁-C₄ alkoxy, C₁-C₄ haloalkyl, C₁-C₄ haloalkoxy, hydroxyl, cyano; A₄′ and B being linked via any atom.

In a still further embodiment the present invention provides a compound of formula (IID)′, or a pharmaceutically acceptable salt thereof, in which:
R is 2-pyridinyl, 3-pyridinyl, 4-pyridinyl or 3-pyridazinyl, which may be substituted by one or more: fluorine, C₁-C₄ alkyl, C₁-C₄ alkoxy, C₁-C₄ haloalkyl, C₁-C₄ haloalkoxy, cyano;
A₄′ is 3,6-pyridazinyl, which may be substituted by one or more: fluorine, C₁-C₄ alkyl;
B is phenyl or 2-pyridyl, which may be substituted by one or more: fluorine. CF₃, C₁-C₄ alkyl.

In a further embodiment the present invention provides a compound of formula (IIE), or a pharmaceutically acceptable salt thereof:

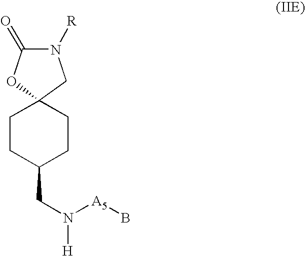

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₅ is benzothiazolyl or benzoisothiazolyl which may be substituted by one or more: halogen, C₁-C₄ alkyl, C₁-C₄ alkoxy, C₁-C₄ haloalkyl, C₁-C₄ haloalkoxy, cyano; A₅ and B being linked via any atom;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, cyano.

In a further embodiment the present invention provides a compound of formula (IIE), or a pharmaceutically acceptable salt thereof, in which
R is pyridyl or pyridazinyl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₅ is benzothiazolyl or benzoisothiazolyl which may be substituted by one or more: fluorine, bromine, chlorine, C₁-C₄ alkyl, C₁-C₄ alkoxy, C₁-C₄ haloalkyl, C₁-C₄ haloalkoxy, cyano;
B is hydrogen.

In a further embodiment the present invention provides a compound of formula (IIF), or a pharmaceutically acceptable salt thereof:

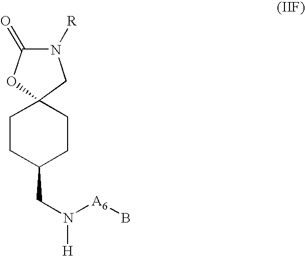

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₆ is benzimidazolyl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, cyano; A₆ and B being linked via any atom.

In a further embodiment the present invention provides a compound of formula (IIF), or a pharmaceutically acceptable salt thereof, in which:
R is pyridyl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₆ is benzimidazolyl, which may be substituted by one or more: fluorine, bromine, chlorine, C₁-C₄ alkyl, C₁-C₄ alkoxy, C₁-C₄ haloalkyl, C₁-C₄ haloalkoxy, cyano;
B is hydrogen.

In a further embodiment the present invention provides a compound of formula (IIG), or a pharmaceutically acceptable salt thereof:

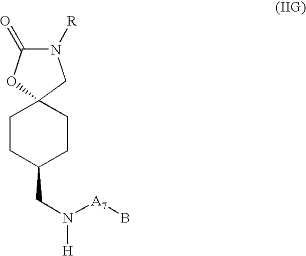

wherein
R is an aryl or heteroaryl, which may be substituted by one or more: halogen, C₁-C₄ alkyl, C₁-C₄ alkoxy, C₁-C₄ haloalkyl, C₁-C₄ haloalkoxy, cyano;
A₇ is imidazo[1,2-b]pyridazinyl, which may be substituted by one or more: halogen, C₁-C₄ alkyl, C₁-C₄ alkoxy, C₁-C₄ haloalkyl, C₁-C₄ haloalkoxy, cyano;
B is hydrogen or is a 5-6 membered heteroaryl, or a 5-6 membered heterocycle, or phenyl, which may be substituted by one or more: halogen, C₁-C₄ alkyl, C₁-C₄ alkoxy, C₁-C₄ haloalkyl, C₁-C₄ haloalkoxy, cyano; A₇ and B being linked via any atom.

In a further embodiment the present invention provides a compound of formula (IIG), or a pharmaceutically acceptable salt thereof, in which:
R is 3-pyridizanyl;
A₇ is 2-imidazo[1,2-b]pyridazinyl-6-yl, which may be substituted by one or more: fluorine, bromine, chlorine, C₁-C₄ alkyl, C₁-C₄ alkoxy, C₁-C₄ haloalkyl, C₁-C₄ haloalkoxy, cyano;
B is hydrogen; phenyl or pyridyl which may be substituted by one or more:

fluorine, bromine, chlorine, C₁-C₄ alkyl, C₁-C₄ alkoxy, C₁-C₄ haloalkyl, C₁-C₄ haloalkoxy, cyano.

In general, the compounds of formula (I) may be made according to the organic synthesis techniques known to those skilled in this field, as well as by the representative methods set forth in the Examples.

Compounds of formula (I), and salts and solvates thereof, may be prepared by the general methods outlined hereinafter. In the following description, the groups R, Z, Z₁, A and B have the meanings as previously defined for compounds of formula (I) unless otherwise stated.

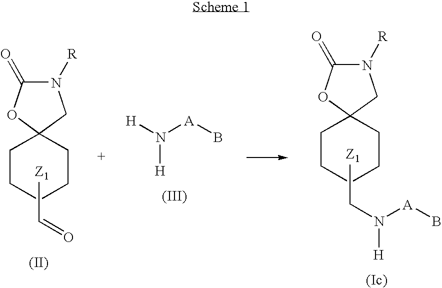

Compounds of formula (Ic), corresponding to compounds of formula (I) where Z=CH₂ can be prepared by reaction of aldehydes of formula (II) and amines of formula (III) in the presence of a reducing agent, for example sodium cyanoborohydride, sodium borohydride or sodium triacetoxyborohydride, optionally in the presence of a reagent, such as titanium tetraisopropoxide, titianium chloro-tri-isopropoxide and/or acetic acid, in a non-protic solvent such as dichloromethane. Compounds of formula (III) are commercially available e.g. 2-amino-5-trifluoromethylpyridine is available from, for example Sigma-Aldrich Chemicals.; 2-amino-5-phenylpyrazine is available from Tokyo Chemical Industry Co., Ltd. Other amines can be prepared according to literature procedures or analogous procedures thereof e.g. 5-(2-fluorophenyl)-2-pyrimidinamine can be prepared as described in WO 2003010175.

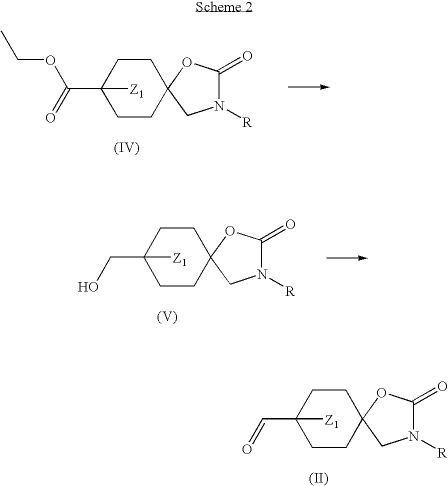

Aldehydes of formula (II) can be prepared by oxidation of alcohols of formula (V) using a reagent such as Dess-Martin periodinane, resin-supported IBX amide, DMPX, TPAP or ‘Swern’ oxidation conditions (oxalyl chloride/dimethyl sulfoxide in the presence of an amine base e.g. triethylamine or Hunig's base). Alcohols of formula (V) can be prepared from esters of formula (IV) via reduction with a reagent such as lithium aluminium hydride at a temperature below 0° C. in an aprotic solvent such as THF.

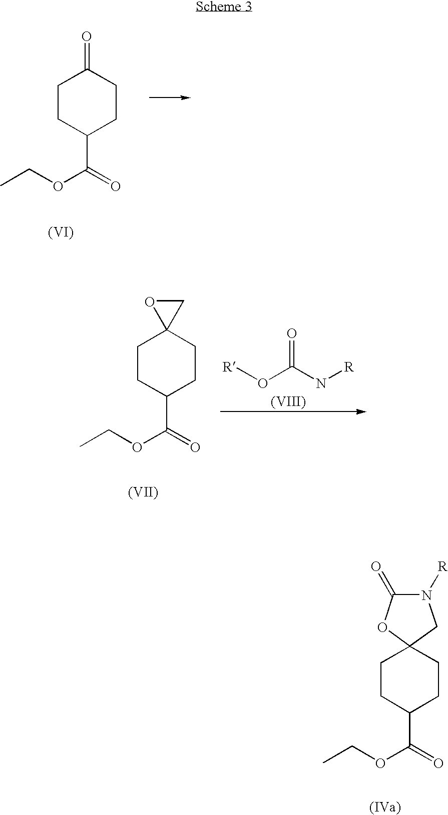

Esters of formula (IVa) can be prepared from an epoxide of formula (VII) and a carbamate of formula (VIII) in a solvent such as HPMA, DMPU, DMF or NMP in the presence of a base such as sodium tertiary-butoxide, sodium hydride or BEMP, preferably at a temperature greater than 100° C. An epoxide of formula (VII) can be prepared from a ketone (VI), which is commercially available from e.g. Sigma-Aldrich Chemicals, by treatment with trimethylsulphoxonium iodide or thrimethylsulphonium iodide in an aprotic solvent such as DMSO or acetonitrile in the presence of a base such as sodium hydride, potassium tertiary-butoxide or 2,8,9-triisobutyl-2,5,8,9-tetraaza-1-phosphabicyclo-[3.3.3]undecane. Carbamates of formula (VIII) are commercially available from e.g. Sigma-Aldrich Chemicals. Alternatively, carbamates of formula (XXVIII) can be prepared from amines of formula RNH2 by procedures described in “Protective Groups in Organic Synthesis (Third Edition)”, Theodora W. Greene, Peter G. M. Wuts, John Wiley & Sons, 2002, Chapter 7.

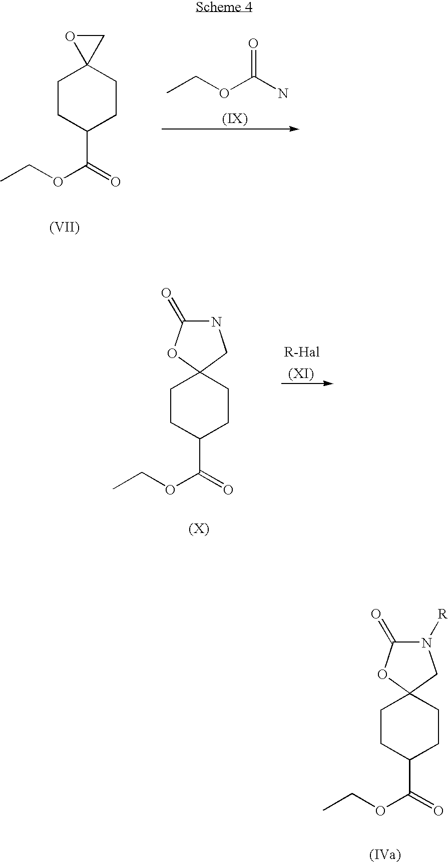

Esters of formula (IVa) can be prepared from esters of formula (X) and an aryl or heteroaryl halide of formula (XI). Suitable reactions conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Aryl or heteroaryl halides of formula (XI) are commercially available from e.g. Sigma-Aldrich Chemicals; 3-chloropyridazine can be prepared as described in WO/0107416. Esters of formula (X) can be prepared from an epoxide of formula (VII) and a carbamate of formula (IX) in a solvent such as HPMA, DMPU, DMF or NMP in the presence of a base such as potassium tertiary-butoxide, sodium hydride or BEMP, preferably at a temperature greater than 100° C. A carbamate of formula (IX) is commercially available from e.g. Sigma-Aldrich Chemicals.

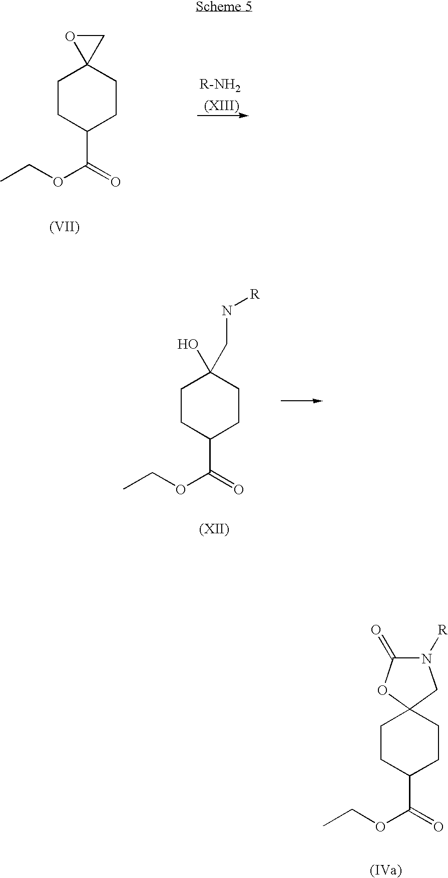

Alternatively, esters of formula (IVa) can be prepared from amino-alcohols of formula (XII) and a reagent such as phosgene, triphosgene, carbonyl di-imidazole, disuccinimidyl carbonate, carbon dioxide, an alkylchloroformate e.g. benzyl chloroformate or ethyl chloroformate, an aryl chloroformate e.g. phenyl chloroformate or a dialkyl pyrocarbonate e.g. di-tertiary-butyl di-carbonate (Boc anhydride), optionally in the presence of a base such as triethylamine in a solvent such as dichloromethane. Amino-alcohols of formula (XII) can be prepared from an epoxide of formula (VII) and amines of formula (XIII) in a protic solvent such as tertiary-butanol or ethoxyethanol at temperatures greater than 100° C. Amines of formula (XIII), such as aniline, are commercially available from e.g. Sigma-Aldrich Chemicals.

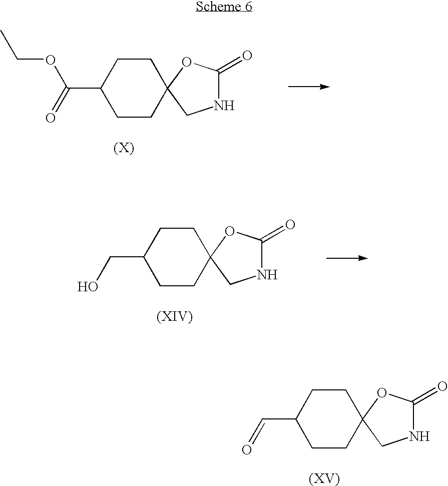

Aldehydes of formula (XV) can be prepared by oxidation of alcohols of formula (XIV) using a reagent such as Dess-Martin periodinane, resin-supported IBX amide, DMPX, TPAP or ‘Swern’ oxidation conditions (oxalyl chloride/dimethyl sulfoxide in the presence of an amine base e.g. triethylamine or Hunig's base). Alcohols of formula (XIV) can be prepared from esters of formula (X) via reduction with a reagent such as lithium aluminium hydride at a temperature below 0° C. in an aprotic solvent such as THF.

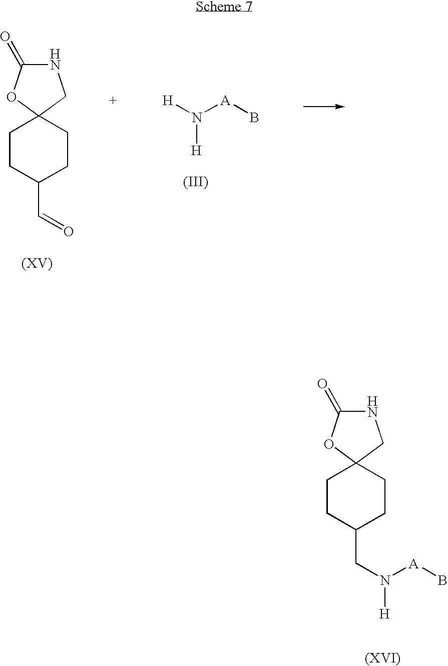

Compounds of formula (XVI) can be prepared by reaction of aldehydes of formula (XV) and amines of formula (III) in the presence of a reducing agent, for example sodium cyanoborohydride, sodium borohydride or sodium triacetoxyborohydride, optionally in the presence of a reagent, such as titanium tetraisopropoxide, titanium chloro-tri-isopropoxide and/or acetic acid, in a non-protic solvent such as dichloromethane. Compounds of formula (III) are commercially available e.g. 2-amino-5-trifluoromethylpyridine is available from, for example Sigma-Aldrich Chemicals.; 2-amino-5-phenylpyrazine is available from Tokyo Chemical Industry Co., Ltd. Other amines can be prepared according to literature procedures or analogous procedures thereof e.g. 5-(2-fluorophenyl)-2-pyrimidinamine can be prepared as described in WO 2003010175.

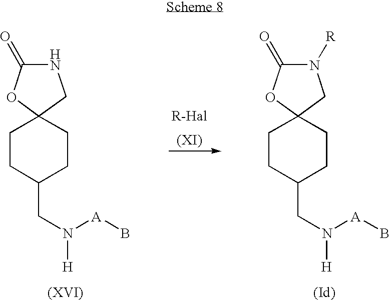

Compounds of formula (Id), corresponding to compounds (1c) where Z₁=H, can be prepared from compounds of formula (XVI) and an aryl halide of formula (XI). Suitable reaction conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Aryl halides of formula (XI) are commercially available from e.g. Sigma-Aldrich Chemicals; 3-chloropyridazine can be prepared according to WO/0107416.

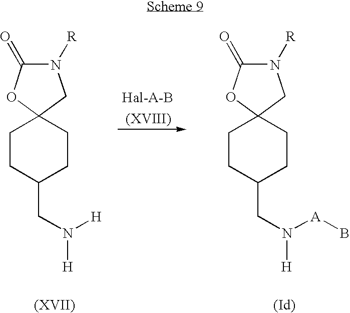

Compounds of formula (Id), corresponding to compounds (Ic) where Z₁=H, can be prepared from amines of formula (XVII) and an aryl halide of formula (XVIII). Suitable reaction conditions have been described in ‘Practical Palladium Catalysts for C—N and C-Q Bond Formation”, Topics in Current Chemistry, 2002, Vol 219, 133-209 and the references therein. Aryl halides of formula (XVIII) are commercially available from e.g. Sigma-Aldrich Chemicals. Alternatively, with some aryl halides of formula (XVIII) suitable reactions conditions consist of heating together in the presence of a base, such as triethylamine, at a temperature >100° C.

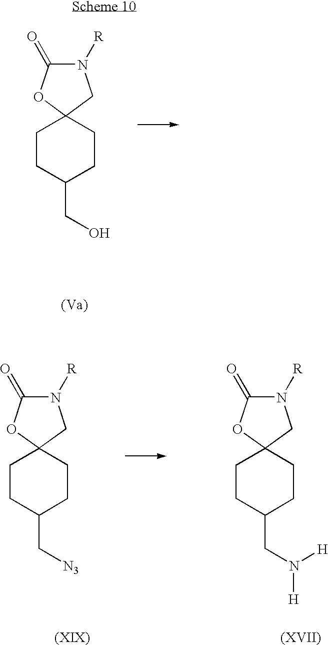

Amines of formula (XVII) can be prepared by reduction of azides of formula (XIX) using reagents such as triphenylphosphine in THF/water or a hydrogen source, such as hydrogen gas or ammonium formiate, in the presence of a palladium catalyst, such as palladium on charcoal or palladium black. Azides of formula (XIX) can be prepared from alcohols of formula (Va) by reaction with diphenyl azidophosphate, optionally in the presence of diisopropyl azodicarboxylate and triphenylphosphine. Alternatively, azides of formula (XIX) can be prepared from alcohols of formula (Va) by conversion to a sulphonate or halide intermediate, such as the methanesulfonate or a bromide, followed by reaction with sodium azide.

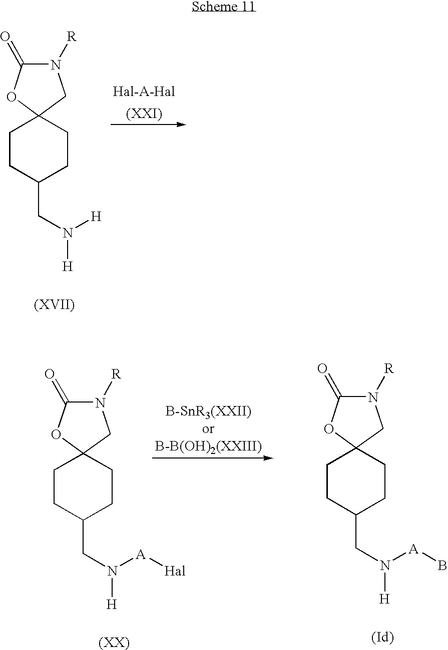

Compounds of formula (Id) can be prepared from aryl halides of formula (XX) by reaction with an organostannane of formula (XXII) or a boronic acid of formula (XXIII). Suitable reaction conditions have been described in “The Stille Reaction”, Organic Reactions (New York) (1997), 50 1-652 and “Transition Metals for Organic Synthesis” (2nd Edition) (2004), 1, 211-229, and the references therein. Organostannanes of formula (XXII) and boronic acids of formula (XXIII) are commercially available from e.g. Sigma-Aldrich Chemicals. Compounds of formula (XX) can be prepared from amines of formula (XVII) and an aryl halide of formula (XXI). Suitable reaction conditions consist of heating together in the presence of a base, such as triethylamine, at a temperature >100° C. Aryl halides of formula (XXI) are commercially available from e.g. Sigma-Aldrich Chemicals.

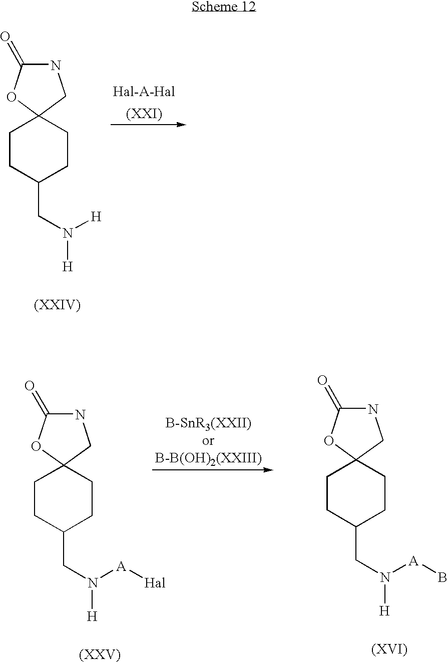

Compounds of formula (XVI) can also be prepared from aryl halides of formula (XXV) by reaction with an organostannane of formula (XXII) or a boronic acid of formula (XXIII). Suitable reaction conditions have been described in “The Stille Reaction”, Organic Reactions (New York) (1997), 50 1-652 and “Transition Metals for Organic Synthesis” (2nd Edition) (2004), 1, 211-229, and the references therein. Organostannanes of formula (XXII) and boronic acids of formula (XXIII) are commercially available from e.g. Sigma-Aldrich Chemicals. Compounds of formula (XXV) can be prepared from amines of formula (XXIV) and an aryl halide of formula (XXI). Suitable reaction conditions consist of heating together in the presence of a base, such as triethylamine or DIPEA, at a temperature >100° C. Aryl halides of formula (XXI) are commercially available from e.g. Sigma-Aldrich Chemicals.

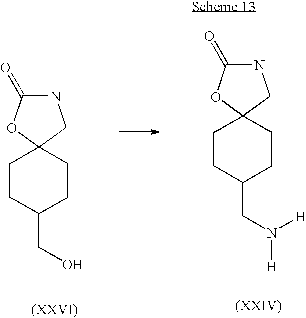

Amines of formula (XXIV) can be prepared via azide by reaction of alcohols of formula (XXVI) with diphenylazidophosphate in the presence of diisopropyl azodicarboxylate and triphenylphosphine, using THF as solvent followed by reduction of the non-isolated azide intermediate by means of water and triphenylphosphine.

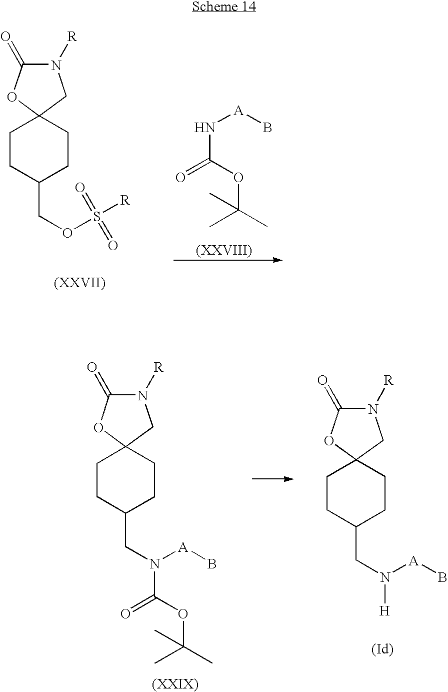

Compounds of formula (Id) can be prepared from compounds of formula (XXIX) by treatment with an acid such as trifluoroacteic acid or hydrochloric acid in a solvent such as dichloromethane, 1,4-dioxane or ethyl acetate. Compounds of formula (XXIX) can be prepared by heating a sulphonate ester of formula (XXVII) with a carbamate of formula (XXVIII) in the presence of a base such as sodium tertiary-butoxide or sodium hydride at a temperature >40° C. in an aprotic solvent such as THF or DMF. Preferably, the sulphonate ester is a methane sulphonate (R=Me).

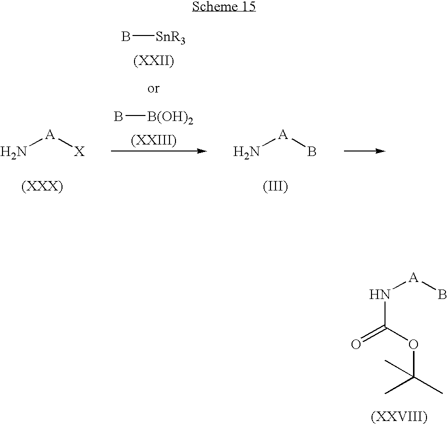

Carbamates of formula (XXVIII) can be prepared from amines of formula (III) by procedures described in “Protective Groups in Organic Synthesis (Third Edition)”, Theodora W. Greene, Peter G. M. Wuts, John Wiley & Sons, 2002, page 518 or by heating to >50° C. with di-tertiary-butyl dicarbonate in an alcohol such as tertiary-butanol. Amines of formula (III) are commercially available e.g. 2-amino-5-phenylpyrazine is available from Tokyo Chemical Industry Co., Ltd. or they may be prepared from an aryl halide of formula (XXX) by reaction with an organostannane of formula (XXII) or a boronic acid of formula (XXIII). Suitable reaction conditions have been described in “The Stille Reaction”, Organic Reactions (New York) (1997), 50 1-652 and “Transition Metals for Organic Synthesis” (2nd Edition) (2004), 1, 211-229, and the references therein. Organostannanes of formula (XXII), boronic acids of formula (XXIII) and aryl halides of formula (XXI) are commercially available from e.g. Sigma-Aldrich Chemicals.

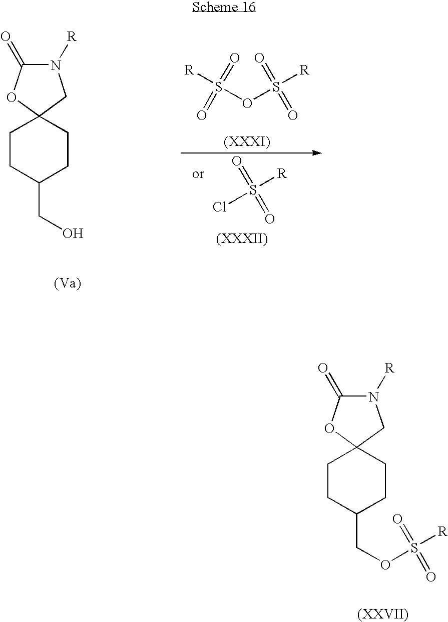

Sulphonate esters of formula (XXVII) can be prepared from alcohols of formula (Va) by reaction with a sulphonic acid anhydride of formula (XXXI) or a sulphonyl chloride of formula (XXXII) in the presence of a base such as triethylamine in an aprotic solvent such as dichloromethane. Sulphonic acid anhydrides of formula (XXXI) and sulphonyl chlorides of formula (XXXII) are commercially available from e.g. Sigma-Aldrich Chemicals.

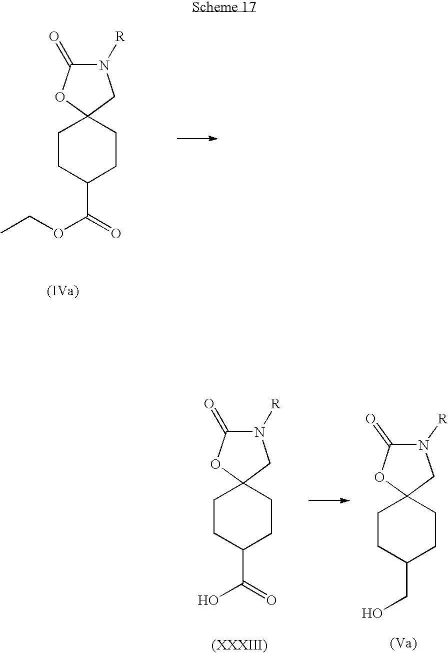

Alcohols of formula (Va) can be prepared from acids of formula (XXIII) by reaction with a reducing agent such as borane tetrahydrofuran complex. Acids of formula (XXXIII) can be prepared from esters of formula (IVa) by hydrolysis with an aqueous acid, such as dilute hydrochloric acid, in a water miscible solvent such as tetrahydrofuran. Alternatively, acids of formula (XXXIII) can be prepared from esters of formula (IVa) by hydrolysis with an alkali metal hydroxide, such as lithium hydroxide, in an alcohol, such as methanol.

Those skilled in the art will appreciate that in the preparation of the compounds of the invention it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl).

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Ci, ¹²³I and ¹²⁵I.

Compounds of the present invention that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹³F isotopes are particularly useful in PET (positron emission tomography), and ¹²⁵I isotopes are particularly useful in SPECT (single photon emission computerized tomography), all useful in brain imaging. Isotopically labelled compounds of formula I and following of this invention can generally be prepared by carrying out the procedures disclosed in the Schemes and/or in the Examples below, by substituting a readily available isotopically labelled reagent for a non-isotopically labelled reagent.

Compounds of the present invention are antagonists of the NPY Y5 receptor and as such are useful for the prevention and treatment of disorders or diseases associated with the NPY Y5 receptor sub-type, preferably for the treatment of eating disorders such as obesity, anorexia nervosa and bulimia nervosa, and other abnormal conditions, such as diabetes, hypertension, hyperlipemia, hypercholesterolemia, congestive heart failure, renal dysfunction, sexual/reproductive disorders, depression, anxiety, shock, epileptic seizure, memory loss, sleep disturbance, pain, migraine, cerebral hemorrhage, nasal congestion, gastrointestinal disorders, arthritis and immunodeficiency syndrome.

The compounds of the present invention may also be used in combination with other anti-obesity agents for increased efficacy in the prevention and treatment of obesity. Such agents would include, but not be limited to: sibutramine; dexfenfluramine; leptin; growth hormone secretagogue antagonists such as those disclosed and specifically described in U.S. Pat. No. 5,536,716; melanocortin agonists such as elanotan II; Beta-3 agonists such as those disclosed and specifically described in patent publications WO94/18161, WO95/29159, WO97/46556, WO98/04526 and WO98/32753; 5HT-2 agonists; orexin antagonists; melanin concentrating hormone antagonists; galanin antagonists; CCK agonists; GLP-1 agonists; corticotrophin releasing hormone agonists; Y1 antagonists, and CB1 antagonists.

More particularly, compounds of the present invention are useful as agents for the treatment and/or prophylaxis of eating disorders such as a binge eating disorder.

The method of treatment of this invention comprises a method of antagonizing the NPY Y5 receptor and treating NPY Y5 receptor mediated diseases by administering to a patient in need of such treatment a non-toxic therapeutically effective amount of a compound of this invention that selectively antagonizes the NPY Y5 receptor in preference to the other NPY receptors.

Within the context of the present invention, the terms describing some indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):

Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9);

In a further embodiment the invention provides the use of a compound of formula (I), or a pharmaceutically acceptable salt thereof, in the preparation of a medicament for the treatment of a binge eating disorder.

In a further embodiment the present invention provides a method of treatment of a mammal suffering from a binge eating disorder, which comprises administering to said subject an effective amount of a compound of formula (I) or a pharmaceutically acceptable salt thereof.

In a further embodiment the invention provides the use of a compound of formula (I), or a pharmaceutically acceptable salt thereof, in the preparation of a medicament for the treatment of obesity.

In a further embodiment the present invention provides a method of treatment of a mammal suffering from obesity, which comprises administering to said subject an effective amount of a compound of formula (I) or a pharmaceutically acceptable salt thereof.

Compounds of formula (I) can be administered orally or parenterally and may be formulated in the form suitable for administration to provide an agent for treatment of various diseases related to NPY, which include, for example, cardiovascular disorders (for example hypertension, nephropathy, heart disease, vasospasm, arteriosclerosis), central nervous system disorders (for example bulimia, depression, anxiety, seizure, epilepsy, dementia, pain, alcoholism, drug withdrawal), metabolic diseases (for example obesity, diabetes, hormone abnormality, hypercholesterolemia, hyperlipidemia), sexual and reproductive dysfunction, gastro-intestinal motility disorder, respiratory disorder, inflammation or glaucoma and the like, preferably, bulimia, obesity, diabetes and the like.

While it is possible that, for use in therapy a therapeutically effective amount of a compound of formula (I), or a pharmaceutically acceptable salt thereof, may be administered as the raw chemical, it is preferable to present the active ingredient as a pharmaceutical composition. Thus, in a further embodiment the invention provides a pharmaceutical composition comprising a compound of formula (I), or a pharmaceutically acceptable salt thereof, in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In a further embodiment the invention also provides a process for the preparation of a pharmaceutical composition including admixing a compound of formula (I), or a pharmaceutically acceptable salt thereof, with one or more pharmaceutically acceptable carriers, diluents or excipients.

In a further embodiment the invention provides a pharmaceutical composition for the treatment of a condition in a human for which modulation of NPY Y5 receptor is beneficial comprising a compound of the invention.

Pharmaceutical compositions of the invention may be formulated for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Therefore, the pharmaceutical compositions of the invention may be formulated, for example, as tablets, capsules, powders, granules, lozenges, creams or liquid preparations, such as oral or sterile parenteral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations adapted for rectal administration may be presented as suppositories or as enemas.

Pharmaceutical formulations adapted for nasal administration wherein the carrier is a solid may include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

It should be understood that in addition to the ingredients particularly mentioned above, the formulations may include other agents conventional in the art having regard to the type of formulation in question.

The compounds of the present invention can be used in combination with other agents useful for treating metabolic and/or eating disorders. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating metabolic and/or eating disorders includes in principle any combination with any pharmaceutical composition useful for treating metabolic and/or eating disorders.

A therapeutically effective amount of a compound of formula (I), or a pharmaceutically acceptable salt thereof will depend upon a number of factors including, for example, the age and weight of the human or other mammals, the precise condition requiring treatment and its severity, the nature of the formulation, and the route of administration, and will ultimately be at the discretion of the attendant physician or veterinarian. However, an effective amount of a compound of formula (I) for the treatment of disorders mediated by the NPY Y5 receptor will generally be in the range of 0.1 to 100 mg/kg body weight of recipient (mammal) per day and more usually in the range of 1 to 10 mg/kg body weight per day. Thus, for a 70 kg human adult, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt thereof may be determined as a proportion of the effective amount of the compound of formula (I) per se.

A compound of formula (I), or a pharmaceutically acceptable salt thereof for use in the instant invention may be used in combination with one or more other therapeutic agents. The invention thus provides in a further embodiment a combination comprising a compound of formula (I), or a pharmaceutically acceptable salt thereof together with a further therapeutic agent, which may be for example an additional anti-obesity agent. In a yet further embodiment the invention also provides the use of a combination comprising a compound of formula (I), or a pharmaceutically acceptable salt thereof with a further therapeutic agent in the treatment of disorders mediated by the NPY Y5 receptor.

When a compound of formula (I), or a pharmaceutically acceptable salt thereof is used in combination with one or more other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further embodiment of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

The following Examples describe the laboratory synthesis of specific compounds of the invention and are not meant to limit the scope of the invention in any way with respect to compounds or processes. It is understood that, although specific reagents, solvents, temperatures and time periods are used, there are many possible equivalent alternatives that can be used to produce similar results. This invention is meant to include such equivalents.

### EXPERIMENTAL

The invention is illustrated by the Compounds described below.

### Abbreviations

DMAP 4-dimethylaminopyridine
DIPEA N,N-diisopropylethylamine
TEA triethylamine
TFA trifluoroacetic acid
EtOAc ethyl acetate
EDC.HCl N-(3-dimethylaminopropyl)-N′-ethylcarbodiimide hydrochloride
HOBt.H₂O 1-hydroxybenzyltriazole hydrate
DMSO dimethylsulfoxide
DCM dichloromethane
DMF N,N-dimethylformamide
HATU (O-7-azabenzotriazol-1-yl)-N,N,N′,N′-tetramethyluroniumhexafluorophosphate)
THF tetrahydrofuran
MDAP mass-directed autopurification
Cv Column volume
r.t. room temperature

Compounds were named using ACD/Name PRO6.02 chemical naming software (Advanced Chemistry Development Inc., Toronto, Ontario, M5H2L3, Canada) with the stereochemical designations (5r, 8r) and (5s, 8s) being replaced, respectively, by the more widely used “trans” and “cis” designations.

Where reactions are described as having been carried out in a similar manner to earlier, more completely described reactions, the general reaction conditions used were essentially the same. Work up conditions used were of the types standard in the art, but may have been adapted from one reaction to another. The starting material may not necessarily have been prepared from the batch referred to. Unless otherwise stated, all compounds with chiral centre(s) are racemic. Compounds synthesised may have various purities ranging from for example 85% to 98%. However, calculations of number of moles and yield are generally not adjusted for this. In the assays used and described herein, the compounds of the present invention were not necessarily from the same batch described above. A test compound from one batch may have been combined with other batch(es) for the assay(s).

### Analytical Equipment

Proton Magnetic Resonance (NMR) spectra were recorded either on Varian instruments at 300, 400, 500 or 600 MHz, or on Bruker instruments at 300 or 400 MHz. Chemical shifts are reported in ppm (δ) using the residual solvent line as internal standard. Splitting patterns are designated as: s, singlet; d, doublet; t, triplet; q, quartet; m, multiplet; br, broad. The NMR spectra were recorded at a temperature ranging from 25 to 90° C. When more than one conformer was detected the chemical shifts for the most abundant one are reported.

Mass spectra (MS) were taken on a 4 II triple quadrupole Mass Spectrometer (Micromass UK) or on a Agilent MSD 1100 Mass Spectrometer, operating in ES(+) and ES(−) ionization mode. The usage of this methodology is indicated by “MS”.

HPLC-Mass spectra (HPLC-MS) were taken on a Agilent LC/MSD 1100 Mass Spectrometer, operating in ES(+) and ES(−) ionization mode coupled with HPLC instrument Agilent 1100 Series [LC/MS-ES (+): analysis performed on a Supelcosil ABZ+Plus (33×4.6 mm, 3 m) (mobile phase: 100% [water+0.1% formic acid] for 1 min, then from 100% [water+0.1% formic acid] to 5% [water+0.1% formic acid] and 95% [acetonitrile] in 5 min, finally under these conditions for 2 min; T=40° C.; flow=1 ml/min; LC/MS-ES (−): analysis performed on a Supelcosil ABZ+Plus (33×4.6 mm, 3 m) (mobile phase: 100% [water+0.05% ammonia] for 1 min, then from 100% [water+0.05% ammonia] to 5% [water+0.05% ammonia] and 95% [acetonitrile] in 5 min, finally under these conditions for 2 min; T=40° C.; flow=1 ml/min]. In the mass spectra only one peak in the molecular ion cluster is reported. The usage of this methodology is indicated by “HPLC-MS1” in the analytical characterization of the described compounds.

Alternatively, HPLC-MS measurements were carried out using a Platform LCZTM single quadrupole Mass Spectrometer (Micromass—Waters), coupled with an HPLC system Agilent 1100 Series. The experimental conditions were: column XBridge C18, (5 mm 4.6×50 mm), column temperature 30° C., mobile phase, A=water+0.1% TFA and B=MeCN, gradient, t=0 min 0% (B) to 60% (B) in 1.5 min to 95% (B) in 3.5 min lasting for 1.5 min (t=6.60 min 0% B stop time=7.0 min), flow rate 2 ml/min, DAD UV range 210 to 350 nm, MS ionisation mode, positive electrospray (ES+), MS range 110 to 1100 atomic mass unit. The usage of this methodology is indicated by “HPLC-MS 2” in the analytical characterization of the described compounds.

Total ion current (TIC) and DAD UV chromatographic traces together with MS and UV spectra associated with the peaks were taken also on a UPLC/MS Acquity™ system equipped with 2996 PDA detector and coupled to a Waters Micromass ZQTM mass spectrometer operating in positive or negative electrospray ionisation mode. [LC/MS-ES (+/−): analyses performed using an Acquity™ UPLC BEH C18 column (50×21 mm, 1.7 μm particle size), column temperature 40° C. (mobile phase: A-water+0.1% formic acid/B-acetonitrile+0.075% formic acid, Flow rate: 1.0 ml/min, Gradient: t=0 min 3% B, t=0.05 min 6% B, t=0.57 min 70% B, t=1.4 min 99% B, t=1.45 min 3% B)]. The usage of this methodology is indicated by “UPLC-MS” in the analytic characterization of the described compounds.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Mega Be—Si pre-packed cartridges or over pre-packed Biotage silica or KP-NH cartridges.

SPE-SCX cartridges are ion exchange solid phase extraction columns supplied by Varian. The eluent used with SPE-SCX cartridges is methanol followed by 2N ammonia solution in methanol.

In a number of preparations, purification was performed using either Biotage manual flash chromatography (Flash+) or automatic flash chromatography (Biotage Horizon, Biotage SP1 and Isco Companion) systems. All these instruments work with standard Biotage Silica cartridges.

SPE-Si cartridges are silica solid phase extraction columns supplied by Varian.

In a number of preparations, purification was performed on a Mass-Directed Autopurification (MDAP) system FractionlynX™ equipped with Waters 2996 PDA detector and coupled with ZQT mass spectrometer (Waters) operating in positive and negative electrospray ionisation mode ES+, ES−. (mass range 100-1000)

A set of acidic as well as basic semi-preparative gradients have been used:

METHOD A: Chromatographic Acidic conditions for up to 30 mg of crude:
Column: 100×21.2 mm Supelcosil™ ABZ+Plus (5 μm particle size)
Mobile phase: A[water+0.1% formic acid]/B[acetonitrile+0.1% formic acid]
Flow rate: 20 ml/min
Gradient: 5% B for 1 min, 95% B in 9 min, 100% B in 3.5 min
METHOD B: Chromatographic Acidic conditions for up to 100 mg of crude:
Column: 150×30 mm XTerra Prep MS C18 (10 μm particle size)
Mobile phase: A[water+0.1% formic acid]/B [acetonitrile+0.1% formic acid]
Flow rate: 40 ml/min
Gradient: 1% B to 100% B in 7 min lasting for 7.5 min.
METHOD C: Chromatographic Basic conditions for up to 100 mg of crude
Column: 150×30 mm XTerra Prep MS C18 (10 μm particle size)
Mobile phase: A-water+10 mM ammonium carbonate (adjusted to pH 10 with ammonia)/B-acetonitrile
Flow rate: 40 ml/min
Gradient: 10% B for 0.5 min, 95% B in 12.5 min
METHOD D: Chromatographic Acidic conditions for up to 100 mg of crude:
Column: 100×19 mm XBridge PREP C18 (5 μm particle size)
Mobile phase: A[water+0.1% TFA]/B[acetonitrile]
Flow rate: 17 ml/min
Gradient: 10% B for 1 min, 10% to 95% B in 12 min, 95% B for 1.5 min

All reactions were monitored by thin-layer chromatography on 0.25 mm E. Merck silica gel plates (either 60F-254 or 60NH₂ F-254S), visualised with UV light, iodine, 5% ethanolic phosphomolybdic acid, ninhydrin solution or vanillin solution.

### Supporting Compounds and Intermediates

### Intermediate 1

### Ethyl 1-oxaspiro[2.5]octane-6-carboxylate

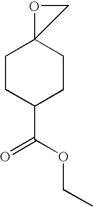

2,8,9-Triisobutyl-2,5,8,9-tetraaza-1-phosphabicyclo[3.3.3]undecane (commercially available, 52.3 g, 153 mmol) in acetonitrile (250 ml) was added dropwise to a mixture of ethyl 4-oxocyclohexanecarboxylate (20.0 g, 118 mmol) and trimethylsulfonium iodide (28.8 g, 141 mmol) in 800 ml of acetonitrile at r.t. The mixture was stirred at this temperature for 90 min, then the solvent was removed from the reaction mixture under vacuum and the crude obtained was tritured with Et₂O (1000 ml) and the mixture was filtered. The filter cake was washed again with Et₂O (800 ml). The combined organic phases were concentrated under reduced pressure to give a crude which was purified on silica gel column (Flash 65i using SP1 eluting with cyclohexane: AcOEt from 9:1 to 7:3) to afford a ˜60:40 trans:cis mixture of the title compound (19 g).

¹H NMR (400 MHz, CDCl₃): δ 1.26 (t, 3H both isomers), 1.35-1.57 (m, 2H both isomers), 1.67-2.14 (m, 6H both isomers), 2.33-2.47 (m, 1H both isomers), 2.61 (s, 2H cis isomer), 2.64 (s, 2H trans isomer), 4.15 (q, 2H both isomers).

### Intermediates 2 and 3

### Ethyl (trans)-2-oxo-1-oxa-3-azaspiro[4.5]decane-8-carboxylate (Intermediate 2) and ethyl (cis)-2-oxo-1-oxa-3-azaspiro[4.5]decane-8-carboxylate (Intermediate 3)

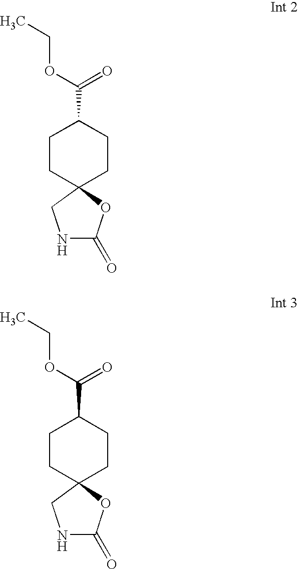

Procedure 2a and 3a

Potassium tert-butoxide (23.14 g, 206 mmol) was added portion-wise to a stirred solution of ethyl carbamate (27.6 g, 309 mmol) in DMF (200 ml) at room temperature. The resulting cloudy mixture was stirred for 1 hour then a solution of ethyl 1-oxaspiro[2.5]octane-6-carboxylate (Intermediate 1, 19 g, 103 mmol) in DMF (50 ml) was added. The reaction mixture was heated to 130° C. overnight (˜18 hours). The mixture was cooled and diluted with saturated NaCl solution (20 ml) and extracted with AcOEt (4×100 ml). The combined organic layers were dried (Na₂SO₄), filtered and concentrated to a pale yellow oil. The residue was purified via Biotage (cyclohexane: AcOEt starting from 1:1 to AcOEt pure; 65i column) to give Intermediate 2 (8.24 g) and intermediate 3 (4.36 g); Intermediate 2

¹H NMR (400 MHz, CDCl₃): δ 5.39 (1H, br s), 4.15 (2H, q), 3.37 (2H, s), 2.47 (1H, sept), 2.01-2.11 (2H, m), 1.80-1.95 (4H, m), 1.62-1.74 (2H, m), 1.27 (3H, t).

### Intermediate 3.

¹H NMR (400 MHz, CDCl₃): δ 5.27 (1H, br s), 4.15 (2H, q), 3.32 (2H, s), 2.28-2.37 (1H, m), 2.13 (2H, br d), 1.85-2.05 (4H, m), 1.53 (2H, td), 1.27 (3H, t).

### Procedure 2b and 3b

In a 1 L four-necked round bottom flask, to an ice-bath cooled solution of ethyl carbamate (41.2 g, 462 mmol) in dry DMPU (190 ml) potassium tert-butoxide (34.6 g, 308 mmol) was added portion-wise over 15 mins. The cooling bath was removed and the mixture was stirred at r.t. for 45 min under nitrogen atmosphere. The solution became cloudy. Ethyl 1-oxaspiro[2.5]octane-6-carboxylate (prepared in a similar fashion to the preparation of Intermediate 1, 28.4 g, 154 mmol) was added by a syringe and the mixture was heated at 130° C. for 22 hours. The mixture was allowed to cool to r.t. and quenched at 0° C. with saturated aqueous NH₄Cl (200 ml). The ice-bath was removed and further sat aq NH₄Cl was added (1000 ml) followed by brine (600 ml) until clear solution. The aqueous solution was extracted with DCM (3×800 ml). The combined organic extracts were dried (Na₂SO₄), filtered and DCM evaporated under reduced pressure. The remaining DMPU was stripped off in speedvacuum system (80° C., 0.1 Torr) for 20 hours affording a yellowish solid as crude material (33 g). Purification by silica gel chromatography (75 L Biotage column) eluting with cyclohexane\EtOAc\MeOH 6:3.5:0.5 afforded:
ethyl (trans)-2-oxo-1-oxa-3-azaspiro[4.5]decane-8-carboxylate (Intermediate 2, 13.7 g, 54.3 mmol)

¹H NMR_(400 MHz, CDCl₃): δ 1.28 (t, 3H), 1.70 (dd, 2H), 1.78-2.00 (m, 4H), 2.01-2.14 (m, 2H), 2.34-2.56 (m, 1H), 3.38 (s, 2H), 4.16 (q, 2H), 5.35 (br s, 1H); UPLC\MS: 0.51 min, 228 [M+H]+ and 455 [2M+H]+.

and
ethyl (cis)-2-oxo-1-oxa-3-azaspiro[4.5]decane-8-carboxylate (Intermediate 3, 15.g, 68.6 mmol)

¹H NMR (400 MHz, CDCl₃): δ 1.26 (t, 3H), 1.45-1.61 (m, 2H), 1.84-2.04 (m, 4H), 2.11 (d, 2H), 2.32 (s, 1H), 3.32 (s, 2H), 4.15 (q, 2H), 5.98 (br s, 1H); UPLC\MS: 0.50 min, 455 [2M+H]+.

### Intermediate 4

### (Trans)-8-(hydroxymethyl)-1-oxa-3-azaspiro[4.5]decan-2-one

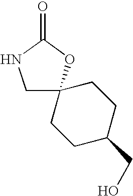

Lithium aluminium hydride (1.0M in THF, 22.00 ml, 22.00 mmol) was added to ethyl (trans)-2-oxo-1-oxa-3-azaspiro[4.5]decane-8-carboxylate (Intermediate 2, procedure 2a, 2500 mg, 11.00 mmol) dissolved in tetrahydrofuran (THF) (50 ml) cooled to 0° C. Evolution of gas was observed adding first equivalent. The resulting mixture was allowed to warm up to room temperature. Na₂SO₄×10 (20 g) was added at −20° C. and left on standing for 1 hour, allowing to warm up to room temperature. The resulting mixture was filtered washing with dichloromethane (500 ml) and dichloromethane/MeOH 90/10 (150 ml). Solvents were removed affording the title product as a colourless solid (2.4 g).

¹H NMR (400 MHz, DMSO-d₆): δ 4.60 (1H, br s), 3.11-3.27 (4H, m), 1.65-1.80 (4H, m), 1.51 (2H, td), 1.29-1.41 (1H, m), 0.90-1.04 (2H, m); UPLC-MS: 0.35 min, 186 [M+H]+.

### Intermediate 5

### (Trans)-2-oxo-1-oxa-3-azaspiro[4.5]decane-8-carbaldehyde

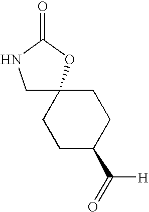

### Procedure 5a

(Trans)-8-(hydroxymethyl)-1-oxa-3-azaspiro[4.5]decan-2-one (prepared in a similar fashion to Intermediate 4, 1.2 g, 5.51 mmol) and PS-IBX amide (11.01 g, 11.01 mmol) were shaken in dichloromethane (100 ml) at room temperature for 24 hours. A further 1.0 equiv. of PS-IBX amide was added and the reaction left for a further 24 hours. The reaction was filtered washing with plenty of dichloromethane (500 ml). The collected organic phases were concentrated affording ca 1.3 g of crude oil. This was purified with Biotage SP1, over a 25M silica cartridge pre-conditioned with 100% EtOAc, eluting with EtOAc (100%). The title compound (240 mg) was recovered as a colourless solid.

¹H NMR (400 MHz, CDCl₃): δ 9.73 (1H, s), 5.34 (1H, br s), 3.32 (2H, s), 2.48 (1H, pentet), 2.06-2.15 (2H, m), 1.88-1.96 (2H, m), 1.71-1.82 (4H, m).

### Procedure 5b

(Trans)-8-(hydroxymethyl)-1-oxa-3-azaspiro[4.5]decan-2-one (prepared in a similar fashion to Intermediate 4, 1.6 g, 8.64 mmol) was dissolved in dry dichloromethane (100 ml) at r.t. under nitrogen atmosphere. Dess-Martin Periodinane (3.66 g, 8.64 mmol) was added and the mixture was stirred at r.t. for 1 hour. Then a saturated aqueous solution of Na₂SO₃ (30 ml), that was degassed with a flow of nitrogen gas for ca 30 min, was added. The reaction was diluted with DCM (100 ml) and the mixture was stirred for 1 hour. The phases were separated and the organic phase was treated with K₂CO₃ (30 ml) (a white aqueous suspension was formed). The aqueous phase was extracted with DCM (2×50 ml) then the organics were combined and dried over Na₂SO₄ and evaporated under reduced pressure to afford the title compound (1.53 g, 97%) as a pale yellow solid which was used in the subsequent reactions without further purification.

¹H NMR (400 MHz, DMSO-d₆): δ 1.44-1.57 (m, 2H), 1.61-1.77 (m, 4H), 1.84-1.94 (m, 2H), 2.34-2.42 (m, 1H), 2.49-2.52 (m, 1H), 3.20 (d, 2H), 9.60 (d, 1H); UPLC-MS: 0.38 min, 184 [M+H]+

### Intermediates 6 and 7

### Ethyl (trans)-2-oxo-3-phenyl-1-oxa-3-azaspiro[4.5]decane-8-carboxylate (Intermediate 6) and Ethyl (cis)-2-oxo-3-phenyl-1-oxa-3-azaspiro[4.5]decane-8-carboxylate (Intermediate 7)

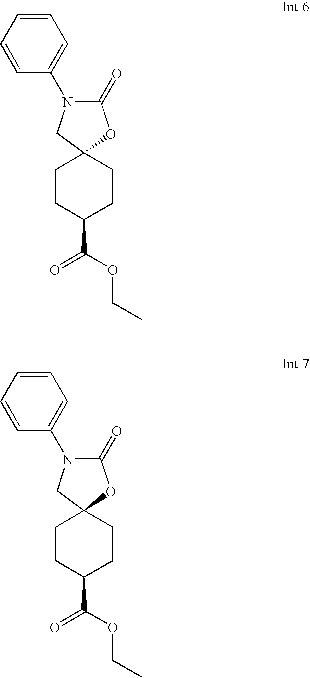

In a round bottom flask ethyl (trans)-2-oxo-1-oxa-3-azaspiro[4.5]decane-8-carboxylate (prepared in a similar fashion to Intermediate 2, procedure 2b, 0.21 g, 0.924 mmol) was dissolved in toluene (2.1 ml). Iodobenzene (0.207 ml, 1.848 mmol), cesium carbonate (0.753 g, 2.310 mmol), copper(I) iodide (8.80 mg, 0.046 mmol) and trans-1,2-diaminocyclohexane (0.011 ml, 0.092 mmol) were added and the mixture was stirred at 80° C. overnight (overall 24 hours). The mixture was allowed to cool to room temperature and partitioned between water (20 ml) and ethyl acetate (2×20 ml). The combined organics were washed (water), filtered through a phase separator filter and concentrated under vacuum. The crude was purified by column chromatography (silica gel; cyclohexane/ethyl acetate, 1:0 to 10:1 to 6:1, stepped gradient) to give Intermediate 6 (0.165 g, 59%) and Intermediate 7 (0.017 g, 7%).

### Intermediate 6:

¹H NMR (400 MHz, CDCl₃): δ 7.56 (2H, d), 7.39 (2H, t), 7.15 (1H, t), 4.17 (2H, q), 3.78 (2H, s), 2.48-2.57 (1H, m), 2.07-2.18 (2H, m), 1.85-2.03 (4H, m), 1.70-1.83 (2H, m), 1.29 (3H, t); UPLC-MS: 0.75 min, 304 [M+H]+.

### Intermediate 7:

¹H NMR (400 MHz, CDCl₃): δ 7.55 (2H, d), 7.39 (2H, t), 7.15 (1H, t), 4.17 (2H, q), 3.75 (2H, s), 2.32-2.43 (1H, m), 2.12-2-22 (2H, m), 1.90-2.10 (4H, m), 1.58-1.70 (2H, m), 1.29 (3H, t); UPLC-MS: 0.74 min, 304 [M+H]+.

### Intermediate 8

### (Trans)-8-(hydroxymethyl)-3-phenyl-1-oxa-3-azaspiro[4.5]decan-2-one

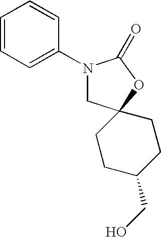

Ethyl (trans)-2-oxo-3-phenyl-1-oxa-3-azaspiro[4.5]decane-8-carboxylate (prepared in a similar fashion to Intermediate 6, 0.50 g, 1.648 mmol) was dissolved in dry tetrahydrofuran (20 ml) under a nitrogen atmosphere. The mixture was cooled to −78° C. and lithium aluminum hydride (1M solution in THF, 1.236 ml, 1.236 mmol) was slowly added. The mixture was stirred at −78° C. for 1 hour then a further 0.5 ml of 1M lithium aluminum hydride solution was added and the mixture was stirred at −78° C. for another 1 hour. The mixture was diluted with diethylether (20 ml), two spatulas of sodium sulfate decahydrate were added and the mixture was vigorously stirred while the temperature reached r.t. (ca 2 hours) then it was left to stand at r.t. overnight. The precipitate was filtered off through a separatory tube washing with Et₂O and the mixture was evaporated under reduced pressure to give 480 mg of a crude that was purified by column chromatography (Silica 25+M column, Biotage SP1) eluting with cyclohexane/EtOAc 9:1 to 1:1 to afford the title compound (396 mg) as a white solid.

¹H NMR (400 MHz, CDCl₃): δ 1.13-1.25 (2H, m), 1.34 (1H, t), 1.53-1.69 (1H, m), 1.82-1.92 (2H, m), 1.93-2.06 (4H, m), 3.57 (2H, t), 3.81 (2H, s), 7.15 (1H, t), 7.39 (2H, t), 7.58 (2H, d); UPLC-MS: 0.60 min, 262 [M+H]+

### Intermediate 9

### (Trans)-2-oxo-3-phenyl-1-oxa-3-azaspiro[4.5]decane-8-carbaldehyde

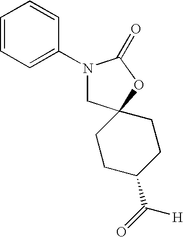

Dess-Martin Periodinane (0.771 g, 1.818 mmol) was added in two portions over 15 min to a solution of (trans)-8-(hydroxymethyl)-3-phenyl-1-oxa-3-azaspiro[4.5]decan-2-one (Intermediate 8, 0.396 g, 1.515 mmol) in dry dichloromethane (15 ml) and the resulting mixture was stirred at r.t. for 3.5 hours. The mixture was diluted with DCM (ca 20 ml) and 20 ml of a 5% solution Na₂SO₃ in saturated aquesous NaHCO₃ solution was added and the mixture was stirred for 45 minutes. The mixture was extracted with DCM (3×10 ml). The combined organic phases were evaporated under reduced pressure to give the title compound (237 mg) as a yellow solid which was used in the subsequent reactions without further purification.

¹H NMR (400 MHz, CDCl₃): δ 1.77-2.04 (6H, m), 2.10-2.22 (2H, m), 2.48-2.57 (1H, m), 3.73 (2H, s), 7.15 (1H, t), 7.39 (2H, t), 7.55 (2H, d), 9.76 (1H, s); UPLC-MS: 0.66 min, 260 [M+H]+

### Intermediate 10

### Ethyl (trans)-2-oxo-3-(2-pyridinyl)-1-oxa-3-azaspiro[4.5]decane-8-carboxylate

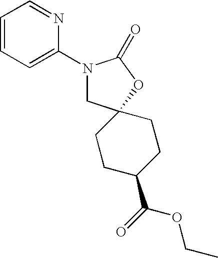

Ethyl (trans)-2-oxo-1-oxa-3-azaspiro[4.5]decane-8-carboxylate (prepared in a similar fashion to Intermediate 2, procedure 2b, 700 mg, 3.08 mmol) was dissolved in 7 ml of toluene and 2-iodopyridine (1263 mg, 6.16 mmol), copper(I) iodide (29.3 mg, 0.154 mmol), (+/−)-trans-1,2-diaminocyclohexane (0.037 ml, 0.308 mol) and cesium carbonate (2509 mg, 7.70 mmol) were added and the mixture was heated at 80° C. and stirred vigorously for 18 hours under a nitrogen atmosphere in a sealed tube. The mixture was cooled to room temperature and partitioned between water (70 ml) and ethyl acetate (2×100 ml). The combined organic extracts were washed (dilute hydrochloric acid, water), dried over Na₂SO₄, filtered and concentrated under vacuum. The crude was purified with SP1 silica gel column eluting with cyclohexane/ethyl acetate (93:7 to 50:50 gradient) to give the title compound (823.7 mg, 97%).

¹H NMR (500 MHz, CDCl₃): δ 8.33 (1H, d), 8.25 (1H, d), 7.68-74 (1H, m), 7.04 (1H, dd), 4.16 (2H, q), 4.03 (2H, s), 2.44-2.52 (1H, m), 2.04-2.15 (2H, m), 1.94-2.02 (2H, m), 1.84-1.92 (2H, m), 1.72-1.83 (2H, m), 1.28 (3H, t); UPLC-MS: 0.74 min, 305 [M+H]+

### Intermediate 11

### (Trans)-8-(hydroxymethyl)-3-(2-pyridinyl)-1-oxa-3-azaspiro[4.5]decan-2-one

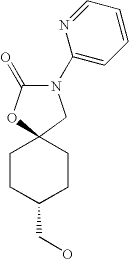

### Procedure 11a

The title compound was made in a similar fashion to the preparation of Intermediate 8 using ethyl (trans)-2-oxo-3-(2-pyridinyl)-1-oxa-3-azaspiro[4.5]decane-8-carboxylate (Intermediate 10, 820 mg, 2.69 mmol) to give the title compound (416.8 mg, 59%).

¹H NMR (400 MHz, CDCl₃): δ 8.29-8.25 (1H, m), 8.20 (1H, td), 7.66 (1H, td), 7.01-6.97 (1H, m), 4.00-3.98 (2H, m), 3.48-3.43 (2H, m), 2.48-2.43 (1H, m), 1.99-1.86 (4H, m), 1.84-1.73 (2H, m), 1.61-1.50 (1H, m), 1.109-1.09 (2H, m).

### Procedure 11b

1.0M Lithium aluminium hydride in THF (6.21 ml, 6.21 mmol) was added dropwise to a stirred solution of ethyl (trans)-2-oxo-3-(2-pyridinyl)-1-oxa-3-azaspiro[4.5]decane-8-carboxylate (prepared in a similar fashion to the preparation of Intermediate 10, 1.89 g, 6.21 mmol) in THF (45 ml) at −78° C. It was allowed to stir and warm to −30° C. over 1 hour then stirred at −30° C. for 30 mins. It was quenched with Na₂SO₄.10H₂O (˜10g), diluted with diethyl ether (150 ml) and stirred vigorously allowing warming to room temperature over 30 minutes. It was filtered washing filter cake with EtOAc (3×50 ml). The combined organic phases were evaporated and the residue was purified via Biotage (50%-100% EtOAc/cyclohexane; 40+M SiO₂ column) to give the title compound as a white solid (1.41 g, 5.38 mmol, 87%).

¹H NMR (400 MHz, CDCl₃): δ 1.15-1.29 (m, 2H), 1.55-1.69 (m, 1H), 1.81-1.92 (m, 2H), 1.92-2.08 (m, 4H), 3.54 (t, 2H), 4.06 (s, 2H), 7.05 (ddd, 1H), 7.69-7.77 (m, 1H), 8.29 (d, 1H), 8.33-8.37 (m, 1H); UPLC-MS: 0.53 min, 263 [M+H]+

### Procedure 11c

(Trans)-8-(hydroxymethyl)-1-oxa-3-azaspiro[4.5]decan-2-one (prepared in a similar fashion to the Intermediate 4, 740 mg, 4.00 mmol), 2-iodopyridine (819 mg, 4.00 mmol), copper(I) iodide (76 mg, 0.400 mmol), trans-1,2-diaminocyclohexane (0.096 ml, 0.799 mmol) and K₃PO₄ (848 mg, 4.00 mmol) were collected and shaken in 1,4-Dioxane (2 ml) at 120° C. for 8 hours. The reaction was diluted with DCM (200 ml) and washed with water (2×50 ml). The resulting organic phase was dried (Na₂SO₄), filtered and concentrated to give a crude which was purified by silica gel chromatography (Biotage SP1, 25M silica column) eluting with a gradient of cyclohexane and EtOAc The required compound was eluted with ca 60% EtOAc and recovered as a colourless solid (830 mg, 3.16 mmol, 79%).

¹H NMR (400 MHz, CDCl₃): δ 1.15-1.30 (m, 2H), 1.55-1.69 (m, 1H), 1.81-1.92 (m, 2H), 1.92-2.08 (m, 4H), 3.55 (t, 2H), 4.05 (s, 2H), 7.05 (ddd, 1H), 7.7-7.8 (m, 1H), 8.29 (d, 1H), 8.33-8.37 (m, 1H); UPLC-MS: 0.53 min, 263 [M+H]+

### Intermediate 12

### (Trans)-2-oxo-3-(2-pyridinyl)-1-oxa-3-azaspiro[4.5]decane-8-carbaldehyde

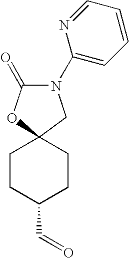

### Procedure 12a

To a solution of (trans)-8-(hydroxymethyl)-3-(2-pyridinyl)-1-oxa-3-azaspiro[4.5]decan-2-one (Intermediate 11, 252 mg, 0.961 mmol) in DCM (45 ml), were added TPAP (42.9 mg, 0.122 mmol) and NMO (169 mg, 1.441 mmol) sequentially and the reaction mixture left at r.t. under stirring until complete disappearance of the starting material as monitored by TLC (Cyclohexane: EtOAc 1:1, R_{f}=0.49). The reaction was diluted with DCM (10 ml) and filtered through celite. The crude was purified on a Biotage 12M silica column with SP1 system eluting with cyclohexane/EtOAc 1:1 to give the title compound (133.7 mg, 54%).

¹H NMR (400 MHz, CDCl₃): δ 9.72 (1H, s), 8.33-8.27 (1H, m), 8.23 (1H, td), 7.73-7.67 (1H, m) 7.06-7.00 (1H, m), 3.96 (2H, s), 2.51-2.43 (1H, m), 2.18-2.08 (2H, m), 1.97-1.72 (6H, m).

### Procedure 12b

(Trans)-8-(hydroxymethyl)-3-(2-pyridinyl)-1-oxa-3-azaspiro[4.5]decan-2-one (Intermediate 11 procedure 11c, 800 mg, 3.05 mmol) and Dess-Martin Periodinane (1294 mg, 3.05 mmol) were collected and stirred in dichloromethane (15 ml) under nitrogen at r.t. for 3 hours. The suspension was then reacted under nitrogen with deareated saturated aqueous solution of Na₂SO₃ (5 ml). The mixture was filtered over a filter tube, rinsing with DCM (10 ml). The resulting organic phase was washed with saturated aqueous K₂CO₃ solution (2×5 ml) and then concentrated under vacuum to afford the title compound (800 mg) as a yellow oil.

¹H NMR (400 MHz, CDCl₃): δ 9.74 (1H, s), 8.36-8.30 (1H, m), 8.25 (1H, d), 7.75-7.68 (1H, m) 7.08-7.02 (1H, m), 3.98 (2H, s), 2.53-2.44 (1H, m), 2.21-2.10 (2H, m), 1.99-1.75 (6H, m); UPLC-MS: 0.61 min, 261 [M+H]+

### Intermediate 13

### Ethyl (trans)-2-oxo-3-(2-pyrazinyl)-1-oxa-3-azaspiro[4.5]decane-8-carboxylate

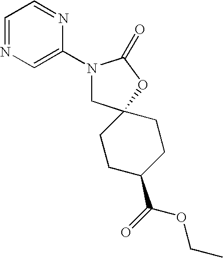

(Trans)-ethyl 2-oxo-1-oxa-3-azaspiro[4.5]decane-8-carboxylate (Intermediate 2, procedure 2a, 300 mg, 1.320 mmol), 2-chloropyrazine (0.236 ml, 2.64 mmol), N,N′-dimethylethylenediamine (0.042 ml, 0.396 mmol), copper(I) iodide (75 mg, 0.396 mmol) and potassium carbonate (657 mg, 4.75 mmol) were suspended in 1,4-dioxane (12 ml) in a microwave vial and irradiated at 130° C. for 30 min, then at 150° C. for 2×30 min. The reaction mixture was diluted with EtOAc (150 ml) and washed with water (40 ml), NaHCO₃ (40 ml) and brine (40 ml). The organic phase was dried (Na₂SO₄), filtered and evaporated. The crude was columned from silica eluting with cyclohexane/EtOAc:9/1 to 1/1 to afford the title compound (345 mg), which eluted with cyclohexane/EtOAc:6/4.

¹H NMR (400 MHz, CDCl₃): δ 9.60 (1H, dd), 8.34 (1H, d), 8.28 (1H, dd), 4.18 (2H, qua), 3.98 (2H, s), 2.52 (1H, sept), 2.07-2.18 (2H, m), 1.96-2.04 (2H, m), 1.87-1.96 (2H, m), 1.73-1.85 (2H, m), 1.30 (3H, t); UPLC-MS: 0.66 min, 306 [M+H]+.

### Intermediate 14

### (Trans)-8-(hydroxymethyl)-3-(2-pyrazinyl)-1-oxa-3-azaspiro[4.5]decan-2-one

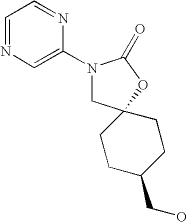

The title compound was made in a similar fashion to the preparation of Intermediate 8 using ethyl (trans)-2-oxo-3-(2-pyrazinyl)-1-oxa-3-azaspiro[4.5]decane-8-carboxylate (Intermediate 13, 325 mg, 1.064 mmol) to give the title compound (152 mg).

¹H NMR (400 MHz, CDCl₃): δ 9.62 (1H, dd), 8.34 (1H, dd), 8.28 (1H, dd), 4.00 (2H, s), 3.55 (2H, t), 1.94-2.08 (4H, m), 1.82-1.94 (2H, m), 1.61-1.70 (1H, m), 1.40 (1H, t), 1.15-1.30 (2H, m); UPLC-MS: 0.49 min, 264 [M+H]+.

### Intermediate 15

### (Trans)-2-oxo-3-(2-pyrazinyl)-1-oxa-3-azaspiro[4.5]decane-8-carbaldehyde

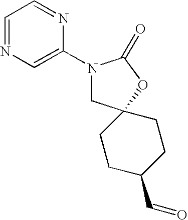

(Trans)-8-(hydroxymethyl)-3-(2-pyrazinyl)-1-oxa-3-azaspiro[4.5]decan-2-one (Intermediate 14, 150 mg, 0.570 mmol) was dissolved in dichloromethane (6.0 ml) and cooled to 0° C. Dess-Martin Periodinane (290 mg, 0.684 mmol) was added in portions. The mixture was stirred for 2 hours while warming to 15° C. The mixture was diluted with DCM (60 ml) and was washed with NaHCO₃ (20 ml) and brine (20 ml). The organic phase was passed through a PTFE frit and was evaporated. The crude was taken up in DCM and filtered to remove insoluble particles. The filtrate was evaporated and the obtained oil columned from silica (Isolute) eluting with DCM/EtOAc 5/1 to afford the title compound (91 mg).

¹H NMR (400 MHz, CDCl₃): δ 9.72 (1H, s), 8.60 (1H, dd), 8.34 (1H, d), 8.28 (1H, dd), 3.92 (2H, s), 2.47-2.55 (1H, m), 2.11-2.22 (2H, m), 1.92-2.02 (2H, m), 1.79-1.91 (4H, m); UPLC-MS: 0.57 min, 262 [M+H]+.

### Intermediate 16

### (Trans)-8-({[5-(trifluoromethyl)-2-pyridinyl]amino}methyl)-1-oxa-3-azaspiro[4.5]decan-2-one

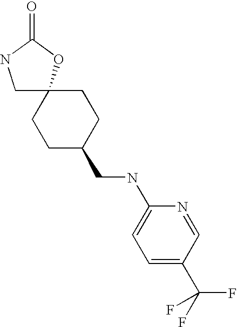

(Trans)-2-oxo-1-oxa-3-azaspiro[4.5]decane-8-carbaldehyde (prepared in a similar fashion to Intermediate 5 procedure 5b, 435 mg, 2.374 mmol) and 5-(trifluoromethyl)-2-pyridinamine (385 mg, 2.374 mmol) were stirred in DCM (8 ml). Titanium(IV) isopropoxide (1.392 ml, 4.75 mmol) was added and the mixture was stirred under nitrogen atmosphere at room temperature overnight. NaBH₄ (269 mg, 7.12 mmol) was added followed by slow addition of ethanol (2 ml) (caution: gas evolution!) and the mixture was stirred at room temperature for 4 hours. The mixture was diluted with DCM (20 ml) and an aqueous saturated solution of NaHCO₃ (20 ml) and stirred at r.t. for 30 minutes. Then the aqueous phase was extracted with DCM (3×10 ml). The organics were combined, dried (Na₂SO₄) and evaporated under reduced pressure to afford a yellow oil that was purified by Biotage-KP-NH column chromatography (Biotage SP1, 25+M column) eluting with cyclohexane/EtOAc 100:0 to 0:100 (ca 20 cv then EtOAc 100% for 15 cv) to afford the title compound as a white solid (141 mg, 18%).

¹H NMR (400 MHz, DMSO-d₆) δ 0.95-1.17 (m, 2H), 1.48-1.69 (m, 3H), 1.69-1.95 (m, 4H), 3.13-3.21 (m, 2H), 3.26 (s, 2H), 6.58 (d, 1H), 7.34-7.41 (m, 1H), 7.42-7.48 (m, 1H), 7.57-7.63 (m, 1H), 8.22-8.30 (m, 1H); UPLC-MS: 0.58 min, 330 [M+H]+

### Intermediate 17

### (Trans)-8-{[(5-phenyl-2-pyridinyl)amino]methyl}-1-oxa-3-azaspiro[4.5]decan-2-one

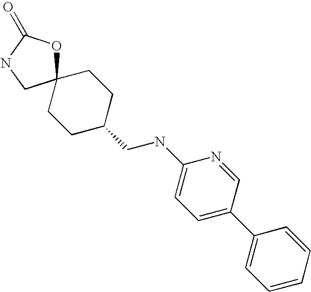

5-phenyl-2-pyridinamine (46.0 mg, 0.270 mmol) and (trans)-2-oxo-1-oxa-3-azaspiro[4.5]decane-8-carbaldehyde (prepared in a similar fashion to Intermediate 5 procedure 5b, 100 mg, 0.246 mmol) were dissolved in dry dichloromethane (8 ml) at r.t. and to the solution was added titanium tetraisopropoxide (0.144 ml, 0.491 mmol). The reaction was stirred overnight. After this time sodium borohydride (27.9 mg, 0.737 mmol) and ethanol (1 ml) were added. The crude was poured into a saturated solution of NaHCO₃ (5 ml) and extracted with DCM (3×50 ml), the combined organic phases were filtered using a phase separator tube and the organic phase was concentrated under vacuo. The crude was purified using a 25M NH column eluting with ethyl acetate to give the title compound (35 mg, 38%).

¹H NMR (400 MHz, CDCl₃): δ 1.66-1.87 (m, 5H), 1.95-2.04 (m, 4H), 3.27 (t, 2H), 3.41 (s, 2H), 5.31 (br s, 1H), 6.47 (d, 1H), 7.26-7.36 (m, 2H), 7.39-7.54 (m, 4H), 7.68 (dd, 1H), 8.34 (d, 1H).

### Intermediate 18

### (Trans)-8-(azidomethyl)-3-(2-pyridinyl)-1-oxa-3-azaspiro[4.5]-decan-2-one

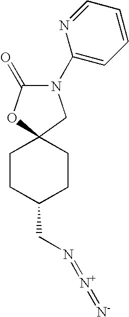

(Trans)-8-(hydroxymethyl)-3-(2-pyridinyl)-1-oxa-3-azaspiro[4.5]decan-2-one (prepared in a similar fashion to Intermediate 11, 200 mg, 0.762 mmol) was dissolved in tetrahydrofuran (13 ml) and diphenyl azidophosphate (0.493 ml, 2.287 mmol), diisopropyl azodicarboxylate (0.148 ml, 0.762 mmol) and triphenylphosphine (600 mg, 2.287 mmol) were added at 0° C. The mixture was stirred for 3 hours at 0° C. to room temperature. The reaction was quenched with saturated aqueous sodium hydrogencarbonate and diluted with ethyl acetate (100 ml). The organic phase was washed with saturated aqueous sodium hydrogencarbonate (20 ml), saturated aqueous ammonium chloride (20 ml), saturated aqueous sodium hydrogencarbonate (20 ml) and brine (20 ml), then passed through a hydrophobic PTFE frit and evaporated. The crude was purified on silica using cyclohexane/ethylacetate:9/1 to 1/1 as eluent to afford the title compound (180 mg), which eluted with cyclohexane/ethyl acetate: 3/1.

¹H NMR (400 MHz, CDCl₃): δ 8.35 (1H, ddd), 8.28 (1H, dt), 7.73 (1H, ddd), 7.06 (1H, ddd), 4.05 (2H, s), 3.24 (2H, d), 1.81-2.07 (6H, m), 1.63-1.76 (1H, m), 1.17-1.34 (2H, m); UPLC-MS: 0.75 min, 288 [M+H]+; TLC: R_{f}=0.47 (cyclohexane/ethyl acetate: 1/1, silica).

### Intermediate 19

### (Trans)-8-(aminomethyl)-3-(2-pyridinyl)-1-oxa-3-azaspiro[4.5]decan-2-one

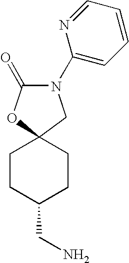

(Trans)-8-(azidomethyl)-3-(2-pyridinyl)-1-oxa-3-azaspiro[4.5]decan-2-one (Intermediate 18, 175 mg, 0.609 mmol) and triphenylphosphine (320 mg, 1.218 mmol) were stirred in dichloromethane (6 ml) at room temperature for 3 hours. The solvent was evaporated and the crude taken up in tetrahydrofuran (6 ml) and water (1 ml). The reaction mixture was stirred at room temperaute for 16 hours. The reaction mixture was transferred onto an SCX resin and eluted with methanol followed by 2M ammonia in methanol. The basic fractions were evaporated to give the title compound (125 mg).

¹H NMR (400 MHz, MeOD-d₄): δ 8.34 (1H, ddd), 8.16 (1H, d), 7.80 (1H, ddd), 7.11 (1H, ddd), 4.08 (2H, s), 2.57 (2H, d), 1.89-2.07 (4H, m), 1.81 (2H, td), 1.39-1.55 (1H, m), 1.18 (2H, quad); UPLC-MS: 0.40 min, 262 [M+H]+.

### Intermediate 20

### 2-bromo-5-[(difluoromethyl)oxy]pyridine

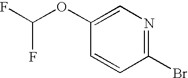

A suspension of 6-bromo-3-pyridinol (0.5 g, 2.87 mmol), chloro(difluoro)acetic acid (0.882 g, 5.75 mmol) and potassium carbonate (0.477 g, 3.45 mmol) in DMF/water (10:1, 5.5 ml) was heated to 125° C. in a sealed microwave vessel for 30 minutes. [CAUTION: pressure generated during reaction]. After cooling reaction mixture was diluted with EtOAc (100 ml) and washed with water/brine mixture (1:1, 3×100 ml), then NaOH solution (3N, 2×50 ml), then brine (50 ml). The organic phase was dried over Na₂SO₄ and evaporated. The residue was purified via Biotage (5%-20% EtOAc/cyclohexane; 12M column) to give the title compound (154 mg) as colourless oil.

¹H NMR (400 MHz, CDCl₃): δ 8.28 (1H, d), 7.51 (1H, d), 7.39 (1H, dd), 6.56 (1H, t); UPLC-MS: 0.67 min, 224 and 226 [M+H]+

### Intermediate 21

### 2-Bromo-5-[(cycloproylmethyl)oxy]pyridine

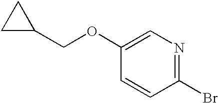

6-Bromo-3-pyridinol (1 g, 5.75 mmol), (bromomethyl)cyclopropane (0.613 ml, 6.32 mmol) and potassium carbonate (1.589 g, 11.49 mmol) were mixed in tetrahydrofuran (11.5 ml) at room temperature for 18 hours, then heated to reflux for 9 hours. The reaction was diluted with ethyl acetate (120 ml) and washed with water (20 ml) and brine (20 ml). The organic phase was passed through a hydrophobic PTFE frit and evaporated. The crude was purified on silica eluting with cyclohexane/ethyl acetate: 19/1 to 4/1 to afford the title compound (810 mg), which eluted with cyclohexane/ethyl acetate: 9/1.

¹H NMR (400 MHz, CDCl₃): δ 8.07 (1H, d), 7.37 (1H, dd), 7.11 (1H, dd), 3.85 (2H, d), 1.22-1.33 (1H, m), 0.65-0.72 (2H, m), 0.35-0.41 (2H, m); UPLC-MS: 0.74 min, 228, 230 [M+H]+; TLC: R_{f}=0.63 (cyclohexane/ethyl acetate: 3/1, silica).

### Intermediate 22

### 2-bromo-5-(cyclobutyloxy)pyridine

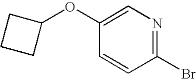

6-Bromo-3-pyridinol (1 g, 5.75 mmol), bromocyclobutane (0.812 ml, 8.32 mmol) and potassium carbonate (1.589 g, 11.49 mmol) were mixed in N,N-dimethylformamide (11.5 ml) and were stirred at 60° C. for 5 hours then at 80° C. for 9 hours. The reaction was diluted with ethyl acetate (120 ml) and washed with water (40 ml), saturated aqueous sodium hydrogencarbonate (30 ml) and brine (30 ml). The organic phase was passed through a hydrophobic PTFE frit and evaporated. The crude was purified on silica eluting with cyclohexane/ethyl acetate: 99/1 to 9/1 to afford the title compound (820 mg), which eluted at cyclohexane/ethyl acetate: 95/5.

¹H NMR (400 MHz, CDCl₃): δ 7.91 (1H, d), 7.35 (1H, d), 7.02 (1H, dd), 4.64 (2H, qui), 2.39-2.53 (2H, m), 2.11-2.25 (2H, m), 1.84-1.97 (1H, m), 1.64-1.80 (1H, m); UPLC-MS: 0.78 min, 228, 230 [M+H]+; TLC: R_{f}=0.74 (cyclohexane/ethyl acetate: 3/1, silica).

### Intermediate 23

### (Trans)-8-{[(6-chloro-3-pyridazinyl)amino]methyl}-3-(2-pyridinyl)-1-oxa-3-azaspiro[4.5]decan-2-one

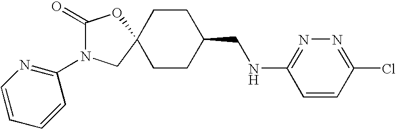

(Trans)-8-(aminomethyl)-3-(2-pyridinyl)-1-oxa-3-azaspiro[4.5]decan-2-one (prepared in a similar fashion to Intermediate 19, 100 mg, 0.383 mmol) was dissolved in butanol (1 ml). TEA (0.059 ml, 0.421 mmol) was added followed by 3,6-dichloropyridazine (57.0 mg, 0.383 mmol). The resulting mixture was heated under microwave irradiation for 1 hour at 150° C. A further 57 mg (0.383 mol) of 3,6-dichloropyridazine were added and the resulting mixture was heated under microwave irradiation for 1 hour at 150° C. Then the mixture was heated under microwave irradiation for 1 hour at 180° C. Solvents were removed under reduced pressure and the resulting crude compound was purified by flash chromatography (ISCO COMPANION, 2×12g silica gel column) with the following eluent: A: Cyclohexane/B: EtOAc: 0% B for 2.1 min, 0% to 65% B in 21.4 min, 65% B for 4.3 min. Evaporation of the solvents gave the title compound as a white solid (90 mg, 62%).

¹H NMR (400 MHz, DMSO-d₆): δ 8.37 (dt, 1H), 8.05-8.15 (m, 1H), 7.84 (ddd, 1H), 7.35 (d, 1H), 7.10-7.22 (m, 2H), 6.92 (d, 1H), 3.97-4.05 (m, 2H), 3.25 (t, 2H), 1.93-2.04 (m, 2H), 1.78-1.89 (m, 2H), 1.62-1.75 (m, 3H), 1.14-1.31 (m, 2H); UPLC-MS: 0.59 min, 187.60 [M+2H]2+, 208.11, 374.02 [M+H]+.

### Intermediate 24

### (Trans)-8-{[(5-bromo-2-pyrimidinyl)amino]methyl}-3-(2-pyridinyl)-1-oxa-3-azaspiro[4.5]decan-2-one

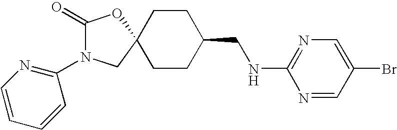

To a suspension of (trans)-8-(aminomethyl)-3-(2-pyridinyl)-1-oxa-3-azaspiro[4.5]decan-2-one (prepared in a similar fashion to Intermediate 19, 50 mg, 0.191 mmol) in isopropanol (2 ml) were added 2-chloro-5-bromopyrimidine (37.0 mg, 0.191 mmol) and DIPEA (49.5 mg, 0.383 mmol). The suspension was irradiated in a microwave oven (120° C. 3 cycles of 15 min each) then the solvent was evaporated and the product was purified by chromatography on SP1 system (10 g SNAP silica cartridge, DCM/MeOH 100/0 to 97/3) to give the title compound as a white solid (52 mg, 65%).

¹H NMR (400 MHz, CDCl₃): δ 8.34 (1H, dt), 8.28 (3H, m), 7.72 (1H, ddd), 7.05 (1H, ddd), 5.22 (1H, t), 4.06 (2H, br s), 3.34 (2H, t), 1.98 (4H, m), 1.79 (3H, m), 1.23 (3H, m); UPLC-MS: 0.74 min, 418 and 420 [M+H]⁺

### Intermediate 25

### 5-(2-Fluorophenyl)-2-pyrazinamine

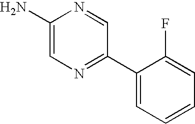

To degassed 1,4-dioxane (17 ml) were added (2-fluorophenyl)boronic acid (0.920 g, 6.57 mmol), 5-bromo-2-pyrazinamine (1.04 g, 5.98 mmol) and bis(triphenylphosphinyl)palladium(II) chloride (0.210 g, 0.299 mmol). The mixture was stirred at room temperature under nitrogen for 30 minutes. A degassed aqueous solution of sodium carbonate (17.93 ml, 17.93 mmol) was added and the mixture was degassed 3 times (vacuum/nitrogen cycles), heated to reflux and stirred under nitrogen for 3 hours. The mixture was cooled to room temperature and partitioned between water and ethyl acetate. The aqueous phase was re-extracted with ethyl acetate. The combined organics were washed (water, brine), filtered through a hydrophobic membrane (phase separator) and concentrated under vacuum. The crude was purified by column chromatography on silica gel eluting with cyclohexane/ethyl acetate (1:0 to 4:1 to 1:1 gradient then isocratic) to afford a first batch of the title compound (0.278 g). A second batch of the title compound (0.555 g) was isolated by recovering an undissolved solid residue from the top of the column, dissolving it in an ethyl acetate/dichloromethane mixture, filtering and concentrating the filtrate under vacuum. The two batches were dissolved in a small volume of hot methanol, combined and concentrated under a stream of nitrogen while heating at 40° C. (block temperature). The residue was dried under vacuum at 40° C. for 2 hours to afford the title compound (0.6087 g).

¹H NMR (400 MHz, DMSO-d₆): δ 8.33 (1H, dd), 8.02 (1H, d), 7.82-7.89 (1H, m), 7.34-7.42 (1H, m), 7.25-7.32 (2H, m), 6.68 (2H, brs); UPLC-MS: 0.57 min, 190 [M+H]+

### Intermediate 26

### 5-(2-Pyrimidinyl)-2-pyridinamine

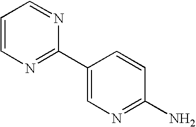

2-bromopyrimidine (144 mg, 0.909 mmol), 5-(4,4,5,5-tetramethyl-1,3,2-dioxaborolan-2-yl)-2-pyridinamine (200 mg, 0.909 mmol), PdCl₂(dppf) (74.2 mg, 0.091 mmol) and sodium carbonate (2.73 ml, 2.73 mmol) were collected in DME (2 ml) and shaken at 90° C. overnight. Then, solvent was removed under vacuum and the residue was taken up with DCM (10 ml) filtering over a filter tube. The resulting crude was then purified with Biotage SP1, over a 25M KP-NH column, with a gradient of DCM/EtOH to afford the title compound as a solid (40 mg, 25%).

¹H NMR (400 MHz, CDCl₃): δ 9.18-9.15 (m, 1H), 8.73 (d, 2H), 8.46 (dd, 1H), 7.13 (t, 1H), 6.59 (dd, 1H), 4.80 (brs, 2H); UPLC-MS: 0.30 min, 173 [M+H]+.

### Intermediate 27

### 5-(1-Methyl-1H-imidazol-5-yl)-2-pyridinamine

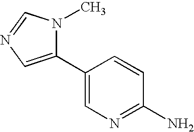

The title compound was made in a similar fashion to the preparation of Intermediate 26 replacing 2-bromopyrimidine with 5-bromo-1-methyl-1H-imidazole (146 mg, 0.909 mmol) to afford the title compound (100 mg, 63%).

¹H NMR (400 MHz, CDCl₃): δ 8.12 (br s, 1H), 7.52 (br s, 1H), 7.46 (dd, 1H), 7.05 (br s, 1H), 4.60 (brs, 2H), 3.63 (s, 3H); UPLC-MS: 0.16 min, 175 [M+H]+.

### Intermediate 28

### 5-(1,3-Thiazol-5-yl)-2-pyridinamine

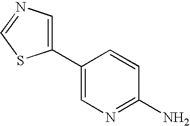

The title compound was made in a similar fashion to the preparation of Intermediate 26 replacing 2-bromopyrimidine with 5-bromo-1,3-thiazole (149 mg, 0.909 mmol) to afford the title compound (110 mg, 68%).

¹H NMR (400 MHz, CDCl₃): δ 8.72 (s, 1H), 8.31 (br s, 1H), 7.96 (s, 1H), 7.63 (dd, 1H), 6.56 (d, 1H), 4.67 (br s, 2H); UPLC-MS: 0.32 min, 178 [M+H]+.

### Intermediate 29

### 5-(5-Methyl-1,3,4-thiadiazol-2-yl)-2-pyridinamine

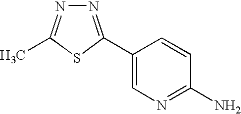

The title compound was made in a similar fashion to the preparation of Intermediate 26 replacing 2-bromopyrimidine with 2-bromo-5-methyl-1,3,4-thiadiazole (163 mg, 0.909 mmol) to afford the title compound (65 mg, 37%).

¹H NMR (400 MHz, CDCl₃): δ 8.56 (d, 1H), 8.07 (dd, 1H), 6.59 (dd, 1H), 4.81 (br s, 2H), 2.81 (s, 3H); UPLC-MS: 0.32 min, 193 [M+H]+.

### Intermediate 30

### 5-(1,3-Thiazol-2-yl)-2-pyridinamine

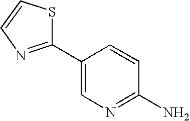

The title compound was made in a similar fashion to the preparation of Intermediate 26 replacing 2-bromopyrimidine with 2-bromo-1,3-thiazole (149 mg, 0.909 mmol) to afford the title compound (100 mg, 62%).

¹H NMR (400 MHz, CDCl₃): δ 8.67 (br s, 1H), 8.03 (dd, 1H), 7.82 (d, 1H), 7.26 (d, 1H), 6.57 (dd, 1H), 4.77 (brs, 2H); UPLC-MS: 0.33 min, 178 [M+H]+.

### Intermediate 31

### 5-(3,5-Dimethyl-4-isoxazolyl)-2-pyridinamine

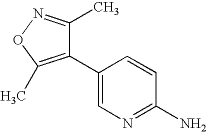

The title compound was made in a similar fashion to the preparation of Intermediate 26 replacing 2-bromopyrimidine with 4-bromo-3,5-dimethylisoxazole (160 mg, 0.909 mmol) to afford the title compound (33 mg, 19%).

¹H NMR (400 MHz, CDCl₃): δ 7.99-7.96 (m, 1H), 7.33 (dd, 1H), 6.60-6.56 (m, 1H), 4.64 (br s, 1H), 2.38 (s, 3H), 2.24 (s, 3H); UPLC-MS: 0.32 min, 190 [M+H]+.

### Intermediate 32

### 5-(2-Methyl-1,3-thiazol-4-yl)-2-pyridinamine

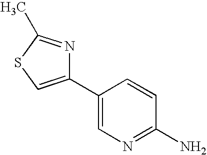

The title compound was made in a similar fashion to the preparation of Intermediate 26 replacing 2-bromopyrimidine with 4-bromo-2-methyl-1,3-thiazole (162 mg, 0.909 mmol) to afford the title compound (150 mg, 86%).

¹H NMR (400 MHz, CDCl₃): δ 8.60 (br s, 1H), 7.95 (dd, 1H), 7.16 (s, 1H), 6.56 (dd, 1H), 4.55 (br s, 2H), 2.77 (s, 3H); UPLC-MS: 0.36 min, 192 [M+H]+.

### Intermediate 33

### 5-(4-Pyridazinyl)-2-pyridinamine

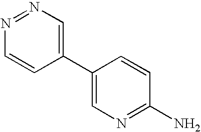

The title compound was made in a similar fashion to the preparation of Intermediate 26 replacing 2-bromopyrimidine with 4-bromopyridazine (144 mg, 0.909 mmol) to afford the title compound (40 mg, 25%).

¹H NMR (400 MHz, CDCl₃): δ 9.42 (br s, 1H), 9.18 (dd, 1H), 8.46 (d, 1H), 7.77 (dd, 1H), 7.64-7.39 (m, 1H), 6.66 (d, 1H), 4.85 (brs, 2H); UPLC-MS: 0.24 min, 173 [M+H]+.

### Intermediate 34

### 5-(1,3,5-Trimethyl-1H-pyrazol-4-yl)-2-pyridinamine

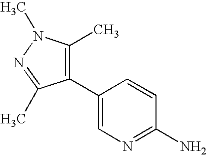

The title compound was made in a similar fashion to the preparation of Intermediate 26 replacing 2-bromopyrimidine with 4-bromo-1,3,5-trimethyl-1H-pyrazole (172 mg, 0.909 mmol) to afford the title compound (60 mg, 32%).

¹H NMR (400 MHz, CDCl₃): δ 7.98-7.95 (m, 1H), 7.34 (dd, 1H), 6.58 (dd, 1H), 4.45 (br s, 2H), 3.78 (s, 3H), 2.22 (s, 6H); UPLC-MS: 0.35 min, 203 [M+H]+.

### Intermediate 35

### 5-(3-Methyl-1H-pyrazol-4-yl)-2-pyridinamine

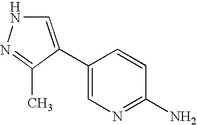

The title compound was made in a similar fashion to the preparation of Intermediate 26 replacing 2-bromopyrimidine with 4-bromo-3-methyl-1H-pyrazole (146 mg, 0.909 mmol) to afford the title compound (30 mg, 19%).

¹H NMR (400 MHz, CDCl₃): δ 8.14 (br s, 1H), 7.61 (br s, 1H), 7.49 (dd, 1H), 6.57 (d, 1H), 4.51 (br s, 2H), 2.41 (s, 3H); UPLC-MS: 0.30 min, 175 [M+H]+.

### Intermediate 36

### 1,1-Dimethylethyl 3-pyridazinylcarbamate

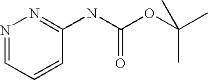

To a stirred suspension of 3-pyridazinamine hydrochloride (100 g, 760 mmol) in DCM (1000 ml) TEA (212 ml, 1520 mmol) was added dropwise under argon flow. The mixture was stirred for 10 minutes then DMAP (13.93 g, 114 mmol) was added portion-wise and the resulting mixture was stirred for 5 minutes then Boc₂O (194 ml, 836 mmol) was added and the mixture was stirred at room temperature overnight. Water (1000 ml) and DCM (500 ml) were added, phases were separated and the organic phase was washed with H₂O/saturated NH₄Cl solution (1/1) (2×1.5 l).The organic phase was filtered, washed with H₂O (500 ml) and dried over Na₂SO₄. Solvent was removed and the resulting crude was purified by flash chromatography eluting with 100% EtOAc to afford the title compound (113 g).

¹H NMR (400 MHz, CDCl₃): δ 8.88 (dd, 1H), 8.24 (d, 1H), 7.79 (br s, 1H), 7.44 (dd, 1H), 1.55 (s, 9H).

### Intermediate 37

### Ethyl (trans)-2-oxo-3-(3-pyridazinyl)-1-oxa-3-azaspiro[4.5]decane-8-carboxylate

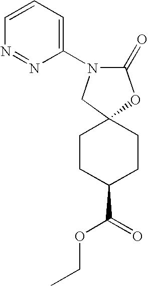

### Procedure 37a

Ethyl (cis)-2-oxo-1-oxa-3-azaspiro[4.5]decane-8-carboxylate and ethyl (trans)-2-oxo-1-oxa-3-azaspiro[4.5]decane-8-carboxylate mixture (˜85/15 mixture) (obtained from combination of four different batches prepared in a similar fashion to preparation of Intermediates 2 and 3, procedures a and b, 10 g, 44.0 mmol), K₃PO₄ (28.0 g, 132 mmol), copper(I) iodide (0.838 g, 4.40 mmol), 3-chloropyridazine (prepared according to WO/0107416, 6.05 g, 52.8 mmol) were collected into a 250 ml reaction flask, deareated, and then suspended in 1,4-dioxane (150 ml) under nitrogen. Trans-1,2-diaminocyclohexane (1.058 ml, 8.80 mmol) was added to the resulting mixture. The reaction was then warmed up to reflux (ext temp. 130° C., int temp 105° C.). The reaction mixture was stirred at that temperature for overall 24 hours. The reaction mixture was taken up with DCM (1000 ml) and poured into water (300 ml) containing 10 ml of ammonium hydroxide, and left under stirring for 10 min. Then, the resulting organic phase was washed with water (2×100 ml) and brine (2×100 ml), dried over Na₂SO₄, filtered and then concentrated. The resulting crude was purified with Biotage SP1, over 65i silica column, using cyclohexane and EtOAc as eluent to afford two different batches of the title compound.

Batch 1: Ethyl (trans)-2-oxo-3-(3-pyridazinyl)-1-oxa-3-azaspiro[4.5]decane-8-carboxylate (3.4 g) was isolated as a single isomer, colourless solid, contaminated by ca 30% of starting chloropyridazine. This batch left at room temperature, in the light, after some days, resulted in a strongly dark coloured and degradated residue.

Batch 2: A ˜80/20 mixture of trans/cis isomers was isolated as a colourless solid (2.2 g) and repurified on Biotage SP1, with a Biotage 65i silica column, using cyclohexane and ethyl acetate as eluent to give isomerically pure ethyl (trans)-2-oxo-3-(3-pyridazinyl)-1-oxa-3-azaspiro[4.5]decane-8-carboxylate (1.6 g)

¹H NMR (400 MHz, CDCl₃): δ 8.96 (dd, 1H), 8.56 (dd, 1H), 7.50 (dd, 1H), 4.20 (s, 2H), 4.18 (q, 2H), 2.51 (quint, 1H), 2.17-1.75 (m, 8H), 1.29 (t, 3H); UPLC-MS: 0.60 min, 306 [M+H]+.

From these two purifications two batches of cis isomer were also isolated:

Batch 1: ethyl (cis)-2-oxo-3-(3-pyridazinyl)-1-oxa-3-azaspiro[4.5]decane-8-carboxylate as single isomer and colourless solid (4.7 g)

Batch 2: ethyl (cis)-2-oxo-3-(3-pyridazinyl)-1-oxa-3-azaspiro[4.5]decane-8-carboxylate (0.5 g).

¹H NMR (400 MHz, CDCl₃): δ 8.96 (dd, 1H), 8.57 (dd, 1H), 7.50 (dd, 1H), 4.18 (q, 3H), 4.14 (s, 2H), 2.51 (quint, 1H), 2.46-2.37 (m, 1H), 2.21-1.66 (m, 8H), 1.29 (t, 3H); UPLC-MS: 1.04 min, 306 [M+H]+.

### Procedure 37b

Ethyl 4-oxocyclohexanecarboxylate (55 g, 323 mmol) was dissolved in DMSO (300 ml). Trimethylsulfoxonium iodide (71.1 g, 323 mmol) was added followed by potassium tert-butoxide (36.3 g, 323 mmol), portion-wise and at 0° C. The resulting mixture was stirred for 1 hour, then a pre-stirred solution of 1,1-dimethylethyl 3-pyridazinylcarbamate (Intermediate 36, 31.5 g, 162 mmol) and potassium tert-butoxide (18.15 g, 162 mmol) in DMSO (100 ml) was added dropwise. The reaction mixture was stirred at 85° C. for 3 hours, then an additional solution of 1,1-dimethylethyl 3-pyridazinylcarbamate (Intermediate 36, 9.46 g) and potassium tert-butoxide (5.44 g) in DMSO (40 ml) was added at r.t and the mixture was heated at 85° C. for further 7 hours then it was allowed to reach r.t. and quenched with a saturated NH₄Cl solution (400 ml), diluted with water (300 ml) and extracted with ethyl acetate (3×500 ml). Organic phase was washed with brine (600 ml), dried (Na₂SO₄) and concentrated. in vacuo to obtain a crude material (84 g), which was processed together with the crude reaction product from Intermediate 37 Procedure 37c below.

### Procedure 37c

Ethyl 4-oxocyclohexanecarboxylate (100 g, 588 mmol) was dissolved in DMSO (700 ml) then trimethylsulfoxonium iodide (129 g, 588 mmol) and potassium tert-butoxide (65.9 g, 588 mmol) were added portionwise. The resulting mixture was stirred for 90 minutes then it was cooled down to 0° C., quenched with a saturated NH₄Cl solution (800 ml) and extracted with Et₂O (3×1000 ml). Combined organic extracts were washed with brine (1000 ml), dried over Na₂SO₄, concentrated in vacuo and then taken up in DMSO (250 ml).

In a separate flask 1,1-dimethylethyl 3-pyridazinylcarbamate (Intermediate 36, 44.5 g, 228 mmol) was dissolved in DMSO (300 ml) and potassium tert-butoxide (25.6 g, 228 mmol) was added portion wise at r.t. The resulting mixture was stirred for 1 hour then it was added dropwise to the above solution.

The resulting mixture was stirred at 85° C. for 3 hours then a pre-stirred solution of 1,1-dimethylethyl 3-pyridazinylcarbamate (Intermediate 36, 13.35 g) and potassium tert-butoxide (7.67 g) in DMSO (100 ml) was added at r.t. and the mixture was heated to 85° C. for a further 7 hours. Then it was allowed to reach r.t. and quenched with a saturated NH₄Cl solution (800 ml), diluted with water (300 ml) and extracted with ethyl acetate (3×800 ml). Organic phase was washed with brine (1000 ml), dried over Na₂SO₄ and concentrated in vacuo to give a residue. The residue was combined with the crude material of Intermediate 37 procedure 37b (84 g) and purified by flash chromatography eluting in gradient with 50%-100% EtOAc\cyclohexane to afford the title compound (24.5 g).

¹H NMR (400 MHz, CDCl₃): δ 8.96 (dd, 1H), 8.56 (dd, 1H), 7.50 (dd, 1H), 4.20 (s, 2H), 4.18 (q, 2H), 2.51 (quint, 1H), 2.20-1.65 (m, 8H), 1.29 (t, 3H); UPLC-MS: 1.08 min, 306 [M+H]+.

Moreover a batch of correspondent cis isomer (48.3 g) and a mixture of cis and trans isomers (11.5 g) were recovered. The mixture was purified by flash chromatography eluting in gradient with 50%-100% EtOAc\cyclohexane to afford an additional batch of the title compound (1.8 g)

¹H NMR (400 MHz, CDCl₃): δ 8.96 (d, 1H), 8.55 (d, 1H), 7.50 (dd, 1H), 4.19 (s, 2H), 4.18 (q, 2H), 2.51 (quint, 1H), 2.18-1.74 (m, 8H), 1.29 (t, 3H); UPLC-MS: 1.08 min, 306 [M+H]+

### Intermediate 38

### (Trans)-2-oxo-3-(3-pyridazinyl)-1-oxa-3-azaspiro[4.5]decane-8-carboxylic acid

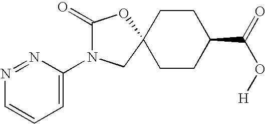

Ethyl (trans)-2-oxo-3-(3-pyridazinyl)-1-oxa-3-azaspiro[4.5]decane-8-carboxylate (Intermediate 37 procedure 37c, two combined batches, 26.3 g, 68.9 mmol) was dissolved in THF (300 ml) and 3.0M HCl (300 ml, 900 mmol) was added. The resulting mixture was stirred at r.t. overnight and then for further 5 days. The mixture was cooled down to 0° C., basified to pH 8 with 3N NaOH and extracted with DCM. The aqueous solution was treated with 1.0M HCl until pH 5.5 and extracted with DCM. This organic phase was dried over Na₂SO₄ and concentrated in vacuo to afford the title compound (16.5 g).

¹H NMR (400 MHz, DMSO-d₆): δ 12.26 (s, 1H), 9.01 (dd, 1H), 8.38 (dd, 1H), 7.73 (dd, 1H), 4.12 (s, 2H), 2.4 (m, 1H), 1.96 (m, 4H), 1.79 (m, 2H), 1.65 (m, 2H); UPLC-MS: 0.46 min, 278 [M+H]+

### Intermediate 39

### (Trans)-8-(hydroxymethyl)-3-(3-pyridazinyl)-1-oxa-3-azaspiro[4.5]decan-2-one

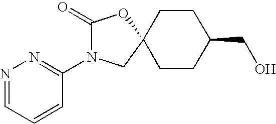

### Procedure 39a

Ethyl (trans)-2-oxo-3-(3-pyridazinyl)-1-oxa-3-azaspiro[4.5]decane-8-carboxylate (Intermediate 37 procedure 37a, 1.6 g, 5.24 mmol) was dissolved in tetrahydrofuran (50 ml), under nitrogen atmosphere, then cooled to −78° C. 1M Lithium aluminum hydride in THF (5.24 ml, 5.24 mmol) was added portion-wise to the resulting cloudy mixture. The resulting milky mixture was stirred at that temperature for 15 min, and then gently warmed up to −40° C. over 5 minutes. The resulting clean and colourless solution was left under stirring at −40° C. for ca 15 minutes. Then, it was cooled to −78° C. and quenched with Na₂SO₄×10H₂O (ca 10 g). It was stirred allowing to warm up to r.t., then filtered with a filter tube, washing the solid with DCM (3×50 ml). The resulting organic phase was concentrated under vacuum to afford 1.6 g of s colourless oil. The crude was purified with Biotage SP1, over a SNAP 40 g silica column, using EtOAc as eluent to afford the title compound as a colourless solid (1.1 g).

¹H NMR (400 MHz, CDCl₃): δ 8.95 (dd, 1H), 8.57 (dd, 1H), 7.50 (dd, 1H), 4.21 (s, 2H), 3.54 (br s, 2H), 2.03-1.13 (m, 9H). UPLC-MS: 0.44 min, 264 [M+H]+.

### Procedure 39b

To an ice cooled suspension of (trans)-2-oxo-3-(3-pyridazinyl)-1-oxa-3-azaspiro[4.5]decane-8-carboxylic acid (Intermediate 38, 16.5 g, 59.5 mmol) in THF (300 ml) borane tetrahydrofuran complex (179 ml, 179 mmol) was added dropwise and the resulting mixture was allowed to reach r.t and stirred for 2 hours. The reaction mixture was quenched at 0° C. with MeOH (40 ml) and 10% aq HCl (120 ml) was added dropwise. The mixture was stirred overnight then it was heated at 40° C. for 6 hours and left at r.t. for 60 hours. The mixture was basified to pH 8 at 0° C. with 3N NaOH and concentrated under reduced pressure to obtain a residue which was diluted with water (200 ml) and extracted with DCM (3×500 ml). Organic phase was dried (Na₂SO₄), filtered and concentrated in vacuo to give a crude which was purified by flash chromatography eluting with 100% ethyl acetate to afford the title compound (10 g, 38 mmol, 64%).

¹H NMR (400 MHz, CDCl₃): δ 8.97 (d, 1H), 8.58 (d, 1H), 7.51 (dd, 1H), 4.22 (s, 2H), 3.56 (d, 2H), 2.10-1.15 (m, 9H). UPLC-MS: 0.44 min, 264 [M+H]+.

### Intermediate 40

### (Trans)-8-(azidomethyl)-3-(3-pyridazinyl)-1-oxa-3-azaspiro[4.5]decan-2-one

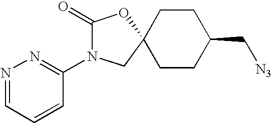

(Trans)-8-(hydroxymethyl)-3-(3-pyridazinyl)-1-oxa-3-azaspiro[4.5]decan-2-one (two combined batches: Intermediate 39 procedure 39a and a batch prepared in a similar fashion, 1.29 g, 4.90 mmol), DIAD (0.953 ml, 4.90 mmol) and diphenyl azidophosphate (4.05 g, 14.70 mmol) were dissolved in tetrahydrofuran (50 ml) and cooled to 0° C. Triphenylphosphine (3.86 g, 14.70 mmol) was added portion-wise. The resulting solution was then stirred at 0° C. for overall 6 hours. Further DIAD (0.2 equiv.) and diphenyl azidophosphate (1.0 equiv) were added. The reaction was left under stirring overnight at low temperature (from 0° C. to 10° C.). It was then taken up with EtOAc (200 ml) and quenched with sat NaHCO₃ (50 ml). The resulting organic phase was washed with sat NH₄Cl (50 ml) and brine. It was then dried over Na₂SO₄, filtered and concentrated. The resulting crude was purified with Biotage SP1, over a Biotage 65i silica cartridge, with a gradient of ethyl acetate and cyclohexane to isolate the title compound as a colourless solid (1.5 g) in a low chemical purity (50% by UPLC\MS). It was decided to use this compound in the next step without any further purification.

¹H NMR (400 MHz, CDCl₃): δ 8.96 (dd, 1H), 8.56 (dd, 1H), 7.50 (dd, 1H), 4.21 (s, 2H), 3.25 (d, 2H), 2.09-1.32 (m, 9H). UPLC-MS: 0.63 min, 289 [M+H]+.

### Intermediate 41

### (Trans)-8-(aminomethyl)-3-(3-pyridazinyl)-1-oxa-3-azaspiro[4.5]decan-2-one

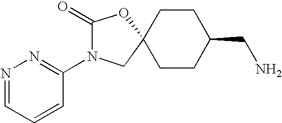

### Procedure 41a

(Trans)-8-(azidomethyl)-3-(3-pyridazinyl)-1-oxa-3-azaspiro[4.5]decan-2-one (Intermediate 40, 1.5 g, 5.20 mmol) and triphenylphosphine (2.73 g, 10.41 mmol) were collected and stirred in a mixture of tetrahydrofuran (20 ml) and water (5 ml) at room temperature overnight. The reaction mixture was then purified with a pre-conditioned Varian 10 g SCX cartridge, washing with MeOH and eluting with 2M ammonia in MeOH to recover the title compound as a colourless solid (0.9 g).

¹H NMR (400 MHz, CDCl₃): δ 8.96 (dd, 1H), 8.58 (dd, 1H), 7.50 (dd, 1H), 4.21 (s, 2H), 2.63 (d, 2H), 2.07-1.14 (m, 9H); UPLC-MS: 0.33 min, 263 [M+H]+.

### Procedure 41b

(Trans)-8-(hydroxymethyl)-3-(3-pyridazinyl)-1-oxa-3-azaspiro[4.5]decan-2-one (prepared in a similar fashion to Intermediate 39 procedure 39a but purifying the crude by SCX cartridge, 2.0 g, 6.84 mmol), DIAD (1.329 ml, 6.84 mmol) and diphenyl azidophosphate (5.64 g, 20.51 mmol) were dissolved in tetrahydrofuran (50 ml) and cooled to 10° C. Triphenylphosphine (5.38 g, 20.51 mmol) was added portion-wise. The resulting mixture was allowed to warm up to r.t. and stirred for 2 hours. Water (30 ml) and triphenylphosphine (1.793 g, 6.84 mmol) were added and the mixture was stirred at r.t. overnight. Further triphenylphosphine (1.793 g, 6.84 mmol) was added, and the mixture was stirred at 40° C. (int temp) for 2 hours. The reaction mixture was purified by a Varian SCX 20 g cartridge washing with MeOH and eluting with 2M ammonia in MeOH to give the title compound as a brown gum (1.85 g) contaminated by minor unknown impurities. This was used in the next step without any further purification.

¹H NMR (400 MHz, CDCl₃): δ 1.09-1.25 (m, 2H), 1.40-1.55 (m, 1H), 1.78-1.94 (m, 2H), 1.94-2.07 (m, 4H), 2.65 (d, 2H), 4.21 (s, 2H), 7.50 (dd, 1H), 8.58 (dd, 1H), 8.96 (dd, 1H); UPLC-MS: 0.33 min, 263 [M+H]+.

### Procedure 41c

(Trans)-8-(hydroxymethyl)-3-(3-pyridazinyl)-1-oxa-3-azaspiro[4.5]decan-2-one (prepared in a similar fashion to Intermediate 39 procedure 39b, 2.501 g, 9.50 mmol), DIAD (1.847 ml, 9.50 mmol) and diphenyl azidophosphate (7.84 g, 28.5 mmol) were collected in tetrahydrofuran (50 ml) and cooled to 0° C. Triphenylphosphine (7.48 g, 28.5 mmol) was added portion-wise. The resulting mixture was then allowed to warm up to r.t. and stirred overnight. Water (30 ml) and triphenylphosphine (2.492 g, 9.50 mmol) were added (caution: evolution of nitrogen!) at r.t. and the mixture was stirred overnight. The required product was present with the intermediate azide in ratio around 1/1. Further amount of THF (50 ml) was added to dilute the reaction mixture which was stirred for further 5 hours then purified over a strong ion exchange SCX cartrudge (50g, Varian) washing with MeOH and eluting with 2M ammonia in MeOH. Solvent was removed and the resulting crude (3.2 g as a mixture of required amine and correspondent azide) was reacted with further triphenylphosphine (2.492 g, 9.50 mmol) in THF (50.0 ml) and water (15 ml) overnight. The reaction mixture was then purified over a strong ion exchange SCX cartrudge (20g, Varian) washing with MeOH and eluting with 2M ammonia in MeOH to give the title compound as a colourless solid (1.5 g).

UPLC-MS analysis indicates the presence of ˜10% residual (trans)-8-(hydroxymethyl)-3-(3-pyridazinyl)-1-oxa-3-azaspiro[4.5]decan-2-one in the title compound. It is used in following reactions without further purification.

¹H NMR (400 MHz, CDCl₃): δ 1.08-1.20 (m, 2H), 1.33-1.51 (m, 1H), 1.81-2.08 (m, 6H), 2.63 (d, 2H), 4.21 (s, 2H), 7.46-7.54 (m, 1H), 8.58 (dd, 1H), 8.96 (dd, 1H); UPLC-MS: 0.33 min, 263 [M+H]+

### Intermediate 42

### (Trans)-8-{[(6-chloro-3-pyridazinyl)amino]methyl}-3-(3-pyridazinyl)-1-oxa-3-azaspiro[4.5]decan-2-one

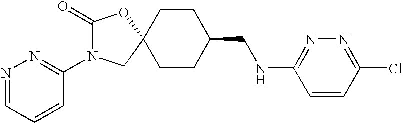

### Procedure 42a

(Trans)-8-(aminomethyl)-3-(3-pyridazinyl)-1-oxa-3-azaspiro[4.5]decan-2-one (Intermediate 41 procedure 41b, 1.85 g, 7.05 mmol), 3,6-dichloropyridazine (1.051 g, 7.05 mmol) and DIPEA (6.16 ml, 35.3 mmol) were dissolved in isopropanol (15 ml) in a closed microwave vial and heated at 130° C. (ext. temp.) overnight then for additional 8 hours. Further 3,6-dichloropyridazine (250 mg) and DIPEA (3 ml, 17.18 mmol) were added. The reaction was stirred at 130° C. for further 24 hours. It was then taken up with DCM (500 ml) and washed with sat NaHCO₃ (3×50 ml) and water (2×50 ml). The organic phase was concentrated under vacuum and the resulting crude was purified with Biotage SP1, over a KP-NH 40+M cartridge, eluting with a gradient of cyclohexane and ethyl acetate. The title compound was eluted with EtOAc and recovered as a colourless solid (1.0 g, 2.67 mmol, 38%).

¹H NMR (400 MHz, CDCl₃): δ 8.96 (dd, 1H), 8.56 (dd, 1H), 7.50 (dd, 1H), 7.18 (d, 1H), 6.68 (d, 1H), 5.05 (brt, 1H), 4.21 (s, 2H), 3.38 (t, 2H), 2.10-1.20 (m, 9H); UPLC-MS: 0.51 min, 375 [M+H]+.

### Procedure 42b

In a 2 ml sealed vial, (trans)-8-(aminomethyl)-3-(3-pyridazinyl)-1-oxa-3-azaspiro[4.5]decan-2-one (prepared in a similar fashion to Intermediate 41, procedure 41a but purifying the product on KP-NH column with 0%-50% MeOHEtOAc, 149 mg, 0.568 mmol), 3,6-dichloropyridazine (85 mg, 0.568 mmol) and TEA (0.238 ml, 1.704 mmol) were suspended in isopropanol (2 ml) and shaken at 130° C. for 72 hours. (Caution high pressure inside!). The reaction mixture was passed through an ion exchange SCX cartridge (Varian, 2g) washing with MeOH and eluting with 2M ammonia in MeOH. The recovered material was purified with Biotage SP1, over a 25M Varian NH₂ cartridge, eluting in gradient with 0%-50% MeOHEtOAc to afford the title compound as a colourless solid (87.7 mg, 0.234 mmol, 41%).

UPLC-MS: 0.51 min, 375 [M+H]+

### Intermediate 43

### (Trans)-8-(aminomethyl)-1-oxa-3-azaspiro[4.5]decan-2-one

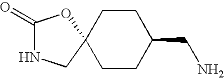

(Trans)-8-(hydroxymethyl)-1-oxa-3-azaspiro[4.5]decan-2-one (prepared in a similar fashion to Intermediate 4, 2.5 g, 13.50 mmol) DIAD (2.66 ml, 13.50 mmol) and diphenyl azidophosphate (8.73 ml, 40.5 mmol) were stirred in tetrahydrofuran (120 ml) at 0° C. Triphenylphosphine (10.62 g, 40.5 mmol) was added portion-wise and the resulting mixture was stirred at 0° C. for 1 hour, then the cooling bath was removed, the temperature was allowed to rise to r.t. and the mixture was stirred for around 3 hours. DIAD (0.5 ml) was further added and the mixture was stirred at r.t. overnight. Then the mixture was cooled to 0° C., triphenylphosphine (4.43 g, 16.87 mmol) was added followed by slow addition of water (40.0 ml) and the mixture was stirred at r.t. for 8 hours then left standing at r.t. over the weekend. The solution was filtered through and SCX cartridge (50g) eluting with MeOH and 2M NH₃ in MeOH. Ammonia fractions were combined and dried to afford the title compound as a yellow solid contaminated by some residual aromatic impurities (1.69 g).

¹H NMR (400 MHz, DMSO-d₆): δ 0.88-1.04 (m, 2H), 1.14-1.31 (m, 1H), 1.48-1.60 (m, 2H), 1.67-1.87 (m, 4H), 2.41 (d, 2H), 3.24 (s, 2H); UPLC-MS: 0.18 min, 185 [M+H]+.

### Intermediate 44

### (Trans)-8-{[(6-chloro-3-pyridazinyl)amino]methyl}-1-oxa-3-azaspiro[4.5]decan-2-one

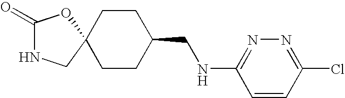

### Procedure 44a

(Trans)-8-(aminomethyl)-1-oxa-3-azaspiro[4.5]decan-2-one (Intermediate 43, 1 g, 5.43 mmol), 3,6-dichloropyridazine (0.809 g, 5.43 mmol) and DIPEA (4.74 ml, 27.1 mmol) were suspended in sulfolane (20 ml) in a closed vial. The resulting mixture was stirred at 140° C. for 3 hours. The reaction was further stirred overnight at 140° C. Then it was cooled to r.t. and purified by means of an ion exchange cartridge (SCX, Strata 10g), washing with MeOH, and eluting with 2M ammonia in MeOH. Solvent was removed to give the title compound as a brown solid (1.2 g).

¹H NMR (400 MHz, DMSO-d₆): δ 7.44 (br s, 1H), 7.34 (d, 1H), 7.14 (br t, 1H), 6.90 (d, 1H), 3.27 (s, 2H), 3.22-3.15 (m, 2H), 1.88-1.02 (m, 9H). UPLC-MS: 0.57 min, 297 [M+H]+.

### Procedure 44b

(Trans)-8-(aminomethyl)-1-oxa-3-azaspiro[4.5]decan-2-one (prepared in a similar fashion to Intermediate 43, 1.024 g, 5.56 mmol), 3,6-dichloropyridazine (0.828 g, 5.56 mmol) and TEA (3.10 ml, 22.23 mmol) in isopropanol (12 ml) were stirred at 130° C. in a closed vial for 20 hours. Then it was cooled to room temperature, the solvent was removed under vacuum and the resulting crude was purified over an ion exchange cartridge (20g, SCX) washing with MeOH and eluting with 2M NH₃ in MeOH. Ammonia fractions were combined and dried (vacuo) to afford the title compound as a beige solid (1.172 g, 71%).

¹H NMR (400 MHz, DMSO-d₆): δ 1.00-1.18 (m, 2H), 1.45-1.89 (m, 7H), 3.14-3.22 (m, 2H), 3.26 (s, 2H), 6.90 (d, 1H), 7.12 (t, 1H), 7.32 (s, 1H), 7.44 (br s, 1H); UPLC-MS: 0.41 min, 297 [M+H]+

### Intermediate 45

### (Trans)-8-({[6-(2-methylphenyl)-3-pyridazinyl]amino}methyl)-1-oxa-3-azaspiro[4.5]decan-2-one

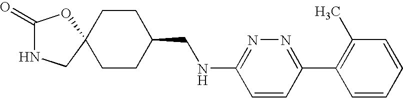

(Trans)-8-{[(6-chloro-3-pyridazinyl)amino]methyl}-1-oxa-3-azaspiro[4.5]decan-2-one (Intermediate 44 procedure 44a, 200 mg, 0.674 mmol), (2-methylphenyl)boronic acid (92 mg, 0.674 mmol), PdCl₂(PPh₃)₂ (23.65 mg, 0.034 mmol), potassium carbonate (186 mg, 1.348 mmol) were collected in a vial, deareated and suspended in deareated water (1 ml) and acetonitrile (2 ml). The resulting mixture was shaken in a closed vial at 100° C. for 3 hours. Solvent was then removed under vacuum. The crude was taken up with DCM (20 ml) and washed with water, filtering over a separation tube. The resulting organic phase was concentrated and the crude was purified with Biotage SP1, over a SNAP 120 g C18 column, using a gradient of water and ACN (made up 0.1% HCOOH). Two batches of fractions were collected, partially evaporated, and neutralised over a 5g Varian SCX cartridge, washing with MeOH and eluting with 2M ammonia in MeOH. The title compound was recovered as a colourless solid (80 mg).

¹H NMR (400 MHz, CDCl₃): δ 7.42-7.39 (m, 1H), 7.34-7.28 (m, 4H), 6.73 (d, 1H), 5.34 (s, 1H), 5.03 (br s, 1H), 3.50 (s, 2H), 3.46-3.39 (m, 2H), 2.41 (s, 3H), 2.06-1.10 (m, 9H); UPLC-MS: 0.47 min, 353 [M+H]+.

### Intermediate 46

### (Trans)-8-({[6-(3,5-dimethylphenyl)-3-pyridazinyl]amino}methyl)-1-oxa-3-azaspiro[4.5]decan-2-one

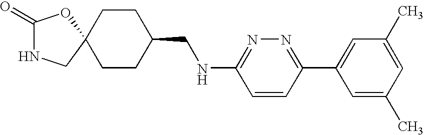

2-(3,5-dimethylphenyl)-4,4,5,5-tetramethyl-1,3,2-dioxaborolane (62.6 mg, 0.27 mmol), (trans)-8-{[(6-chloro-3-pyridazinyl)amino]methyl}-1-oxa-3-azaspiro[4.5]decan-2-one (Intermediate 44 procedure 44a, 80 mg, 0.270 mmol) and tetrakis(triphenylphosphine)palladium(0) (31.2 mg, 0.027 mmol) were collected in a closed vial and suspended in N,N-dimethylformamide (3 ml) and K₃PO₄ (0.270 ml, 0.539 mmol). The resulting mixture was shaken at 90° C. (ext temp) overnight. It was then concentrated under vacuum, taken up with DCM (20 ml) and filtered over a separation tube. The resulting organic solution was concentrated and then purified with Biotage SP1, over a KP-C18 50 g column, eluting with a gradient of water and ACN (made up with 0.1% HCOOH). Fractions containing the required compound were collected, partially concentrated, and then neutralised with a 2g SCX cartridge (Varian) washing with MeOH and eluting with 2M ammonia in MeOH. The title compound was recovered as a colourless liquid (20 mg).

¹H NMR (400 MHz, CDCl₃): δ 7.62-7.58 (m, 3H), 7.06 (br s, 1H), 6.72 (d, 1H), 5.75 (br s, 1H), 5.03 (br s, 1H), 3.47-3.38 (m, 4H), 2.40 (s, 6H), 2.05-1.07 (m, 8H); UPLC-MS: 0.76, 367 [M+H]+.

### Intermediate 47

### (Trans)-8-({[6-(2-fluorophenyl)-3-pyridazinyl]amino}methyl)-1-oxa-3-azaspiro[4.5]decan-2-one

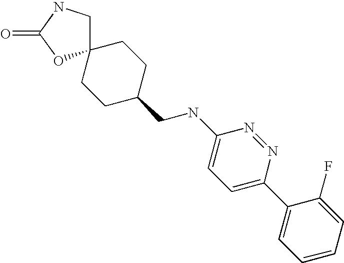

(Trans)-8-{[(6-chloro-3-pyridazinyl)amino]methyl}-1-oxa-3-azaspiro[4.5]decan-2-one (Intermediate 44 procedure 44b, 250 mg, 0.842 mmol), (2-fluorophenyl)boronic acid (141 mg, 1.011 mmol), PdCl₂(PPh₃)₂ (29.6 mg, 0.042 mmol) and K₂CO₃ (233 mg, 1.685 mmol) were suspended in acetonitrile (4 ml) and water (2 ml) in a microwave vial. The mixture was irradiated at 120° C. for 30 min (normal heating). The mixture was filtered through a separating tube washing with DCM. The organic was concentrated (vacuo) to afford a crude that was purified by chromatography (KP-NH column, 25+M, Biotage SP1) eluting in gradient with 0%-90% MeOHDCM to afford the title compound as a pale yellow solid (205 mg, 68%).

¹H NMR (400 MHz, DMSO-d₆): δ 1.01-1.22 (m, 2H), 1.49-1.64 (m, 2H), 1.64-1.76 (m, 1H), 1.76-1.92 (m, 4H), 3.12-3.20 (m, 2H), 3.26-3.31 (m, 2H), 6.83-6.95 (m, 1H), 7.04-7.20 (m, 1H), 7.25-7.36 (m, 2H), 7.36-7.53 (m, 2H), 7.53-7.65 (m, 1H), 7.78-7.94 (m, 1H); UPLC-MS: 0.46 min, 357 [M+H]+.

### Intermediate 48

### (Trans)-8-({[6-(3-fluorophenyl)-3-pyridazinyl]amino}methyl)-1-oxa-3-azaspiro[4.5]decan-2-one

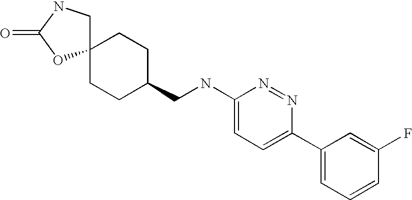

(Trans)-8-{[(6-chloro-3-pyridazinyl)amino]methyl}-1-oxa-3-azaspiro[4.5]decan-2-one (Intermediate 44 procedure 44b, 82 mg, 0.276 mmol), (3-fluorophenyl)boronic acid (38.7 mg, 0.276 mmol), K₂CO₃ (76 mg, 0.553 mmol), PdCl₂(PPh₃)₂ (9.70 mg, 0.014 mmol) in acetonitrile (2 ml) and water (1 ml) were placed in a 5 ml microwave vial and the mixture was irradiated at 120° C. for 40 min (normal heating). The mixture was dried (vacuo) then it was taken up with DCM and few drops of MeOH and filtered through a separating tube washing with DCM. The organic was combined and dried (vacuo) to afford a crude that was purified by SCX (5g) loading with DCM/MeOH and eluting with 2M NH₃ in MeOH. Ammonia fractions were combined and dried to afford the title compound as a yellow solid (80 mg, 81%)

¹H NMR (400 MHz, DMSO-d₆): δ 1.01-1.22 (m, 2H), 1.49-1.74 (m, 3H), 1.76-1.92 (m, 4H), 3.22-3.32 (m under H₂O peak, 4H), 6.83-6.95 (m, 1H), 7.04-7.18 (m, 1H), 7.18-7.28 (m, 1H), 7.39-7.47 (m, 1H), 7.47-7.57 (m, 1H), 7.73-7.91 (m, 2H); UPLC-MS: 0.48 min, 357 [M+H]+

### Intermediate 49

### 3-Chloro-6-(2-pyridinyl)pyridazine

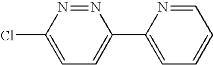

A mixture of 3,6-dichloropyridazine (596 mg, 4 mmol), 6-phenyl-2-(2-pyridinyl)tetrahydro-4H-1,3,6,2-dioxazaborocine (1823 mg, 6.80 mmol), bis(triphenylphosphine)palladium(II) chloride (140 mg, 0.200 mmol), potassium phosphate tribasic (1698 mg, 8.00 mmol) and copper (I) iodide (381 mg, 2.000 mmol) was placed in a carosel reaction tube and placed under nitrogen. DMF (20 ml) was added and the resulting mixture stirred and heated to 100° C. for 90 minutes. The reaction mixture was cooled, diluted with EtOAc (100 ml) and washed with water (200 ml). The aqueous phase was back-extracted with EtOAc (100 ml). The combined organics were washed with water (100 ml), dried over Na₂SO₄ and evaporated. The residue was chromatographed three times on the Biotage (50%-100% EtOAc/cyclohexane; 40+M NH column then 20% EtOAc/dichloromethane; 25M SiO₂ column then 25% diethyl ether/cyclohexane; 25M NH column) to give the title compound (120 mg) as a white solid.

¹H NMR (400 MHz, CDCl₃): δ 8.73 (ddd, 1H), 8.67 (dt, 1H), 8.58 (d, 1H), 7.88-7.95 (m, 1H), 7.65 (d, 1H), 7.44 (ddd, 1H); UPLC-MS: 0.56 min, 192 and 194 [M+H]+

### Intermediate 50

### 6-(Tetrahydro-2H-pyran-4-yloxy)-3-pyridazinamine

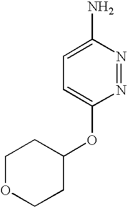

A freshly cut lump of sodium (100 mg, 4.35 mmol) was added to tetrahydro-2H-pyran-4-ol (5 g, 49.0 mmol) in a screw top vial. The mixture was shaken in a PLS reactor and was warmed gradually, checking that the rate of hydrogen liberation was not excessive, until a temperature of 105° C. was reached. After ˜1 hour the sodium has been consumed to give a brown solution. 6-Chloro-3-pyridazinamine (0.451 g, 3.48 mmol) was added and shaking was continued at 105° C. overnight. It was cooled and diluted with methanol (10 ml) and the reaction mixture was neutralised with dilute hydrochloric acid (˜2 ml of 0.5M HCl required). It was loaded onto a pre-conditioned SCX cartridge; eluted first with MeOH to remove the excess tetrahydro-2H-pyran-4-ol then with 2M NH₃ in MeOH. The basic fractions containing product were evaporated then the residue was purified via Biotage (5%-10% EtOAc/CH₂Cl₂; 40+M NH column) to give the title compound (140 mg) as a pale yellow solid.

¹H NMR (400 MHz, CDCl₃): δ 6.79-6.84 (AB quartet, 2H), 5.34 (sept, 1H), 4.39 (br s, 2H), 3.95-4.05 (m, 2H), 3.56-3.67 (m, 2H), 2.10-2.20 (m, 2H), 1.75-1.87 (m, 2H); UPLC-MS: 0.32 min, 196 [M+H]+.

### Intermediate 51

### 6-({2-[(1,1-dimethylethyl)oxy]ethyl}oxy)-3-pyridazinamine

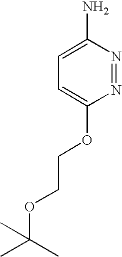

60% sodium hydride in mineral oil (102 mg, 2.54 mmol) was washed with anhydrous hexane (3 ml) and then suspended in DME (1 ml). A solution of 2-[(1,1-dimethylethyl)oxy]ethanol (200 mg, 1.692 mmol) in DME (2 ml) was added dropwise to the stirred suspension of NaH. After addition and hydrogen liberation was complete, the torbid mixture was taken up in a syringe and filtered through a millipore syringe filter into a screw-topped vial to give a limpid solution. 6-chloro-3-pyridazinamine (175 mg, 1.354 mmol) was added and the mixture was heated to 130° C. for 45 mins. The temperature was lowered to 90° C. and left overnight at this temperature, then heated again to 130° C. for 3 hours. The reaction mixture was cooled and neutralised with dilute hydrochloric acid solution (˜2 ml, 1.0M). The reaction mixture was loaded onto a pre-conditioned SCX cartridge (20 g) and eluted with MeOH and then 2M NH₃ in MeOH. The basic fractions containing product were evaporated and the residue was purified by reverse phase chromatography on the Biotage (eluent MeCN/water/formic acid 5/94.9/0.1, SNAP 60g C18 column). The fractions containing the desired product were loaded onto a pre-conditioned SCX cartridge (10 g) and eluted with MeOH and then 2M NH₃ in MeOH. The basic fractions containing product were evaporated to give 90 mg of a yellow oil.

¹H NMR (400 MHz, CDCl₃): δ 6.89 (d, 1H), 6.80 (d, 1H), 4.51 (t, 2H), 4.47 (brs, 2H), 3.75 (t, 2H), 1.24 (s, 9H); UPLC-MS: 0.53 min, 212 [M+H]+ and 156 [M-butene+H]+.

### Intermediate 52

### 6-{[(3-methyl-3-oxetanyl)methyl]oxy}-3-pyridazinamine

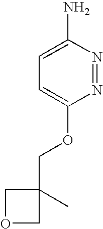

A solution of (3-methyl-3-oxetanyl)methanol (259 mg, 2.53 mmol) in toluene (3 ml) was added dropwise to a stirred suspension of 60% sodium hydride in mineral oil (95 mg, 2.364 mmol) in toluene (1 ml) at room temperature. The mixture was stirred for ˜30 minutes when no further gas evolution was observed. 6-chloro-3-pyridazinamine (175 mg, 1.351 mmol) and NMP (1 ml) were added and the mixture was heated to 110° C. for 2 hours. This was cooled to 70° C. and left overnight (˜18 hours). The mixture was cooled and neutralised with dilute hydrochloric acid solution (˜2 ml, 1.0M). The reaction mixture was loaded onto a pre-conditioned SCX cartridge (10 g) and eluted with MeOH and then 2M NH₃ in MeOH. The basic fractions containing product were evaporated and the residue was purified on the Biotage (10-40% EtOAc/CH₂Cl₂, 25M NH column) to give the title compound (79 mg) as a brown oil.

¹H NMR (400 MHz, CDCl₃): δ 6.89 (d, 1H), 6.82 (d, 1H), 4.68 (d, 2H), 4.40-4.50 (m, 6H), 1.44 (s, 3H); UPLC-MS: 0.33 min, 196 [M+H]+.

### Intermediate 53

### 6-(cyclopentyloxy)-3-Pyridazinamine

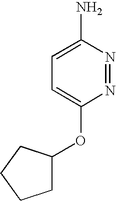

Freshly cut sodium (127 mg, 5.52 mmol) was added to cyclopentanol (5 g, 58.1 mmol) in a screw-topped pressure tube at room temperature. The mixture was stirred overnight at room temperature then heated to ˜80° C. The sodium reacts very slowly over the course of several hours. The mixture was heated further until the solid sodium became molten—at this point the evolution of hydrogen was visibly faster and the last remaining sodium reacted within 1 hour. The mixture was cooled, at which point solidified, and 6-chloro-3-pyridazinamine (573 mg, 4.42 mmol) was added and then the mixture was heated to 140° C. Purification via SCX (10g) was tried but some material of the desired molecular weight eluted with MeOH washings whereas some stuck to the resin and was only eluted with NH₃ in MeOH. All product containing fractions were recombined and evaporated. The dark brown residue was purified via Biotage (5%-10% EtOAc/CH₂Cl₂; 40+M NH column) to give the title compound (411 mg) as a yellow oil, which solidified on standing.

¹H NMR (400 MHz, CDCl₃): δ 6.77 (s, 2H), 5.50 (sept, 1H), 4.34 (br s, 2H), 1.96-2.09 (m, 2H), 1.72-1.87 (m, 4H), 1.56-1.70 (m, 2H); UPLC-MS: 0.44 min, 180 [M+H]+.

### Intermediate 54

### 6-(3,5-Difluorophenyl)-3-pyridazinamine

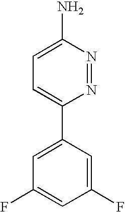

Potassium carbonate (427 mg, 3.09 mmol), (3,5-difluorophenyl)boronic acid (366 mg, 2.316 mmol), 6-chloro-3-pyridazinamine (200 mg, 1.544 mmol) and PdCl₂(PPh₃)₂ (54.2 mg, 0.077 mmol) were weighed into a microwave vial and placed under nitrogen. De-aereated acetonitrile/water (3:1, 12 ml) was added and the resulting biphasic mixture was heated to 110° C. in a microwave reactor for 45 min. This was evaporated under reduced pressure then the residue was partitioned between dichloromethane (50 ml) and water (50 ml). This was filtered through a hydrophobic frit (Phase Seperator cartridge) washing with more dichloromethane (2×30 ml). The combined organic phases were evaporated under reduced pressure then loaded onto an SCX cartridge (5g) which was eluted with MeOH then 2M NH₃ in MeOH. The basic fractions containing product were evaporated to give a beige solid which was purified via Biotage (EtOAc; 25M NH column) to give the title compound (189 mg) as a white solid.

¹H NMR (400 MHz, CDCl₃): δ 7.60 (d, 1H), 7.48-7.58 (m, 2H), 6.82-6.90 (m, 2H), 4.92 (s, 2H); UPLC-MS: 0.60 min, 208 [M+H]+

### Intermediate 55

### 1,1-Dimethylethyl [6-(3,5-difluorophenyl)-3-pyridazinyl]-carbamate

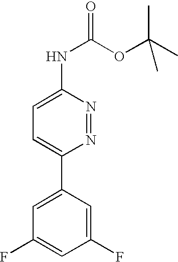

A solution of 6-(3,5-difluorophenyl)-3-pyridazinamine (Intermediate 54, 100 mg, 0.483 mmol) and Boc₂O (0.168 mL, 0.724 mmol) in tert-butanol (3 ml) was shaken at 80° C. in a screw topped vial for 2 hours then at 60° C. overnight. Further Boc2O (0.84 mL, 0.362 mmol) was added and shaken at 80° C. for another 2 hours. The t-butanol was evaporated then the residue suspended in methanol (3 ml), potassium carbonate (˜200 mg) and a few drops of water were added and shaken at 80° C. for 1 hour. This was diluted with water (10 ml) and dichloromethane (10 ml), and filtered through a hydrophobic frit (Phase Seperator cartridge). The organic phase was evaporated and the residue purified via Biotage (gradient from 50% DCM/50% cyclohexane to 10% EtOAc/45% dichloromethane/45% cyclohexane; SNAP 10 SiO2 column) to give the title compound (129 mg) as a white solid.

¹H NMR (400 MHz, CDCl₃): δ 8.35 (d, 1H), 7.85 (brs, 1H), 7.81 (d, 1H), 7.55-7.65 (m, 2H), 6.92 (tt, 1H), 1.58 (s, 9H); UPLC-MS: 0.92 min, 252 [M+H—C₄H₈]+ and 308 [M+H]+

### Intermediate 56

### [(Trans)-2-oxo-3-(3-pyridazinVl)-1-oxa-3-azaspiro[4.5]dec-8-yl]methyl methanesulfonate

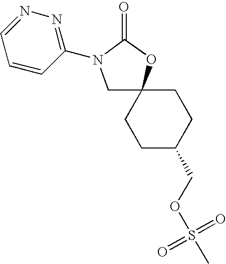

A solution of methanesulfonic anhydride (331 mg, 1.899 mmol) in dichloromethane (2 ml) was added dropwise at 0° C. to a stirred solution of (trans)-8-(hydroxymethyl)-3-(3-pyridazinyl)-1-oxa-3-azaspiro[4.5]decan-2-one (prepared in a similar fashion to Intermediate 39 procedure 39b, 250 mg, 0.950 mmol) and triethylamine (0.291 ml, 2.089 mmol) in dichloromethane (10 ml). After addition was complete, the reaction was allowed to warm to room temperature and stirred for 1 hour. It was quenched with saturated sodium hydrogen carbonate solution (15 ml) and stirred vigorously for 30 minutes, then filtered through a hydrophobic frit (Phase Seperator cartridge). The organic layer was shaken with dilute aqueous hydrochloric acid (0.5M, 15 ml) and filtered through a hydrophobic frit (Phase Seperator cartridge). The organic layer was evaporated under reduced pressure to give an oily residue which was purified via Biotage (100% EtOAc; 25M SiO₂ column) to give the title compound (231 mg) as a white solid.

¹H NMR (400 MHz, CDCl₃): δ 8.98 (dd, 1H), 8.58 (dd, 1H), 7.52 (dd, 1H), 4.23 (s, 2H), 4.13 (d, 2H), 3.07 (s, 3H), 1.99-2.12 (m, 4H), 1.86-1.98 (m, 3H), 1.25-1.38 (m, 2H); UPLC-MS: 0.59 min, 342 [M+H]+

### Intermediate 57

### 1,1-Dimethylethyl [6-(3,5-difluorophenyl)-3-pyridazinyl]{[(trans)-2-oxo-3-(3-pyridazinyl)-1-oxa-3-azaspiro[4.5]dec-8-yl]methyl}carbamate

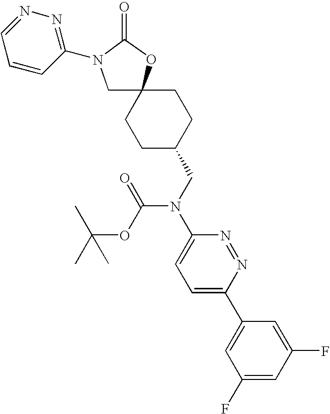

Potassium tert-butoxide (44.4 mg, 0.395 mmol) was added to a stirred solution of 1,1-dimethylethyl [6-(3,5-difluorophenyl)-3-pyridazinyl]carbamate (Intermediate 55, 122 mg, 0.395 mmol) in DMF (2 ml) at room temperature under nitrogen. This was stirred for 30 min then [(trans)-2-oxo-3-(3-pyridazinyl)-1-oxa-3-azaspiro[4.5]dec-8-yl]methyl methanesulfonate (Intermediate 56, 90 mg, 0.264 mmol) added and heated to 80° C. for 1 hour. This was cooled and poured into a saturated aqueous solution of NH4Cl (20 ml). It was extracted with EtOAc (3×20 ml), then the combined organic phases were washed with water (50 ml) and brine (50 ml), dried (Na2SO4) and evaporated under reduced pressure. The residue was purified via Biotage (50%-100% EtOAc/cyclohexane; 25M SiO2 column) to give 45 mg of recovered 1,1-dimethylethyl [6-(3,5-difluorophenyl)-3-pyridazinyl]carbamate and the title compound in a ˜2:1 mixture with its regioisomer 1,1-dimethylethyl [(3E)-6-(3,5-difluorophenyl)-2-{[(trans)-2-oxo-3-(3-pyridazinyl)-1-oxa-3-azaspiro[4.5]dec-8-yl]methyl}-3(2H)-pyridazinylidene]carbamate (145 mg) as a pale yellow solid.

¹H NMR (400 MHz, CDCl₃): δ 8.98 (dd, 1H Intermediate 57+1H regioisomer), 8.57 (dd, 1H Intermediate 57+1H regioisomer), 8.31 (d, 1H regioisomer), 8.18 (d, 1H Intermediate 57), 7.77 (d, 1H Intermediate 57), 7.60-7.69 (m, 2H Intermediate 57), 7.55 (d, 1H regioisomer), 7.50 (dd, 1H Intermediate 57+1H regioisomer), 7.31-7.38 (m, 2H regioisomer), 6.88-6.98 (m, 1H Intermediate 57+1H regioisomer), 4.37 (d, 2H regioisomer), 4.18-4.27 (m, 4H Intermediate 57+2H regioisomer), 2.22-2.37 (m, 1H regioisomer), 1.20-2.10 (m, 9H Intermediate 57+8H regioisomer), 1.45 (s, 9H Intermediate 57+9H regioisomer); UPLC-MS: 1.00 min, 553 [M+H]+ and 453 [M+H-Boc]+ (Intermediate 57) and 0.96 min, 553 [M+H]+ and 453 [M+H-Boc]+(regioisomer)

### Intermediate 58

### 3,6-Diiodopyridazine

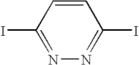

3,6-dichloropyridazine (1 g, 6.71 mmol) was suspended in HI 67% (40 ml). The mixture was stirred at r.t. for 19 hours 50 minutes. The solution was poured into 100 ml of water and the aqueous solution was extracted first with 100 ml of EtOAc and then 6 times with 50 ml of DCM. The combined organic layers were washed with 50 ml of brine, dried over Na₂SO₄, filtered and evaporated to dryness. The resulting crude compound was purified by flash chromatography on silica gel (Biotage SP1, 100 g SNAP column) with the following gradient: A: cyclohexane/B: EtOAc: 0% B for 6 min, 0% to 10% B in 15 min, 10% B for 5 min to give the title compound as a white solid (1.046 g, 45%).

¹H NMR (400 MHz, CDCl₃): δ 7.52 (s, 2H); UPLC-MS: 0.56 min, 332.71 [M+H]+.

### Intermediate 59

### 2-Bromo-5-(cyclopentyloxy)pyridine

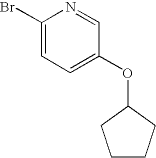

6-bromo-3-pyridinol (500 mg, 2.87 mmol) and K₂CO₃ (794 mg, 5.75 mmol) were suspended in N,N-dimethylformamide (6 ml). Bromocyclopentane (0.308 ml, 2.87 mmol) was added and the mixture was stirred at 80° C. for 5 hours, then it was left standing at r.t. overnight and heated again at 80° C. for 8 hours. The mixture was cooled to r.t. filtered on a filter tube washing with EtOAc. The organic was washed with ice/water (3×2 ml) then dried (vacuo) to afford 630 mg of a brown oil that was purified by silica gel chromatography (Biotage SP1 25+M column), eluting with Cyclohexane: 10% EtOAc\cyclohexane. Product fractions were combined and dried (vacuo) to afford the title compound as a colourless oil (408 mg, 58%).

¹H NMR (400 MHz, CDCl₃): δ 1.63-1.72 (m, 2H), 1.75-2.00 (m, 6H), 4.73-4.80 (m, 1H), 7.08 (dd, 1H), 7.36 (d, 1H), 8.04 (d, 1H); UPLC-MS: 0.80 min, 242, 244 [M+H]+.

### Intermediate 60

### (Trans)-2-oxo-3-(3-pyridazinyl)-1-oxa-3-azaspiro[4.5]decane-8-carbaldehyde

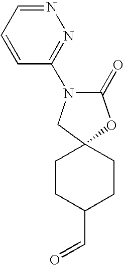

The title compound was made in a similar fashion to Intermediate 5 procedure 5b, replacing (trans)-8-(hydroxymethyl)-1-oxa-3-azaspiro[4.5]decan-2-one with (trans)-8-(hydroxymethyl)-3-(3-pyridazinyl)-1-oxa-3-azaspiro[4.5]decan-2-one (Intermediate 39 procedure 39a, 500 mg, 1.899 mmol) to afford the title compound as a colourless solid (490 mg).

¹H NMR (400 MHz, CDCl₃): δ 9.73 (s, 1H), 8.96 (dd, 1H), 8.54 (dd, 1H), 7.50 (dd, 1H), 4.13 (s, 2H), 2.52-2.46 (m, 1H), 2.20-1.78 (m, 8H); UPLC-MS: 0.49 min, 262 [M+H]+.

### Intermediate 61

### 5-(Cyclopentyloxy)-2-pyridinamine

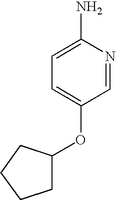

6-amino-3-pyridinol (200 mg, 1.816 mmol) and K₂CO₃ (502 mg, 3.63 mmol) were suspended in N,N-dimethylformamide (2.5 ml). Bromocyclopentane (0.195 ml, 1.816 mmol) was added and the mixture was stirred at 80° C. for 5 hours. The mixture was allowed to cool to r.t., filtered on a filter tube washing with EtOAc. The organic was washed with ice/water (3×2 ml) then dried (vacuo) to afford a brown oil that was purified by silica gel chromatography (Biotage SP1, 25+M column) eluting in gradient with 0%-10% MeOH\DCM to afford the title compound as a brown oil (11 mg, 3%).

UPLC-MS: 0.46 min, 179 [M+H]+; TLC (DCM:MeOH 9:1) R_{f} 0.63.

### Intermediate 62

### 5-(Cyclohexyloxy)-2-pyridinamine

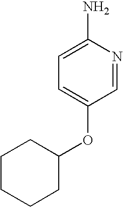

6-amino-3-pyridinol (300 mg, 2.72 mmol), bromocyclohexane (0.333 ml, 2.72 mmol) and K₂CO₃ (753 mg, 5.45 mmol) were suspended in N,N-dimethylformamide (3 ml). The resulting mixture was stirred at r.t. overnight then it was filtered and washing with EtOAc. The organic solution was dried (vacuo) to afford a crude that was purified by silica gel chromatography (Biotage SP1, 25+M column) eluting first with 5% cyclohexaneDCM and then with 5% MeOHDCM to afford the title compound (17 mg, 3%).

¹H NMR (400 MHz, CDCl₃): δ 1.21-1.42 (m, 3H), 1.42-1.68 (m, 3H), 1.68-1.88 (m, 2H), 1.88-2.08 (m, 2H), 3.97-4.09 (m, 1H), 4.21 (br s., 2H), 6.48 (dd, 1H), 7.12 (dd, 1H), 7.81 (dd, 1H); MS: 193 [M+H]+.

### Intermediate 63

### (Trans)-8-({[6-(3,5-difluorophenyl)-3-pyridazinyl]amino}methyl)-1-oxa-3-azaspiro[4,5-]decan-2-one

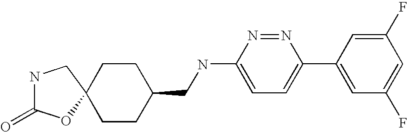

The title compound was made in a similar fashion to the preparation of Intermediate 48 replacing (3-fluorophenyl)boronic acid with (3,5-difluorophenyl)boronic acid (319 mg, 2.022 mmol) to afford the title compound as a yellow solid (263 mg, 52%).

UPLC-MS: 0.51 min, 375 [M+H]+.

### Intermediate 64

### (Trans)-8-({[6-(cyclopentyloxy)-3-pyridazinyl]amino}methyl)-1-oxa-3-azaspiro[4.5]decan-2-one

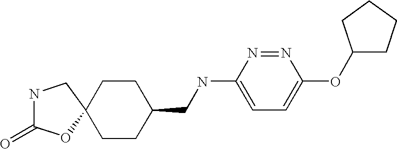

(Trans)-2-oxo-1-oxa-3-azaspiro[4.5]decane-8-carbaldehyde (prepared in a similar fashion to Intermediate 5 procedure 5b, 250 mg, 1.365 mmol), 6-(cyclopentyloxy)-3-pyridazinamine (Intermediate 53, 245 mg, 1.365 mmol) and titanium(IV) isopropoxide (0.800 ml, 2.73 mmol) in dichloromethane (20 ml) were stirred at r.t. overnight. Titanium(IV) isopropoxide (0.2 ml) was further added and the mixture was stirred at r.t. for hours then NaBH₄ (258 mg, 6.82 mmol) followed by ethanol (6.67 ml) were added (caution: gas evolution!) and the mixture was stirred at r.t. for 5 hours. Then a saturated NaHCO₃ solution was added. The mixture was stirred for 30 minutes at r.t. then it was extracted with DCM (3×20 ml). The organics were combined, filtered through a separatory tube and dried (vacuo) to afford a crude that was purified by reverse phase flash chromatography (Biotage SP1, C18 120 g column) eluting with a gradient of acetonitrile and water containing 0.1% HCOOH. Fractions containing the required compound were collected and passed through a SCX cartridge (10g, Varian) washing with MeOH and eluting with 2M ammonia in MeOH. The ammonia fractions were dried (vacuo) to afford the title compound as a white solid (222 mg, 47%).

¹H NMR (400 MHz, CDCl₃): δ 1.04-1.19 (m, 2H), 1.54-1.70 (m, 2H), 1.70-1.90 (m, 7H), 1.91-2.09 (m, 6H), 3.30-3.44 (m, 4H), 4.19-4.27 (m, 1H), 4.91-4.98 (m, 1H), 5.45-5.52 (m, 1H), 6.60-6.70 (d, 1H), 6.70-6.80 (d, 1H); UPLC-MS: 0.49 min, 347 [M+H]+.

### Intermediate 65

### (Trans)-2-oxo-3-(3-pyridinyl)-1-oxa-3-azaspiro[4.5]decane-8-carbaldehyde

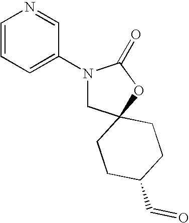

Dess-Martin Periodinane (231 mg, 0.545 mmol) and (trans)-8-(hydroxymethyl)-3-(3-pyridinyl)-1-oxa-3-azaspiro[4.5]decan-2-one (prepared in a similar fashion to Intermediate 11 procedure 11b, 130 mg, 0.496 mmol) were collected, deareated and then suspended in deareated DCM (10 ml). The resulting solution was stirred at r.t. for 4 hours. The reaction mixture was taken up with DCM (10 ml) and treated with a saturated Na₂SO₃ aq solution (2 ml) deareated for 30 min. Then, the reaction was filtered over a filter tube. DCM was treated with sat K₂CO₃ (4 ml) and filtered over a filter tube. The resulting organic phase was concentrated to afford the title compound (140 mg).

¹H NMR (400 MHz, CDCl₃): δ 9.76 (s, 1H), 8.59 (d, 1H), 8.40 (dd, 1H), 8.20 (dq, 1H), 7.33 (ddd, 1H), 3.75 (s, 2H), 2.54 (quint, 1H), 2.22-1.79 (m, 8H).

### Intermediate 66

### 2-(6-Fluoro-3-pyridinyl)pyrimidine

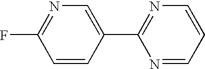

In 20 ml sealed vial a mixture of 2-bromopyrimidine (300 mg, 1.887 mmol), 2-fluoro-5-(4,4,5,5-tetramethyl-1,3,2-dioxaborolan-2-yl)pyridine (421 mg, 1.887 mmol), PdCl₂(dppf) (154 mg, 0.189 mmol) and sodium carbonate (5.66 ml, 5.66 mmol) in DME (2 ml) was stirred at 100° C. for 5 hours. Solvent was removed under vacuum; the residue was taken up with DCM (100 ml) and washed with water (2×20 ml). The resulting organic phase was concentrated and the crude (500 mg) was purified by silica gel chromatography (Biotage SP1) over a 25M silica cartridge, eluting with a gradient of cyclohexane and EtOAc. The title compound was eluted with ca 40% EtOAc and recovered as colourless solid (300 mg).

¹H NMR (400 MHz, CDCl₃): δ 9.32 (d, 1H), 8.87-8.80 (m, 3H), 7.29-7.27 (m, 1H), 7.07 (ddd, 1H); UPLC-MS: 0.54 min, 176 [M+H]+.

### Intermediate 67

### 3-Chloro-6-(2-fluorophenyl)pyridazine

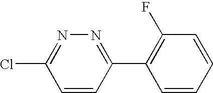

3,6-dichloropyridazine (0.555 g, 3.72 mmol), (2-fluorophenyl)boronic acid (0.25 g, 1.787 mmol), potassium carbonate (0.412 g, 2.98 mmol), PdCl₂(PPh₃)₂ (0.105 g, 0.149 mmol), were collected into a 20 ml microwave vial and suspended in acetonitrile (10 ml) and water (5.00 ml). The reaction mixture was irradiated at 120° C. for 10 min then diluted with DCM (200 ml) and washed with brine (2×50 ml). Aqueous phase was further extracted with EtOAc (2×100 ml), Et₂O (2×100 ml) and DCM (2×100 ml). The collected organic phases were dried over Na₂SO₄ and concentrated to afford 650 mg of crude oil. The crude was purified with Biotage SP1, on a silica SNAP 100 g cartridge, eluting in gradient with cyclohexane and EtOAc. The title compound was eluted with ca 30% EtOAc and recovered as a colourless solid (130 mg).

¹H NMR (400 MHz, CDCl₃): δ 8.18 (dt, 1H), 7.96 (dd, 1H), 7.60 (d, 1H), 7.55-7.46 (m, 1H), 7.37-7.33 (m, 1H), 7.25-7.20 (m, 1H). UPLC-MS: 0.65 min, 209 [M+H]+.

### Intermediate 68

### 3-Fluoro-6-(2-fluorophenyl)pyridazine

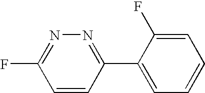

3-chloro-6-(2-fluorophenyl)pyridazine (Intermediate 67, 50 mg, 0.240 mmol), potassium fluoride (20.89 mg, 0.360 mmol), 18-crown-6 (6.33 mg, 0.024 mmol) were dissolved in sulfolane (2 ml, 21.14 mmol) in a 2 ml microwave vial and irradiated at 200° C. for overall 60 min. The reaction mixture was diluted with DCM (50 ml) and washed with water (5×30 ml). The organic phase was then concentrated under vacuum and the resulting crude was purified with Biotage SP1 over a 10 g SNAP cartridge, using cyclohexane and ethyl acetate as eluent. The required compound was eluted with ca 20% EtOAc and recovered as colourless solid (15 mg).

¹H NMR (400 MHz, CDCl₃): δ 8.17-8.07 (m, 2H), 7.56-7.45 (m, 1H), 7.38-7.27 (m, 2H), 7.27-7.17 (m, 1H). UPLC-MS: 0.61 min, 193 [M+H]+.

### Intermediate 69

### 6-(1-Piperidinyl)-3-pyridazinamine

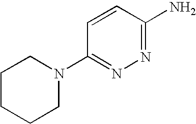

6-chloro-3-pyridazinamine (250 mg, 1.930 mmol) and piperidine (0.382 ml, 3.86 mmol) were suspended in N,N-dimethylacetamide (3 ml) and irradiated at 150° C. for 30 min. The reaction was further irradiated at 250° C. for 1 hour. The reaction mixture was passed through an ion exchange cartridge (SCX, 5g, Varian) washing with methanol and eluting with 2M ammonia in methanol. The recovered crude (250 mg) was purified with Biotage SP1, over a KP-NH 40M cartridge, eluting with a gradient of cyclohexane and ethyl acetate. The required compound was recovered as brown oil (100 mg) after elution with 10% MeOHDCM. This material was further purified by RP-flash chromatography (Biotage SP1) over a C18 12g cartridge eluting with a gradient of water and ACN (made up with 0.1% HCOOH). The collected fractions were passed through an ion exchange cartridge (SCX, 2g Varian), eluting with 2M ammonia in MeOH to give the title compound (35 mg) as yellow oil.

¹H NMR (400 MHz, CDCl₃): δ 6.94 (d, 1H), 6.71 (d, 1H), 4.32 (br s, 2H), 3.48-3.40 (m, 4H), 1.73-1.50 (m, 4H); UPLC-MS: 0.37 min, 179 [M+H]+.

### Intermediate 70

### 2-Fluoro-6-(4,4,5,5-tetramethyl-1,3,2-dioxaborolan-2-yl)pyridine

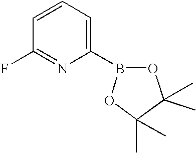

2-bromo-6-fluoropyridine (200 mg, 1.136 mmol), 4,4,4′,4′,5,5,5′,5′-octamethyl-2,2′-bi-1,3,2-dioxaborolane (317 mg, 1.250 mmol), potassium acetate (335 mg, 3.41 mmol), PdCl₂(dppf) (83 mg, 0.114 mmol) were collected in a vial, deareated, and then suspended in DMSO (10 ml) under nitrogen atmosphere. The resulting mixture was stirred at 80° C. for 3 hours, diluted with DCM (500 ml) and washed with water (5×100 ml). The resulting organic phase was concentrated under vacuum to give 250 mg of a crude residue which was used in the next step without any further purification.

UPLC-MS: 0.38 min, 142 [M-C₆H₁₀+H]+

### Intermediate 71

### (Trans)-8-[({6-[3-(trifluoromethyl)phenyl]-3-pyridazinyl}amino)methyl]-1-oxa-3-azaspiro[4.5]decan-2-one

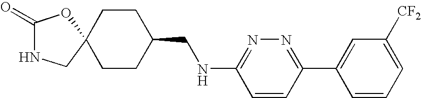

The title compound was made in a similar fashion to the preparation of Intermediate 45 replacing (2-methylphenyl)boronic acid with [3-(trifluoromethyl)phenyl]boronic acid (128 mg, 0.674 mmol) to give the title compound as a colourless solid (100 mg).

¹H NMR (400 MHz, CDCl₃): δ 1.08-1.24 (m, 2H), 1.75-1.88 (m, 3H), 1.94-2.05 (m, 4H), 3.41 (s, 2H), 3.48 (t, 2H), 4.90-4.99 (m, 1H), 6.75 (d, 1H), 7.56-7.70 (m, 3H), 8.17 (d, 1H), 8.27 (s, 1H); UPLC-MS: 0.54 min, 407 [M+H]+.

### Intermediate 72

### (Trans)-8-[({6-[3,5-bis(trifluoromethyl)phenyl]-3-pyridazinyl}amino)methyl]-1-oxa-3-azaspiro[4.5]decan-2-one

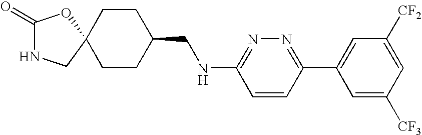

The title compound was made in a similar fashion to the preparation of Intermediate 45 replacing (2-methylphenyl)boronic acid with [3,5-bis(trifluoromethyl)phenyl]boronic acid (174 mg, 0.674 mmol) to give the title compound as a colourless solid (100 mg).

¹H NMR (400 MHz, CDCl₃): δ 1.08-1.26 (m, 2H), 1.75-1.91 (m, 3H), 1.94-2.07 (m, 4H), 3.42 (s, 2H), 3.50 (t, 2H), 4.96-5.14 (m, 1H), 6.78 (d, 1H), 7.69 (d, 1H), 7.90 (s, 1H), 8.47 (s, 2H); UPLC-MS: 0.65 min, 475 [M+H]+.

### Intermediate 73

### (Trans)-8-{[(6-chloro-4-methyl-3-pyridazinyl)amino]methyl}-3-(3-pyridazinyl)-1-oxa-3-azaspiro[4.5]decan-2-one-(Trans)-8-{[(6-chloro-5-methyl-3-pyridazinyl)amino]methyl}-3-(3-pyridazinyl)-1-oxa-3-azaspiro[4.5]decan-2-one (1:1)

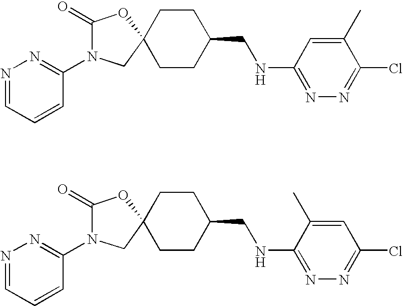

(Trans)-8-(aminomethyl)-3-(3-pyridazinyl)-1-oxa-3-azaspiro[4.5]decan-2-one (Intermediate 41 procedure 41c, 300 mg, 1.144 mmol), 3,6-dichloro-4-methylpyridazine (186 mg, 1.144 mmol), DIPEA (0.999 ml, 5.72 mmol) were collected in a closed vial and suspended in sulfolane (4 ml). The resulting yellow solution was stirred at 150° C. overnight then DIPEA was removed under vacuum and the resulting oil was passed through a SCX cartridge (10 g), washing with MeOH and eluting with 2M ammonia in MeOH. The resulting crude (500 mg) was purified by RP-flash chromatography with Biotage SP1 over a SNAP C18 column, using water and ACN (made up with 0.1% HCOOH) as eluent. Fractions containing the required product were partially evaporated, and then passed through a 5g Varian SCX cartridge, washing with MeOH and eluting with 2M ammonia in MeOH. The title compound was recovered as a yellow oil (150 mg) and as a mixture ca 1/1 of two different regioisomers.

¹H NMR (400 MHz, CDCl₃): δ 8.97 (dd, 2H), 8.58 (dq, 2H), 7.50 (ddd, 2H), 7.06 (d, 1H), 6.55 (d, 1H), 4.85 (br s, 1H), 4.46 (br s, 1H), 4.22 (s, 4H), 3.53 (t, 2H), 3.35 (t, 2H), 2.32 (d, 3H), 2.16 (d, 3H), 2.09-1.04 (m, 16H); UPLC-MS: 0.51 and 0.53 min, 389 [M+H]+.

### Intermediate 74

### 6-Chloro-2-(trifluoromethyl)imidazo[1,2-b]pyridazine

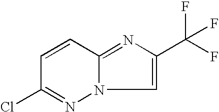

3-chloro-1,1,1-trifluoro-2-propanone (0.5 g, 3.41 mmol) and 6-chloro-3-pyridazinamine (0.442 g, 3.41 mmol) were dissolved in ethanol (10 ml) in a closed vial and the reaction mixture was stirred at 100° C. overnight. Solvent was removed under vacuum. The crude was taken up with DCM (300 ml) and washed with saturated NaHCO₃ aq solution (100 ml) and water (2×100 ml), dried over Na₂SO₄, filtered and concentrated to give 350 mg of yellow solid. The residue was purified by silica gel chromatography (Biotage SP1, 40M silica column) eluting with cyclohexane and ethyl acetate. The title compound was eluted with ca 50% EtOAc and recovered as a brown solid (220 mg).

¹H NMR (400 MHz, CDCl₃): δ 8.22 (br s, 1H), 8.00 (dd, 1H), 7.22 (d, 1H); UPLC-MS: 0.66 min, 222 [M+H]+.

### Intermediate 75

### 6-Chloro-2-(2-pyridinyl)imidazo[1,2-b]pyridazine

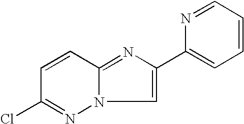

2-Bromo-1-(2-pyridinyl)ethanone hydrobromide (1.084 g, 3.86 mmol) and 6-chloro-3-pyridazinamine (0.5 g, 3.86 mmol) were collected in a closed vial and suspended in ethanol (10 ml). The resulting mixture was stirred at 120° C. (ext temp.) for 6 hours. Solvent was removed under vacuum, and the crude was taken up with DCM (300 ml), washing with a saturated NaHCO₃ aq solution (2×100 ml) and filtering over a separation tube. The resulting organic phase was concentrated and the residue was purified by silica gel chromatography (Biotage SP1, silica 25M Biotage cartridge), eluting with a gradient of cyclohexane and ethyl acetate. The title compound was eluted with ca 70% EtOAc and recovered as a yellowish solid (320 mg, 1.39 mmol, 36%).

¹H NMR (400 MHz, CDCl₃): δ 7.10 (d, 1H), 7.26-7.33 (m, 1H), 7.83 (td, 1H) 7.95 (d, 1H), 8.17-8.22 (m, 1H), 8.59 (s, 1H), 8.66-8.72 (m, 1H); UPLC-MS: 0.65 min, 231 [M+H]+.

### Intermediate 76

### 6-Chloro-2-(3-fluorophenyl)imidazo[1,2-b]pyridazine

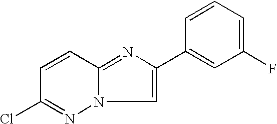

The title compound was made in a similar fashion to the Intermediate 75 replacing 2-bromo-1-(2-pyridinyl)ethanone hydrobromide with 2-bromo-1-(3-fluorophenyl)ethanone (0.838 g, 3.86 mmol) and purifying the crude by KP-NH chromatography to afford the title compound as a brown solid (700 mg, 2.54 mmol, 66%).

¹H NMR (400 MHz, CDCl₃): δ 7.04-7.14 (m, 2H), 7.40-7.50 (m, 1H), 7.66-7.77 (m, 2H), 7.93 (d, 1H), 8.23 (s, 1H); UPLC-MS: 0.74 min, 248 [M+H]+.

### Intermediate 77

### 2-Bromo-1-(2-fluorophenyl)ethanone

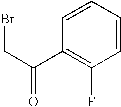

1-(2-fluorophenyl)ethanone (1 g, 7.24 mmol) was dissolved in Et₂O (30 ml). Bromine (0.373 ml, 7.24 mmol) was added portion wise, and the resulting red solution was stirred at r.t. for 2 hours. The reaction mixture was taken up with Et₂O (200 ml), washed with a saturated NaHCO₃ aq solution (3×100 ml), dried over Na₂SO₄, filtered and concentrated to give a crude which was used in the next step without any further purification (1.8 g).

¹H NMR (400 MHz, CDCl₃): δ 4.56 (d, 2H), 7.16-7.24 (m, 1H), 7.28-7.33 (m, 1H), 7.57-7.65 (m, 1H), 7.93-8.00 (m, 1H).

### Intermediate 78

### 6-Chloro-2-(2-fluorophenyl)imidazo[1,2-b]pyridazine

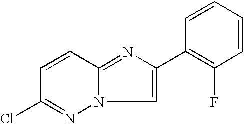

The title compound was made in a similar fashion to the preparation of Intermediate 76 replacing 2-bromo-1-(3-fluorophenyl)ethanone with 2-bromo-1-(2-fluorophenyl)ethanone (Intermediate 77, 1 g, 4.61 mmol) to afford the title compound as a yellow solid (700 mg, 61%).

¹H NMR (400 MHz, CDCl₃): δ 7.09 (d, 1H), 7.16-7.24 (m, 1H), 7.27-7.33 (m, 1H), 7.33-7.41 (m, 1H), 7.93 (d, 1H), 8.32 (td, 1H), 8.41 (d, 1H); UPLC-MS: 0.76 min, 248 [M+H]+.

### Intermediate 79

### 2-Bromo-1-(3,5-difluorophenyl)ethanone

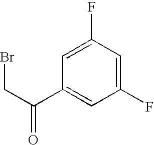

The title compound was made in a similar fashion to the preparation of Intermediate 77 replacing 1-(2-fluorophenyl)ethanone with 1-(3,5-difluorophenyl)ethanone (1 g, 6.40 mmol) to give the title compound as a colourless oil (1.6 g).

¹H NMR (400 MHz, CDCl₃): δ 4.40 (s, 2H), 7.06-7.13 (m, 1H), 7.50-7.54 (m, 2H).

### Intermediate 80

### 6-Chloro-2-(3,5-difluorophenyl)imidazo[1,2-b]pyridazine

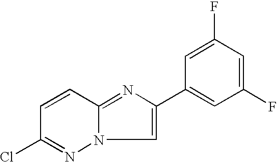

2-Bromo-1-(3,5-difluorophenyl)ethanone (1 g, 4.25 mmol) and 6-chloro-3-pyridazinamine (0.551 g, 4.25 mmol) were collected in a closed vial and suspended in ethanol (10 ml). The mixture was stirred at 100° C. overnight. Solvent was then removed under vacuum, and the crude was taken up with DCM (300 ml), washed with a saturated NaHCO₃ solution (3×100 ml), dried over Na₂SO₄, filtered and concentrated. The resulting crude was purified by flash chromatography with Biotage SP1, over a 40M column, using a gradient of cyclohexane and DCM to afford the title compound as a colourless solid (0.6 g).

¹H NMR (400 MHz, CDCl₃): δ 6.79-6.89 (m, 1H), 7.12 (d, 1H), 7.45-7.54 (m, 2H), 7.93 (d, 1H), 8.22 (s, 1H); UPLC-MS: 0.77 min, 266 [M+H]+.

### Intermediate 81

### Methyl 6-amino-3-pyridinecarboxylate

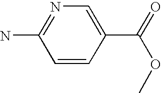

6-Aminonicotinic acid (1 g, 7.24 mmol) was dissolved in methanol (10 ml) and cooled to 0° C. in an ice-water bath. Thionyl chloride (1.74 ml, 23.9 mmol) was added to the solution and the reaction mixture was refluxed for 5 hours. The mixture was cooled to r.t., concentrated in vacuo, diluted with EtOAc, washed with a saturated NaHCO₃ solution and extracted with EtOAc (150 ml). The separated organic phase was dried (Na₂SO₄), filtered and concentrated in vacuo to give the title compound (890 mg, 5.85 mmol, 81%) which was used in the next step without any further purification.

¹H NMR (400 MHz, CDCl₃): δ 8.75 (s, 1H), 8.02 (d, 1H), 6.5 (d, 1H), 4.9 (br s, 2H), 3.88 (s, 3H); UPLC-MS: 0.27 min, 153 [M+H]+.

### Intermediate 82

### (Trans)-8-({[5-(2-pyrimidinyl)-2-pyridinyl]amino}methyl)-1-oxa-3-azaspiro[4.5]decan-2-one

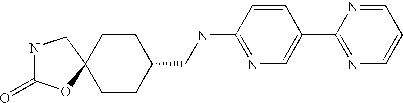

(Trans)-2-oxo-1-oxa-3-azaspiro[4.5]decane-8-carbaldehyde (prepared in a similar fashion to the Intermediate 5 procedure 5b, 433 mg, 2.363 mmol) and 5-(2-pyrimidinyl)-2-pyridinamine (prepared in a similar fashion to the Intermediate 26 but purifying the crude by silica gel chromatography with 0%-10% MeOHDCM, 407 mg, 2.363 mmol) were dissolved in anhydrous DCM (8 ml). Titanium(IV) isopropoxide (1.385 ml, 4.73 mmol) was added and the reaction mixture was stirred at r.t. overnight. Sodium borohydride (268 mg, 7.09 mmol) and ethanol (2 ml) were added and the resulting mixture was stirred, then it was poured into a saturated NaHCO₃ solution (5 ml) and extracted with DCM (3×50 ml) through a phase separator tube. Combined organic extracts were concentrated under vacuum and the resulting crude was purified by KP-NH chromatography (Biotage 25M column) eluting with 100% EtOAc to afford the title compound (230 mg, 0.678 mmol, 29

¹H NMR (400 MHz, CDCl₃): δ 0.83-2.23 (m, 10H), 3.08-3.58 (m, 3H), 5.51 (br s., 1H), 6.44 (br s, 1H), 6.59 (br s, 1H), 7.06 (br s, 1H), 7.55 (br s, 1H), 8.31-8.46 (m, 1H), 8.67 (br s, 1H), 9.09 (br s, 1H); UPLC-MS: 0.39 min, 340 [M+H]+.

### Intermediate 83

### 4-Bromo-1-(tetrahydro-2H-pyran-2-yl)-1H-pyrazole

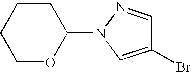

TFA (2.62 μl, 0.034 mmol) was added to a mixture of 4-bromo-1H-pyrazole (100 mg, 0.680 mmol) and 3,4-dihydro-2H-pyran (86 mg, 1.021 mmol) and the resulting mixture was shaken at 80° C. for 16 hours. The reaction mixture was cooled to r.t. then partitioned between DCM (5 ml) and 1M NaOH aq solution (2 ml). This was filtered through a hydrophobic frit (Phase Seperator cartridge) washing with dichloromethane. The combined organics were evaporated under reduced pressure. The residue was purified via Biotage (10%-30% EtOAc/cyclohexane; 25M silica column) to afford the title compound as a colourless oil (136 mg, 0.589 mmol, 86% yield).

UPLC-MS: 0.65 min, 232.9 [M+H]+.

### Intermediate 84

### (Trans)-8-({[5-(2-pyrimidinyl)-2-pyridinyl]amino}methyl)-3-[1-(tetrahydro-2H-pyran-2-yl)-1H-pyrazol-4-yl]-1-oxa-3-azaspiro[4.5]decan-2-one

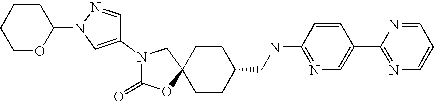

To a solution of (trans)-8-({[5-(2-pyrimidinyl)-2-pyridinyl]amino}methyl)-1-oxa-3-azaspiro[4.5]decan-2-one (Intermediate 82, 46 mg, 0.136 mmol) and 4-bromo-1-(tetrahydro-2H-pyran-2-yl)-1H-pyrazole (Intermediate 83, 39 mg, 0.169 mmol) in 1,4-dioxane (2 ml), copper(I) iodide (25.8 mg, 0.136 mmol), (+/−)-trans-1,2-diaminocyclohexane (0.033 ml, 0.271 mmol) and K₃PO₄ (144 mg, 0.678 mmol) were added and the resulting mixture was irradiated at 80° C. for 30 min in microwave system then it was stirred at 150° C. overnight. This was cooled to room temperature and the solvent evaporated. Dichloromethane (5 ml) was added to the residue and which was then filtered washing with more dichloromethane (2×1 ml). The organic phase was washed with a pH 3 citrate buffer solution (5 ml) then filtered through a hydrophobic frit (Phase Separator cartridge) and the organic solution was evaporated. The residue was purified by flash chromatography (Biotage SP1) eluting in gradient with 30%-100% EtOAc/cyclohexane to give the title compound (14.8 mg, 0.030 mmol, 22%).

¹H NMR (400 MHz, CDCl₃): δ 1.58-1.76 (m, 8H), 1.95-2.20 (m, 8H), 3.35 (t, 2H), 3.68 (s, 3H), 4.01-4.12 (m, 1H), 4.86-4.96 (m, 1H), 6.46-6.52 (m, 1H), 7.11 (t, 1H), 7.53-7.59 (m, 1H), 7.94 (dd, 1H), 8.45 (dd, 1H), 8.73 (d, 2H), 9.17-9.20 (m, 1H); UPLC-MS: 0.51 min, 490 [M+H]+.

### Intermediate 85

### 6-({[Trans-2-oxo-3-(2-pyridinyl)-1-oxa-3-azaspiro[4.5]dec-8-yl]methyl}amino)-3-pyridinecarboxylic Acid

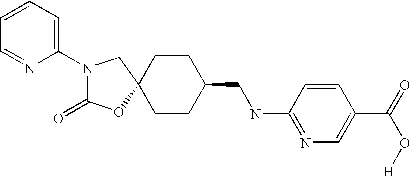

(Trans)-8-(aminomethyl)-3-(2-pyridinyl)-1-oxa-3-azaspiro[4.5]decan-2-one (prepared in a similar fashion to Intermediate 19, 235 mg, 0.899 mmol) was dissolved DMF. (3 ml). TEA (0.251 ml, 1.799 mmol) and 6-fluoro-3-pyridinecarboxylic acid (127 mg, 0.899 mmol) were added and the mixture was irradiated at 150° C. for 30 min and then it was allowed heating at 150° C. for two days. The reaction mixture was loaded onto a SCX cartridge (20g) washed with MeOH (20 ml) and eluted with 2M NH₃ in MeOH (40 ml) to give a residue. The residue was purified by silca gel chromatography (25M column) eluting in gradient with 0%-100% MeOHDCM to afford the title compound (70 mg, 0.046 mmol, 5%).

¹H NMR (400 MHz, MeOD-d₄) δ 1.20-1.37 (m, 3H), 1.81 (br s, 3H), 1.95-2.10 (m, 4H), 3.33-3.41 (m, 3H), 4.10 (s, 2H), 6.50-6.59 (m, 1H), 7.12 (ddd, 1H), 7.73-7.85 (m, 1H), 7.90-7.97 (m, 1H), 8.13-8.20 (m, 1H), 8.32-8.39 (m, 1H), 8.59-8.63 (m, 1H); UPLC-MS: 0.49 min, 383 [M+H]+.

### Intermediate 86

### 2-Fluoro-5-(1-pyrrolidinylcarbonyl)pyridine

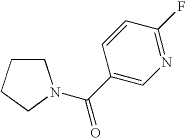

6-Fluoro-3-pyridinecarboxylic acid (150 mg, 1.063 mmol) was dissolved in acetonitrile (2 ml). HOBt (195 mg, 1.276 mmol) and EDC.HCl (245 mg, 1.276 mmol) were added and the mixture was stirred for 15 minutes. Then pyrrolidine (0.088 ml, 1.063 mmol) was added and the resulting suspension was stirred at r.t. overnight. The reaction mixture was poured into a saturated NaHCO₃ solution (5 ml) and extracted with EtOAc. Combined organic extracts were washed with a pH 3 buffer solution, dried (Na₂SO₄), filtered and concentrated in vacuo to give a residue. The residue was purified by KP-NH flash chromatography (Biotage SP1, 12M NH column) eluting in gradient with 40%-100% EtOAc\cyclohexaane to afford the title compound (176.6 mg, 0.818 mmol, 77%).

¹H NMR (400 MHz, CDCl₃): δ 1.85-2.05 (m, 4H), 3.33-3.50 (m, 2H), 3.52-3.70 (m, 2H), 6.90-7.08 (m, 1H), 7.89-8.03 (m, 1H), 8.32-8.50 (m, 1H); UPLC-MS: 0.47 min, 195 [M+H]+.

### Intermediate 87

### 3-Fluoro-2,3′-biPyridin-6′-amine

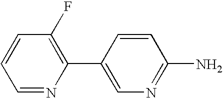

The title compound was made in a similar fashion to the Intermediate 26 replacing 2-bromopyrimidine with 2-bromo-3-fluoropyridine (600 mg, 3.41 mmol) and purifying the crude with 50%-100% EtOAc\cyclohexane to afford the title compound (580 mg, 3.07 mmol, 90%).

¹H NMR (400 MHz, CDCl₃): δ 4.50-4.87 (m, 2H), 6.57-6.65 (m, 1H), 7.15-7.24 (m, 1H), 7.44-7.51 (m, 1H), 8.11-8.21 (m, 1H), 8.44-8.52 (m, 1H), 8.69-8.80 (m, 1H); UPLC-MS: 0.34 min, 190 [M+H]+.

### Intermediate 88

### (Trans)-8-{[(3-fluoro-2,3′-bipyridin-6′-yl)amino]methyl}-oxa-3-azaspiro[4.5]decan-2-one

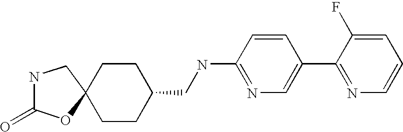

The title compound was made in a similar fashion to the preparation of Intermediate 82 replacing 5-(2-pyrimidinyl)-2-pyridinamine with 3-fluoro-2,3′-bipyridin-6′-amine (Intermediate 87, 367 mg, 1.938 mmol) and trying to purify the crude twice by KP-NH chromatography (Biotage 25M NH column; eluents: first 0%-50% MeOHEtOAc then 0%-50% Et₂ODCM) plus a washing with a pH 3 buffer solution to afford the title compound (200 mg, 0.561 mmol) which was used in the next step without any further purification.

¹H NMR (400 MHz, CDCl₃): δ 1.60-2.32 (m, 8H), 3.14-3.59 (m, 3H), 4.92 (br s, 1H), 5.31 (br s, 1H), 7.14-7.27 (m, 1H), 7.38-7.54 (m, 2H), 8.05-8.21 (m, 1H), 8.39-8.53 (m, 1H), 8.73 (d, 1H); UPLC-MS: 0.43 min, 357 [M+H]+.

### Intermediate 89

### 2-Fluoro-5-(1-piperidinylcarbonyl)pyridine

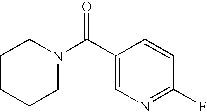

The title compound was made in a similar fashion to the Intermediate 86 replacing pyrrolidine with piperidine (0.105 ml, 1.063 mmol) to afford the title compound (215 mg, 1.033 mmol, 97%).

¹H NMR (400 MHz, CDCl₃): δ 1.38-1.79 (m, 6H), 3.19-3.47 (m, 2H), 3.68 (d, 2H) 6.90-7.02 (m, 1H), 7.79-7.91 (m, 1H), 8.18-8.34 (m, 1H); UPLC-MS: 0.53 min, 209 [M+H]+.

### Intermediate 90

### 6′-Fluoro-6-(trifluoromethyl)-2,3′-bipyridine

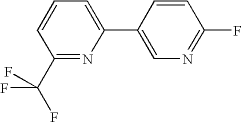

The title compound was made in a similar fashion to the preparation of Intermediate 66 replacing 2-bromopyrimidine with 2-bromo-6-(trifluoromethyl)pyridine (203 mg, 0.897 mmol) to afford the title compound (200 mg, 0.897 mmol).

¹H NMR (400 MHz, CDCl₃): δ 7.07-7.12 (m, 1H), 7.70 (dd, 1H), 7.91-7.96 (m, 1H), 7.98-8.05 (m, 1H), 8.58 (ddd, 1H), 8.86 (m, 1H); UPLC-MS: 0.74 min, 243 [M+H]+.

### Intermediate 91

### 2-Fluoro-5-[2-(trifluoromethyl)phenyl]pyridine

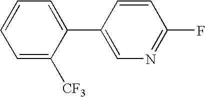

The title compound was made in a similar fashion to the preparation of Intermediate 66 replacing 2-bromopyrimidine with 1-bromo-2-(trifluoromethyl)benzene (0.122 ml, 0.897 mmol) to afford the title compound (118 mg, 0.489 mmol, 55%).

¹H NMR (400 MHz, CDCl₃): δ 6.97-7.05 (m, 1H), 7.32-7.38 (m, 1H), 7.52-7.58 (m, 1H), 7.60-7.66 (m, 1H), 7.74-7.84 (m, 2H), 8.19 (dd, 1H); UPLC-MS: 0.76 min, 242 [M+H]+.

### Intermediate 92

### 5-(2-Pyridinyloxy)-2-pyridinamine

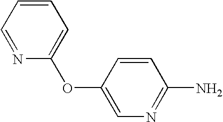

To a solution of 6-amino-3-pyridinol (300 mg, 2.72 mmol) in sulfolane (2 ml) TEA (0.759 ml, 5.45 mmol) and 2-fluoropyridine (0.236 ml, 2.72 mmol) were added and the resulting mixture was irradiated at 200° C. for 30 minutes (3 cycles). Then it was loaded onto SCX cartridge (20g), washed with MeOH (20 ml) and eluted with 2M NH₃ in MeOH to give a residue. The residue was purified on Biotage 25M KP-NH column eluting in gradient with 50%-100% EtOAc\cyclohexane to afford the title compound (75 mg, 0.401 mmol, 15%).

¹H NMR (400 MHz, CDCl₃): δ 4.32-4.69 (m, 2H), 6.52-6.59 (m, 1H), 6.88-6.96 (m, 1H), 6.97-7.04 (m, 1H), 7.23-7.33 (m, 1H), 7.64-7.73 (m, 1H), 7.94-8.01 (m, 1H), 8.12-8.22 (m, 1H); UPLC-MS: 0.36 min, 188 [M+H]+.

### Intermediate 93

### 4-Phenyl-2-pyridinamine

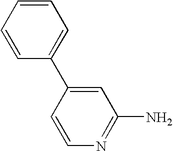

Phenylboronic acid (141 mg, 1.156 mmol), 4-bromo-2-pyridinamine (200 mg, 1.156 mmol) and PdCl₂(dppf)CH₂Cl₂ adduct (94 mg, 0.116 mmol) were collected and suspended in 1,2-dimethoxyethane (3 ml) and 1M aq sodium carbonate solution (3.47 ml, 3.47 mmol). The resulting mixture was stirred at 90° C. for 5 hours then it was cooled to r.t. and filtered over a celite pad washing with DCM. The solvent was evaporated (in vacuo) and the crude was purified by silica gel chromatography (Biotage 40M column) and eluting in gradient with 0%-10% MeOHDCM to afford the title compound (195 mg, 1.146 mmol, 99%).

¹H NMR (400 MHz, CDCl₃): δ 4.49 (dd, 2H), 6.69-6.78 (m, 1H), 6.90 (dd, 1H), 7.39-7.52 (m, 3H), 7.56-7.68 (m, 2H), 8.10-8.17 (m, 1H); UPLC-MS: 0.42 min, 171 [M+H]+.

### Intermediate 94

### (Trans)-8-({[6-(4-morpholinyl)-3-pyridinyl]amino}methyl)-1-oxa-3-azaspiro[4.5]decan-2-one

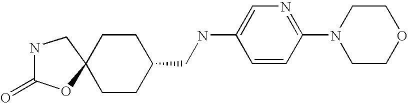

The title compound was made in a similar fashion to the preparation of Intermediate 82 replacing 5-(2-pyrimidinyl)-2-pyridinamine with 6-(4-morpholinyl)-3-pyridinamine (44.0 mg, 0.246 mmol) to afford the title compound (49.8 mg, 0.144 mmol, 58%).

¹H NMR (400 MHz, CDCl₃): δ 1.03-1.16 (m, 1H), 1.23-1.30 (m, 7H), 1.62-1.73 (m, 1H), 1.76-1.87 (m, 1H), 1.90-2.01 (m, 3H), 2.93-3.05 (m, 1H), 3.28-3.47 (m, 4H), 3.81-3.88 (m, 3H), 4.11-4.19 (m, 2H), 6.58-6.64 (m, 1H), 6.93 (dd, 1H), 7.68-7.75 (m, 1H); UPLC-MS: 0.40 min, 347 [M+H]+.

### Intermediate 95

### 6-(2-Fluorophenyl)-3-pyridinamine

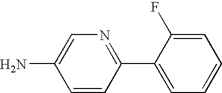

The title compound was made in a similar fashion to the preparation of Intermediate 93 replacing 4-bromo-2-pyridinamine with 6-bromo-3-pyridinamine (500 mg, 2.89 mmol) and phenylboronic acid with (2-fluorophenyl)boronic acid (404 mg, 2.89 mmol) to afford the title compound (430 mg, 2.28 mmol, 79%).

¹H NMR (400 MHz, CDCl₃): δ 3.75-3.95 (m, 2H), 7.00-7.08 (m, 1H), 7.11-7.18 (m, 1H), 7.19-7.25 (m, 1H), 7.26-7.34 (m, 1H), 7.58-7.64 (m, 1H), 7.85-7.99 (m, 1H), 8.19-8.24 (m, 1H); UPLC-MS: 0.38 min, 189 [M+H]+.

### Intermediate 96

### (Trans)-8-({[6-(2-fluorophenyl)-3-pyridinyl]amino}methyl)-1-oxa-3-azaspiro[4.5]decan-2-one

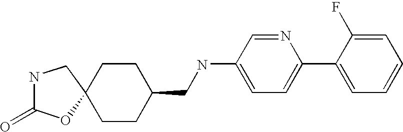

The title compound was made in a similar fashion to the preparation of Intermediate 82 replacing 5-(2-pyrimidinyl)-2-pyridinamine with 6-(2-fluorophenyl)-3-pyridinamine (205 mg, 1.092 mmol) to afford (trans)-8-({[6-(2-fluorophenyl)-3-pyridinyl]amino}methyl)-1-oxa-3-azaspiro[4.5]decan-2-one (287.5 mg, 0.809 mmol, 74%).

¹H NMR (400 MHz, CDCl₃): δ 1.04-1.20 (m, 2H), 1.69-1.84 (m, 4H), 1.95-2.03 (m, 3H), 3.09 (t, 2H), 3.37-3.44 (m, 2H), 3.92 (br s, 1H), 5.55 (s, 1H), 6.94 (dd, 1H), 7.10-7.19 (m, 1H), 7.20-7.37 (m, 2H), 7.60-7.69 (m, 1H), 7.88-7.98 (m, 1H), 8.10-8.19 (m, 1H); UPLC-MS: 0.50 min, 356 [M+H]+.

### Intermediate 97

### 6-(Cyclohexyloxy)-3-pyridazinamine

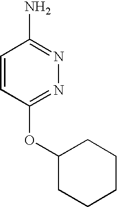

Freshly cut sodium (200 mg, 8.70 mmol) was added to cyclohexanol (12 g, 120 mmol) in a screw-topped pressure tube at room temperature. Evolution of hydrogen was very slow and the surfaces of the sodium quickly became opaque. This was stirred and heated overnight at 100° C. The sodium continued to react very slowly over the course of several hours [NB if the reaction was allowed to cool the mixture became a gel presumably due to low solubility of the sodium alkoxide in cyclohexanol]. Heating was continued at 140° C. until the solid sodium became molten—at this point the evolution of hydrogen was visibly faster and the last remaining sodium reacted. 6-chloro-3-pyridazinamine (763 mg, 5.89 mmol) was added and then the mixture was heated to 140° C. Upon heating the mixture began to turn yellow then when everything dissolved it rapidly became dark brown, the reaction was left at 140° C. overnight. It was added a solution of NH₄Cl in MeOH and then concentrated under vacuum before to apply on SCX however some material of the desired molecular weight eluted with MeOH washings whereas some stuck to the resin and was only eluted with NH₃ in MeOH. All product containing fractions were recombined and evaporated. The dark brown residue was purified via Biotage (5%-10% EtOAc/CH₂Cl₂; 40+M NH column) to give 6-(cyclohexyloxy)-3-pyridazinamine (1.38 g, 7.14 mmol, 82%).

¹H NMR (400 MHz, CDCl₃): δ 1.25-1.31 (m, 2H), 1.36-1.68 (m, 3H), 1.70-1.81 (m, 2H), 1.85-1.96 (m, 2H), 2.03-2.13 (m, 1H), 3.57-3.69 (m, 1H), 4.34-4.47 (m, 1H), 5.09-5.17 (m, 1H), 6.73-6.81 (m, 1H), 7.28 (s, 1H); UPLC-MS: 0.49 min, 194 [M+H]+.

### Intermediate 98

### 1-(6-Amino-3-pyridazinyl)-2(1H)-pyridinone

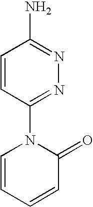

A microwave vial was charged with copper(I) iodide (10.01 mg, 0.053 mmol) and potassium carbonate (291 mg, 2.103 mmol), evacuated and backfilled with nitrogen. 2(1H)-pyridinone (100 mg, 1.052 mmol), 6-bromo-3-pyridazinamine (183 mg, 1.052 mmol) and N,N-Dimethylformamide (4 ml) was added under nitrogen. The vial was sealed and the reaction mixture was stirred at 130° C. for 3 hours. The solvent was evaporated. Dichloromethane (5 ml) was added to the residue and it was filtered washing with more dichloromethane (2×1 ml). The residue was purified by silica gel chromatography via Biotage (10%-50% DCMMeOH) to give 1-(6-amino-3-pyridazinyl)-2(1H)-pyridinone (51 mg, 0.271 mmol, 26%).

¹H NMR (400 MHz, CDCl₃): δ 4.93 (br s, 1H), 5.05 (br s, 1H), 5.46 (br s, 1H), 6.04 (br s, 2H), 6.22 (brs, 1H); UPLC-MS: 0.36 min, 189 [M+H]+.

### Intermediate 99

### 2,3′-Bipyridin-6′-amine

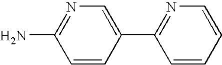

To a mixture of 2-bromopyridine (86 mg, 0.545 mmol) and 5-(4,4,5,5-tetramethyl-1,3,2-dioxaborolan-2-yl)-2-pyridinamine (0.100 g, 0.454 mmol) in dry DMF (2 ml), PdCl₂(dppf)-CH₂Cl₂ adduct (30 mg, 0.036 mmol) and aqueous 2.0M sodium carbonate (0.192 g, 1.816 mmol) (1 ml) were added and the resulting mixture was shaken at 80° C. overnight. The mixture was diluted with DCM, filtered through filter tube and concentrated under vacuum to give a brown residue which was dissolved in MeOH, loaded into SCX cartridge (2g) and eluted with 2.0M NH₃ in MeOH to give a crude. The crude was purified by flash chromatography on KP-NH 12+M column, by isocratic elution with 50% EtOAc\cyclohexane to give the title compound as a white solid (34 mg).

UPLC\MS: 0.34 min, 172 [M+H]+.

### Intermediate 100

### 5-Methyl-2,3′-bipyridin-6′-amine

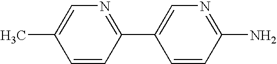

The title compound was made in a similar fashion to the preparation of Intermediate 99 replacing 2-bromopyridine with 2-bromo-5-methylpyridine (94 mg, 0.545 mmol) to give the title compound as a white solid (41 mg).

UPLC\MS: 0.37 min, 186 [M+H]+.

### Intermediate 101

### 6-Fluoro-2,3′-bipyridin-6′-amine

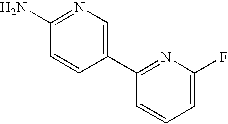

The title compound was made in a similar fashion to the preparation of Intermediate 99 replacing 2-bromopyridine with 2-bromo-6-fluoropyridine (96 mg, 0.545 mmol) to give the title compound as a white solid (48 mg).

UPLC\MS: 0.38 min, 190 [M+H]+.

### Intermediate 102

### 3-Fluoro-6-methyl-2,3′-bipyridin-6′-amine

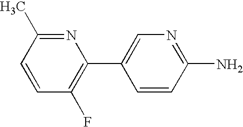

The title compound was made in a similar fashion to the preparation of Intermediate 99 replacing 2-bromopyridine with 2-bromo-3-fluoro-6-methylpyridine (104 mg, 0.545 mmol) to give the title compound as a white solid (35 mg).

UPLC\MS: 0.40 min, 204 [M+H]+.

### Intermediate 103

### 5-Fluoro-2,3′-bipyridin-6′-amine

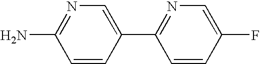

The title compound was made in a similar fashion to the preparation of Intermediate 99 replacing 2-bromopyridine with 2-bromo-5-fluoropyridine (96 mg, 0.545 mmol) to give the title compound as a white solid (34 mg).

UPLC\MS: 0.37 min, 190 [M+H]+.

### Intermediate 104

### 5-{3-[(Trifluoromethyl)oxy]phenyl}-2-pyridinamine

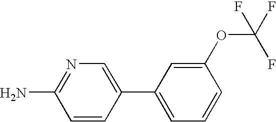

To a solution of 5-(4,4,5,5-tetramethyl-1,3,2-dioxaborolan-2-yl)-2-pyridinamine (100 mg, 0.454 mmol) and 1-bromo-3-[(trifluoromethyl)oxy]benzene (131 mg, 0.545 mmol) in dry DMF\EtOH\H₂O (2:1:1) tetrakis(triphenylphosphine)palladium(0) (42.0 mg, 0.036 mmol) and potassium acetate (89 mg, 0.909 mmol) were added and the mixture was shaken at 100° C. overnight. The mixture was diluted with DCM, filtered through filter tube and concentrated in vacuo to give a brown residue which was dissolved in methanol and loaded into SCX cartridge (2g), washed with MeOH and eluted with 2.0M ammonia in MeOH. The resulting ammonia\methanolic solution was concentrated in vacuo to give the title compound (87 mg, 0.329 mmol, 72%).

UPLC\MS: 0.55 min, 255 [M+H]+.

### Intermediate 105

### 5-(2-Fluorophenyl)-2-pyridinamine

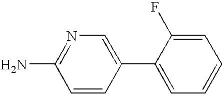

The title compound was made in a similar fashion to the preparation of Intermediate 104 replacing 1-bromo-3-[(trifluoromethyl)oxy]benzene with 2-bromofluorobenzene (95 mg, 0.545 mmol) to give the title compound (62 mg, 0.316 mmol, 70%).

UPLC\MS: 0.43 min, 189 [M+H]+.

### Intermediate 106

### 3,3′-Bipyridin-6-amine

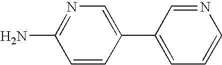

To a mixture of 5-(4,4,5,5-tetramethyl-1,3,2-dioxaborolan-2-yl)-2-pyridinamine (100 mg, 0.454 mmol), 3-bromopyridine (53.8 μl, 0.545 mmol) and potassium carbonate (126 mg, 0.909 mmol) in DMF (0.600 ml) and H2O (0.200 ml) tetrakis(triphenylphosphine)palladium(0) was added (26.3 mg, 0.023 mmol) and the resulting suspension was irradiated in Initiator microwave system at 150° C. for 10 min. Then the mixture was diluted with EtOAc, filtered through IST filter tube and concentrated in vacuo to give the title compound as a yellowish solid without any further purification (77 mg, 0.450 mmol).

UPLC\MS: 0.20 min, 172 [M+H]+.

### Intermediate 107

### 6′-Fluoro-3,3′-bipyridin-6-amine

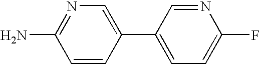

The title compound was made in a similar fashion to the preparation of Intermediate 106 replacing 3-bromopyridine with 5-bromo-2-fluoropyridine (96 mg, 0.545 mmol) to give the title compound as a yellowish solid (83 mg, 0.333 mmol).

UPLC\MS: 0.34 min, 190 [M+H]+.

### Intermediate 108

### 2′-Fluoro-3,3′-bipyridin-6-amine

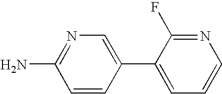

The title compound was made in a similar fashion to the preparation of Intermediate 106 replacing 3-bromopyridine with 3-bromo-2-fluoropyridine (96 mg, 0.545 mmol) to give the title compound as yellowish solid (61.5 mg, 0.293 mmol).

UPLC\MS: 0.34 min, 190 [M+H]+.

### Intermediate 109

### 3-Fluoro-2,3′-bipyridin-6′-amine

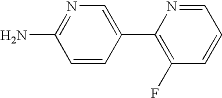

PdCl₂(dppf)-CH₂Cl₂ adduct (29.7 mg, 0.036 mmol) and aqueous 2.0M sodium carbonate (0.909 ml, 1.818 mmol) were added to a mixture of 2-bromo-3-fluoropyridine (96 mg, 0.545 mmol) and 5-(4,4,5,5-tetramethyl-1,3,2-dioxaborolan-2-yl)-2-pyridinamine (100 mg, 0.454 mmol) in dry DMF (3 ml) and the resulting mixture was shaken at 80° C. overnight. The mixture was diluted with DCM, filtered through filter tube and concentrated under vigorous nitrogen flow (Radleys blowdown system, at 45° C.) to obtain a residue which was taken into DCM and washed with water. After separation, the organic phase was concentrated under vacuum to give a brown residue. The crude was purified by flash chromatography on KP-NH 12+M column eluting in gradient with 20%-80% EtOAc\cyclohexane in 10 cv to give the title compound as yellow solid (56 mg, 0.263 mmol, 58%).

¹H NMR (400 MHz, DMSO-d₆) δ 6.36 (br s, 2H), 6.55 (d, 1H), 7.28-7.38 (m, 1H), 7.68-7.79 (m, 1H), 7.90-8.01 (m, 1H), 8.41-8.48 (m, 1H), 8.49-8.56 (m, 1H); UPLC\MS: 0.35 min, 190 [M+H]+.

### Intermediate 110

### 3-(6-Fluoro-3-pyridinyl)benzonitrile

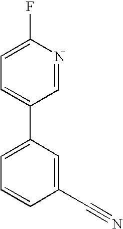

De-aerated acetonitrile/water (3:1, 12 ml) was added to a mixture of 5-bromo-2-fluoropyridine (0.5 g, 2.84 mmol), (3-cyanophenyl)boronic acid (0.626 g, 4.26 mmol), potassium carbonate (0.785 g, 5.68 mmol) and bis(triphenylphosphine)palladium(II) chloride (0.100 g, 0.142 mmol) at room temperature in a 20 ml microwave vial. The vial was capped and heated with shaking to 100° C. in a PLS reaction station for 2 hours. This was allowed to cool to room temperature—a large amount of product crystalised from the acetonitrile layer. The reaction mixture was filtered, washing the solids with diethyl ether (20 ml). The solids were taken up in dichloromethane (40 ml), filtered through a hydrophobic frit (Phase Separator cartridge) and evaporated under reduced pressure to give a dark grey solid. This was passed through a plug of SiO2 eluting with dichloromethane (this removed much of the colour but some black colour passed through the SiO2) and evaporated to give a grey solid. This was dissolved in dichloromethane (30 ml) and treated with Fluorosil (1g) which immobilised the remaining coloured impurities. This was filtered and evaporated under reduced pressure to give a white solid which was then recrystallised from acetonitrile to give the title compound (297 mg) as white crystals.

¹H NMR (400 MHz, CDCl₃): δ 8.44 (d, 1H), 7.99 (td, 1H), 7.85 (s, 1H), 7.80 (d, 1H), 7.73 (d, 1H), 7.63 (t, 1H), 7.10 (dd, 1H); UPLC-MS: 0.66 min, 199 [M+H]+.

### Intermediate 111

### [6-({[(Trans)-2-oxo-3-(3-pyridazinyl)-1-oxa-3-azaspiro[4.5]dec-8-yl]methyl}amino)-3-pyridinyl]boronic Acid

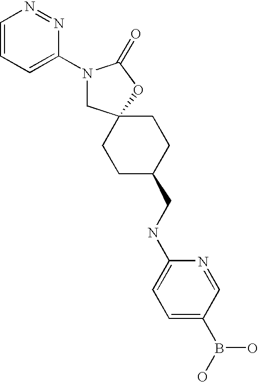

In a round bottom flask, to a suspension of (trans)-2-oxo-3-(3-pyridazinyl)-1-oxa-3-azaspiro[4.5]decane-8-carbaldehyde (prepared in a similar fashion to Intermediate 60 but purifying the crude by silica gel chromatography on SNAP 50 g column eluting in gradient with 60%-100% EtOAc\cyclohexane in 10 cv then 100% EtOAc\cyclohexane 3 cv, 1.31 g, 5.01 mmol) and 5-(4,4,5,5-tetramethyl-1,3,2-dioxaborolan-2-yl)-2-pyridinamine (1.25 g, 5.68 mmol) in dry THF (50 ml) was added acetic acid (0.574 ml, 10.03 mmol) and the mixture was stirred at room temperature 3 hours. Dichloromethane (5.0 ml) was added to get starting material into solution completely. The resulting solution was stirred at room temperature overnight then further acetic acid (0.290 ml, 5 mmol) was added followed by addition of sodium cyanoborohydride (1.11 g, 17.66 mmol) and stirring at room temperature for 1.5 hours. The mixture was cooled to 0° C., diluted with DCM and quenched with a saturated NaHCO₃ solution (50 ml). The organic phase was separated and the aq one was back-extracted with DCM (2×30 ml). Combined organic extracts were dried (Na₂SO₄), filtered and concentrated in vacuo to give a crude as a yellowish solid. Purification by reverse phase chromatography (SNAP KP-C18 120 g column, SP1 Biotage system), eluting in gradient with 100% phase A (H₂O+5% acetonitrile+0.1% HCO₂H) (2 cv), 0%-50% phase B (acetonitrile)\phase A (in 10 cv), then 50% phase B (3 cv), 50%-95% phase B (in 6 cv), 95% phase B (3 cv) and 95%-0% phase B (in 2 cv). Fractions containing target product (by mass) were collected and concentrated in vacuo to give the title compound as a yellowish solid (454 mg).

UPLC\MS: 0.50 min, 384 [M+H]+

### Intermediate 112

### 6-[(2-MethylPhenyl)oxy]-3-pyridazinamine

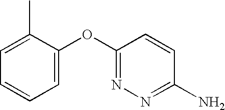

In a 5 ml microwave vial 2.0M aq sodium hydroxide (1.158 ml, 2.316 mmol) was added to a mixture of 6-chloro-3-pyridazinamine (100 mg, 0.772 mmol) and 2-methylphenol (209 mg, 1.93 mmol) in dry acetonitrile (2.5 ml) and the reaction mixture was irradiated at 180° C. for 30 minutes then at 170° C. for 1 hour. The suspension was concentrated under nitrogen flow and the resulting residue was taken into DCM and water. The organic phase was separated through IST phase separator tube and concentrated under vacuum to obtain a brown residue. The residue was dissolved in MeOH, loaded onto SCX cartridge (2g) washed with methanol and eluted with 2.0M NH₃ in MeOH to give, after concentration of ammonia\methanolic solution, a crude. The crude was purified by reverse phase flash chromatography on SP1 Biotage system (KP-C18 HS10 g column) eluting in gradient with 0% phase B (acetonitrile+0.1% HCO₂H) (1 cv), 0%-90% phase B\phase A (H₂O+0.5% acetonitrile 0.1% HCO₂H) (in 12 cv), 90% phase B\phase A (3 cv), 90%-0% phase B\phase A (in 2 cv) to yield the title compound as a white solid (8 mg, 0.040 mmol).

UPLC\MS: 0.46 min, 202 [M+H]+

### Intermediate 113

### 6-[(3-Fluorophenyl)oxy]-3-pyridazinamine

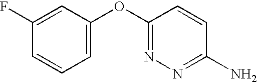

The title compound was made in a similar fashion to the preparation of Intermediate 112 replacing 2-methylphenol with 3-fluorophenol (216 mg, 1.93 mmol) to afford the title compound as a white solid (9.5 mg, 0.046 mmol).

UPLC\MS: 0.44 min, 206 [M+H]+.

### Intermediate 114

### 6-{[2-(Trifluoromethyl)phenyl]oxy}-3-pyridazinamine

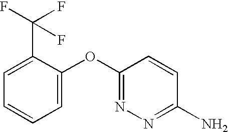

The title compound was made in a similar fashion to the preparation of Intermediate 112 replacing 2-methylphenol with 2-(trifluoromethyl)phenol (313 mg, 1.93 mmol) and irradiating the mixture at 180° C. for 30 minutes then at 200° C. for 40 min to yield the title compound as a white solid (10 mg, 0.039 mmol).

UPLC\MS: 0.50 min, 256 [M+H]+.

### Intermediate 115

### 6-[(4-Fluorophenyl)oxy]-3-pyridazinamine

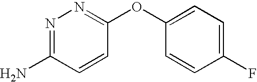

The title compound was made in a similar fashion to the preparation of Intermediate 112 replacing 2-methylphenol with 4-fluorophenol (216 mg, 1.93 mmol) and irradiating the mixture once at 180° C. for 30 minutes to yield the title compound as a white solid (8.1 mg, 0.040 mmol, 5%).

UPLC\MS: 0.43 min, 206 [M+H]+.

### Intermediate 116

### 6-[(2,4-Dimethylphenyl)oxy]-3-pyridazinamine

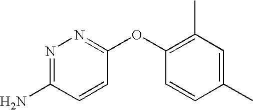

The title compound was made in a similar fashion to the preparation of Intermediate 114 replacing 2-(trifluoromethyl)phenol with 2,4-dimethylphenol (236 mg, 1.93 mmol) to afford the title compound as a white solid (11 mg, 0.051 mmol, 7%).

UPLC-MS: 0.51 min, 216 [M+H]+.

### Intermediate 117

### 6-({2-[(Trifluoromethyl)oxy]phenyl}oxy)-3-pyridazinamine

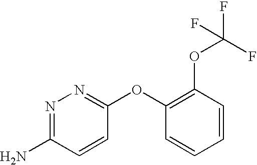

The title compound was made in a similar fashion to the preparation of Intermediate 115 to afford the title compound as a white solid (9.1 mg, 0.034 mmol, 4%).

UPLC-MS: 0.51 min, 272 [M+H]+.

### Examples

### Example 1

### Preparation of Compounds of Formula (IIA)

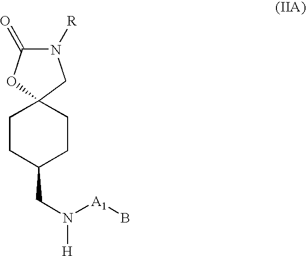

### Example 1-1

### (Trans)-3-phenyl-8-({[5-(trifluoromethyl)-2-pyridinyl]amino}methyl)-1-oxa-3-azaspiro[4.5]decan-2-one

(Trans)-2-oxo-3-phenyl-1-oxa-3-azaspiro[4.5]decane-8-carbaldehyde (Intermediate 9, 30 mg, 0.116 mmol) and (trifluoromethyl)-2-pyridinamine (20.63 mg, 0.127 mmol) were combined and dissolved in dichloromethane (2 ml). The solution was stirred at r.t. for 3.5 hours then cyanoborohydride on polymer support (99 mg, 0.347 mmol) and acetic acid (0.066 ml, 1.157 mmol) were added and the mixture was stirred at r.t. for 3 hours then left standing at r.t. over the week-end (ca 60 hours). Then the resin was filtered off washing with DCM. The DCM layer was washed with NaHCO₃ saturated solution and the organic was dried to give a crude that was purified by column chromatography (Silica 12+M, on Biotage SP1) eluting with cyclohexane/EtOAc from 100:0 to 50:50 to afford 18 mg that were further purified by preparative MDAP. The product fraction was dried under vacuo then treated with sat. NaHCO₃ solution and extracted with DCM (3×1 ml). DCM extracts were combined and dried to afford a solid (UPLC-MS: 0.79 min, 406 [M+H]+) that was dissolved again in DCM and dried to afford the title compound as a white solid (2.2 mg, 4.6%);

### Example 1-2

### (Trans)-3-(2-pyridinyl)-8-({[5-(trifluoromethyl)-2-pyridinyl]amino}methyl)-1-oxa-3-azaspiro[4.5]decan-2-one

(Trans)-2-oxo-3-(2-pyridinyl)-1-oxa-3-azaspiro[4.5]decane-8-carbaldehyde (prepared in a similar fashion to Intermediate 12 procedure 12a, 98.5 mg, 0.378 mmol) and 5-(trifluoromethyl)-2-pyridinamine (90 mg, 0.555 mmol) were dissolved in tetrahydrofuran (3 ml) at r.t and titanium(IV) isopropoxide (0.222 ml, 0.757 mmol) was added. The reaction was stirred overnight then sodium borohydride (43.0 mg, 1.135 mmol) and 500 μl of ethanol were added. The crude was poured into a saturated aqueous solution of NaHCO₃ (5 ml) and extracted with DCM (3×50 ml), the solution was filtered using a phase separator tube and the organic phase was concentrated under vacuo. The crude was purified using a 25M NH column eluting in gradient with DCM/Et₂O from 100:0 to 70:30 to give (trans)-3-(2-pyridinyl)-8-({[5-(trifluoromethyl)-2-pyridinyl]amino}methyl)-1-oxa-3-azaspiro[4.5]decan-2-one (49 mg).

¹H NMR (500 MHz, CDCl₃): δ 1.18-1.30 (m, 2H), 1.70-1.80 (m, 1H), 1.80-1.89 (m, 2H), 1.95-2.07 (m, 4H), 3.30 (t, 2H), 4.05 (s, 2H), 4.87-4.92 (m, 1H), 6.39-6.42 (m, 1H), 7.03-7.07 (m, 1H), 7.57-7.60 (m, 1H), 7.69-7.74 (m, 1H), 8.25-8.29 (m, 1H), 8.32-8.35 (m, 2H); UPLC-MS: 0.75 min, 407 [M+H]+.

The above compound was dissolved in DCM (1 ml) and a 1M solution of HCl in Et₂O was added dropwise under stirring. The solution was left at r.t. under stirring for 30 min and then the precipitate was separated, triturated with Et₂O, dried under a flow of nitrogen and then for 18 hours under high vacuum at 40° C. to give the title compound (53.2 mg);

### Example 1-3

### (Trans)-3-(2-pyridinyl)-8-({[5-(2-pyrimidinyl)-2-pyridinyl]amino}methyl)-1-oxa-3-azaspiro[4.5]decan-2-one Hydrochloride

(Trans)-2-oxo-3-(2-pyridinyl)-1-oxa-3-azaspiro[4.5]decane-8-carbaldehyde (Intermediate 12 procedure 12b, 45.3 mg, 0.174 mmol), 5-(2-pyrimidinyl)-2-pyridinamine (Intermediate 26, 30 mg, 0.174 mmol) and titanium(IV) isopropoxide (0.102 ml, 0.348 mmol) were collected and stirred in dichloromethane (2 ml) at r.t. for 24 hours. Then, sodium borohydride (19.77 mg, 0.523 mmol) and ethanol (2.000 ml) were added. The resulting mixture was stirred for a further 5 hours, then quenched with a saturated aqueous solution of NaHCO₃ and filtering over a filter tube. The resulting crude was purified by means of Biotage SP1, over a C18 12M column, eluting with a gradient of 5 mM (NH₄)₂CO₃ aq and acetonitrile to afford (trans)-3-(2-pyridinyl)-8-({[5-(2-pyrimidinyl)-2-pyridinyl]amino}methyl)-1-oxa-3-azaspiro[4.5]decan-2-one as a colourless solid (8.5 mg).

¹H NMR (400 MHz, CDCl₃): δ 9.18 (s, 1H), 8.73 (d, 1H), 8.45 (dd, 1H), 8.36-8.32 (m, 1H), 8.29-8.25 (m, 1H), 7.75-7.69 (m, 1H), 7.10 (t, 1H), 7.07-7.02 (m, 1H), 6.48 (d, 1H), 4.98 (brs, 1H), 4.06 (s, 2H), 2.08-1.17 (m, 9H); UPLC-MS: 0.56 min, 417 [M+H]+.

The above compound was dissolved in DCM (2 ml) and reacted with 1.0 equiv. of 1M HCl in Et₂O to afford the title compound (8.0 mg) as a colourless solid;

### Example 1-4

### (Trans)-8-({[5-(1-methyl-1H-imidazol-5-yl)-2-pyridinyl]amino}methyl)-3-(2-pyridinyl)-1-oxa-3-azaspiro[4.5]decan-2-one hydrochloride

The title compound was made in a similar fashion to the preparation of Example 1-3 replacing 5-(2-pyrimidinyl)-2-pyridinamine with 5-(1-methyl-1H-imidazol-5-yl)-2-pyridinamine (Intermediate 27, 30 mg, 0.172 mmol) and purifying with a KP-NH 12M column using a gradient of DCM and MeOH to afford the title compound (23 mg);

### Example 1-5

### (Trans)-3-(2-pyridinyl)-8-({[5-(1,3-thiazol-5-yl)-2-pyridinyl]amino}methyl)-1-oxa-3-azaspiro[4.5]decan-2-one Hydrochloride

The title compound was made in a similar fashion to the preparation of Example 1-3 replacing 5-(2-pyrimidinyl)-2-pyridinamine with 5-(1,3-thiazol-5-yl)-2-pyridinamine (Intermediate 28, 30 mg, 0.169 mmol) and purifying with a KP-NH 12M column using EtOAc as eluent to afford the title compound (17.4 mg);

### Example 1-6

### (Trans)-8-({[5-(5-methyl-1,3,4-thiadiazol-2-yl)-2-pyridinyl]amino}methyl)-3-(2-pyridinyl)-1-oxa-3-azaspiro[4.5]decan-2-one Hydrochloride

The title compound was made in a similar fashion to the preparation of Example 1-3 replacing 5-(2-pyrimidinyl)-2-pyridinamine with 5-(5-methyl-1,3,4-thiadiazol-2-yl)-2-pyridinamine (Intermediate 29, 30 mg, 0.156 mmol) and purifying with MDAP to afford the title compound (12.5 mg);

### Example 1-7

### (Trans)-8-[({5-[(difluoromethyl)oxy]-2-pyridinyl}amino)methyl]-3-(2-pyridinyl)-1-oxa-3-azaspiro[4.5]decan-2-one Dihydrochloride

(Trans)-8-(aminomethyl)-3-(2-pyridinyl)-1-oxa-3-azaspiro[4.5]decan-2-one (Intermediate 19, 121 mg, 0.464 mmol), 2-bromo-5-[(difluoromethyl)oxy]pyridine (Intermediate 20, 52 mg, 0.232 mmol), tris(dibenzylideneacetone)dipalladium (4.25 mg, 4.64 μmol), 2,2′-bis(diphenylphosphino)1,1′-binapthyl (5.78 mg, 9.29 μmol), and sodium tert-butoxide (31.2 mg, 0.325 mmol) were dissolved in toluene (2.1 ml) under nitrogen atmosphere. The mixture was irradiated in a microwave at 80° C. for 20 minutes, then at 100° C. for two cycles of 25 minutes. The mixture was diluted with ethyl acetate (30 ml) and washed with brine (2×10 ml). The organic phase was passed through a hydrophobic frit and evaporated. The crude was transferred onto a SCX resin and eluted with dichloromethane, methanol and 2M ammonia in methanol. The obtained semi-solid was purified on silica gel eluting with dichloromethane to dichloromethane/methanol 9/1 and then dichloromethane/2M ammonia in methanol 9/1 to 4/1 to afford (trans)-8-[({5-[(difluoromethyl)oxy]-2-pyridinyl}amino)methyl]-3-(2-pyridinyl)-1-oxa-3-azaspiro[4.5]decan- 2-one, which eluted at dichloromethane/2M ammonia in methanol 4/1, (26.2 mg).

¹H NMR (400 MHz, CDCl₃): δ 8.34 (1H, ddd), 8.27 (1H, d), 7.98 (1H, d), 7.72 (1H, ddd), 7.25-7.30 (1H, m), 7.05 (1H, ddd), 6.40 (1H, t), 6.37 (1H, d), 4.56 (1H, t), 4.05 (2H, s), 3.22 (2H, t), 1.89-2.07 (4H, m), 1.86 (2H, td), 1.67-1.79 (1H, m), 1.16-1.31 (2H, m); UPLC-MS: 0.60 min, 405 [M+H]+.

The above compound was dissolved in DCM (2 ml) and 1M hydrogen chloride in diethyl ether (0.5 ml) was added. The solvent was stripped off and the hygroscopic yellow solid dried under vacuum to afford the title compound (31.5 mg);

### Example 1-8

### (Trans)-8-[({5-[(cyclopropylmethyl)oxy]-2-pyridinyl}amino)methyl]-3-(2-pyridinyl)-1-oxa-3-azaspiro[4.5]decan-2-one dihydrochloride

(Trans)-8-(aminomethyl)-3-(2-pyridinyl)-1-oxa-3-azaspiro[4.5]decan-2-one (prepared in a similar fashion to Intermediate 19, 154 mg, 0.587 mmol), 2-bromo-5-[(cyclopropylmethyl)oxy]pyridine (Intermediate 21, 67 mg, 0.294 mmol), 2,2′-bis(diphenylphosphino)1,1′-binapthyl (18.29 mg, 0.029 mmol), tris(dibenzylideneacetone)dipalladium (13.45 mg, 0.015 mmol) and sodium tert-butoxide (39.5 mg, 0.411 mmol) were dissolved in toluene (3.0 ml) and shaken on the PLS at 95° C. for 18 hours. The mixture was diluted with ethyl acetate (30 ml) and washed with brine (2×10 ml). The organic phase was passed through a hydrophobic PTFE frit and evaporated. The crude was transferred onto an SCX resin and eluted with dichloromethane, methanol and 2M ammonia in methanol. The basic fractions were evaporated and the solid obtained was purified on NH₂-modified silica gel eluting with cyclohexane/ethyl acetate: 9/1 to 3/7 to afford trans 8-[({5-[(cyclopropylmethyl)oxy]-2-pyridinyl}amino)methyl]-3-(2-pyridinyl)-1-oxa-3-azaspiro[4.5]decan-2-one, which elutes at cyclohexane/ethyl acetate: 6/4 to 5/5, (46.5 mg).

¹H NMR (400 MHz, CDCl₃): δ 8.34 (1H, ddd), 8.28 (1H, d), 7.82 (1H, d), 7.72 (1H, ddd), 7.14 (1H, dd), 7.04 (1H, ddd), 6.37 (1H, d), 4.28 (1H, t), 4.05 (2H, s), 3.76 (2H, d), 3.18 (2H, t), 1.95-2.06 (4H, m), 1.85 (2H, td), 1.68-1.78 (1H, m), 1.15-1.31 (3H, m), 0.64 (2H, dqua), 0.34 (2H, qua); UPLC-MS: 0.56 min, 409 [M+H]+.

The above compound was dissolved in dichloromethane (1 ml) and treated with 1M hydrogen chloride in diethyl ether (1 ml). The solvents were evaporated and the solid dried at 42° C. under high vacuum for 6 hours to afford the title compound (50.0 mg);

### Example 1-9

### (Trans)-8-({[5-(cyclobutyloxy)-2-pyridinyl]amino}methyl)-3-(2-pyridinyl)-1-oxa-3-azaspiro[4.5]decan-2-one dihydrochloride

The title compound was made in a similar fashion to the preparation of Example 1-8 replacing 2-bromo-5-[(cyclopropylmethyl)oxy]pyridine with 2-bromo-5-(cyclobutyloxy)pyridine (Intermediate 22, 67 mg, 0.294 mmol) to afford the title compound (46.0 mg);

### Example 1-10

### (Trans)-5-phenyl-2-pyridinyl)amino]methyl}-3-(2-pyrazinyl)-1-oxa-3-azaspiro[4.5]decan-2-one Dihydrochloride

5-phenyl-2-pyridinamine (16.29 mg, 0.096 mmol) and (trans)-2-oxo-3-(2-pyrazinyl)-1-oxa-3-azaspiro[4.5]decane-8-carbaldehyde (Intermediate 15, 25 mg, 0.096 mmol) were dissolved in dry dichloromethane (2.000 ml) at r.t. and to the solution was added titanium tetraisopropoxide (0.056 ml, 0.191 mmol). The reaction was stirred overnight then sodium borohydride (10.86 mg, 0.287 mmol) and ethanol (0.5 ml) were added. The crude was poured into a saturated solution of NaHCO₃ (5 ml) and extracted with DCM (3×50 ml), the solution was filtered using a phase separator tube and the organic phase was concentrated under vacuo. The crude was purified using a 12M NH column eluting with cyclohexane/ethyl acetate (from 80:20 to 0:100) to give trans-8-{[(5-phenyl-2-pyridinyl)amino]methyl}-3-(2-pyrazinyl)-1-oxa-3-azaspiro[4.5]decan-2-one (16 mg).

¹H NMR (400 MHz, CDCl₃): δ 1.10-1.24 (m, 3H), 1.64-1.76 (m, 1H), 1.77-1.87 (m, 2H), 1.92-2.02 (m, 4H), 3.23 (t, 2H), 3.92-3.93 (m, 2H), 6.39 (dd, 1H), 7.19-7.26 (m, 1H), 7.32-7.38 (m, 2H), 7.41-7.46 (m, 2H), 7.58-7.63 (m, 1H), 8.19-8.21 (m, 1H), 8.23-8.29 (m, 2H), 9.53 (dd, 1H).

The above compound was dissolved in DCM (1 ml) and a 1M solution of hydrochloric acid in diethyl ether (0.085 ml, 0.085 mmol) was added dropwise under stirring. The solution was left at r.t. under stirring for 30 min and then the precipitate was separated, triturated with diethyl ether (1.0 ml), concentrated under a flow of nitrogen and dried for 18 hours under high vacuum at 40° C. to give the title compound (18 mg, 31%);

### Example 1-11

### (Trans)-8-{[(5-phenyl-2-pyridinyl)amino]methyl}-3-(3-pyridazinyl)-1-oxa-3-azaspiro[4.5]decan-2-one dihydrochloride

To a solution of (trans)-8-{[(5-phenyl-2-pyridinyl)amino]methyl}-1-oxa-3-azaspiro[4.5]decan-2-one (Intermediate 17, 35 mg, 0.104 mmol) and 3-chloropyridazine (14.26 mg, 0.124 mmol) in 1,4-dioxane (3 ml) were added copper(I) iodide (19.76 mg, 0.104 mmol), (+/−)-trans-1,2-diaminocyclohexane (0.025 ml, 0.207 mmol) and potassium phosphate (110 mg, 0.519 mmol). The mixture was stirred at 120° C. for 8 hours. 1,4-Dioxane was evaporated and the crude was dissolved in ethyl acetate (50 ml) and washed with a pH 3 citrate buffer solution (10 ml), the organic phase was dried on Na₂SO₄ and concentrated under vacuo. The crude was purified on a NH cartridge eluting with a gradient of cyclohexane/ethyl acetate (from 10% to 100%) to give trans-8-{[(5-phenyl-2-pyridinyl)amino]methyl}-3-(3-pyridazinyl)-1-oxa-3-azaspiro[4.5]decan-2-one (23.9 mg).

¹H NMR (400 MHz, CDCl₃): δ 1.60-1.96 (m, 6H), 2.01-2.11 (m, 4H), 3.27-3.33 (m, 2H), 4.22-4.25 (m, 2H), 6.50 (dd, 1H), 7.29-7.36 (m, 1H), 7.40-7.47 (m, 2H), 7.47-7.56 (m, 3H), 7.70 (dd, 1H), 8.36 (dd, 1H), 8.58 (dd, 1H), 8.97 (dd, 1H); UPLC-MS: 0.59 min, 416 [M+H]+

The above compound was dissolved in DCM (2 ml) a 1M solution of hydrochloric acid in diethyl ether (0.127 ml, 0.127 mmol) was added dropwise under stirring. The solution was left at r.t. under stirring for 30 min and then the precipitate was separated, triturated with diethyl ether (2 ml), concentrated under a flow of nitrogen and dried for 6 hours under high vacuum at 40° C. to give the title compound (9 mg, 32%);

### Example 1-12

### (Trans)-3-(2-fluoro-3-pyridinyl)-8-({[5-(trifluoromethyl)-2-pyridinyl]amino}methyl)-1-oxa-3-azaspiro[4.5]decan-2-one hydrochloride

(Trans)-8-({[5-(trifluoromethyl)-2-pyridinyl]amino}methyl)-1-oxa-3-azaspiro[4.5]decan-2-one (Intermediate 16, 35 mg, 0.106 mmol), 3-bromo-2-fluoropyridine (18.70 mg, 0.106 mmol), copper(I) iodide (20.24 mg, 0.106 mmol), trans-1,2-diaminocyclohexane (0.013 ml, 0.106 mmol) and potassium phosphate (113 mg, 0.531 mmol) were combined in 1,4-dioxane (2 ml) in a closed vial and the resulting mixture was stirred at 120° C. for 5 hours then it was left standing at r.t. overnight. Then further copper(I) iodide (10 mg) and trans-1,2-diaminocyclohexane (0.006 ml) were added and the mixture was stirred at 120° C. for 7 hours then it was left at r.t. overnight. Solvent was evaporated under vacuo then the mixture was taken up with DCM and filtered over a filter cartridge washing with more DCM (20 ml total). The mixture was dried and purified by Biotage KP-NH column chromatography (Biotage SP1, 12+M) eluting with cyclohexane (lcv), cyclohexane:EtOAc from 100:0 to 0:100 (15 cv). Product fractions were combined and dried to afford (trans)-3-(2-fluoro-3-pyridinyl)-8-({[5-(trifluoromethyl)-2-pyridinyl]amino}methyl)-1-oxa-3-azaspiro[4.5]decan-2-one as a pale yellow solid (23.4 mg, 52%).

¹H NMR (400 MHz, CDCl₃): δ 1.10-1.25 (m, 2H), 1.70-1.83 (m, 1H), 1.83-1.96 (m, 2H), 1.97-2.11 (m, 4H), 3.33 (t, 2H), 3.90 (s, 2H), 4.86-4.97 (m, 1H), 6.38-6.45 (m, 1H), 7.25-7.31 (m, 1H), 7.56-7.62 (m, 1H), 8.07-8.12 (m, 1H), 8.13-8.22 (m, 1H), 8.28-8.39 (m, 1H); UPLC-MS: 0.71 min, 425 [M+H]+

The above compound was suspended in Et₂O (0.5 ml) and HCl 1M in Et₂O (1.2 eq, 0.066 ml, 0.066 mmol) were added. The resulting solid was triturated with Et₂O (3×0.3 ml) and dried to afford the title compound as a pale yellow solid (22 mg, 45%);

### Example 1-13

### (Trans)-3-(3-pyridazinyl)-8-({[5-(trifluoromethyl)-2-pyridinyl]amino}methyl)-1-oxa-3-azaspiro[4.5]decan-2-one Hydrochloride

The title compound was made in a similar fashion to the preparation of Example 1-12 replacing 3-bromo-2-fluoropyridine with 3-chloropyridazine (12.17 mg, 0.106 mmol) to afford the title compound as a white solid (17.2 mg, 37%);

### Example 1-14

### (Trans)-3-(1-methyl-1H-pyrazol-3-yl)-8-({[5-(trifluoromethyl)-2-pyridinyl]amino}methyl)-1-oxa-3-azaspiro[4.5]decan-2-one hydrochloride

The title compound was made in a similar fashion to the preparation of Example 1-12 replacing 3-bromo-2-fluoropyridine with 3-iodo-1-methyl-1H-pyrazole (22.11 mg, 0.106 mmol) to afford the title compound as a white solid (28 mg, 59%);

### Example 1-15

### (Trans)-3-(2-pyridinyl)-8-({[5-(1,3-thiazol-2-yl)-2-pyridinyl]amino}methyl)-1-oxa-3-azaspiro[4.5]decan-2-one Hydrochloride

The title compound was made in a similar fashion to the preparation of Example 1-3 replacing 5-(2-pyrimidinyl)-2-pyridinamine with 5-(1,3-thiazol-2-yl)-2-pyridinamine (Intermediate 30, 34 mg, 0.192 mmol) and purifying with a KP-NH 12 M column and a silica gel column using a gradient of DCM and diethyl ether and then MDAP to afford the title compound (13.5 mg);

### Example 1-16

### (Trans)-8-({[5-(3,5-dimethyl-4-isoxazolyl)-2-pyridinyl]amino}methyl)-3-(2-pyridinyl)-1-oxa-3-azaspiro[4.5]decan-2-one

The title compound was made in a similar fashion to the preparation of Example 1-3 replacing 5-(2-pyrimidinyl)-2-pyridinamine with 5-(3,5-dimethyl-4-isoxazolyl)-2-pyridinamine (Intermediate 31, 33 mg, 0.192 mmol) and purifying with a 12 M silica gel column using a gradient of DCM and diethyl ether and then MDAP to afford the title compound (3.2 mg);

### Example 1-17

### (Trans)-8-({[5-(2-methyl-1,3-thiazol-4-yl)-2-pyridinyl]amino}methyl)-3-(2-pyridinyl)-1-oxa-3-azaspiro[4.5]decan-2-one

In a closed vial 5-(2-methyl-1,3-thiazol-4-yl)-2-pyridinamine (Intermediate 32, 36.7 mg, 0.192 mmol) and (trans)-2-oxo-3-(2-pyridinyl)-1-oxa-3-azaspiro[4.5]decane-8-carbaldehyde (Intermediate 12 procedure 12b, 50 mg, 0.192 mmol) were dissolved in dichloromethane (2 ml). Titanium (IV) isopropoxide (0.113 ml, 0.384 mmol) was added and the mixture was stirred at r.t. for ca 24 hours then NaBH₄ (21.80 mg, 0.576 mmol) and ethanol (0.5 ml) (caution: gas evolution) were added and the mixture was stirred at r.t. for 8 hours. Then the mixture was diluted with DCM (1 ml), NaHCO₃ aqueous saturated solution (ca 3 ml) was added and the mixture was extracted with DCM (3×5 ml). The organics were combined and dried to afford a crude that was purified by MDAP (Preparative Column: AXIA 50×21 mm; Method: Focus 01: 30% (B) to 35% (B) in 1 min; 35% (B) to 65% (B) in 7 min; 65% (B) to 100% (B) in 1 min; 100% (B) for 1.5 min; where Mobile phasse are A: NH₄HCO₃ so1.10 mM, pH10; B: ACN). Product fractions were combined and dried to afford a mixture that was further purified by Biotage silica gel column chromatography (Biotage SP1, 12+M) eluting with DCM/Et₂O 100: to 60:40 (15 cv) to afford impure title compound as a white solid (24.4 mg, purity 83%). 23.3 mg (0.05 mmol) of this material were dissolved in DCM (2 ml). 17 mg (0.04 mmol) of polymer supported p-toluenesulfonyl hydrazide, (nucleofilic scavenger from Polymer Labs, loading 2.27 mmol/g) were added and the mixture was shaked on a PLS at r.t. for 17 hours. Then the resin was filtered off on a separatory cartridge rinsing with DCM. The solvent was evaporated under reduced pressure to afford a mixture that was purified by silica column chromatography (Biotage SP1, 12+M) eluting with DCM/Et₂O 100:0 to 60:40 (25 cv) to afford the title compound as a pale yellow solid (12.4 mg, 15%);

### Example 1-18

### (Trans)-8-({[5-(4-pyridazinyl)-2-pyridinyl]amino}methyl)-3-(2-pyridinyl)-1-oxa-3-azaspiro[4.5]decan-2-one Hydrochloride

The title compound was made in a similar fashion to the preparation of Example 1-3 replacing 5-(2-pyrimidinyl)-2-pyridinamine with 5-(4-pyridazinyl)-2-pyridinamine (Intermediate 33, 33.1 mg, 0.192 mmol) and purifying with MDAP to afford the title compound (6 mg);

### Example 1-19

### (Trans)-3-(2-pyridinyl)-8-({[5-(1,3,5-trimethyl-1H-pyrazol-4-yl)-2-pyridinyl]amino}methyl)-1-oxa-3-azaspiro[4.5]decan-2-one

In a closed vial 5-(1,3,5-trimethyl-1H-pyrazol-4-yl)-2-pyridinamine (Intermediate 34, 31.1 mg, 0.154 mmol) and (trans)-2-oxo-3-(2-pyridinyl)-1-oxa-3-azaspiro[4.5]decane-8-carbaldehyde (Intermediate 12 procedure 12b, 40 mg, 0.154 mmol) were dissolved in 1,2-dichloroethane (1 ml). Titanium (IV) isopropoxide (0.090 ml, 0.307 mmol) was added and the mixture was stirred at 60° C. for 7 hours. Then it was cooled to r.t. and NaBH₄ (17.44 mg, 0.461 mmol) followed by ethanol (0.5 ml) were added and the mixture was left stirring at r.t. overnight. 2 props of acetic acid were added and the mixture was stirred at r.t. for 4 hours. Then 2 ml of a saturated aqueous solution of K₂CO₃ were added and the mixture was stirred at r.t. for 30 minutes then it was left standing at r.t. for 5 days. The mixture was extracted with DCM (3×5 ml), the organic layers were combined and dried under vacuo to afford 73 mg of a colourless oil that was dissolved in DCM (1 ml), NaBH₄ (17 mg, 0.45 mmol) followed by 2 drops of acetic acid were added and the mixture was stirred at r.t. overnight. Then 2 ml of a saturated aqueous solution of K₂CO₃ were added and the mixture was stirred at r.t. for 2 hours then it was extracted with DCM (3×5 ml), the organic layers were combined and dried under vacuo to afford 70 mg of a colourless oil that was purified by Biotage silica column chromatography (Biotage SP1, 12+M) eluting with DCM/Et₂O 100:0 to 60:40 (25 cv) (column performed twice) to afford 22 mg of a mixture that was further purified by MDAP (Preparative Column: AXIA 50×21 mm; Method: Focus 00:10% (B) to 15% (B) in 1 min; 15% (B) to 70% (B) in 7 min; 70% (B) to 100% (B) in 1 min; 100% (B) for 1.5 min; where Mobile phase are A: NH₄HCO₃ so1.10 mM, pH10; B: ACN). Product fractions were combined and dried to afford the title compound as a yellow solid (0.9 mg, 1.3%);

### Example 1-20

### (Trans)-8-({[5-(3-methyl-1H-pyrazol-4-yl)-2-pyridinyl]amino}methyl)-3-(2-pyridinyl)-1-oxa-3-azaspiro[4.5]decan-2-one

The title compound was made in a similar fashion to the preparation of Example 1-3 replacing 5-(2-pyrimidinyl)-2-pyridinamine with 5-(3-methyl-1H-pyrazol-4-yl)-2-pyridinamine (Intermediate 35, 30 mg, 0.172 mmol) and purifying with MDAP to afford the title compound (2.5 mg);

### Example 1-21

### (Trans)-8-({[5-(4-morpholinyl)-2-pyridinyl]amino}methyl)-3-(2-pyridinyl)-1-oxa-3-azaspiro[4.5]decan-2-one Hydrochloride

In a closed vial (trans)-2-oxo-3-(2-pyridinyl)-1-oxa-3-azaspiro[4.5]decane-8-carbaldehyde (Intermediate 12 procedure 12b, 29.0 mg, 0.112 mmol), and 5-(4-morpholinyl)-2-pyridinamine (can be commercially available, 20 mg, 0.112 mmol) were dissolved in dichloromethane (2 ml). Titanium(IV) isopropoxide (0.065 ml, 0.223 mmol) was added and the mixture was stirred at r.t. overnight (ca 15 hours). Then NaBH₄ (16.89 mg, 0.446 mmol) followed by ethanol (0.5 ml) were added and the resulting mixture was stirred at r.t. for 6 hours. The mixture was diluted with DCM (2 ml), K₂CO₃ saturated solution (2 ml) was added and the mixture was stirred at r.t. for 30 minutes followed by extraction with DCM (3×3 ml). The organics were combined, filtered through a filtering tube and dried (vacuo) to afford a crude that was purified by Fraction Lynx (Preparative Column: AXIA 50×21 mm; Focus 00:10% (B) to 15% (B) in 1 min; 15% (B) to 70% (B) in 7 min; 70% (B) to 100% (B) in 1 min; 100% (B) for 1.5 min; where Mobile phase are A: NH₄HCO₃ so1.10 mM, pH10; B: ACN). Product fractions were combined and dried to afford (trans)-8-({[5-(4-morpholinyl)-2-pyridinyl]amino}methyl)-3-(2-pyridinyl)-1-oxa-3-azaspiro[4.5]decan-2-one as a whitish foam (17.2 mg, 0.041 mmol).

¹H NMR (400 MHz, CDCl₃): δ 1.15-1.31 (m, 2H), 1.65-1.77 (m, 1H), 1.77-1.92 (m, 2H), 1.94-2.07 (m, 4H), 2.96-3.07 (m, 4H), 3.14-3.24 (m, 2H), 3.82-3.92 (m, 4H), 4.05 (s, 2H), 4.28-4.38 (m, 1H), 6.36-6.44 (m, 1H), 7.00-7.08 (m, 1H), 7.15-7.22 (m, 1H), 7.67-7.76 (m, 1H), 7.79-7.84 (m, 1H), 8.24-8.30 (m, 1H), 8.31-8.37 (m, 1H); UPLC-MS: 0.50 min, 424 [M+H]+.

The above compound (17.2 mg, 0.041 mmol) was suspended in Et₂O (0.5 ml) and 1M HCl in Et₂O (0.049 ml, 0.049 mmol) were added. The resulting solid was triturated with Et₂O (3×0.3 ml) and dried (vacuo at 40° C. overnight) to afford the title compound as a pale yellow powder (16.5 mg, 32%).

### Example 1-22

### (Trans)-8-({[5-(cyclopentyloxy)-2-pyridinyl]amino}methyl)-3-(2-pyridinyl)-1-oxa-3-azaspiro[4.5]decan-2-one hydrochloride

(Trans)-8-(aminomethyl)-3-(2-pyridinyl)-1-oxa-3-azaspiro[4.5]decan-2-one (prepared in a similar fashion to Intermediate 19, 37.8 mg, 0.145 mmol), 2-bromo-5-(cyclopentyloxy)pyridine (Intermediate 59, 35 mg, 0.145 mmol), BINAP (9.00 mg, 0.014 mmol), Pd₂ dba₃ (6.62 mg, 7.23 μmol) and sodium tert-butoxide (19.45 mg, 0.202 mmol) were mixed in toluene (1.5 ml) in a closed vial and the resulting mixture was shaken in a PLS instrument at 95° C. for ca 18 hours. The mixture was allowed to cool to r.t. and it was partitioned between EtOAc/brine. The organics were filtered over a separatory cartridge and dried (vacuo). The crude mixture was purified by KP-NH column (Biotage SP1, 12+M) eluting with cyclohexane:EtOAc 9:1 to 3:7 to afford a mixture that was further purified by silica gel chromatography (cartridge 2g) eluting with cyclohexane:EtOAc 9:1 to 1:1 (TLC on silica plate, cyclohexane:EtOAc 1:1; R_{f} 0.23). Product fractions were combined and dried to afford (trans)-8-({[5-(cyclopentyloxy)-2-pyridinyl]amino}methyl)-3-(2-pyridinyl)-1-oxa-3-azaspiro[4.5]decan-2-one as a yellowish solid (7.6 mg).

¹H NMR (400 MHz, CDCl₃): δ 1.12-1.35 (m, 2H), 1.56-1.77 (m, 3H), 1.77-1.94 (m, 8H), 1.94-2.02 (m, 4H), 3.13-3.23 (m, 2H), 4.06 (s, 2H), 4.64 (t, 1H), 6.38 (d, 1H), 7.00-7.09 (m, 1H), 7.12 (dd, 1H), 7.67-7.80 (m, 2H), 8.28 (d, 1H), 8.35 (dt, 1H); HPLC-MS: 1.709 min, 423 [M+H]+.

The above compound (7.6 mg, 0.018 mmol) was suspended in Et₂O (0.5 ml). 1M HCl in Et₂O (0.022 mmol, 0.022 ml) was added and the resulting solid was triturated with Et₂O (3×0.3 ml) then dried (vacuo, 40° C. ca 16 hours) to afford the title compound as a white solid (5.3 mg, 8%).

### Example 1-23

### (Trans)-8-({[5-(cyclopentyloxy)-2-pyridinyl]amino}methyl)-3-(3-pyridazinyl)-1-oxa-3-azaspiro[4.5]decan-2-one

(Trans)-2-oxo-3-(3-pyridazinyl)-1-oxa-3-azaspiro[4.5]decane-8-carbaldehyde (Intermediate 60, 16.13 mg, 0.062 mmol) and 5-(cyclopentyloxy)-2-pyridinamine (Intermediate 61, 11 mg, 0.062 mmol) were suspended in dichloromethane (1 ml) in a closed vial. Titanium(IV) isopropoxide (0.036 ml, 0.123 mmol) was added and the resulting solution was stirred at room temperature overnight. Further titanium(IV) isopropoxide (0.036 ml, 0.123 mmol) was added and the mixture was stirred for 4 hours, then NaBH₄ (11.67 mg, 0.309 mmol) followed by ethanol (1 ml) were added and the mixture was stirred at r.t overnight (ca 18 hours). Saturated NaHCO₃ solution (2 ml) was added and the mixture was stirred at r.t. for 30 min then the aqueous phase was extracted with DCM (3×3 ml). The organic extracts were combined and dried to afford a crude that was purified by KP-NH cartridge (Biotage SP1 system) eluting in gradient with 0%-50% Et₂O\DCM to afford the title compound (7 mg, 27%).

### Example 1-24

### (Trans)-8-({[5-(cyclohexyloxy)-2-pyridinyl]amino}methyl)-3-(3-pyridazinyl)-1-oxa-3-azaspiro[4.5]decan-2-one Hydrochloride

(Trans)-2-oxo-3-(3-pyridazinyl)-1-oxa-3-azaspiro[4.5]decane-8-carbaldehyde (Intermediate 60, 20.38 mg, 0.078 mmol) and 5-(cyclohexyloxy)-2-pyridinamine (Intermediate 62, 15 mg, 0.078 mmol) were suspended in dichloromethane (1 ml) in a closed vial. Titanium(IV) isopropoxide (0.046 ml, 0.156 mmol) was added and the resulting solution was stirred at room temperature overnight. Then NaBH₄ (14.76 mg, 0.390 mmol) followed by ethanol (0.5 ml) were added and the mixture was stirred at r.t. for 5 hours. Saturated NaHCO₃ solution (2 ml) was added and the mixture was stirred at r.t. for 30 min. then the aqueous was extracted with DCM (3×3 ml). The organic extracts were combined and dried to afford a crude which was purified on KP-NH cartridge (Biotage SP1) eluting in gradient with 0%-50% Et₂ODCM to afford a mixture that was further purified by silica gel chromatography (Biotage SP1) eluting in gradient with 0%-50% Et₂ODCM then with 5%-10% MeOHDCM to afford (trans)-8-({[5-(cyclohexyloxy)-2-pyridinyl]amino}methyl)-3-(3-pyridazinyl)-1-oxa-3-azaspiro[4.5]decan-2-one as a yellowish solid (8.5 mg).

¹H NMR (400 MHz, CDCl₃): δ 1.14-1.42 (m, 6H), 1.42-1.65 (m, 3H), 1.67-2.13 (m, 10H), 3.14-3.27 (m, 2H), 3.96-4.08 (m, 1H), 4.22 (s, 2H), 4.32-4.45 (m, 1H), 6.37 (d, 1H), 7.14 (dd, 1H), 7.50 (dd, 1H), 7.84 (d, 1H), 8.58 (dd, 1H), 8.97 (dd, 1H); HPLC-MS: 1.665 min, 438 [M+H]+

The above compound (8.5 mg) was dissolved in Et₂O (0.5 ml) and DCM (0.5 ml). 1M HCl in Et₂O (0.023 mml, 0.023 ml) was added, the mixture was dried (vacuo) and the resulting solid was triturated with Et₂O (3×0.3 ml) then dried (vacuo, 40° C., 2 hours) to afford the title compound as a pale yellow solid (7.6 mg, 20%).

### Example 1-25

### (Trans)-3-(3-pyridinyl)-8-({[6-(trifluoromethyl)-2-pyridinyl]amino}methyl)-1-oxa-3-azaspiro[4.5]decan-2-one Dihydrochloride

6-(Trifluoromethyl)-2-pyridinamine (24.91 mg, 0.154 mmol), (trans)-2-oxo-3-(3-pyridinyl)-1-oxa-3-azaspiro[4.5]decane-8-carbaldehyde (Intermediate 65, 40 mg, 0.154 mmol) and titanium(IV) isopropoxide (0.090 ml, 0.307 mmol) were collected in dichloromethane (2 ml) and shaken at r.t. overnight. Then sodium borohydride (17.44 mg, 0.461 mmol) and ethanol (2.0 ml) were added and the resulting mixture was shaken for 5 hours. It was then rinsed with DCM (20 ml), treated with NaHCO₃ (5 ml) and filtered over a filter tube washing with DCM (2×5 ml). The collected organic phases were concentrated to afford 50 mg of crude compound. This was purified by MDAP system Fraction Lynx (method D) to afford (trans)-3-(3-pyridinyl)-8-({[6-(trifluoromethyl)-2-pyridinyl]amino}methyl)-1-oxa-3-azaspiro[4.5]decan-2-one (8.5 mg).

¹H NMR (400 MHz, CDCl₃): δ 8.62 (d, 1H), 8.40 (dd, 1H), 8.24 (dq, 1H), 7.54 (t, 1H), 7.32 (ddd, 1H), 6.93 (d, 1H), 6.54 (d, 1H), 4.82 (br s, 1H), 3.83 (s, 2H), 3.34 (t, 2H), 2.10-1.72 (m, 7H), 1.30-1.18 (m, 2H); UPLC-MS: 0.72 min, 407 [M+H]+.

The above compound was dissolved in DCM (2 ml) and converted into the corresponding salt by reaction with 1M HCl in Et₂O (2 eq) to afford the title compound as a colourless solid (12 mg).

### Example 1-26

### (Trans)-3-(3-pyridazinyl)-8-({[5-(2-pyrimidinyl)-2-pyridinyl]amino}methyl)-1-oxa-3-azaspiro[4.5]decan-2-one Hydrochloride

(Trans)-8-(aminomethyl)-3-(3-pyridazinyl)-1-oxa-3-azaspiro[4.5]decan-2-one (Intermediate 41 procedure 41a, 30 mg, 0.114 mmol), 2-(6-fluoro-3-pyridinyl)pyrimidine (Intermediate 66, 20.0 mg, 0.114 mmol) were dissolved in N,N-dimethylacetamide (2 ml) in a microwave vial and irradiated at 140° C. for 30 min, then 150° C. for 30 min, and finally at 1600 for 30 min. The mixture was passed through an ion exchange cartridge (SCX 2g, Varian) washing with MeOH and eluting with 2M ammonia in MeOH. The resulting crude was further purified by reverse phase flash chromatography (Biotage SP1, 12M KP-C18 column) eluting with ACN and water (made-up with 0.1% HCOOH). The required compound was recovered as a colourless solid (18 mg) and it was further passed through an ion exchange cartridge (SCX Varian 1 g) washing with methanol and eluting with 2M ammonia in methanol. Solvent was removed in vacuo to afford (trans)-3-(3-pyridazinyl)-8-({[5-(2-pyrimidinyl)-2-pyridinyl]amino}methyl)-1-oxa-3-azaspiro[4.5]decan-2-one as a colourless solid (16 mg).

¹H NMR (400 MHz, CDCl₃): δ 9.17 (dd, 1H), 8.95 (dd, 1H), 8.72 (d, 2H), 8.56 (dd, 1H), 8.43 (dd, 1H), 7.50 (ddd, 1H), 7.10 (t, 1H), 6.48 (dd, 1H), 5.14 (br s, 1H), 4.22 (s, 2H), 3.22 (t, 2H), 2.13-1.13 (m, 9H); UPLC-MS: 0.46 min, 418 [M+H]+.

The above compound was dissolved in DCM (2 ml) and reacted with 1.0 equiv. of 1M HCl in Et₂O to give the title compound as a colourless solid (15 mg).

### Example 1-27

### (Trans)-8-{[(5-bromo-2-pyridinyl)amino]methyl}-3-(2-pyridinyl)-1-oxa-3-azaspiro[4.5]decan-2-one

In a 2 ml microwave vial, (trans)-8-(aminomethyl)-3-(2-pyridinyl)-1-oxa-3-azaspiro[4.5]decan-2-one (prepared in a similar fashion to Intermediate 19, 60 mg, 0.230 mmol) was dissolved in N,N-dimethylformamide (0.5 ml) and TEA (0.064 ml, 0.459 mmol) and 5-bromo-2-fluoropyridine (0.028 ml, 0.276 mmol) were added. The resulting mixture was irradiated at 130° C. for 30 min (3 cycles), then stirred at 130° C. overnight. The reaction mixture was loaded onto a SCX cartridge (5g), washed with MeOH and eluted with 2.0M NH₃ in MeOH to give the title compound (30.7 mg, 0.074 mmol, 32%).

### Example 1-28

### Methyl 6-({[2-oxo-3-(3-pyridazinyl)-1-oxa-3-azaspiro[4.5]dec-8-yl]methyl}amino)-3-Pyridinecarboxylate

To a mixture of methyl 6-amino-3-pyridinecarboxylate (Intermediate 81, 23.3 mg, 0.153 mmol) and trans-2-oxo-3-(4-pyridazinyl)-1-oxa-3-azaspiro[4.5]decane-8-carbaldehyde (Intermediate 60, 40 mg, 0.153 mmol) in DCM (3 ml) was added chlorotitanium triisopropoxide (0.110 ml, 0.459 mmol) dissolved in DCM (1.0 ml) and the mixture was stirred at r.t. overnight. Sodium triacetoxyborohydride (162 mg, 0.765 mmol) and acetic acid (8.76 μl, 0.153 mmol) were added and the reaction mixture was stirred for 2 hours followed by a further addition of acetic acid (5 eq). The crude was poured into a saturated NaHCO₃ solution (20 ml) and extracted with DCM (50 ml). To the resulting emulsion 2M NaOH (3 ml) was added and the organic phase was separated by a phase separator tube then concentrated under vacuum to give a crude. The crude was purified by KP-NH flash-chromatography (Biotage SP1, 12M NH column) eluting in gradient with 20%-100% EtOAc\cyclohexane to afford the title compound (14 mg, 0.035 mmol, 23%).

### Example 1-29

### (Trans)-8-({[5-(1H-pyrazol-1-yl)-2-pyridinyl]amino}methyl)-3-(2-pyridinyl)-1-oxa-3-azaspiro[4.5]decan-2-one.dihydrochloride

Copper(I) iodide (1.295 mg, 6.80 μmol), 1H-pyrazole (4.63 mg, 0.068 mmol), potassium carbonate (19.7 mg, 0.143 mmol), trans-8-{[(5-bromo-2-pyridinyl)amino]methyl}-3-(2-pyridinyl)-1-oxa-3-azaspiro[4.5]decan-2-one (prepared in a similar fashion to the Example 1-27, 34.4 mg, 0.082 mmol) and (+/−)-trans-N,N-dimethyl-1,2-diaminocyclohexane (2.145 μl, 0.014 mmol) were dissolved in toluene (1 ml) in a sealed tube. The mixture was stirred at 110° C. for 48 hours. Solvent was removed under vacuum, the residue was taken up with DCM and filtered over a filter tube; the organic phase was concentrated under vacuum to give a crude. The crude was purified on a 25M NH cartridge eluting in gradient with 20%-100% EtOAc\cyclohexane to give trans-8-({[5-(1H-pyrazol-1-yl)-2-pyridinyl]amino}methyl)-3-(2-pyridinyl)-1-oxa-3-azaspiro[4.5]decan-2-one (14.1 mg, 0.035 mmol, 51%).

¹H NMR (400 MHz, CDCl₃): δ 1.58-1.91 (m, 5H), 1.98-2.05 (m, 4H), 3.26-3.31 (m, 2H), 4.06 (s, 2H), 4.66-4.73 (m, 1H), 6.44-6.51 (m, 2H), 7.05 (ddd, 1H), 7.69-7.80 (m, 4H), 8.28 (dt, 1H), 8.33-8.39 (m, 2H); UPLC-MS: 0.56 min, 405 [M+H]+.

The above compound (14.1 mg, 0.035 mmol) was dissolved in DCM (2 ml) and treated with 1.0M HCl in Et₂O (0.087 ml, 0.087 mmol) and some drops of Et₂O for 30 minutes under stirring. Solvent was evaporated under reduced pressure and the residue was triturated with Et₂O (2.0 ml) and filtered. The solid was dried under vacuum at 60° C. to afford the title compound (14.3 mg, 0.030 mmol, 86%).

### Example 1-30

### (Trans)-3-(2-fluoro-3-pyridinyl)-8-({[5-(2-pyrimidinyl)-2-pyridinyl]amino}methyl)-1-oxa-3-azaspiro[4.5]decan-2-one.dihydrochloride

To a solution of (trans)-8-({[5-(2-pyrimidinyl)-2-pyridinyl]amino}methyl)-1-oxa-3-azaspiro[4.5]decan-2-one (Intermediate 82, 40 mg, 0.118 mmol) and 2-fluoro-3-iodopyridine (31.5 mg, 0.141 mmol) in 1,4-dioxane (2 ml) were added copper(I) iodide (22.45 mg, 0.118 mmol), (+/−)-trans-1,2-diaminocyclohexane (0.028 ml, 0.236 mmol) and K₃PO₄ (125 mg, 0.589 mmol) in a sealed microwave tube and the mixture was irradiated at 80° C. for 10 min (four cycles). The solvent was evaporated. DCM (5 ml) was added to the resulting residue and this was filtered washing with more DCM (2×1 ml). The organic solution was concentrated to give a residue which was purified by KP-NH flash chromatography (via Biotage SP1, 12M NH column) eluting in gradient with 30%-100% EtOAc/cyclohexane to give 16.4 mg of desired compound contaminated by 5-(2-pyrimidinyl)-2-pyridinamine. This residue was dissolved in DCM and washed with a pH 3 citrate buffer solution (3 ml) to afford clean (trans)-3-(2-fluoro-3-pyridinyl)-8-({[5-(2-pyrimidinyl)-2-pyridinyl]amino}methyl)-1-oxa-3-azaspiro[4.5]decan-2-one (13.4 mg, 0.031 mmol, 26%).

¹H NMR (400 MHz, CDCl₃): δ 1.69-1.84 (m, 3H), 1.84-1.94 (m, 2H), 2.02-2.10 (m, 4H), 3.26-3.39 (m, 2H), 3.85-3.96 (m, 2H), 4.95 (s, 1H), 6.44-6.52 (m, 1H), 7.06-7.16 (m, 1H), 7.21-7.33 (m, 2H), 8.08 (dt, 1H), 8.16-8.21 (m, 1H), 8.40-8.50 (m, 1H), 8.70-8.80 (m, 1H), 9.15-9.22 (m, 1H); UPLC-MS: 0.47 min, 435 [M+H]+.

The above compound (13.4 mg, 0.031 mmol) was dissolved in DCM (2 ml) and treated with 1.0M HCl in diethyl ether (0.077 ml, 0.077 mmol) for 30 minutes. Solvent was evaporated under reduced pressure and the solid was triturated with diethyl ether (2.0 ml) then dried under vacuum at 60° C. for 7 hours to afford the title compound (12.1 mg, 0.024 mmol, 77%).

### Example 1-31

### (Trans)-3-(1H-pyrazol-4-yl)-8-({[5-(2-pyrimidinyl)-2-pyridinyl]amino}methyl)-1-oxa-3-azaspiro[4.5]decan-2-one.dihydrochloride

1M HCl in diethyl ether (0.882 ml, 0.882 mmol) was added to a solution of trans-8-({[5-(2-pyrimidinyl)-2-pyridinyl]amino}methyl)-3-[1-(tetrahydro-2H-pyran-2-yl)-1H-pyrazol-4-yl]-1-oxa-3-azaspiro[4.5]decan-2-one (two combined batches: Intermediate 84, 14.8 mg, 0.044 mmol and a batch prepared in a similar fashion to Intermediate 84 but stirring the reaction mixture at 120° C. for ca 64 hours, 6.8 mg) in ethanol (2.0 ml) at room temperature under nitrogen and the resulting solution was left to stand for 1 hour. Volatiles were evaporated under reduced pressure. The residue was dissolved in MeOH (1 ml) and loaded onto a 2g SCX cartridge. Washed with MeOH and eluted with 2M NH₃ in MeOH. The basic fractions were combined and evaporated under reduced pressure. The residue was purified by KP-NH chromatography (Biotage SP1, 12M NH column) eluting in gradient with 5%-20% MeO H/CH₂Cl₂ to give (trans)-3-(1H-pyrazol-4-yl)-8-({[5-(2-pyrimidinyl)-2-pyridinyl]amino}methyl)-1-oxa-3-azaspiro[4.5]decan-2-one (7.3 mg, 0.018 mmol, 41%).

UPLC-MS: 0.41 min, 406 [M+H]+.

The above compound (7.3 mg, 0.018 mmol) was dissolved in dichloromethane (1 ml) and MeOH (0.2 ml) and treated with 1.0M HCl in diethyl ether (0.045 ml, 0.045 mmol). The resulting solution was left to stand for 30 minutes then evaporated under reduced pressure. The residue was triturated with diethyl ether (3 ml) and the solid was dried under vacuum at 60° C. for 7 hours to afford the title compound (8 mg, 0.017 mmol, 38%).

### Example 1-32

### N-Cyclohexyl-6-({[(trans)-2-oxo-3-(2-pyridinyl)-1-oxa-3-azaspiro[4.5]dec-8-yl]methyl}amino)-3-pyridinecarboxamide

6-({[Trans-2-oxo-3-(2-pyridinyl)-1-oxa-3-azaspiro[4.5]dec-8-yl]methyl}amino)-3-pyridinecarboxylic acid (Intermediate 85, 35.2 mg, 0.023 mmol) was dissolved in acetonitrile (2 ml) and HOBt (3.52 mg, 0.023 mmol) and EDC hydrochloride (4.41 mg, 0.023 mmol) were added and the mixture was stirred for 15 min followed by addition of cyclohexylamine (2.288 μl, 0.02 mmol). The resulting suspension was stirred at r.t. overnight then DMF (500 μl) was added to try to dissolve the suspension. Further HOBt (5.2 mg 0.034 mmol) and EDC hydrochloride (6.6 mg, 0.034 mmol) were added to the mixture which was left at r.t. overnight then heated at 38° C. for 24 hours. The reaction mixture was poured into a saturated NaHCO₃ solution (5 ml) and extracted with EtOAc. The separated organic phase was washed with a pH 3 buffer solution, dried (Na₂SO₄), filtered and concentrated under vacuum to give a residue. The residue was purified by KP-NH chromatography (Biotage SP1, 12M NH column) eluting in gradient with 40%-100% EtOAc\cyclohexane to afford the title compound (4.6 mg, 9.92 μmol, 50%).

### Example 1-33

### (Trans)-3-(2-pyridinyl)-8-({[5-(1-pyrrolidinylcarbonyl)-2-pyridinyl]amino}methyl)-1-oxa-3-azaspiro[4.5]decan-2-one Hydrochloride

(Trans)-8-(aminomethyl)-3-(2-pyridinyl)-1-oxa-3-azaspiro[4.5]decan-2-one (prepared in a similar fashion to the Intermediate 19 but purifying the crude by KP-NH chromatography with 0%-50% MeOHEtOAc as eluent, 40.2 mg, 0.154 mmol) was dissolved in sulfolane (2 ml) and TEA (0.043 ml, 0.308 mmol) and 2-fluoro-5-(1-pyrrolidinylcarbonyl)pyridine (Intermediate 86, 29.9 mg, 0.154 mmol) were added. The resulting mixture was irradiated at 150° C. for 30 min (three cycles), then at 150° C. for 1 hour, at 170° C. (1 hour) and finally at 200° C. (1 hour). The reaction mixture was loaded onto SCX cartridge, washed with MeOH and eluted with 2.0M NH₃ in MeOH to give a crude which was purified by KP-NH chromatography (Biotage SP1, 25M NH column) eluting in gradient with 50%-100% EtOAc\cyclohexane to afford trans-3-(2-pyridinyl)-8-({[5-(1-pyrrolidinylcarbonyl)-2-pyridinyl]amino}methyl)-1-oxa-3-azaspiro[4.5]decan-2-one (21.9 mg, 0.050 mmol, 33%).

¹H NMR (400 MHz, CDCl₃): δ 1.12-1.32 (m, 3H), 1.60-2.02 (m, 10H), 3.23-3.33 (m, 2H), 3.55-3.69 (m, 3H), 4.02-4.09 (m, 2H), 4.85-4.96 (m, 1H), 6.36-6.42 (m, 1H), 7.01-7.11 (m, 1H), 7.67-7.74 (m, 2H), 8.22-8.28 (m, 1H), 8.31-8.38 (m, 2H); UPLC-MS: 0.50 min, 436 [M+H]+.

(Trans)-3-(2-pyridinyl)-8-({[5-(1-pyrrolidinylcarbonyl)-2-pyridinyl]amino}methyl)-1-oxa-3-azaspiro[4.5]decan-2-one (50.1 mg, 0.115 mmol; two combined batches: the above one and a batch prepared in a similar fashion) was dissolved in DCMMeOH (2 ml2 ml)) and treated with 1.0M HCl in Et₂O (0.161 ml, 0.161 mmol) for 30 min under stirring. Solvent was evaporated under reduced pressure and the residue was triturated with acetone (3×1 ml) to give a white solid which was dried in vacuo at 60° C. overnight to afford the title compound (49.3 mg, 0.104 mmol, 91% yield).

Alternatively, (trans)-3-(2-pyridinyl)-8-({[5-(1-pyrrolidinylcarbonyl)-2-pyridinyl]amino}-methyl)-1-oxa-3-azaspiro[4.5]decan-2-one can be prepared in a similar fashion to the Example 1-32 replacing cyclohexylamine with pyrrolidine (1.654 μl, 0.02 mmol) to afford the desired compound (4.8 mg, 0.011 mmol, 55%).

### Example 1-34

### (Trans)-8-{[(3-fluoro-2,3′-bipyridin-6′-yl)amino]-methyl}-3-(2-fluoro-3-pyridinyl)-1-oxa-3-azaspiro[4.5]decan-2-one.hydrochloride

In a microwave vial copper(I) iodide (26.7 mg, 0.140 mmol), (+/−)-trans-1,2-diaminocyclohexane (0.034 ml, 0.281 mmol) and potassium phosphate tribasic (149 mg, 0.701 mmol) were added to a solution of trans-8-{[(3-fluoro-2,3′-bipyridin-6′-yl)amino]methyl}-1-oxa-3-azaspiro[4.5]decan-2-one (Intermediate 88, 50 mg, 0.140 mmol) and 2-fluoro-3-iodopyridine (37.5 mg, 0.168 mmol) in 1,4-dioxane (2 ml) and the resulting mixture was irradiated at 80° C. for 20 min (two cycles) then at 100° C. for 30 min. The solvent was evaporated. Dichloromethane (5 ml) was added to the residue and this was filtered washing with more dichloromethane (2×1 ml). The organic solution was concentrated in vacuo to give a crude which was purified by KP-NH chromatography (Biotage SP1, 25M NH column) eluting in gradient with 30%-100% EtOAc/cyclohexane to give a residue. The residue was further purified by R^{P} flash-chromatography (Biotage SP1, C18 50 g SNAP cartridge) eluting with a gradient of ACN and water (made-up with 0.1% HCOOH). Fractions containing the required compound were collected and passed through a SCX cartridge (5g, Varian) eluting with 2M ammonia in MeOH to afford trans-8-{[(3-fluoro-2,3′-bipyridin-6′-yl)amino]methyl}-3-(2-fluoro-3-pyridinyl)-1-oxa-3-azaspiro[4.5]decan-2-one (7.8 mg, 0.017 mmol, 12%).

¹H NMR (400 MHz, MeOD-d₄): δ 1.24-1.33 (m, 2H), 1.75-1.91 (m, 3H), 2.01 (br s, 2H), 2.14 (d, 2H), 3.31-3.35 (m, 3H), 3.94-4.01 (m, 2H), 6.68-6.74 (m, 1H), 7.32-7.43 (m, 2H), 7.67 (ddd, 1H), 8.03-8.09 (m, 1H), 8.09-8.17 (m, 2H), 8.43-8.46 (m, 1H), 8.53-8.56 (m, 1H); UPLC-MS: 0.52 min, 452 [M+H]+.

A DCM\MeOH solution (1.0\0.2 ml) of the above compound (7.8 mg, 0.017 mmol) was treated with 1.0M HCl in diethyl ether (0.024 ml, 0.024 mmol) for 30 minutes under stirring. Solvent was removed under reduced pressure and the residue was triturated with acetone (3×1 ml) to give a white solid which was dried under vacuum at 60° C. overnight to afford the title compound (7 mg, 0.014 mmol, 83%).

### Example 1-35

### (Trans)-8-({[5-(1-piperidinylcarbonyl)-2-pyridinyl]amino}methyl)-3-(2-pyridinyl)-1-oxa-3-azaspiro[4.5]decan-2-one.hydrochloride

In a microwave vial (trans)-8-(aminomethyl)-3-(2-pyridinyl)-1-oxa-3-azaspiro[4.5]decan-2-one (prepared in a similar fashion to the Intermediate 19 but purifying the crude by KP-NH chromatography with 0%-50% MeOHEtOAc as eluent, 80 mg, 0.306 mmol) was dissolved in sulfolane (2 ml) and TEA (0.085 ml, 0.612 mmol), 2-fluoro-5-(1-piperidinylcarbonyl)pyridine (Intermediate 89 63.7 mg, 0.306 mmol) were added and the mixture was irradiated at 200° C. for 1 hour. The reaction mixture was loaded onto a SCX cartridge (20g), washed with MeOH and eluted with 2.0M NH₃ in MeOH to give a crude which was purified by KP-NH chromatography eluting in gradient with 50%-100% EtOAc\cyclohexane (Biotage SP1, 25M NH column) to afford trans-8-({[5-(1-piperidinylcarbonyl)-2-pyridinyl]amino}methyl)-3-(2-pyridinyl)-1-oxa-3-azaspiro[4.5]decan-2-one (68.3 mg, 0.152 mmol, 49.6%).

¹H NMR (400 MHz, CDCl₃): δ 1.07-1.32 (m, 3H), 1.51-1.75 (m, 6H), 1.75-1.90 (m, 2H), 1.90-2.08 (m, 5H), 3.25 (t, 2H), 3.44-3.70 (m, 3H), 4.01-4.06 (m, 2H), 4.95 (t, 1H), 6.35-6.43 (m, 1H), 7.01-7.08 (m, 1H), 7.53 (dd, 1H), 7.67-7.75 (m, 1H), 8.17-8.21 (m, 1H), 8.24-8.29 (m, 1H), 8.31-8.35 (m, 1H); UPLC-MS: 0.53 min, 450 [M+H]+.

To a mixture of the above compound (68.3 mg, 0.152 mmol) in DCM (1 ml) 1.0M HCl in diethyl ether (0.213 ml, 0.213 mmol) and methanol (0.2 ml) were added. The resulting solution was stirred for 30 minutes and then evaporated under reduced pressure. The residue was triturated with acetone (3×1 ml) to give a white solid which was dried under vacuum at 60° C. overnight to afford the title compound (73 mg, 0.150 mmol, 99%).

### Example 1-36

### (Trans)-3-(3-pyridazinyl)-8-({[6-(trifluoromethyl)-2,3′-bipyridin-6′-yl]amino}methyl)-1-oxa-3-azaspiro[4.5]decan-2-one.hydrochloride

The title compound was made in a similar fashion to the preparation of Example 1-35 replacing (trans)-8-(aminomethyl)-3-(2-pyridinyl)-1-oxa-3-azaspiro[4.5]decan-2-one with (trans)-8-(aminomethyl)-3-(3-pyridazinyl)-1-oxa-3-azaspiro[4.5]decan-2-one (prepared in a similar fashion to Intermediate 41 procedure 41b, 50 mg, 0.191 mmol) and 2-fluoro-5-(1-piperidinylcarbonyl)pyridine with 6′-fluoro-6-(trifluoromethyl)-2,3′-bipyridine (Intermediate 90, 46.2 mg, 0.191 mmol) to afford the title compound (31.5 mg, 0.060 mmol, 90%).

### Example 1-37

### (Trans)-3-(3-pyridazinyl)-8-[({5-[2-(trifluoromethyl)phenyl]-2-pyridinyl}amino)methyl]-1-oxa-3-azaspiro[4.5]decan-2-one.hydrochloride

The title compound was made in a similar fashion to the preparation of Example 1-35 replacing (trans)-8-(aminomethyl)-3-(2-pyridinyl)-1-oxa-3-azaspiro[4.5]decan-2-one with (trans)-8-(aminomethyl)-3-(3-pyridazinyl)-1-oxa-3-azaspiro[4.5]decan-2-one (prepared in a similar fashion to the Intermediate 41 procedure 41b, 50 mg, 0.191 mmol) and 2-fluoro-5-(1-piperidinylcarbonyl)pyridine with 2-fluoro-5-[2-(trifluoromethyl)phenyl]pyridine (Intermediate 91, 46.0 mg, 0.191 mmol) to afford the title compound (11.5 mg, 0.022 mmol, 68%).

### Example 1-38

### (Trans)-3-(3-pyridazinyl)-8-({[5-(2-pyridinyloxy)-2-pyridinyl]amino}methyl)-1-oxa-3-azaspiro[4.5]decan-2-one.hydrochloride

5-(2-Pyridinyloxy)-2-pyridinamine (Intermediate 92, 35 mg, 0.187 mmol) and (trans)-2-oxo-3-(3-pyridazinyl)-1-oxa-3-azaspiro[4.5]decane-8-carbaldehyde (prepared in a similar fashion to Intermediate 60 but purifying the crude by silica gel chromatography on a 50g SNAP Biotage column eluting in gradient with 60%-100% EtOAC\cyclohexane then 100% EtOAc, 48.9 mg, 0.187 mmol) were dissolved in anhydrous DCM (2 ml) and titanium(IV) isopropoxide (0.164 ml, 0.561 mmol) was added. The reaction mixture was stirred at r.t. overnight then sodium borohydride (21.22 mg, 0.561 mmol) and ethanol (2 ml) were added and stirring for 2 hours. The reaction mixture was poured into a saturated NaHCO₃ solution (5 ml) and extracted with DCM. Combined organic extracts were concentrated under vacuum to give a crude which was purified by KP-NH chromatography (25M column) eluting in gradient with 0%-50% MeOHEtOAc to afford (trans)-3-(3-pyridazinyl)-8-({[5-(2-pyridinyloxy)-2-pyridinyl]amino}methyl)-1-oxa-3-azaspiro[4.5]decan-2-one (79 mg, 0.183 mmol, 98%).

¹H NMR (400 MHz, CDCl₃): δ 1.19-1.24 (m, 2H), 1.71-2.02 (m, 7H), 3.19-3.24 (m, 2H), 4.20 (s, 2H), 4.68 (t, 1H), 6.39-6.47 (m, 1H), 6.87-6.93 (m, 1H), 6.95-7.02 (m, 1H), 7.25-7.32 (m, 1H), 7.45-7.53 (m, 1H), 7.62-7.72 (m, 1H), 7.95-7.99 (m, 1H), 8.12-8.18 (m, 1H), 8.51-8.57 (m, 1H), 8.91-8.97 (m, 1H); UPLC-MS: 0.49 min, 433 [M+H]+.

A solution of the above compound (79 mg, 0.183 mmol) in DCM (2 ml) was treated with a 1.0M HCl in diethyl ether (0.256 ml, 0.256 mmol) for 30 minutes under stirring then evaporated under reduced pressure. The resulting residue was triturated with acetone to give a white solid which was dried under vacuum at 60° C. overnight to afford the title compound (76 mg, 0.162 mmol, 89%).

### Example 1-39

### (Trans)-8-{[(4-phenyl-2-pyridinyl)amino]methyl}-3-(3-pyridazinyl)-1-oxa-3-azaspiro[4.5]decan-2-one hydrochloride

The title compound was made in a similar fashion to the preparation of Example 1-38 replacing 5-(2-pyridinyloxy)-2-pyridinamine with 4-phenyl-2-pyridinamine (Intermediate 93, 35.8 mg, 0.210 mmol) to afford the title compound (44 mg, 0.097 mmol, 89%).

### Example 1-40

### (Trans)-8-[(2,3′-bipyridin-6′-ylamino)methyl]-3-(3-pyridazinyl)-1-oxa-3-azaspiro[4.5]decan-2-one

2,3′-Bipyridin-6′-amine (Intermediate 99, 34 mg, 0.199 mmol) was dissolved in anhydrous DCM (2 ml). (Trans)-2-oxo-3-(3-pyridazinyl)-1-oxa-3-azaspiro[4.5]decane-8-carbaldehyde (prepared in a similar fashion to the Intermediate 60, 57.1 mg, 0.218 mmol) and titanium(IV) isopropoxide (0.175 ml, 0.596 mmol) were added and the resulting mixture was shaken at room temperature for 23 hours. Additional titanium(IV) isopropoxide (0.100 ml) was added to the mixture and shaken for further 2 hours. Ethanol (1 ml) and sodium borohydride (23 mg, 0.596 mmol) were added and the mixture was shaken 3 hours, then diluted with DCM and treated with saturated NaHCO₃ solution (1 ml). The organic phase was separated through phase separator tube and the aqueous phase was back-extracted with DCM. Combined organic extracts were dried (Na₂SO₄), filtered through filter tube and concentrated under nitrogen flow to obtain a residue which was purified by RP-HPLC chromatography to afford the title compound as a white solid (27.1 mg).

### Example 1-41

### (Trans)-8-{[(5-methyl-2,3′-bipyridin-6′-yl)amino]methyl}-3-(3-pyridazinyl)-1-oxa-3-azaspiro[4.5]decan-2-one

The title compound was made in a similar fashion to the preparation of Example 1-40 replacing 2,3′-bipyridin-6′-amine with 5-methyl-2,3′-bipyridin-6′-amine (Intermediate 100, 41 mg, 0.221 mmol) to afford the title compound as a white solid (34.4 mg).

### Example 1-42

### (Trans)-8-{[(6-fluoro-2,3′-bipyridin-6′-yl)amino]-methyl}-3-(3-pyridazinyl)-1-oxa-3-azaspiro[4.5]decan-2-one

The title compound was made in a similar fashion to the preparation of Example 1-40 replacing 2,3′-bipyridin-6′-amine with 6-fluoro-2,3′-bipyridin-6′-amine (Intermediate 101, 48 mg, 0.254 mmol) and purifying with RP-HPLC followed by flash-chromatography on KP-NH column using a gradient 30%-90% ethyl acetate\cyclohexane to afford the title compound as a white solid (18 mg).

### Example 1-43

### (Trans)-8-{[(3-fluoro-6-methyl-2,3′-bipyridin-6′-yl)amino]methyl}-3-(3-pyridazinyl)-1-oxa-3-azaspiro[4.5]decan-2-one

The title compound was made in a similar fashion to the preparation of Example 1-42 replacing 6-fluoro-2,3′-bipyridin-6′-amine with 3-fluoro-6-methyl-2,3′-bipyridin-6′-amine (Intermediate 102, mg, 0.172 mmol) to afford the title compound as a white solid (14.6 mg).

### Example 1-44

### (Trans)-8-{[(5-fluoro-2,3′-bipyridin-6′-yl)amino]methyl}-3-(3-pyridazinyl)-1-oxa-3-azaspiro[4.5]decan-2-one

The title compound was made in a similar fashion to the preparation of Example 1-42 replacing 6-fluoro-2,3′-bipyridin-6′-amine with 5-fluoro-2,3′-bipyridin-6′-amine (Intermediate 103, 34 mg, 0.18 mmol) to afford the title compound as a white solid (13.6 mg).

### Example 1-45

### (Trans)-3-(3-pyridazinyl)-8-{[(5-{3-[(trifluoromethyl)oxy]phenyl}-2-pyridinyl)amino]methyl}-1-oxa-3-azaspiro[4.5]decan-2-one

The title compound was made in a similar fashion to the preparation of Example 1-42 replacing

### 6-fluoro-2,3′-bipyridin-6′-amine with 5-{3-[(trifluoromethyl)oxy]phenyl}-2-pyridinamine (Intermediate 104, 87 mg, 0.342 mmol) to afford the title compound as a white solid (12 mg).

### Example 1-46

### (Trans)-8-({[5-(2-fluorophenyl)-2-pyridinyl]amino}methyl)-3-(3-pyridazinyl)-1-oxa-3-azaspiro[4.5]decan-2-one

The title compound was made in a similar fashion to the preparation of Example 1-42 replacing 6-fluoro-2,3′-bipyridin-6′-amine with 5-(2-fluorophenyl)-2-pyridinamine (Intermediate 105, 62 mg, 0.329 mmol) to afford the title compound as a white solid (7.9 mg).

### Example 1-47

### (Trans)-8-[(3,3′-bipyridin-6-ylamino)methyl]-3-(3-pyridazinyl)-1-oxa-3-azaspiro[4.5]decan-2-one

The title compound was made in a similar fashion to the preparation of Example 1-42 replacing 6-fluoro-2,3′-bipyridin-6′-amine with 3,3′-bipyridin-6-amine (Intermediate 106, 77 mg, 0.450 mmol) to afford the title compound as a white solid (18.3 mg).

### Example 1-48

### (Trans)-8-{[(6′-fluoro-3,3′-bipyridin-6-yl)amino]methyl}-3-(3-pyridazinyl)-1-oxa-3-azaspiro[4.5]decan-2-one

The title compound was made in a similar fashion to the preparation of Example 1-42 replacing 6-fluoro-2,3′-bipyridin-6′-amine with 6′-fluoro-3,3′-bipyridin-6-amine (Intermediate 107, 83 mg, 0.439 mmol) to afford the title compound as a white solid (13.7 mg).

### Example 1-49

### (Trans)-8-{[(2′-fluoro-3,3′-bipyridin-6-yl)amino]methyl}-3-(3-pyridazinyl)-1-oxa-3-azaspiro[4.5]decan-2-one

The title compound was made in a similar fashion to the preparation of Example 1-42 replacing 6-fluoro-2,3′-bipyridin-6′-amine with 2′-fluoro-3,3′-bipyridin-6-amine (Intermediate 108, 61 mg, 0.322 mmol) to afford the title compound as a white solid (15 mg).

### Example 1-50

### (Trans)-8-{[(3-fluoro-2,3′-bipyridin-6′-yl)amino]methyl}-3-(3-pyridazinyl)-1-oxa-3-azaspiro[4.5]decan-2-one hydrochloride

To a solution of 3-fluoro-2,3′-bipyridin-6′-amine (Intermediate 109, 56 mg, 0.296 mmol) in dry 1,2-dichloroethane (2 ml), (trans)-2-oxo-3-(3-pyridazinyl)-1-oxa-3-azaspiro[4.5]decane-8-carbaldehyde (prepared in a similar fashion to Intermediate 60, 70.3 mg, 0.269 mmol) and titanium(IV) isopropoxide (0.118 ml, 0.404 mmol) were added followed by isopropanol (0.4 ml). The resulting solution was shaken at room temperature overnight then sodium borohydride (30.5 mg, 0.807 mmol) and ethanol (2 ml) were added and the mixture was shaken at room temperature 2 hours. The mixture was diluted with DCM and quenched with a saturated NaHCO₃ solution; the organic phase was separated through IST phase separator tube and concentrated in vacuo to give a residue. The crude was purified by flash chromatography on KP-NH column (Biotage 12+M column) eluting in gradient with 0%-80% EtOAc\cyclohexane (in 15 cv) to afford (trans)-8-{[(3-fluoro-2,3′-bipyridin-6′-yl)amino]methyl}-3-(3-pyridazinyl)-1-oxa-3-azaspiro[4.5]decan-2-one as a white solid (22 mg, 0.051 mmol, 19%).

¹H NMR (400 MHz, CDCl₃): δ 1.19-1.38 (m, 2H), 1.75-1.99 (m, 3H), 2.01-2.14 (m, 4H), 3.34 (t, 2H), 4.24 (s, 2H), 4.95 (br s, 1H), 6.53 (d, 1H), 7.17-7.25 (m, 1H), 7.41-7.56 (m, 2H), 8.11-8.20 (m, 1H), 8.45-8.52 (m, 1H), 8.55-8.63 (m, 1H), 8.76-8.82 (m, 1H), 8.95-9.00 (m, 1H); UPLC-MS: 0.50 min, 435 [M+H]+.

To a solution of the above compound (22 mg, 0.051 mmol) in DCM (1 ml) 1.0M HCl in Et₂O (0.061 mmol, 0.061 ml) was added and the resulting solution was shaken at room temperature 15 min, then concentrated in vacuo to give a solid which was triturated with Et₂O, filtrated and dried under vacuum at 40° C. overnight to afford the title compound as a white solid (22 mg).

Alternatively (trans)-8-{[(3-fluoro-2,3′-bipyridin-6′-yl)amino]methyl}-3-(3-pyridazinyl)-1-oxa-3-azaspiro[4.5]decan-2-one can be prepared in a similar fashion to the preparation of Example 1-42 replacing 6-fluoro-2,3′-bipyridin-6′-amine with 3-fluoro-2,3′-bipyridin-6′-amine (prepared in a similar fashion to the Intermediate 109 but performing a SCX cartridge before purification, 51 mg, 0.27 mmol) to afford the desired compound as a white solid (8.2 mg).

### Example 1-51

### (Trans)-8-{[(6-methyl-2,3′-bipyridin-6′-yl)amino]methyl}-3-(3-pyridazinyl)-1-oxa-3-azaspiro[4.5]decan-2-one Dihydrochloride

PdCl₂(dppf)-CH₂Cl₂ adduct (64.4 mg, 0.079 mmol) and aq 2.0M sodium carbonate (0.394 ml, 0.788 mmol) were added to a mixture of 2-bromo-6-methylpyridine (81 mg, 0.473 mmol) and [6-({[(trans)-2-oxo-3-(3-pyridazinyl)-1-oxa-3-azaspiro[4.5]dec-8-yl]methyl}amino)-3-pyridinyl]boronic acid (Intermediate 111, 151 mg, 0.394 mmol) in dry DMF (3 ml) and the resulting mixture was shaken at 80° C. overnight. Then an additional amount of aq 2.0M sodium carbonate (0.394 ml, 0.788 mmol), PdCl₂(dppf)-CH₂Cl₂ adduct (64.4 mg, 0.079 mmol) and 2-bromo-6-methylpyridine (81 mg, 0.473 mmol) was added and the mixture was shaken at 80° C. for further 6 hours. The mixture was filtered through IST filter tube and concentrated under vacuum to give a residue which was dissolved in a mixture of MeOH and DMC and loaded into SCX cartridge (5g), washed with MeOH and eluted with 2.0M NH₃ in MeOH to give a crude. The crude was purified by RP-flash chromatography (SNAP C18 60 g column) eluting in gradient with 0% phase B (acetonitrile), 0%-50% phase B\phase A (H₂O5% acetonitrile\0.1% HCO₂H) in 10 cv, 50%-95% phase B\phase A in 6 cv, 95%-0% phase B in 2 cv. A yellow oil was recovered which was purified by flash chromatography on KP-NH 12+M column eluting in gradient with 50%-100% EtOAc\cyclohexane (in 15 cv) then 100% EtOAc (5 cv) to afford (trans)-8-{[(6-methyl-2,3′-bipyridin-6′-yl)amino]methyl}-3-(3-pyridazinyl)-1-oxa-3-azaspiro[4.5]decan-2-one as a white solid (49 mg, 0.114 mmol, 29%).

¹H NMR (400 MHz, CDCl₃): δ 1.17-1.39 (m, 2H), 1.72-1.99 (m, 3H), 2.07 (s, 4H), 2.61 (s, 3H), 3.31 (t, 2H), 4.24 (s, 2H), 4.83 (t, 1H), 6.50 (d, 1H), 7.05 (d, 1H), 7.40-7.46 (m, 1H), 7.48-7.54 (m, 1H), 7.61 (t, 1H), 8.15 (dd, 1H), 8.58 (dd, 1H), 8.66-8.70 (m, 1H), 8.97 (dd, 1H); UPLC-MS: 0.76 min, mass 431 [M+H]+.

The above compound (49 mg, 0.114 mmol) was dissolved in DCMMeOH (20.5 ml) and treated with 1.0M HCl in Et₂O (0.251 ml, 0.251 mmol) by stirring for 15 minutes. Solvent was removed in vacuo and the resulting pale yellowish solid was dried under vacuum at 40° C. overnight to obtain the title compound as a pale yellow solid (54 mg, 0.107 mmol).

### Example 1-52

### 6′-({[(Trans)-2-oxo-3-(3-pyridazinyl)-1-oxa-3-azaspiro[4.5]dec-8-yl]methyl}amino)-2,3′-bipyridine-6-carbonitrile dihydrochloride

PdCl₂(dppf)-CH₂Cl₂ adduct (64.4 mg, 0.079 mmol) and aqueous 2.0M sodium carbonate (0.394 ml, 0.788 mmol) were added to a mixture of 6-bromo-2-pyridinecarbonitrile (87 mg, 0.473 mmol) and [6-({[(trans)-2-oxo-3-(3-pyridazinyl)-1-oxa-3-azaspiro[4.5]dec-8-yl]methyl}amino)-3-pyridinyl]boronic acid (Intermediate 111, 151 mg, 0.394 mmol) in dry DMF (3 ml) and the resulting mixture was shaken at 80° C. overnight. Additional 2.0M aq sodium carbonate (0.394 ml, 0.788 mmol), PdCl₂(dppf)-CH₂Cl₂ adduct (64.4 mg, 0.079 mmol) and 6-bromo-2-pyridinecarbonitrile (87 mg, 0.473 mmol) were added and the mixture was shaken at 80° C. for further 6 hours then it was cooled down, filtered through IST filter tube and concentrated in vacuo to give a brown residue. The residue was dissolved in MeOHDCM, loaded into SCX cartridge (5 g), washed with MeOH and eluted with 2.0M NH₃ in MeOH. After concentration in vacuo, the resulting crude was purified by RP-flash chromatography (SNAP C18 60 g column) eluting in gradient with 0% phase B (acetonitrile) (2 cv), 0%-50% phase B\phase A (H₂O+5% acetonitrile+0.1% HCO₂H) (in 12 cv), 50% phase B (lcv), 50%-95% phase B (in 6 cv), 95% phase B (1 cv) then 95%-0% phase B (in 2 cv) to afford a residue. The residue was loaded onto SCX cartridge, washed with methanol and eluted with 2.0M NH₃ in MeOH to obtain 6′-({[(trans)-2-oxo-3-(3-pyridazinyl)-1-oxa-3-azaspiro[4.5]dec-8-yl]methyl}amino)-2,3′-bipyridine-6-carbonitrile as a yellowish solid (46 mg, 0.104 mmol, 26%).

¹H NMR (400 MHz, CDCl₃): δ 1.18-1.38 (m, 2H), 1.72-1.99 (m, 3H), 1.99-2.12 (m, 4H), 3.29-3.40 (t, 2H), 4.23 (s, 2H), 4.86-4.99 (m, 1H), 6.48-6.57 (d, 1H), 7.47-7.59 (m, 2H), 7.77-7.91 (m, 2H), 8.15-8.25 (m, 1H), 8.54-8.62 (m, 1H), 8.69-8.76 (m, 1H), 8.92-9.01 (m, 1H); UPLC\MS: 0.75 min, mass 442 [M+H]+.

The above compound (46 mg, 0.104 mmol) was dissolved in acetone\MeOH (2.0\0.5 ml) and treated with 1.0M HCl in Et₂O (0.229 ml, 0.229 mmol) by stirring for 15 min. Solvent was removed under nitrogen flow and the resulting solid was dried under vacuum at 40° C. overnight to afford the title compound (48 mg).

### Example 1-53

### (Trans)-3-(3-pyridazinyl)-8-({[5-(3-pyridazinyl)-2-pyridinyl]amino}methyl)-1-oxa-3-azaspiro[4.5]decan-2-one hydrochloride

In a 8 ml vial, PdCl₂(dppf)-CH₂Cl₂ adduct (64.4 mg, 0.079 mmol) and 2.0M aq sodium carbonate (0.394 ml, 0.788 mmol) were added to a mixture of 3-chloropyridazine (54.2 mg, 0.473 mmol) and [6-({[(trans)-2-oxo-3-(3-pyridazinyl)-1-oxa-3-azaspiro[4.5]dec-8-yl]methyl}amino)-3-pyridinyl]boronic acid (Intermediate 111, 151 mg, 0.394 mmol) in dry DMF (3 ml) and the resulting mixture was shaken at 80° C. overnight. Additional 2.0M aq sodium carbonate (0.394 ml, 0.788 mmol), PdCl₂(dppf)-CH₂Cl₂ adduct (64.4 mg, 0.079 mmol) and 3-chloropyridazine (54.2 mg, 0.473 mmol) were added and the mixture was shaken at 80° C. for further 6 hours. The mixture was cooled down, filtered through a filter tube and concentrated in vacuo (Biotage V10-evaporator system) to give a brown residue. The residue was dissolved into MeOH and DCM and loaded onto SCX cartridge (5 g), washed with MeOH and eluted with 2.0M NH₃ in MeOH. After concentration in vacuo of ammonia\methanolic solution a brown residue was obtained. The crude was partially purified by RP-flash chromatography (Biotage SP1, SNAP C18 60 g column) eluting in gradient with 0%-40% phase B (acetonitrile)\phase A (H₂O+5% acetonitrile+0.1% HCO₂H) (in 15 cv) then 40%-95% phase B\phase A (in 6 cv), 95% phase B (2 cv) and 95%-0% phase B (in 2 cv) to recover a yellow solid. The residue was purified by flash chromatography on KP-NH column (12+M Biotage) eluting in gradient with 70%-100% EtOAc\cyclohexane (in 12 cv) then 100% EtOAc (5 cv) to recover (trans)-3-(3-pyridazinyl)-8-({[5-(3-pyridazinyl)-2-pyridinyl]amino}methyl)-1-oxa-3-azaspiro[4.5]decan-2-one as a white solid (31.4 mg, 0.075 mmol, 19%).

¹H NMR (400 MHz, CDCl₃): δ 1.20-1.37 (m, 2H), 1.85-1.99 (m, 2H), 2.00-2.13 (m, 4H), 3.36 (t, 2H), 4.24 (s, 2H), 4.93-5.03 (m, 1H), 6.57 (d, 1H), 7.48-7.56 (m, 2H), 7.80 (dd, 1H), 8.34 (dd, 1H), 8.59 (dd, 1H), 8.74 (d, 1H), 8.96-8.99 (m, 1H), 9.11 (dd, 1H); UPLC\MS: 0.59 min, 418 [M+H]+.

The above compound (31.4 mg, 0.075 mmol) was dissolved in acetone (2 ml) and treated with 1.0M HCl in Et₂O (0.099 ml, 0.099 mmol) for 15 minutes under stirring. Solvent was removed under nitrogen flow then solid was dried in vacuo overnight at 40° C. to afford the title compound as a white solid (30 mg).

### Example 1-54

### 3-[6-({[(Trans)-2-oxo-3-(3-pyridazinyl)-1-oxa-3-azaspiro[4.5]dec-8-yl]methyl}amino)-3-pyridinyl]benzonitrile Dihydrochloride

A mixture of 3-(6-fluoro-3-pyridinyl)benzonitrile (Intermediate 110, 29.5 mg, 0.149 mmol), (trans)-8-(aminomethyl)-3-(3-pyridazinyl)-1-oxa-3-azaspiro[4.5]decan-2-one (prepared in a similar fashion to the Intermediate 41 procedure 41b but adding triphenylphosphine at 0° C. and stirring the mixture at 0° C. for 3 hours, 30 mg, 0.114 mmol) and DIPEA (0.060 ml, 0.343 mmol) in sulfolane (1 ml) was heated to 120° C. for 1 hour, then to 150° C. for 46 hours. This was cooled and loaded onto a pre-conditioned SCX cartridge (3g), then eluted with MeOH and then 2M NH₃ in MeOH. The basic fractions were evaporated to give a brown gummy residue which was purified via Biotage (50%-100% EtOAc/cyclohexane; 12M NH column) to give 15 mg of a colourless gum. ¹H NMR and UPLC show desired product contaminated with ˜5% (trans)-8-(aminomethyl)-3-(3-pyridazinyl)-1-oxa-3-azaspiro[4.5]decan-2-one. This was dissolved in dichloromethane (10 ml), washed with pH3 buffer solution (10 ml) and filtered through a hydrophobic frit (Phase Separator cartridge). This was evaporated to give 14 mg of 3-[6-({[(trans)-2-oxo-3-(3-pyridazinyl)-1-oxa-3-azaspiro[4.5]dec-8-yl]methyl}amino)-3-pyridinyl]benzonitrile as a colourless gum.

¹H NMR (400 MHz, CDCl₃): δ 8.97 (dd, 1H), 8.58 (dd, 1H), 8.34 (d, 1H), 7.78 (s, 1H), 7.74 (dt, 1H), 7.66 (dd, 1H), 7.57-7.63 (m, 1H), 7.48-7.56 (m, 2H), 6.52 (d, 1H), 4.87 (t, 1H), 4.24 (s, 2H), 3.32 (t, 2H), 1.75-2.12 (m, 7H), 1.20-1.35 (m, 2H); UPLC-MS: 0.52 min, 441 [M+H]+ and 221 [M+2H]2+.

The above product was dissolved in dichloromethane and treated with 1.0M HCl in diethyl ether (0.1 ml, 0.1 mmol). It was stirred for 5 minutes then evaporated under reduced pressure. The residue was triturated with diethyl ether then filtered washing the solids with diethyl ether. The solid residue was dried under reduced pressure at 50° C. overnight (˜18 hours) then under high vacuum at 50° C. for 6 hours to give the title compound (13 mg) as an off white solid.

### Example 1-55

### (Trans)-3-(3-pyridinyl)-8-({[6-(trifluoromethyl)-3-pyridinyl]amino}methyl)-1-oxa-3-azaspiro[4.5]decan-2-one Dihydrochloride

The title compound was made in a similar fashion to the preparation of Example 1-25 replacing 6-(trifluoromethyl)-2-pyridinamine with 6-(trifluoromethyl)-3-pyridinamine (can be commercially available e.g from Aldrich, 24.91 mg, 0.154 mmol) to afford the title compound as a colourless solid (20 mg).

### Example 1-56

### (Trans)-3-(3-pyridinyl)-8-({[5-(trifluoromethyl)-3-pyridinyl]amino}methyl)-1-oxa-3-azaspiro[4.5]decan-2-one dihydrochloride

The title compound was made in a similar fashion to the preparation of Example 1-25 replacing 6-(trifluoromethyl)-2-pyridinamine with 5-(trifluoromethyl)-3-pyridinamine (24.91 mg, 0.154 mmol) to afford the title compound as a colourless solid (16 mg).

### Example 1-57

### (Trans)-8-({[6-(4-morpholinyl)-3-pyridinyl]amino}methyl)-3-(2-pyridinyl)-1-oxa-3-azaspiro[4.5]decan-2-one.dihydrochloride

To a solution of (trans)-8-({[6-(4-morpholinyl)-3-pyridinyl]amino}methyl)-1-oxa-3-azaspiro[4.5]decan-2-one (Intermediate 94, 49.8 mg, 0.144 mmol) and 2-iodopyridine (29.5 mg, 0.144 mmol) in 1,4-dioxane (3 ml) copper(I) iodide (27.4 mg, 0.144 mmol), (+/−)-trans-1,2-diaminocyclohexane (0.035 ml, 0.288 mmol) and potassium phosphate (153 mg, 0.719 mmol) were added and the mixture was stirred at 120° C. for 8 hours. Solvent was evaporated and the residue was dissolved in EtOAc (50 ml) and washed with a pH3 solution (10 ml). The organic phase was dried (Na₂SO₄) and concentrated under vacuum to give a crude which was purified on KP-NH cartridge eluting in gradient with 10%-100% EtOAc\cyclohexane to afford (trans)-8-({[6-(4-morpholinyl)-3-pyridinyl]amino}-methyl)-3-(2-pyridinyl)-1-oxa-3-azaspiro[4.5]decan-2-one (20.2 mg, 0.048 mmol, 33%).

¹H NMR (400 MHz, CDCl₃): δ 1.59-1.90 (m, 5H), 1.96-2.07 (m, 4H), 2.96-3.07 (m, 2H), 3.29-3.43 (m, 5H), 3.82-3.88 (m, 4H), 4.02-4.11 (m, 2H), 6.56-6.65 (m, 1H), 6.94-6.99 (m, 1H), 7.04-7.09 (m, 1H), 7.69-7.78 (m, 2H), 8.27-8.31 (m, 1H), 8.31-8.38 (m, 1H); UPLC-MS: 0.56 min, 424 [M+H]+.

To a solution of the above compound (20.2 mg, 0.048 mol) in DCM (2 ml) 1.0M HCl in Et₂O (0.105 ml, 0.105 mmol) was added and the solution was stirred at r.t for 30 min. The precipitate was separated, triturated with Et₂O (2 ml) and dried for 6 hours under high vacuum at 40° C. to give the title compound (18.1 mg, 0.036 mmol, 76%).

### Example 1-58

### (Trans)-8-({[6-(2-fluorophenyl)-3-pyridinyl]amino}methyl)-3-(2-pyridinyl)-1-oxa-3-azaspiro[4.5]decan-2-one.hydrochloride

To a solution of (trans)-8-({[6-(2-fluorophenyl)-3-pyridinyl]amino}methyl)-1-oxa-3-azaspiro-[4.5]decan-2-one (Intermediate 96, 0.031 ml, 0.169 mmol) and 2-iodopyridine (34.6 mg, 0.169 mmol) in 1,4-dioxane (2 ml) copper(I) iodide (32.2 mg, 0.169 mmol), (+/−)-trans-1,2-diaminocyclohexane (0.041 ml, 0.338 mmol) and potassium phosphate (179 mg, 0.844 mmol) were added and the mixture was irradiated in microwave system at 80° C. for 30 min then at 100° C. (30 min, 2 cycles) and then allowed to stir at 100° C. overnight under normal heating. The solvent was evaporated. Dichloromethane (5 ml) was added to the residue which was then filtered washing with more dichloromethane (2×10 ml). After concentration in vacuo of organic solution, the crude was purified by KP-NH chromatography (Biotage 25M NH column) eluting in gradient with 30%-100% EtOAc/cyclohexane to give (trans)-8-({[6-(2-fluorophenyl)-3-pyridinyl]amino}methyl)-3-(2-pyridinyl)-1-oxa-3-azaspiro[4.5]decan-2-one (48.3 mg, 0.112 mmol, 66%).

¹H NMR (400 MHz, CDCl₃): δ 1.21-1.31 (m, 2H), 1.67-1.92 (m, 4H), 1.98-2.07 (m, 3H), 3.10 (t, 2H), 3.95-4.01 (m, 1H), 4.07 (s, 2H), 6.92-6.97 (m, 1H), 7.03-7.16 (m, 2H), 7.20-7.33 (m, 2H), 7.62-7.67 (m, 1H), 7.70-7.76 (m, 1H), 7.93 (td, 1H), 8.13-8.17 (m, 1H), 8.28 (dt, 1H), 8.34 (m, 1H); UPLC-MS: 0.62 min, 433 [M+H]+.

The above compound (48.3 mg, 0.112 mmol) was dissolved in 1:1 DCM\MeOH (2 ml2 ml) and treated with 1.0M HCl in Et₂O (0.156 ml, 0.156 mmol) for 30 minutes under stirring. Solvent was evaporated under reduced pressure and the residue was triturated with acetone (3×1 ml) to give a white solid which was dried under vacuum at 60° C. overnight to afford the title compound (51 mg, 0.109 mmol, 97%).

### Example 1-59

### (Trans)-8-({[6-(2-fluorophenyl)-3-pyridinyl]amino}methyl)-3-(3-pyridazinyl)-1-oxa-3-azaspiro[4.5-]decan-2-one.hydrochloride

In a sealed tube to a solution of (trans)-8-({[6-(2-fluorophenyl)-3-pyridinyl]amino}methyl)-1-oxa-3-azaspiro[4.5]decan-2-one (Intermediate 96, 0.026 ml, 0.141 mmol) and 3-chloropyridazine (16.11 mg, 0.141 mmol) in 1,4-dioxane (2 ml), copper(I) iodide (26.8 mg, 0.141 mmol), (+/−)-trans-1,2-diaminocyclohexane (0.034 ml, 0.281 mmol) and potassium phosphate (149 mg, 0.703 mmol) were added. The mixture was stirred at 100° C. overnight. The solvent was evaporated. Dichloromethane (5 ml) was added to the residue which was then filtered washing with more dichloromethane (2×1 ml). The organic solution was concentrated in vacuo and the residue was purified by KP-NH chromatography (Biotage SP1, 25M NH column) eluting in gradient with 30%-100% EtOAc/cyclohexane to give (trans)-8-({[6-(2-fluorophenyl)-3-pyridinyl]amino}methyl)-3-(3-pyridazinyl)-1-oxa-3-azaspiro[4.5]decan-2-one (43 mg, 0.099 mmol, 70%).

¹H NMR (400 MHz, CDCl₃): δ 1.64-1.74 (m, 1H), 1.77-1.84 (m, 1H), 1.85-1.96 (m, 2H), 2.02-2.10 (m, 5H), 3.10-3.16 (m, 2H), 4.00 (t, 1H), 4.23 (s, 2H), 6.95 (dd, 1H), 7.09-7.16 (m, 1H), 7.20-7.33 (m, 2H), 7.47-7.55 (m, 1H), 7.62-7.71 (m, 1H). 7.90-7.96 (m, 1H), 8.12-8.20 (m, 1H), 8.53-8.61 (m, 1H), 8.93-9.00 (m, 1H); UPLC-MS: 0.63 min, 433 [M+H]+.

The above compound (43 mg, 0.099 mmol) was dissolved in 1:1 DCMMeOH (2 ml2 ml) and treated with 1.0M HCl in Et₂O (0.139 ml, 0.139 mmol) for 30 minutes under stirring. Solvent was evaporated under reduced pressure and the residue was triturated with acetone (3×1 ml) to give a white solid which was dried under vacuum at 60° C. overnight to afford the title compound (41 mg, 0.087 mmol, 88%).

### Example 1-60

### 5-({[(Trans)-2-oxo-3-(2-pyridinyl)-1-oxa-3-azaspiro[4.5]dec-8-yl]methyl}amino)-2-pyridinecarbonitrile Dihydrochloride

(Trans)-2-oxo-3-(2-pyridinyl)-1-oxa-3-azaspiro[4.5]decane-8-carbaldehyde (prepared in a similar fashion to the Intermediate 12 procedure 12a, 41 mg, 0.158 mmol) and 5-amino-2-pyridinecarbonitrile (18.76 mg, 0.158 mmol) were dissolved at r.t. in dry THF (3 ml) and titanium(IV) isopropoxide (0.092 ml, 0.315 mmol) was added and the reaction mixture was stirred overnight. Sodium borohydride (17.88 mg, 0.473 mmol) and ethanol (500 μl) were added and the mixture was allowed to stir, then it was poured into a saturated NaHCO₃ solution (5 ml), extracted with DCM (3×50 ml), filtered by a phase separator tube and then the organic phase was concentrated under vacuum to give a crude. The crude was purified by KP-NH chromatography (25M NH column) eluting in gradient with 0%-30% Et₂ODCM to give a residue (28 mg). The residue was purified again by silica gel chromatography (12M column) eluting with 100% EtOAc to afford 5-({[(trans)-2-oxo-3-(2-pyridinyl)-1-oxa-3-azaspiro[4.5]dec-8-yl]methyl}amino)-2-pyridinecarbonitrile (22 mg, 0.059 mmol, 38%).

¹H NMR (400 MHz, CDCl₃): δ 1.19-1.34 (m, 2H), 1.70-1.80 (m, 1H), 1.82-1.92 (m, 2H), 1.97-2.07 (m, 4H), 3.12 (t, 2H), 4.06 (s, 2H), 4.35-4.36 (m, 1H), 6.79-6.85 (m, 1H), 7.03-7.08 (m, 1H), 7.49 (d, 1H), 7.71-7.76 (m, 1H), 8.04-8.07 (m, 1H), 8.28 (d, 1H), 8.31-8.35 (m, 1H); UPLC-MS: 0.86 min, 364 [M+H]+.

To a solution of the above compound (22 mg, 0.059 mmol) in DCM (1 ml) 1M HCl in Et₂O was added dropwise and the solution was left at r.t. under stirring for 30 mins. Then the precipitate was separated, triturated with Et₂O, left under nitrogen flow and dried for 18 hours under high vacuum at 40° C. to afford the title compound (20 mg).

All the analytical data are set forth in the following Table 1-1 and in which R, A₁ and B are:

| | |
|---|---|
| | (IIA) |
| 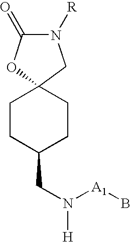 | |

| Cpd. | | | |
|---|---|---|---|
| No. | R | A1—B | Analytical Data |
| 1-1 | Ph | 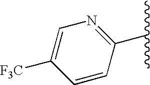 | ¹H NMR (400 MHz, CDCl₃): δ 1.08-1.38 (m, 2 H), 1.68-1.95 (m, 3 H), 1.95-2.13 (m, 4 H), 3.24-3.48 (m, 2 H), 3.81 (s, 2 H), 4.82-4.95 (m, 1 H), 6.35-6.51 (m, 1 H), 7.09-7.22 (m, 1 H), 7.35-7.46 (m, 2 H), 7.52-7.64 (m, 3 H), 8.29-8.39 (m, 1 H); |
| 1-2 | 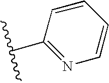 | 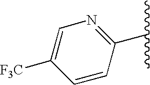 | ¹H NMR (400 MHz, DMSO-d₆): δ 1.11-1.25 (m, 2 H), 1.57-1.69 (m, 3 H), 1.74-1.83 (m, 2 H), 1.94-1.85 (m, 2 H), 3.17-3.26 (m, 2 H), 3.97 (s, 2 H), 6.63-6.68 (m, 1 H), 7.09-7.14 (m, 1 H), 7.63-7.64 (m, 1 H), 7.77-7.83 (m, 1 H), 8.07 (d, 1 H), 8.23-8.27 (m, 1 H), 8.31-8.35 (m, 1 H); UPLC-MS: 0.78 min, 407 [M + H]+ |
| 1-3 | 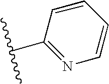 | 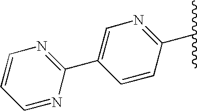 | ¹H NMR (400 MHz, DMSO-d₆): δ 8.90 (d, 2 H), 8.78-8.59 (m, 2 H), 8.37 (dq, 1 H), 8.11 (dt, 1 H), 7.87-7.82 (m, 1 H), 7.48 (t, 1 H), 7.18-7.14 (m, 2 H), 4.04 (s, 2 H), 2.02-1.64 (m, 7 H), 1.36-1.25 (m, 2 H); UPLC-MS: 0.56 min, 417 [M + H]+ |
| 1-4 | 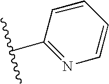 | 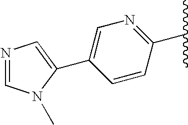 | ¹H NMR (400 MHz, DMSO-d₆): δ 9.16 (s, 1 H), 8.37 (dq, 1 H), 8.15-8.09 (m, 2 H), 7.87-7.79 (m, 4 H), 7.17-7.14 (m, 1 H), 4.02 (s, 2 H), 3.80 (s, 3 H), 3.33-3.27 (m, 1 H), 2.04-1.62 (m, 7 H), 1.31-1.18 (m, 2 H); UPLC-MS: 0.46 min, 419 [M + H]+ |
| 1-5 | 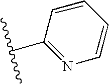 | 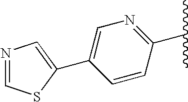 | ¹H NMR (400 MHz, DMSO-d₆): δ 9.12 (s, 1 H), 8.37 (dq, 1 H), 8.30 (br s, 1 H), 8.21-8.02 (m, 3 H), 7.87-7.82 (m, 1 H), 7.18-7.14 (m, 2 H), 4.02 (s, 2 H), 3.30 (br s, 1 H), 2.06-1.67 (m, 7 H), 1.35-1.19 (m, 2 H); UPLC-MS: 0.54 min, 422 [M + H]+ |
| 1-6 | 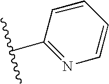 | 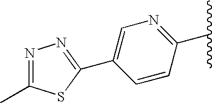 | ¹H NMR (400 MHz, DMSO-d₆): δ 8.45 (br s, 1 H), 8.37 (dq, 1 H), 8.10 (dt, 2 H), 7.87-7.82 (m, 1 H), 7.17-7.13 (m, 1 H), 6.91 (br s, 1 H), 4.02 (s, 2 H), 2.05-1.64 (m, 7 H), 1.33-1.22 (m, 2 H); UPLC-MS: 0.58 min, 437 [M + H]+ |
| 1-7 | 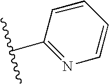 | 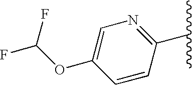 | ¹H NMR (400 MHz, MeOD-d₄): δ 8.37 (1 H, ddd), 8.10 (1 H, d), 7.88-7.92 (1 H, m), 7.84 (1 H, ddd), 7.75-7.81 (1 H, m), 7.16 (1 H, ddd), 7.15 (1 H, t), 7.04-7.11 (1 H, m), 4.02 (2 H, s), 3.30 (2 H, d), 1.94-2.04 (2 H, m), 1.80-1.90 (2 H, m), 1.62-1.75 (3 H, m), 1.18-1.34 (2 H, m). |
| 1-8 | 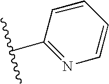 | 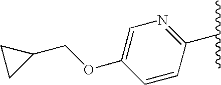 | ¹H NMR (400 MHz, DMSO-d₆): δ 13.31-13.56 (1 H, m), 8.58-8.68 (1 H, m), 8.37 (1 H, ddd), 8.10 (1 H, dt), 7.85 (1 H, ddd), 7.76 (1 H, dd), 7.36-7.43 (1 H, m), 7.16 (1 H, ddd), 7.11 (1 H, d), 4.05 (2 H, s), 3.81 (2 H, d), 3.25-3.32 (2 H, m), 1.95-2.03 (2 H, m), 1.81-1.91 (2 H, m), 1.62-1.74 (3 H, m), 1.13-1.33 (3 H, m), 0.58 (2 H, dqua), 0.33 (2 H, dqua). |
| 1-9 | 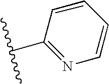 | 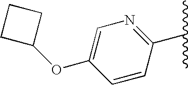 | ¹H NMR (400 MHz, DMSO-d₆): δ 13.27-13.58 (1 H, m), 8.58-8.71 (1 H, m), 8.36 (1 H, ddd), 8.10 (1 H, dt), 7.85 (1 H, ddd), 7.69 (1 H, dd), 7.25-7.31 (1 H, m), 7.16 (1 H, ddd), 7.11 (1 H, d), 6.35 (1 H, d), 4.61-4.73 (2 H, m), 4.02 (2 H, s), 3.22-3.32 (2 H, m), 2.34-2.44 (2 H, m), 1.94-2.10 (4 H, m), 1.74-1.91 (3 H, m), 1.58-1.73 (4 H, m), 1.18-1.33 (2 H, m). |
| 1-10 | 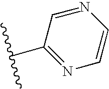 | 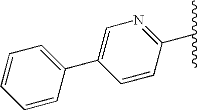 | ¹H NMR (500 MHz, DMSO-d₆): δ 1.21-1.34 (m, 2 H), 1.65-1.78 (m, 3 H), 1.88 (d, 2 H), 2.04 (d, 2 H), 3.28-3.49 (m, 2 H), 4.01 (s, 2 H), 7.16 (d, 1 H), 7.40 (t, 1 H), 7.49 (t, 2 H), 7.66 (d, 2 H), 8.16 (s, 1 H), 8.21 (d, 1 H), 8.39 (d, 1 H), 8.44 (s, 1 H), 9.37 (s, 1 H), 13.73 (br s, 1 H); UPLC-MS: 0.95 min, 416 [M + H]+ |
| 1-11 | 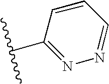 | 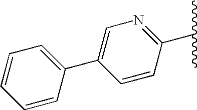 | ¹H NMR (400 MHz, CDCl₃): δ 1.23 (s, 3 H), 1.80-2.02 (m, 4 H), 2.02-2.12 (m, 1 H), 2.15-2.24 (m, 2 H), 3.23-3.34 (m, 2 H), 4.22 (s, 2 H), 6.88-6.99 (m, 1 H), 7.44-7.55 (m, 5 H), 7.70-7.77 (m, 1 H), 7.97-8.01 (m, 1 H), 8.13 (dd, 1 H), 8.81 (dd, 1 H), 9.02 (dd, 1 H); UPLC-MS: 0.59 min, 416 [M + H]+ |
| 1-12 | 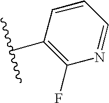 | 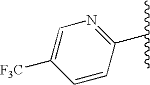 | ¹H NMR (400 MHz, DMSO-d₆): δ 1.04-1.22 (m, 2 H), 1.52-1.75 (m, 3 H), 1.76-1.88 (m, 2 H), 1.92-2.06 (m, 2 H), 3.20 (t, 2 H), 3.90 (s, 2 H), 6.56-6.66 (m, 1 H), 7.38-7.50 (m, 2 H), 7.56-7.65 (m, 1 H), 8.08-8.22 (m, 2 H), 8.22-8.30 (m, 1 H); UPLC-MS: 0.91 min, 425 [M + H]+ |
| 1-13 | 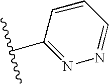 | 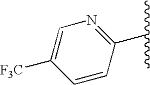 | ¹H NMR (400 MHz, DMSO-d₆): δ 1.25 (q, 2 H), 1.56-1.75 (m, 3 H), 1.81 (d, 2 H), 2.00 (d, 2 H), 3.21-3.29 (m, 2 H), 4.14 (s, 2 H), 6.67 (d, 1 H), 7.57-7.69 (m, 2 H), 7.73 (dd, 1 H), 8.29 (s, 1 H), 8.38 (dd, 1 H), 9.01 (dd, 1 H); UPLC-MS: 0.86 min, 408 [M + H]+ |
| 1-14 | 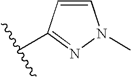 | 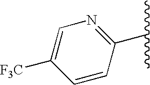 | ¹H NMR (400 MHz, DMSO-d₆): δ 1.11-1.26 (m, 2 H), 1.54-1.70 (m, 3 H), 1.73-1.85 (m, 2 H), 1.89-1.91 (m, 2 H), 3.18-3.27 (m, 2 H), 3.75 (s, 3 H), 3.80 (s, 2 H), 6.34-6.37 (m, 1 H), 6.65-6.73 (m, 1 H), 7.70 (br s, 2 H), 7.59-7.64 (m, 1 H), 7.63-7.71 (m, 1 H), 8.27 (s, 1 H); UPLC-MS: 0.86 min, 410 [M + H]+ |
| 1-15 | 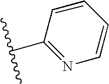 | 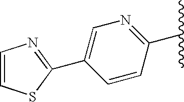 | ¹H NMR (400 MHz, DMSO-d₆): δ 1.15-1.36 (m, 2 H), 1.62-1.76 (m, 3 H), 1.78-1.92 (m, 2 H), 1.92-2.08 (m, 2 H), 3.23-3.34 (m, 2 H), 4.03 (s, 2 H), 6.73-6.91 (br s, 1 H), 7.06-7.21 (m, 1 H), 7.65-7.74 (m, 1 H), 7.78-7.92 (m, 2 H), 7.97-8.16 (m, 2 H), 8.34-8.42 (m, 1 H), 8.44-8.55 (m, 1 H); HPLC-MS: 1.713 min, 422 [M + H]+ |
| 1-16 | 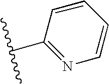 | 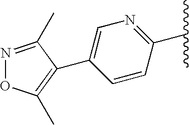 | ¹H NMR (400 MHz, CDCl₃): δ 1.19-1.36 (m, 2 H), 1.71-1.83 (m, 1 H), 1.83-1.96 (m, 2 H), 1.98-2.12 (m, 4 H), 2.27 (s, 3 H), 2.41 (s, 3 H), 3.29 (t, 2 H), 4.08 (s, 2 H), 4.64-4.74 (br s, 1 H), 6.44-6.52 (m, 1 H), 7.03-7.12 (m, 1 H), 7.31-7.37 (m, 1 H), 7.69-7.78 (m, 1 H), 7.98-8.04 (m, 1 H), 8.26-8.32 (m, 1 H), 8.33-8.39 (m, 1 H); UPLC-MS: 0.59 min, 434 [M + H]+ |
| 1-17 | 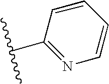 | 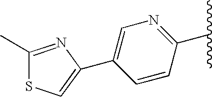 | ¹H NMR (400 MHz, CDCl₃): δ 1.18-1.35 (m, 2 H), 1.68-1.93 (m, 3 H), 1.97-2.08 (m, 4 H), 2.78 (s, 3 H), 3.28 (t, 2 H), 4.06 (s, 2 H), 4.68-4.77 (br t, 1 H), 6.41-6.48 (m, 1 H), 7.05 (ddd, 1 H), 7.14 (s, 1 H), 7.73 (ddd, 1 H), 7.95 (dd, 1 H), 8.28 (dt, 1 H), 8.35 (ddd, 1 H), 8.58-8.62 (m, 1 H); HPLC-MS: 1.617 min, 436 [M + H]+ |
| 1-18 | 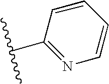 | 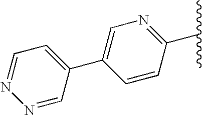 | ¹H NMR (400 MHz, DMSO-d₆): δ 1.18-1.34 (m, 2 H), 1.62-1.77 (m, 3 H), 1.79-1.92 (m, 2 H), 1.94-2.06 (m, 2 H), 3.26-3.36 (m, 2 H), 4.03 (s, 2 H), 6.82-6.97 (br s, 1 H), 7.10-7.20 (m, 1 H), 7.80-7.89 (m, 1 H), 7.99-8.22 (m, 3 H), 8.34-8.41 (m, 1 H), 8.63 (br s, 1 H), 9.24 (d, 1 H), 9.63-9.70 (m, 1 H); HPLC-MS: 1.516 min, 417 [M + H]+ |
| 1-19 | 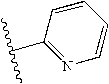 | 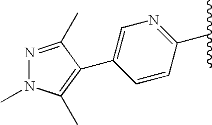 | ¹H NMR (400 MHz, CDCl₃): δ 1.19-1.34 (m, 2 H), 1.72-1.95 (m, 3 H), 1.98-2.10 (m, 4 H), 2.23 (s, 6 H), 3.27 (t, 2 H), 3.80 (s, 3 H), 4.08 (s, 2 H), 4.55-4.64 (m, 1 H), 6.44-6.49 (m, 1 H), 7.03-7.09 (m, 1 H), 7.32-7.37 (m, 1 H), 7.70-7.78 (m, 1 H), 7.96-8.02 (m, 1 H), 8.25-8.31 (m, 1 H), 8.33-8.37 (m, 1 H); HPLC-MS: 1.518 min, 447 [M + H]+ |
| 1-20 | 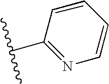 | 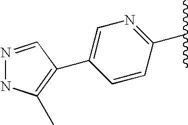 | ¹H NMR (400 MHz, CDCl₃): δ 1.17-1.35 (m, 2 H), 1.71-1.82 (m, 1 H), 1.82-1.95 (m, 2 H), 1.96-2.11 (m, 4 H), 2.43 (s, 3 H), 3.21-3.32 (m, 2 H), 4.07 (s, 2 H), 4.59-4.70 (m, 1 H), 6.47 (d, 1 H), 7.03-7.10 (m, 1 H), 7.49 (dd, 1 H), 7.61 (s, 1 H), 7.70-7.78 (m, 1 H), 8.16 (d, 1 H), 8.29 (d, 1 H), 8.32-8.39 (m, 1 H); HPLC-MS: 1.498 min, 419 [M + H]+ |
| 1-21 | 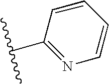 | 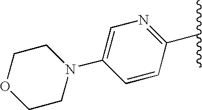 | ¹H NMR (400 MHz, DMSO-d₆): δ 1.15-1.38 (m, 2 H), 1.60-1.76 (m, 3 H), 1.78-1.92 (m, 2 H), 1.93-2.08 (m, 2 H), 2.93-3.08 (m, 4 H), 3.18-3.32 (m, 2 H), 3.65-3.83 (m, 4 H), 4.03 (s, 2 H), 7.03-7.11 (m, 1 H), 7.12-7.22 (m, 2 H), 7.78-7.90 (m, 1 H), 7.91-7.99 (m, 1 H), 8.02-8.17 (m, 1 H), 8.32-8.40 (m, 1 H), 8.41-8.58 (br s, 1 H), 13.04-13.23 (br s, 1 H); HPLC-MS: 1.446 min, 424 [M + H]+ |
| 1-22 | 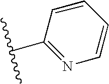 | 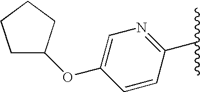 | ¹H NMR (400 MHz, DMSO-d₆): δ 1.16-1.35 (m, 2 H), 1.52-1.77 (m, 9 H), 1.78-1.93 (m, 4 H), 1.94-2.05 (m, 2 H), 3.17-3.26 (m, 2 H), 4.02 (s, 2 H), 4.71-4.82 (m, 1 H), 6.92-7.08 (m, 1 H), 7.12-7.21 (m, 1 H), 7.39-7.48 (m, 1 H), 7.58-7.71 (m, 1 H), 7.80-7.91 (m, 1 H), 8.06-8.17 (m, 1 H), 8.32-8.42 (m, 1 H); HPLC-MS: 1.712 min, 423 [M + H]+. |
| 1-23 | 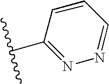 | 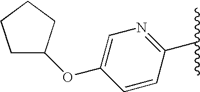 | ¹H NMR (400 MHz, CDCl₃): δ 1.13-1.33 (m, 2 H), 1.55-1.70 (m, 3 H), 1.70-1.97 (m, 8 H), 1.97-2.12 (m, 4 H), 3.15-3.25 (m, 2 H), 4.22 (s, 2 H), 4.24-4.34 (m, 1 H), 4.59-4.71 (m, 1 H), 6.38 (dd, 1 H), 7.10 (dd, 1 H), 7.50 (dd, 1 H), 7.80 (dd, 1 H), 8.58 (dd, 1 H), 8.97 (dd, 1 H); HPLC-MS: 1.599 mm; 424 [M + H]+ |
| 1-24 | 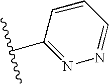 | 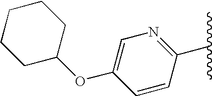 | ¹H NMR (400 MHz, DMSO-d₆): δ 1.18-1.60 (m, 8 H), 1.63-1.80 (m, 5 H), 1.81-1.97 (m, 4 H), 2.00-2.12 (m, 2 H), 3.17-3.29 (m, 2 H), 4.16 (s, 2 H), 4.22-4.34 (m, 1 H), 7.02-7.14 (m, 1 H), 7.48-7.57 (m, 1 H), 7.70-7.82 (m, 2 H), 8.35-8.42 (m, 1 H), 8.42-8.55 (m, 1 H), 8.99-9.06 (m, 1 H); HPLCMS: 1.673 min, 438 [M + H]+ |
| 1-25 | 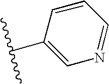 | 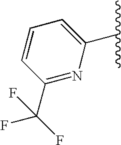 | ¹H NMR (400 MHz, DMSO-d6): δ 8.96 (s, 1 H), 8.50 (br s, 1 H), 8.38 (m, 1 H), 7.77 (m, 1 H), 7.56 (m, 1 H), 6.86 (d, 1 H), 6.75 (d, 1 H), 4.0 (s, 2 H), 3.17 (d, 2 H), 2.05-1.60 (m, 4 H), 1.31-1.15 (m, 5 H); UPLC-MS: 0.72 min, 407 [M + H]+ |
| 1-26 | 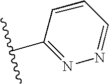 | 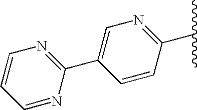 | ¹H NMR (400 MHz, DMSO-d6): δ 9.02 (dd, 1 H), 8.91 (d, 2 H), 8.76-8.63 (m, 2 H), 8.40 (d, 1 H), 7.74 (ddd, 1 H), 7.50 (t, 1 H), 7.22 (br s, 1 H), 4.17 (s, 2 H), 3.40 (br s, 2 H), 2.13-1.20 (m, 9 H); UPLC-MS: 0.47 min, 418 [M + H]+ |
| 1-27 | 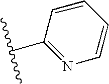 | 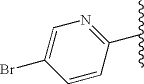 | ¹H NMR (400 MHz, CDCl₃): δ 1.60-1.79 (m, 3 H), 1.80-1.91 (m, 2 H), 1.95-2.06 (m, 4 H), 3.18-3.25 (m, 2 H), 4.05 (s, 2 H), 4.60 (br s, 1 H), 6.30-6.34 (m, 1 H), 7.03-7.08 (m, 1 H), 7.48 (dd, 1 H), 7.70-7.76 (m, 1 H), 8.11-8.13 (m, 1 H), 8.28 (dt, 1 H), 8.34 (m, 1 H); UPLC-MS: 0.67 min, 418 [M + H]+ |
| 1-28 | 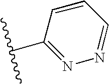 | 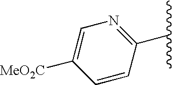 | ¹H NMR (400 MHz, CDCl₃): δ 1.68-1.94 (m, 5 H), 1.97-2.22 (m, 4 H), 3.32 (td, 2 H), 3.89 (s, 3 H), 4.08-4.25 (m, 2 H), 5.20 (d, 1 H), 6.37-6.41 (m, 1 H), 7.46-7.57 (m, 1 H), 7.98-8.05 (m, 1 H), 8.56 (ddd, 1 H), 8.73-8.79 (m, 1 H), 8.94-8.99 (m, 1 H); UPLC-MS: 0.48 min, 398 [M + H]+ |
| 1-29 | 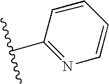 | 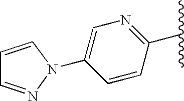 | ¹H NMR (400 MHz, CDCl₃): δ 1.92 (br s, 4 H), 2.00-2.18 (m, 5 H), 3.28-3.42 (m, 2 H), 4.19-4.29 (m, 2 H), 6.55 (t, 1 H), 6.98-7.11 (m, 1 H), 7.13-7.22 (m, 1 H), 7.76 (d, 1 H), 7.83 (d, 2 H), 8.17-8.21 (m, 1 H), 8.30-8.37 (m, 1 H), 8.41 (br s, 2 H); UPLC-MS: 0.54 min, 405 [M + H]+ |
| 1-30 | 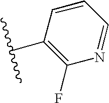 | 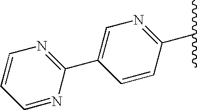 | ¹H NMR (400 MHz, DMSO-d₆): δ 1.20-1.28 (m, 2 H), 1.68-1.79 (m, 3 H), 1.86-1.93 (m, 2 H), 2.01-2.09 (m, 2 H), 3.30-3.36 (m, 2 H), 3.94 (s, 2 H), 7.43-7.48 (m, 2 H), 8.13-8.19 (m, 2 H), 8.54-8.61 (m, 1 H), 8.76-8.82 (m, 1 H), 8.88 (s, 1 H), 8.89 (s, 2 H); UPLC-MS: 0.47 min, 435 [M + H]+. |
| 1-31 | 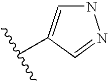 | 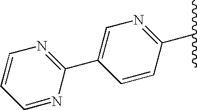 | ¹H NMR (400 MHz, MeOD-d₄): δ 1.80-1.99 (m, 4 H), 2.08 (s, 5 H), 3.40 (d, 3 H), 3.89 (s, 2 H), 7.19-7.30 (m, 1 H), 7.44 (t, 1 H), 7.89 (s, 2 H), 8.88 (d, 4); UPLC-MS: 0.41 min, 406 [M + H]+. |
| 1-32 | 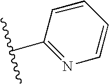 | 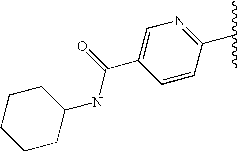 | ¹H NMR (400 MHz, CDCl₃): δ 1.41-1.47 (m, 2 H), 1.59-1.90 (m, 11 H), 1.95-2.05 (m, 6 H), 3.29 (t, 2 H), 3.92-4.02 (m, 1 H), 4.06 (s, 2 H), 4.90 (br s, 1 H), 5.78 (br s, 1 H), 6.35-6.44 (m, 1 H), 7.05 (ddd, 1 H), 7.69-7.76 (m, 1 H), 7.86 (dd, 1 H), 8.28 (dt, 1 H), 8.32-8.36 (m, 1 H), 8.45-8.49 (m, 1 H); UPLC-MS: 0.58 min, 464 [M + H]+. |
| 1-33 | 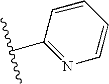 | 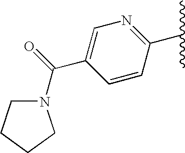 | ¹H NMR (400 MHz, DMSO-d₆): δ 1.70 (d, 4 H), 1.81-1.92 (m, 8 H), 1.99 (br s, 3 H), 3.34 (br s, 2 H), 3.46 (br s, 2 H), 4.03 (s, 2 H), 7.06-7.13 (m, 1 H), 7.16 (ddd, 1 H), 7.82-7.88 (m, 1 H), 7.96-8.03 (m, 1 H), 8.07-8.14 (m, 2 H), 8.35-8.39 (m, 1 H); UPLC-MS: 0.49 min, 436 [M + H]+. |
| 1-34 | 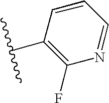 | 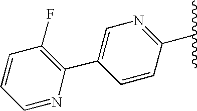 | ¹H NMR (400 MHz, MeOD-d₄): δ 1.85-1.95 (m, 3 H), 2.02-2.10 (m, 2 H), 2.11-2.20 (m, 3 H), 3.34-3.38 (m, 3 H), 3.98 (s, 2 H), 7.13-7.19 (m, 1 H), 7.38-7.44 (m, 1 H), 7.44-7.55 (m, 1 H), 7.70-7.81 (m, 1 H), 8.10-8.21 (m, 2 H), 8.42-8.49 (m, 1 H), 8.49-8.60 (m, 2 H); UPLC-MS: 0.51 min, 452 [M + H]+. |
| 1-35 | 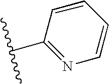 | 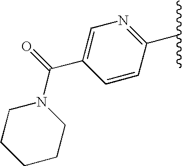 | ¹H NMR (400 MHz, MeOD-d₄): δ 1.35-1.42 (m, 2 H), 1.73-1.81 (m, 2 H), 1.84-1.95 (m, 4 H), 2.01-2.16 (m, 5 H), 3.31-3.34 (m, 7 H), 3.35-3.41 (m, 2 H), 4.16 (s, 2 H), 7.13-7.20 (m, 1 H), 7.26-7.32 (m, 1 H), 7.91-8.05 (m, 3 H), 8.06-8.11 (m, 1 H), 8.37-8.42 (m, 1 H); UPLC-MS: 0.54 min, 450 [M + H]+. |
| 1-36 | 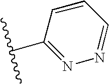 | 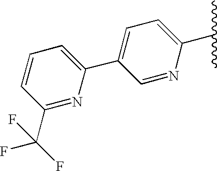 | ¹H NMR (400 MHz, DMSO-d₆): δ 1.26-1.42 (m, 2 H), 1.67-1.82 (m, 3 H), 1.91 (d, 2 H), 2.07 (d, 2 H), 3.38-3.46 (m, 2 H), 4.17 (s, 2 H), 7.25 (d, 1 H), 7.75 (dd, 1 H), 7.90 (d, 1 H), 8.23 (t, 1 H), 8.32 (d, 1 H), 8.40 (dd, 1 H), 8.53-8.65 (m, 2 H), 9.03 (dd, 1 H); UPLC-MS: 0.58 min, 485 [M + H]+ |
| 1-37 | 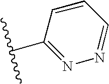 | 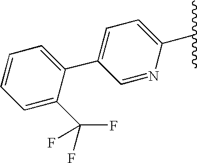 | ¹H NMR (400 MHz, DMSO-d₆): δ 1.65-1.81 (m, 1 H), 1.84-2.14 (m, 7 H), 3.27-3.35 (m, 2 H), 4.12 (s, 1 H), 4.17 (s, 2 H), 4.70-4.86 (m, 1 H), 7.49 (d, 1 H), 7.65-7.83 (m, 4 H), 7.88 (d, 1 H), 8.39 (dd, 2 H), 8.99-9.04 (m, 2 H); UPLC-MS: 0.58 min, 484 [M + H]+ |
| 1-38 | 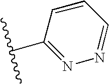 | 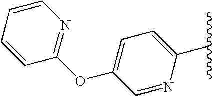 | ¹H NMR (400 MHz, DMSO-d₆): δ 1.69-1.81 (m, 3 H), 1.88-2.09 (m, 6 H), 3.33-3.40 (m, 2 H), 4.16 (s, 2 H), 7.14-7.25 (m, 3 H), 7.77 (dd, 1 H), 7.86-7.98 (m, 3 H), 8.13-8.18 (m, 1 H), 8.41 (dd, 1 H), 9.04 (dd, 1 H); UPLC-MS: 0.48 min, 433 [M + H]+ |
| 1-39 | 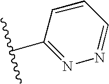 | 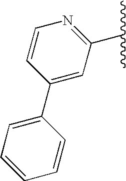 | ¹H NMR (400 MHz, DMSO-d₆): δ 1.29-1.41 (m, 2 H), 1.74 (td, 3 H), 1.89-1.98 (m, 2 H), 2.03-2.09 (m, 2 H), 3.41 (t, 2 H), 4.17 (s, 2 H), 7.21 (dd, 1 H), 7.39-7.42 (m, 1 H), 7.56-7.63 (m, 3 H), 7.73-7.88 (m, 3 H), 8.00 (br s, 1 H), 8.40 (dd, 1 H), 9.03 (dd, 1 H); UPLC-MS: 0.53 min, 416 [M + H]+ |
| 1-40 | 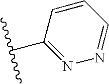 | 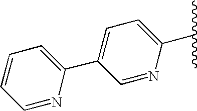 | ¹H NMR (400 MHz, CDCl₃): δ 1.19-1.35 (m, 2 H), 1.73-1.98 (m, 3 H), 2.00-2.13 (m, 4 H), 3.32 (t, 2 H), 4.23 (s, 2 H), 4.72-4.83 (m, 1 H), 6.51 (d, 1 H), 7.12-7.23 (m, 1 H), 7.46-7.55 (m, 1 H), 7.65 (d, 1 H), 7.69-7.77 (m, 1 H), 8.16 (dd, 1 H), 8.54-8.61 (m, 1 H), 8.62-8.69 (m, 1 H), 8.68-8.75 (m, 1 H), 8.93-9.01 (m, 1 H); UPLC-MS: 0.46 min, 417 [M + H]+ |
| 1-41 | 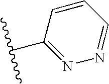 | 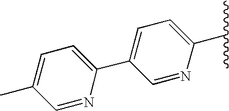 | ¹H NMR (400 MHz, CDCl₃): δ 1.17-1.34 (m, 2 H), 1.70-1.97 (m, 3 H), 1.99-2.12 (m, 4 H), 2.37 (s, 3 H), 3.31 (t, 2 H), 4.23 (s, 2 H), 4.68-4.79 (m, 1 H), 6.49 (d, 1 H), 7.45-7.58 (m, 3 H), 8.12 (dd, 1 H), 8.43-8.52 (m, 1 H), 8.54-8.62 (m, 1 H), 8.65-8.71 (m, 1 H), 8.93-9.01 (m, 1 H); UPLC-MS: 0.49 min, 431 [M + H]+ |
| 1-42 | 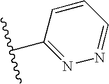 | 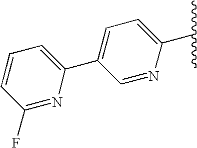 | ¹H NMR (400 MHz, CDCl₃): δ 1.16-1.37 (m, 2 H), 1.73-1.99 (m, 3 H), 2.01-2.12 (m, 4 H), 3.32 (t, 2 H), 4.23 (s, 2 H), 4.82 (t, 1 H), 6.49 (d, 1 H), 6.75-6.83 (m, 1 H), 7.44-7.57 (m, 2 H), 7.73-7.86 (m, 1 H), 8.09-8.19 (m, 1 H), 8.58 (dd, 1 H), 8.69-8.76 (m, 1 H), 8.92-9.00 (m, 1 H); UPLC-MS: 0.52 min, 435 [M + H]+ |
| 1-43 | 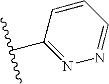 | 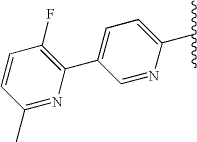 | ¹H NMR (400 MHz, CDCl₃): δ 1.17-1.36 (m, 2 H), 1.74-1.98 (m, 3 H), 1.98-2.13 (m, 4 H), 2.58 (s, 3 H), 3.33 (t, 2 H), 4.23 (s, 2 H), 4.79 (t, 1 H), 6.49 (d, 1 H), 6.97-7.07 (m, 1 H), 7.29-7.40 (m, 1 H), 7.45-7.56 (m, 1 H), 8.05-8.16 (m, 1 H), 8.54-8.62 (m, 1 H), 8.70-8.81 (m, 1 H), 8.93-9.01 (m, 1 H); UPLC\MS: 0.53 min, 449 [M + H]+. |
| 1-44 | 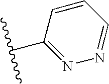 | 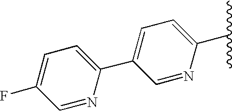 | ¹H NMR (400 MHz, CDCl₃): δ 1.17-1.36 (m, 2 H), 1.72-1.98 (m, 3 H), 1.99-2.12 (m, 4 H), 3.32 (t, 2 H), 4.23 (s, 2 H), 4.78 (t, 1 H), 6.50 (d, 1 H), 7.38-7.56 (m, 2 H), 7.58-7.66 (m, 1 H), 8.04-8.12 (m, 1 H), 8.47-8.53 (m, 1 H), 8.55-8.61 (m, 1 H), 8.62-8.69 (m, 1 H), 8.93-9.01 (m, 1 H); UPLC\MS: 0.50 min, 435 [M + H]+. |
| 1-45 | 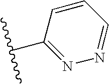 | 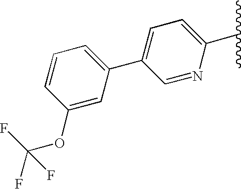 | ¹H NMR (400 MHz, CDCl₃): δ 1.18-1.37 (m, 2 H), 1.73-1.99 (m, 3 H), 2.00-2.13 (m, 4 H), 3.33 (t, 2 H), 4.24 (s, 2 H), 4.65-4.79 (m, 1 H), 6.50 (d, 1 H), 7.25-7.35 (m, 2 H), 7.42-7.57 (m, 3 H), 7.62-7.72 (m, 1 H), 8.32-8.41 (m, 1 H), 8.53-8.64 (m, 1 H), 8.94-9.03 (m, 1 H); UPLC\MS: 0.61 min, 500 [M + H]+. |
| 1-46 | 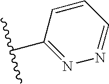 | 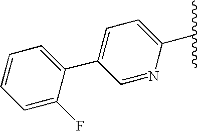 | ¹H NMR (400 MHz, CDCl₃): δ 1.16-1.36 (m, 2 H), 1.75-1.98 (m, 3 H), 2.01-2.14 (m, 4 H), 3.31 (t, 2 H), 4.24 (s, 2 H), 4.65-4.75 (m, 1 H), 6.45-6.54 (m, 1 H), 7.37-7.46 (m, 1 H), 7.46-7.55 (m, 1 H), 7.64-7.72 (m, 2 H), 8.26-8.35 (m, 1 H), 8.53-8.63 (m, 1 H), 8.90-9.03 (m, 1 H); HPLC\MS: 1.851 min, 434 [M + H]+. |
| 1-47 | 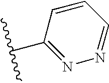 | 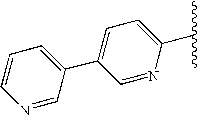 | ¹H NMR (400 MHz, CDCl₃): δ 1.19-1.34 (m, 2 H), 1.75-1.97 (m, 3 H), 2.01-2.11 (m, 4 H), 3.32 (t, 2 H), 4.23 (s, 2 H), 4.71-4.78 (m, 1 H), 6.52 (d, 1 H), 7.33-7.39 (m, 1 H), 7.47-7.54 (m, 1 H), 7.65-7.71 (m, 1 H), 7.78-7.83 (m, 1 H), 8.35 (d, 1 H), 8.53-8.62 (m, 2 H), 8.76-8.82 (m, 1 H), 8.97 (dd, 1 H); HPLC\MS: 1.485 min, 417 [M + H]+. |
| 1-48 | 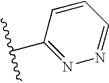 | 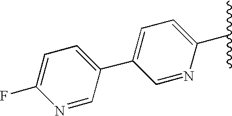 | ¹H NMR (400 MHz, CDCl₃): δ 1.18-1.37 (m, 2 H), 1.72-1.98 (m, 3 H), 2.00-2.14 (m, 4 H), 3.32 (t, 2 H), 4.23 (s, 2 H), 4.68-4.79 (m, 1 H), 6.46-6.57 (m, 1 H), 6.96-7.06 (m, 1 H), 7.46-7.56 (m, 1 H), 7.59-7.67 (m, 1 H), 7.84-7.95 (m, 1 H), 8.27-8.40 (m, 2 H), 8.54 8.63 (m, 1 H), 8.93-9.01 (m, 1 H); HPLC\MS: 1.589 min, 435 [M + H]+. |
| 1-49 | 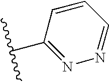 | 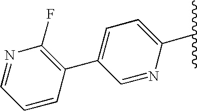 | ¹H NMR (400 MHz, CDCl₃): δ 1.17-1.34 (m, 2 H), 1.73-1.98 (m, 3 H), 1.98-2.13 (m, 4 H), 3.32 (t, 2 H), 4.23 (s, 2 H), 4.74-4.82 (m, 1 H), 6.50 (dd, 1 H), 7.22-7.33 (m, 1 H), 7.45-7.56 (m, 1 H), 7.66-7.74 (m, 1 H), 7.79-7.91 (m, 1 H), 8.13-8.21 (m, 1 H), 8.28-8.37 (m, 1 H), 8.53-8.62 (m, 1 H), 8.94-9.01 (m, 1 H); UPLC-MS: 0.48 min, 435 [M + H]+. |
| 1-50 | 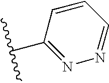 | 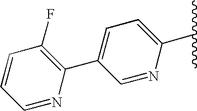 | ¹H NMR (400 MHz, DMSO-d₆): δ 1.26-1.42 (m, 2 H), 1.67-1.83 (m, 3 H), 1.85-1.97 (m, 2 H), 2.02-2.13 (m, 2 H), 3.34-3.41 (m, 2 H), 4.18 (s, 2 H), 7.15-7.28 (m, 1 H), 7.53 (quin, 1 H), 7.72-7.79 (m, 1 H), 7.85-7.96 (m, 1 H), 8.35-8.51 (m, 3 H), 8.54-8.59 (m, 1 H), 9.01-9.05 (m, 1 H); UPLC\MS: 0.49 min, 435 [M + H]+. |
| 1-51 | 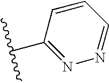 | 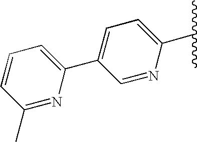 | ¹H NMR (400 MHz, DMSO-d₆): δ 1.25-1.43 (m, 2 H), 1.65-1.83 (m, 3 H), 1.85-1.98 (m, 2 H), 2.02-2.12 (m, 2 H), 2.58 (s, 3 H), 3.36-3.46 (m, 2 H), 4.17 (s, 2 H), 7.13-7.28 (m, 1 H), 7.29-7.41 (m, 1 H), 7.72-7.80 (m, 1 H), 7.81-7.98 (m, 2 H), 8.37-8.45 (m, 1 H), 8.46-8.61 (m, 2 H), 9.01-9.07 (m, 1 H); UPLC\MS: 0.76 min, 431 [M + H]+. |
| 1-52 | 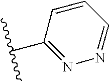 | 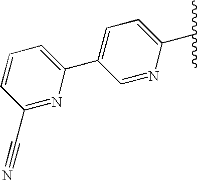 | ¹H NMR (400 MHz, DMSO-d₆): δ 1.26-1.44 (m, 2 H), 1.67-1.84 (m, 3 H), 1.86-1.98 (m, 2 H), 2.02-2.11 (m, 2 H), 3.38-3.46 (m, 2 H), 4.17 (s, 2 H), 7.17-7.29 (m, 1 H), 7.71-7.79 (m, 1 H), 8.03 (d, 1 H), 8.18 (t, 1 H), 8.33 (d, 1 H), 8.37-8.43 (m, 1 H), 8.46-8.65 (m, 2 H), 8.99-9.05 (m, 1 H); UPLC\MS: 0.75 min, 442 [M + H]+. |
| 1-53 | 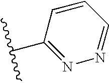 | 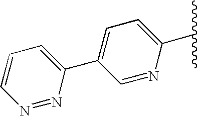 | ¹H NMR (400 MHz, DMSO-d₆): δ 1.26-1.45 (m, 2 H), 1.66-1.85 (m, 3 H), 1.85-1.99 (m, 2 H), 2.01-2.14 (m, 2 H), 3.35-3.47 (m, 2 H), 4.18 (s, 2 H), 7.21-7.30 (m, 1 H), 7.69-7.80 (m, 1 H), 7.81-7.89 (m, 1 H), 8.24-8.35 (m, 1 H), 8.36-8.44 (m, 1 H), 8.55-8.72 (m, 2 H), 8.99-9.06 (m, 1 H), 9.20-9.28 (m, 1 H); UPLC\MS: 0.58 min, 418 [M + H]+. |
| 1-54 | 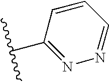 | 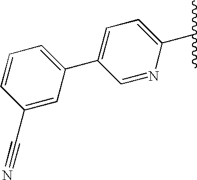 | ¹H NMR (400 MHz, DMSO-d₆): δ 9.02 (dd, 1 H), 8.40 (dd, 1 H), 8.25-8.37 (m, 2 H), 8.24 (s, 1 H), 8.05 (d, 1 H), 7.88 (d, 1 H), 7.76 (dd, 1 H), 7.71 (t, 1 H), 7.25 (d, 1 H), 4.17 (s, 2 H), 3.35-3.43 (m, 2 H), 2.02-2.12 (m, 2 H), 1.85-1.96 (m, 2 H), 1.67-1.79 (m, 3 H), 1.26-1.40 (m, 2 H); UPLC-MS: 0.52 min, 441 [M + H]+ and 221 [M + 2 H]2+ |
| 1-55 | 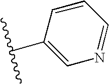 | 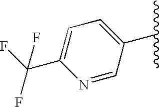 | ¹H NMR (400 MHz, DMSO-d₆): δ 8.94 (s, 1 H), 8.49 (d, 1 H), 8.35 (br s, 1 H), 8.36 (m, 1 H), 7.77-7.66 (m, 1 H), 7.52 (d, 1 H), 7.02 (br s, 1 H), 4.13 (br s, 1 H), 4.0 (s, 2 H), 3.02 (d, 2 H), 2.05-1.84 (m, 4 H), 1.74-0.80 (m, 5 H); UPLC-MS: 0.66 min, 407 [M + H]+. |
| 1-56 | 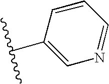 | 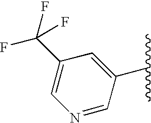 | ¹H NMR (400 MHz, DMSO-d₆): δ 8.96 (s, 1 H), 8.48 (d, 1 H), 8.37 (br s, 1 H), 8.28 (s, 1 H) 7.75 (m, 2 H), 6.72 (m, 1 H), 4.0 (s, 2 H), 3.24 (d, 2 H), 2.05-1.60 (m, 4 H), 1.31-1.15 (m, 5 H); UPLC-MS: 0.63 min, 407 [M + H]+. |
| 1-57 | 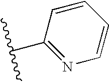 | 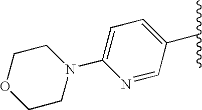 | ¹H NMR (400 MHz, DMSO-d₆): δ 1.18-1.33 (m, 2 H), 1.56-1.73 (m, 3 H), 1.84 (d, 2 H), 1.97 (d, 2 H), 2.94-3.03 (m, 2 H), 3.43-3.50 (m, 4 H), 3.71-3.78 (m, 4 H), 4.01 (s, 2 H), 6.15 (br s, 1 H), 7.12-7.19 (m, 1 H), 7.19-7.36 (m, 2 H), 7.65 (dd, 1 H), 7.84 (td, 1 H), 8.10 (d, 1 H), 8.32-8.39 (m, 1 H), 13.62 (br s, 1 H); UPLC-MS: 0.67 min, 424 [M + H]+ |
| 1-58 | 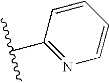 | 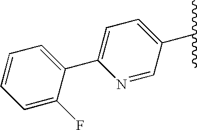 | ¹H NMR (400 MHz, DMSO-d₆): δ 1.21-1.40 (m, 2 H), 1.69 (t, 3 H), 1.86 (d, 2 H), 1.99 (br s, 2 H), 3.16 (d, 2 H), 4.03 (s, 2 H), 7.13-7.19 (m, 1 H), 7.39-7.49 (m, 2 H), 7.57-7.64 (m, 1 H), 7.72-7.80 (m, 2 H), 7.82-7.87 (m, 1 H), 7.88-7.93 (m, 1 H), 8.10 (dt, 1 H), 8.15-8.19 (m, 1 H), 8.35-8.40 (m, 1 H); UPLC-MS: 0.63 min, 433 [M + H]+. |
| 1-59 | 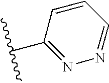 | 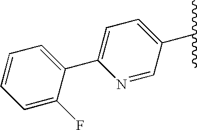 | ¹H NMR (400 MHz, MeOD-d₄): δ 1.41 (br s, 3 H), 1.82-2.00 (m, 3 H), 2.03-2.19 (m, 4 H), 3.23-3.28 (m, 1 H), 4.22 (s, 2 H), 7.36-7.49 (m, 2 H), 7.61-7.74 (m, 2 H), 7.81-7.88 (m, 1 H), 7.92-8.00 (m, 2 H), 8.04-8.09 (m, 1 H), 8.86 (dd, 1 H), 9.10 (dd, 1 H); UPLC-MS: 0.56 min, 433 [M + H]+. |
| 1-60 | 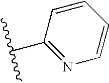 | 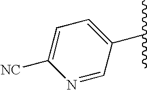 | ¹H NMR (400 MHz, CDCl₃): δ 8.31-8.41 (m, 2 H), 8.07 (d, 1 H), 7.80 (ddd, 1 H), 7.49 (dd, 1 H), 7.09-7.15 (m, 1 H), 6.85 (dd, 1 H), 4.14 (s, 2 H), 3.14 (d, 2 H), 1.74-2.09 (m, 10 H). |

### Example 2

### Preparation of Compounds of Formula (IIb)

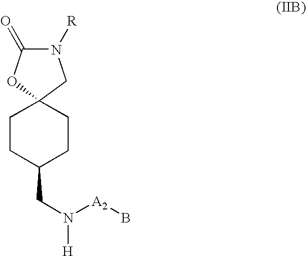

### Example 2-1

### (Trans)-8-({[5-(2-fluorophenyl)-2-pyrazinyl]amino}methyl)-3-(2-pyridinyl)-1-oxa-3-azaspiro[4.5]decan-2 one Hydrochloride

In a closed vial 5-(2-fluorophenyl)-2-pyrazinamine (Intermediate 25, 29.1 mg, 0.154 mmol) and (trans)-2-oxo-3-(2-pyridinyl)-1-oxa-3-azaspiro[4.5]decane-8-carbaldehyde (Intermediate 12 procedure 12b, 40 mg, 0.154 mmol) were dissolved in 1,2-dichloroethane (1 ml). Titanium (IV) isopropoxide (0.090 ml, 0.307 mmol) was added and the mixture was stirred at 60° C. for 3 hours. Then the reaction was allowed to cool to r.t. NaBH₄ (29.1 mg, 0.768 mmol) followed by ethanol (0.5 ml) (caution: gas evolution) were added and the mixture was stirred at r.t. overnight (ca 20 hours). The mixture was diluted with DCM (2 ml), a saturated aqueous solution of K₂CO₃ (2 ml) was added and the mixture was stirred at r.t. for 30 minutes. The mixture was extracted with DCM (3×3 ml). The organic layers were combined, filtered through a filter tube and dried under vacuo to afford a crude that was purified by Biotage silica column chromatography (Biotage SP1, 12+M) eluting with DCM/Et₂O 100:0 to 60:40 (25 cv) to afford a mixture that was further purified by MDAP (Preparative Column: AXIA 50×21 mm; Method: Focus O₂: 40% (B) to 45% (B) in 1 min; 45% (B) to 80% (B) in 7 min; 80% (B) to 100% (B) in 1 min; 100% (B) for 1.5 min; where Mobile phase are A: NH₄HCO₃ so1.10 mM, pH10; B: ACN). Product fractions were combined and dried to afford (trans)-8-({[5-(2-fluorophenyl)-2-pyrazinyl]amino}methyl)-3-(2-pyridinyl)-1-oxa-3-azaspiro[4.5]decan-2 one as a whitish solid (N3113-38-1 18.8 mg, 28%).

¹H NMR (400 MHz, CDCl₃): δ 1.20-1.36 (m, 2H) 1.73-1.94 (m, 3H), 1.97-2.09 (m, 4H), 3.38 (t, 2H), 4.08 (s, 2H), 4.77-4.87 (m, 1H), 7.02-7.09 (m, 1H), 7.13-7.20 (m, 1H), 7.22-7.28 (m, 1H), 7.30-7.37 (m, 1H), 7.67-7.79 (m, 1H), 7.86-7.95 (m, 1H), 7.99-8.06 (m, 1H), 8.24-8.31 (m, 1H), 8.31-8.39 (m, 1H), 8.50-8.58 (m, 1H); UPLC-MS: 0.78 min, 434 [M+H]+ The above compound was suspended in Et₂O (0.5 ml) and HCl 1M in Et₂O (1.2 eq, 0.052 ml, 0.052 mmol) were added. The resulting solid was triturated with Et₂O (3×0.3 ml) and dried under vacuo to afford the title compound as a whitish solid (16.3 mg, 23%);

### Example 2-2

### (Trans)-8-({[5-(2-fluorophenyl)-2-pyrazinyl]amino}methyl)-3-(3-pyridazinyl)-1-oxa-3-azaspiro[4.5]decan-2-one Hydrochloride

(Trans)-2-oxo-3-(3-pyridazinyl)-1-oxa-3-azaspiro[4.5]decane-8-carbaldehyde (Intermediate 60, 40 mg, 0.153 mmol) and 5-(2-fluorophenyl)-2-pyrazinamine (Intermediate 25, 29.0 mg, 0.153 mmol) were suspended in dichloromethane (2 ml) in a closed vial. Titanium (IV) isopropoxide (0.090 ml, 0.306 mmol) was added and the resulting solution was stirred at 45° C. for 5 hours then at r.t. overnight (ca 18 hours). NaBH₄ (29.0 mg, 0.765 mmol) followed by ethanol (1 ml) were added and the mixture was stirred at r.t. for 4 hours then saturated NaHCO₃ solution was added (2 ml) and the mixture was stirred at r.t. for 30 minutes then extracted with DCM (3×2 ml); the organics were combined and dried (vacuo) to afford a crude that was purified by KP-NH chromatography (Biotage SP1; 12+M) eluting in gradient with cyclohexane:EtOAc 100:0 to 50:50 (ca 25 cv) and then it was further purified by silica flash chromatography (2g) eluting in gradient with 0%-50% Et₂ODCM to afford trans)-8-({[5-(2-fluorophenyl)-2-pyrazinyl]amino}methyl)-3-(3-pyridazinyl)-1-oxa-3-azaspiro[4.5]decan-2-one as a white solid (13 mg).

¹H NMR showed the presence of an impurity (singlet at 4.14 ppm) which was assumed to be the corresponding cis isomer (˜10%); ¹H NMR (400 MHz, CDCl₃): δ 1.22-1.35 (m, 2H), 1.77-1.98 (m, 3H), 2.00-2.11 (m, 4H), 3.39 (t, 2H), 4.24 (s, 2H), 4.92 (t, 1H), 7.16 (ddd, 1H), 7.22-7.28 (m, 1H), 7.29-7.37 (m, 1H), 7.51 (dd, 1H), 7.91 (td, 1H), 8.04 (d, 1H), 8.53-8.61 (m, 2H), 8.97 (d, 1H); UPLC-MS: 0.70 min, 435 [M+H]+.

The above compound (11.8 mg, 0.027 mmol) was suspended in Et₂O (0.5 mL) and HCl 1M in Et₂O (1.2 eq, 0.032 mmol, 0.032 mL) were added. The resulting solid was triturated with Et₂O (3×0.3 mL) and dried (vacuo, 40° C. 12 hours) to afford the title compound as a whitish solid (10.3 mg, 14%)

¹H NMR showed the presence of an impurity (singlet at 4.03 ppm) which was assumed to be the corresponding cis isomer of the target compound (˜10%).

All the analytical data are set forth in the following Table 2-1 and in which R, A₂ and B are:

| | |
|---|---|
| | (IIB) |
| 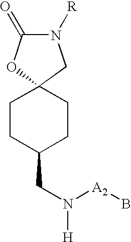 | |

| Cpd. | | | |
|---|---|---|---|
| No. | R | A2-B | Analytical Data |
| 2-1 | 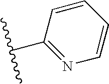 | 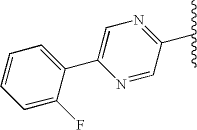 | ¹H NMR (400 MHz, DMSO-d6) δ 1.15- 1.38 (m, 2 H), 1.57-1.77 (m, 3 H), 1.78- 1.92 (m, 2 H), 1.93-2.07 (m, 2 H), 3.18- 3.33 (m, 2 H), 3.96-4.11 (m, 2 H), 7.09- 7.20 (m, 1 H), 7.23-7.32 (m, 2 H), 7.33- 7.41 (m, 1 H), 7.43-7.57 (m, 1 H), 7.79- 7.91 (m, 2 H), 8.06-8.16 (m, 2 H), 8.33- 8.43 (m, 2 H); HPLC-MS: 2.647 min, 434 [M + H]+ |
| 2-2 | 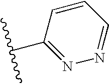 | 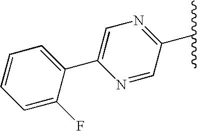 | ¹H NMR (400 MHz, DMSO-d₆) δ 1.19- 1.42 (m, 2 H), 1.62-1.81 (m, 3 H), 1.81- 1.96 (m, 2 H), 1.98-2.12 (m, 2 H), 3.20- 3.34 (m, 2 H), 4.16 (s, 2 H), 7.21-7.33 (m, 2 H), 7.33-7.44 (m, 1 H), 7.44-7.56 (m, 1 H), 7.69-7.79 (m, 1 H), 7.81-7.93 (m, 1 H), 8.08-8.15 (m, 1 H), 8.34-8.44 (m, 2 H), 8.98-9.06 (m, 1 H); UPLC-MS: 0.70 min, 435 [M + H]+. |

### Example 3

### Preparation of Compounds of Formula (IIc)

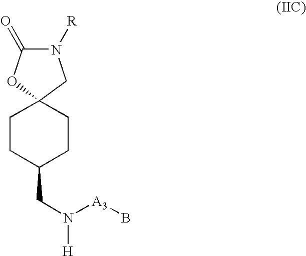

### Example 3-1

### (Trans)-8-({[5-(2-fluorophenyl)-2-pyrimidinyl]amino}methyl)-3-phenyl-1-oxa-3-azaspiro[4.5]decan-2-one

5-(2-fluorophenyl)-2-pyrimidinamine (this can be prepared as described in WO 2003010175, 29.2 mg, 0.154 mmol) and (trans)-2-oxo-3-phenyl-1-oxa-3-azaspiro[4.5]decane-8-carbaldehyde (Intermediate 9, 40 mg, 0.154 mmol) were dissolved in dry tetrahydrofuran (1 ml). Titanium(IV) isopropoxide (0.090 ml, 0.309 mmol) was added and the mixture was stirred at r.t. overnight (ca 17 hours). EtOH (0.150 ml) and sodium borohydride (17.51 mg, 0.463 mmol) were added and the mixture was stirred at r.t. overnight. One drop of water was added and the mixture was evaporated under reduced pressure. Purification by silica gel chromatography (12+M Biotage SP1) eluting with cyclohexane/EtOAc 100:0 to 1:1 was performed followed by a second purification on a NH₂ cartridge (1g) eluting with DCM. Compound fractions were combined and evaporated to afford the title compound as a white solid (0.9 mg, 1%);

### Example 3-2

### (Trans)-8-({[5-(2-fluorophenyl)-2-pyrimidinyl]amino}methyl)-3-(2-pyridinyl)-1-oxa-3-azaspiro[4.5]decan-2-one Hydrochloride

(Trans)-2-oxo-3-(2-pyridinyl)-1-oxa-3-azaspiro[4.5]decane-8-carbaldehyde (prepared in a similar fashion to Intermediate 12 procedure 12a, 76.6 mg, 0.294 mmol) and 5-(2-fluorophenyl)-2-pyrimidinamine (this can be prepared as described in WO 2003010175, 55.7 mg, 0.294 mmol) were dissolved in tetrahydrofuran (3 ml) at r.t. and titanium(IV) isopropoxide (0.172 ml, 0.589 mmol) was added. The reaction was stirred overnight then sodium borohydride (33.4 mg, 0.883 mmol) and 300 μl of ethanol were added. The crude was poured into a saturated aqueous solution of NaHCO₃ (3 ml) and extracted with DCM (3×30 ml), the combined organic phases were filtered using a phase separator tube and the organic phase was concentrated under vacuo. The crude was purified using a 12M NH column eluting in gradient with DCM/Et₂O from 100:0 to 30:70 to give trans-8-({[5-(2-fluorophenyl)-2-pyrimidinyl]amino}methyl)-3-(2-pyridinyl)-1-oxa-3-azaspiro[4.5]decan-2-one (7.2 mg, ˜7%).

¹H NMR (400 MHz, CDCl₃): δ 1.26-1.30 (m, 4H), 1.42-1.46 (m, 2H), 1.75-1.93 (m, 2H), 1.99-2.07 (m, 2H), 3.43 (t, 2H), 4.06-4.08 (m, 2H), 6.99-7.08 (m, 1H), 7.14-7.27 (m, 2H), 7.30-7.42 (m, 2H), 7.69-7.75 (m, 1H), 8.28 (dt, 1H), 8.33-8.36 (m, 1H), 8.51 (d, 1H), 8.53 (d, 1H).

The above compound was dissolved in DCM (1 ml) and a 1M solution of HCl in Et₂O (0.037 ml, 0.037 mmol) was added drop by drop under stirring. The solution was left at r.t. under stirring for 30 min and then the precipitate was separated, triturated with Et₂O, concentrated under a flow of nitrogen and dried for 18 h under high vacuum at 40° C. to give the title compound (4.9 mg, 44%);

### Example 3-3

### (Trans)-3-(2-pyridinyl)-8-({[5-(1,3-thiazol-2-yl)-2-pyrimidinyl]amino}methyl)-1-oxa-3-azaspiro[4.5]decan-2-one

To a solution of (trans)-8-{[(5-bromo-2-pyrimidinyl)amino]methyl}-3-(2-pyridinyl)-1-oxa-3-azaspiro[4.5]decan-2-one (Intermediate 24, 30 mg, 0.072 mmol) in dimethyl sulfoxide (3 ml) were added 2-(tributylstannanyl)-1,3-thiazole (32.2 mg, 0.086 mmol) and Pd(PPh₃)₄ (3.32 mg, 2.87 μmol). The mixture was irradiated in a microwave oven (120° C. 2 cycles of 10 min each one) then partitioned between water (5 ml) and DCM (3×5 ml). The organic phase was dried over Na₂SO₄, filtered and evaporated under reduced pressure. Purification by chromatography (10 g Si—SNAP cartridge, DCM/MeOH 100/0 to 93/7) gave the title compound (16 mg, 52.8%) as a white solid;

### Example 3-4

### (Trans)-3-(2-pyridinyl)-8-({[5-(2-pyridinyl)-2-pyrimidinyl]amino}methyl)-1-oxa-3-azaspiro[4.5]decan-2-one

The title compound was made in a similar fashion to the preparation of Example 3-3 replacing 2-(tributylstannanyl)-1,3-thiazole with 2-(tributylstannanyl)-pyridine (26.4 mg, 0.072 mmol) to afford the title compound (10 mg, 28.5%);

### Example 3-5

### (Trans)-8-({[5-(2-pyrazinyl)-2-pyrimidinyl]amino}methyl)-3-(2-pyridinyl)-1-oxa-3-azaspiro[4.5]decan-2-one

The title compound was made in a similar fashion to the preparation of Example 3-3 replacing 2-(tributylstannanyl)-1,3-thiazole with 2-(tributylstannanyl)-pyrazine (26.5 mg, 0.072 mmol) to afford the title compound (5 mg, 16%);

### Example 3-6

### (Trans)-8-({[5-(1-methyl-1H-imidazol-2-yl)-2-pyrimidinyl]amino}methyl)-3-(2-pyridinyl)-1-oxa-3-azaspiro[4.5]decan-2-one

The title compound was made in a similar fashion to the preparation of Example 3-3 replacing 2-(tributylstannanyl)-1,3-thiazole with 1-methyl-2-(tributylstannanyl)-1H-imidazole (26.6 mg, 0.072 mmol) to afford the title compound (25 mg, 85%);

### Example 3-7

### (Trans)-8-{[(5-bromo-2-pyrimidinyl]amino}methyl)-3-(2-pyridinyl)-1-oxa-3-azaspiro[4.5]decan-2-one

The title compound was made in a similar fashion to the preparation of Intermediate 24 to afford the title compound as a white solid (300 mg, 0.717 mmol, 53%).

### Example 3-8

### (Trans)-8-[(2,5′-bipyrimidin-2′-Vlamino)methyl]-3-(2-pyridinyl)-1-oxa-3-azaspiro[4.5]decan-2-one

To a solution of (trans)-8-{[(5-bromo-2-pyrimidinyl)amino]methyl}-3-(2-pyridinyl)-1-oxa-3-azaspiro[4.5]decan-2-one (Intermediate 24, 30 mg, 0.072 mmol) in dimethylsulfoxide (3 ml) 2-(tributylstannanyl)pyrimidine (26.5 mg, 0.072 mmol) and tetrakis(triphenylphosphine)palladium(0) (3.32 mg, 2.87 μmol) were added and the resulting mixture was irradiated in a microwave oven at 120° C. for 10 min. The crude solution was first eluted on a SCX cartridge (DCM/MeOH/NH₃ (2M soln in MeOH) 100/0/0 to 80/10/10) and evaporated to dryness. Purification by silica gel chromatography (SP1 automated instrument, SNAP 10 g Si cartridge, elution in gradient with 0%-10% MeOHDCM) gave the title compound as a white solid (15 mg, 0.036 mmol, 50%).

### Example 3-9

### (Trans)-8-({[5-(4-pyridazinyl)-2-pyrimidinyl]amino}methyl)-3-(2-pyridinyl)-1-oxa-3-azaspiro[4.5]decan-2-one

The title compound was made in a similar fashion to the preparation of Example 3-8 replacing 2-(tributylstannanyl)pyrimidine with 4-(tributylstannanyl)pyridazine (26.5 mg, 0.072 mmol) to afford the title compound as a white solid (7 mg, 0.015 mmol, 21%).

### Example 3-10

### (Trans)-8-{[(4-phenyl-2-pyrimidinyl)amino]methyl}-3-(3-pyridazinyl)-1-oxa-3-azaspiro[4.5]decan-2-one hydrochloride

In a 2 ml microwave vial 2-chloro-4-phenylpyrimidine (18.17 mg, 0.095 mmol) and (trans)-8-(aminomethyl)-3-(3-pyridazinyl)-1-oxa-3-azaspiro[4.5]decan-2-one (Intermediate 41 procedure 41a, 25 mg, 0.095 mmol) were dissolved in N,N-dimethylacetamide (2 ml). The reaction mixture was irradiated first at 150° C. for 30 min and then at 160° C. for further 30 min. DIPEA (0.033 ml, 0.191 mmol) was added and the mixture was irradiated at 150° C. for 30 min. Solvent was removed under vacuum, the residue was taken up with isopropanol (2.0 ml) and DIPEA (0.033 ml, 0.191 mmol) was added. The reaction mixture was irradiated at 140° C. for 45 min then purified over an ion exchange cartridge (SCX 1g, Varian), washing with MeOH and eluting with 2M ammonia in MeOH. The resulting material was further purified by flash chromatography (Biotage SP1 NH₂ Varian 12M column) eluting with cyclohexane and ethyl acetate to afford (trans)-8-{[(4-phenyl-2-pyrimidinyl)amino]methyl}-3-(3-pyridazinyl)-1-oxa-3-azaspiro[4.5]decan-2-one as a colourless oil (9 mg).

¹H NMR (400 MHz, CDCl₃): δ 8.96 (dd, 1H), 8.55 (dd, 1H), 7.84 (d, 1H), 7.48 (ddd, 1H), 5.90 (d, 1H), 4.91 (br s, 1H), 4.20 (s, 2H), 3.61-3.52 (m, 3H), 3.31 (m, 2H), 2.06-1.14 (m, 14H); UPLC-MS: 0.57 min, 424 [M+H]+.

The above compound was dissolved in DCM (2 ml) and reacted with 1M HCl in Et₂O (1.0 equiv) to give the title compound as a colourless solid (9.9 mg).

### Example 3-11

### (Trans)-8-({[4-(1-pyrimidinyl)-2-pyrimidinyl]amino}methyl)-3-(3-pyridazinyl)-1-oxa-3-azaspiro[4.5]decan-2-one Dihydrochloride

In a 2 ml microwave vial 2-chloro-4-(1-piperidinyl)pyrimidine (18.84 mg, 0.095 mmol) and (trans)-8-(aminomethyl)-3-(3-pyridazinyl)-1-oxa-3-azaspiro[4.5]decan-2-one (Intermediate 41 procedure 41a, 25 mg, 0.095 mmol) were dissolved in N,N-dimethylacetamide (2 ml) and irradiated first at 150° C. for 30 min and then at 160° C. for 30 min. Solvent was removed under vacuum, the residue was taken up with isopropanol (2.0 ml) and DIPEA (0.033 ml, 0.191 mmol) was added. The reaction mixture was irradiated at 150° C. for 1 hour and then at 180° C. for 2 hours. The mixture was purified over an ion exchange SCX cartridge (1g, Varian), washing with MeOH and eluting with 2M ammonia in MeOH. The resulting material was further purified by flash chromatography (Biotage SP1, NH₂ Varian 12M column) eluting with cyclohexane and ethyl acetate to afford (trans)-8-({[4-(1-piperidinyl)-2-pyrimidinyl]amino}methyl)-3-(3-pyridazinyl)-1-oxa-3-azaspiro[4.5]decan-2-one as a colourless oil (4.4 mg).

¹H NMR (400 MHz, CDCl₃): δ 8.96 (dd, 1H), 8.55 (dd, 1H), 7.84 (d, 1H), 7.48 (dd, 1H), 5.90 (d, 1H), 4.91 (br s, 1H), 4.20 (s, 2H), 3.61-3.52 (m, 3H), 3.31 (t, 2H), 2.06-1.14 (m, 14H); UPLC-MS: 0.57 min, 424 [M+H]+.

The above compound was dissolved in DCM (2 ml) and reacted with 1M HCl in Et₂O (2.0 equiv) to give the title compound as a colourless solid (4.4 mg).

### Example 3-12

### (Trans)-8-[({4-[(phenylmethyl)oxy]-2-pyrimidinyl}amino)methyl]-3-(3-pyridazinyl)-1-oxa-3-azaspiro[4.5]decan-2-one hydrochloride

The title compound was made in a similar fashion to the preparation of Example 3-10 replacing 2-chloro-4-phenylpyrimidine with 2-chloro-4-[(phenylmethyl)oxy]pyrimidine (21.0 mg, 0.095 mmol) to afford the title compound as a colourless solid (5.0 mg).

All the analytical data are set forth in the following Table 3-1 and in which R, A₃ and B are:

| | |
|---|---|
| | (IIC) |
| 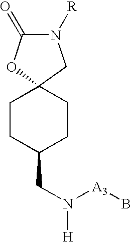 | |

| Cpd. | | | |
|---|---|---|---|
| No. | R | A3-B | Analytical Data |
| 3-1 | Ph | 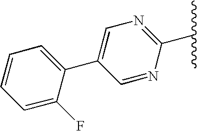 | ¹H NMR (400 MHz, CDCl₃): δ 1.17-1.31 (m, 2 H), 1.73-1.96 (m, 3 H), 1.97-2.11 (m, 4 H), 3.46 (t, 2 H), 3.82 (s, 2 H), 5.25-5.34 (m, 1 H), 7.12-7.27 (m, 2 H), 7.31-7.46 (m, 4 H), 7.52-7.63 (m, 3 H), 8.48-8.54 (m, 2 H); UPLC-MS: 0.82 min, 433 [M + H]+. |
| 3-2 | 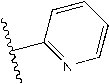 | 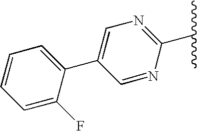 | ¹H NMR (500 MHz, DMSO-d₆): δ 1.17- 1.27 (m, 2 H), 1.58-1.75 (m, 3 H), 1.76- 1.87 (m, 2 H), 1.92-2.00 (m, 2 H), 3.21- 3.28 (m, 2 H), 4.00 (s, 2 H,) 7.11-7.17 (m, 1 H), 7.24 -7.43 (m, 3 H), 7.51-7.62 (m, 2 H), 7.80-7.86 (m, 1 H), 8.07-8.09 (m, 1 H), 8.34-8.38 (m, 1 H), 8.46-8.50 (m, 2 H); UPLC-MS: 0.89 min, 434 [M + H]+. |
| 3-3 | 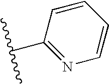 | 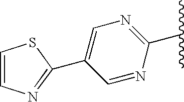 | ¹H NMR (400 MHz, CDCl₃): δ 8.85 (2 H, s), 8.33 (2 H, m), 7.86 (1 H, d), 7.73 (1 H, m), 7.31 (1 H, d), 7.06 (1 H, td), 5.59 (1 H, t), 4.07 (2 H, t), 3.46 (2 H, t), 2.02 (4 H, m), 1.83 (3 H, m), 1.70 (3 H, br s), 1.28 (3 H, m); UPLC-MS: 0.68 min, 423 [M + H]⁺ |
| 3-4 | 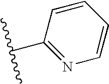 | 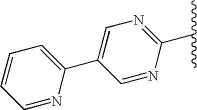 | ¹H NMR (400 MHz, CDCl₃): δ 8.93 (1 H, s), 8.67 (1 H, m), 8.32 (2 H, m), 7.74 (2 H, m), 7.61 (1 H, d), 7.23 (1 H, ddd), 7.05 (1 H, m), 5.52 (1 H, t), 4.06 (2 H, br s), 3.42 (2 H, m), 2.02 (5 H, m), 1.84 (3 H, m), 1.27 (2 H, m); UPLC-MS: 0.62 min, 417 [M + H]⁺ |
| 3-5 | 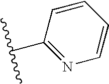 | 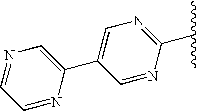 | ¹H NMR (400 MHz, CDCl₃): δ 8.96 (2 H, br s), 8.93 (1 H, d), 8.61 (1 H, dd), 8.50 (1 H, d), 8.35 (1 H, dd), 8.28 (1 H, dt), 7.73 (1 H, ddd), 7.06 (1 H, ddd), 5.53 (1 H, t), 4.06 (2 H, br s), 3.47 (2 H, t), 2.03 (4 H, m), 1.84 (3 H, m), 1.28 (3 H, m); UPLC-MS: 0.63 min, 418 [M + H]⁺ |
| 3-6 | 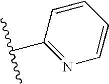 | 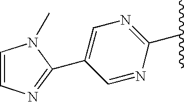 | ¹H NMR (400 MHz, CDCl₃): δ 8.32 (4 H, m), 7.73 (1 H, ddd), 7.56 (1 H, s), 7.06 (2 H, m), 5.37 (1 H, t), 4.06 (2 H, br s), 3.64 (3 H, s), 3.42 (2 H, t), 2.03 (4 H, m), 1.83 (3 H, m), 1.30 (3 H, m); UPLC-MS: 0.50 min, 420 [M + H]⁺ |
| 3-7 | 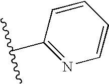 | 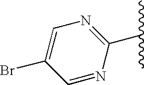 | ¹H NMR (400 MHz, CDCl₃): δ 8.34 (1 H, dt) 8.28 (3 H, m), 7.72 (1 H, ddd), 7.05 (1 H, ddd), 5.22 (1 H, t), 4.06 (2 H, br s), 3.34 (2 H, t), 1.98 (4 H, m), 1.79 (3 H, m), 1.23 (3 H, m); UPLC-MS: 0.74 min, 418 and 420 [M + H]⁺ |
| 3-8 | 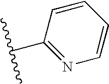 | 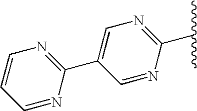 | ¹H NMR (400 MHz, CDCl₃): δ 9.29 (2 H, s), 8.75 (2 H, d), 8.31-8.41 (1 H, m), 8.27 (1 H, d), 7.72 (1 H, ddd), 7.16 (1 H, t), 7.00- 7.08 (1 H, m), 5.55 (1 H, t), 4.06 (2 H, s), 3.48 (2 H, t), 2.02 (4 H, d), 1.70-1.93 (3 H, m), 1.17-1.35 (2 H, m); UPLC-MS: 0.65 min, 418 [M + H]⁺ |
| 3-9 | 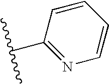 | 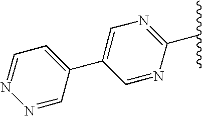 | ¹H NMR (400 MHz, CDCl₃): δ 9.42 (1 H, dd), 9.21 (1 H, dd), 8.65 (2 H, br s.), 8.31- 8.37 (1 H, m), 8.27 (1 H, d), 7.72 (1 H, ddd), 7.58 (1 H, dd), 7.05 (1 H, ddd), 5.61 (1 H, t), 4.02-4.10 (2 H, m), 3.46 (2 H, t), 1.95-2.08 (4 H, m), 1.73-1.93 (3 H, m), 1.22-1.33 (2 H, m); UPLC-MS: 0.57 418 [M + H]⁺ |
| 3-10 | 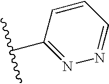 | 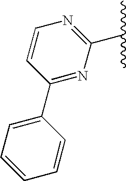 | ¹H NMR (400 MHz, DMSO-d6): δ 9.01 (dd, 1 H), 8.39 (dd, 1 H), 7.99 (br s, 1 H), 7.79 (br s, 1 H), 7.74 (ddd, 1 H), 6.53 (br s, 1 H), 4.14 (s, 2 H), 3.95-3.57 (m, 10 H), 3.30 (br s, 1 H), 2.13-1.17 (m, 11 H); UPLC-MS: 0.57 min, 424 [M + H]+ |
| 3-11 | 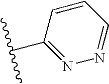 | 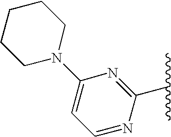 | ¹H NMR (400 MHz, DMSO-d6): δ 9.01 (dd, 1 H), 8.39 (dd, 1 H), 7.99 (br s, 1 H), 7.74 (dd, 1 H), 6.53 (brs, 1 H), 4.14 (s, 2 H), 3.95-3.57 (m, 4 H), 3.30 (br s, 2 H), 2.13-1.17 (m, 16 H); UPLC-MS: 0.57 min, 424 [M + H]+ |
| 3-12 | 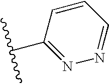 | 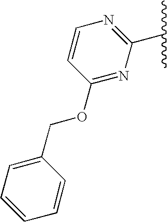 | ¹H NMR (400 MHz, DMSO-d6): δ 9.01 (dd, 1 H), 8.70 (m, 1 H), 8.38 (dd, 1 H), 8.16 (m, 1 H), 7.74 (ddd, 1 H), 7.51-7.36 (m, 5 H), 6.47 (m, 1 H), 5.46 (m, 2 H), 4.2 (s, 2 H), 3.36 (m, 2 H), 2.08-1.14 (m, 9 H); UPLC-MS: 0.60 min, 446 [M + H]+ |

### Example 4

### Preparation of Compounds of Formula (IID)

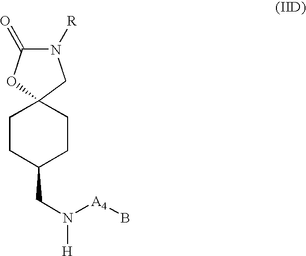

### Example 4-1

### (Trans)-3-(2-pyridinyl)-8-({[6-(1-pyrrolidinyl)-3-pyridazinyl]amino}methyl)-1-oxa-3-azaspiro[4.5]decan-2-one Hydrochloride

The title compound was made in a similar fashion to the preparation of Example 1-3 replacing 5-(2-pyrimidinyl)-2-pyridinamine with 6-(1-pyrrolidinyl)-3-pyridazinamine (this may be prepared as described in U.S. Pat. No. 4,104,385 or DE2737542, 30 mg, 0.183 mmol) and purifying with MDAP to afford the title compound (6 mg);

### Example 4-2

### (Trans)-8-({[6-(2-fluorophenyl)-3-pyridazinyl]amino}methyl)-3-(2-pyridinyl)-1-oxa-3-azaspiro[4.5]decan-2-one

(Trans)-8-{[(6-chloro-3-pyridazinyl)amino]methyl}-3-(2-pyridinyl)-1-oxa-3-azaspiro[4.5]decan-2-one (Intermediate 23, 82 mg, 0.219 mmol), (2-fluorophenyl)boronic acid (33.8 mg, 0.241 mmol), Pd(PPh₃)₄ (12.67 mg, 10.97 μmol) and sodium carbonate (59.3 mg, 0.559 mmol) were placed in a vial under nitrogen. Then toluene (430 μl), ethanol (430 μl) and water (430 μl) were added and the mixture was heated at 110° C. for 6 h. The mixture was allowed to cool to r.t. and 20 ml of AcOEt were added followed by 10 ml of a saturated solution of K₂CO₃. The aqueous phase was extracted with 20 ml of AcOEt. The combined organic layers were washed with 20 ml of brine, dried over Na₂SO₄, filtered and evaporated to dryness. The resulting compound was purified by flash chromatography (Biotage SP1, 50 g SNAP silica gel column) with the following eluent: A: cyclohexane/B: AcOEt: 0% B for 3 min, 0% to 100% B in 20 min, 60% B for 6 min, 60% B to 100% B in 5 min, 100% B for 5 min. Evaporation of the solvents gave the title compound as a slightly yellow solid (76 mg, 76%);

### Example 4-3

### (Trans)-8-({[6-(3,5-difluorophenyl)-3-pyridazinyl]amino}methyl)-3-(3-pyridazinyl)-1-oxa-3-azaspiro[4.5]decan-2-one Dihydrochloride

### Procedure a

Potassium carbonate (253 mg, 1.830 mmol), (trans)-8-{[(6-chloro-3-pyridazinyl)amino]methyl}-3-(3-pyridazinyl)-1-oxa-3-azaspiro[4.5]decan-2-one (Intermediate 42 procedure 42a, 343 mg, 0.915 mmol), PdCl₂(PPh₃)₂ (32.1 mg, 0.046 mmol) and (3,5-difluorophenyl)boronic acid (289 mg, 1.830 mmol) were weighed into a microwave reaction vial and placed under nitrogen. De-aereated acetonitrile (9 ml) and de-aereated water (3 ml) were added and the resulting biphasic system was stirred and heated to 120° C. for 45 minutes in a microwave reactor. The mixture was evaporated under reduced pressure and the resulting mixture was partitioned between dichloromethane (75 ml) and water (75 ml) and filtered through a hydrophobic frit (phase separator cartridge). The aqueous was further extracted with dichloromethane (2×50 ml). The combined organic phases were evaporated under reduced pressure and the resulting residue was purified via reverse phase chromatography on the Biotage (Phase A, water 95%, acetonitrile 4.9%, formic acid 0.1%; Phase B acetonitrile 99.9%, formic acid 0.1%; elution began with 100% A then a gradient to 5% A/95% B then isocratic at 5% A/95% B then back to 100% A; SNAP 100 g C-18 column). The fractions containing the desired product were partially evaporated under reduced pressure then loaded onto an SCX (10g) cartridge. The cartridge was eluted with MeOH and then 2M NH₃ in MeOH. The product containing fractions were evaporated to give (trans)-8-({[6-(3,5-difluorophenyl)-3-pyridazinyl]amino}methyl)-3-(3-pyridazinyl)-1-oxa-3-azaspiro[4.5]decan-2-one (198 mg) as a white solid.

¹H NMR (400 MHz, CDCl₃): δ 8.97 (dd, 1H), 8.58 (dd, 1H), 7.48-7.62 (m, 4H), 6.86 (tt, 1H), 6.76 (d, 1H), 4.91 (t, 1H), 4.24 (s, 2H), 3.49 (t, 2H), 2.01-2.14 (m, 4H), 1.86-2.00 (m, 3H), 1.21-1.35 (m, 2H); UPLC-MS: 0.56 min, 453 [M+H]+

The above product was dissolved in dichloromethane/MeOH (3:1, 8 ml) and treated with 1M HCl in diethyl ether (1 ml, 1 mmol). This was stirred for 10 minutes then the volatiles were evaporated on V-10 evaporator. Acetone (4 ml) was added then evaporated. The residue was triturated with acetone and filtered, washing the filter cake with more acetone (2 ml). The white solid was collected and dried under reduced pressure at 40° C. overnight to give the title compound as a white solid (223 mg);

### Procedure b

TFA (1 ml, 12.98 mmol) was added to a stirred solution of 1,1-dimethylethyl [6-(3,5-difluorophenyl)-3-pyridazinyl]{[(trans)-2-oxo-3-(3-pyridazinyl)-1-oxa-3-azaspiro[4.5]dec-8-yl]methyl}carbamate (Intermediate 57, 135 mg, 0.159 mmol) in dichloromethane (2 ml) at room temperature. The mixture was stirred for 1 hour. The volatiles were evaporated under reduced pressure and the residue loaded onto a preconditioned SCX cartridge (1g). This was eluted with MeOH and then 2M NH₃ in MeOH. The basic fractions containing product were evaporated to give a residue which was purified via Biotage (30%-100% EtOAc/cyclohexane; 12M NH column) to give (trans)-8-({[6-(3,5-difluorophenyl)-3-pyridazinyl]amino}methyl)-3-(3-pyridazinyl)-1-oxa-3-azaspiro[4.5]decan-2-one (72 mg) as a white solid.

¹H NMR (400 MHz, CDCl₃): δ 8.98 (dd, 1H), 8.58 (dd, 1H), 7.47-7.62 (m, 4H), 6.86 (tt, 1H), 6.78 (d, 1H), 5.06 (brs, 1H), 4.24 (s, 2H), 3.49 (t, 2H), 2.02-2.13 (m, 4H), 1.86-2.01 (m, 3H), 1.20-1.35 (m, 2H); UPLC-MS: 0.80 min, 453 [M+H]+

71 mg of this material (0.157 mmol) together with another 2 batches of (trans)-8-({[6-(3,5-difluorophenyl)-3-pyridazinyl]amino}methyl)-3-(3-pyridazinyl)-1-oxa-3-azaspiro[4.5]decan-2-one prepared in a similar fashion (279 mg, 0.617 mmol) and (96 mg, 0.212 mmol) were dissolved in dichloromethane/MeOH and treated with 1.0M HCl in diethyl ether (2 ml, 2 mmol). This was stirred for 10 minutes then the volatiles were evaporated and the residue was triturated with diethyl ether. The solids were filtered, washing with diethyl ether and dried at 50° C. under reduced pressure overnight then dried further under high vacuum at 50° C. for 6 hours then at 70° C. for 48 hours to give the title compound (444 mg) as an off white solid.

### Example 4-3 A

### Characterisation of Form 1 of (trans)-8-({[6-(3,5-difluorophenyl)-3-pyridazinyl]amino}-methyl)-3-(3-pyridazinyl)-1-oxa-3-azaspiro[4.5]decan-2-one

Free base of (trans)-8-({[6-(3,5-difluorophenyl)-3-pyridazinyl]amino}methyl)-3-(3-pyridazinyl)-1-oxa-3-azaspiro[4.5]decan-2-one, obtained according to Example 4-3 procedure b, has been characterised by X-Ray powder diffraction spectrometry, Raman spectrometry, Differential Scanning Calorimetry (DSC) and FT-IR. Free base of (trans)-8-({[6-(3,5-difluorophenyl)-3-pyridazinyl]amino}methyl)-3-(3-pyridazinyl)-1-oxa-3-azaspiro[4.5]decan-2-one, obtained according to Example 4-3, procedure b, shows the XPRD pattern of FIG. 1 and has been identified as Form 1.

Table 1 shows the XRPD angles and d spacings for Form 1 of the free base of (trans)-8-({[6-(3,5-difluorophenyl)-3-pyridazinyl]amino}methyl)-3-(3-pyridazinyl)-1-oxa-3-azaspiro[4.5]decan-2-one.

TABLE 1

| Angle | d value |
|---|---|
| 2-Theta ° | Angstrom |
| 4.9 | 18.0 |
| 9.8 | 9.0 |
| 10.2 | 8.6 |
| 11.1 | 8.0 |
| 12.3 | 7.2 |
| 14.0 | 6.3 |
| 14.7 | 6.0 |
| 15.3 | 5.8 |
| 16.0 | 5.6 |
| 16.2 | 5.4 |
| 17.7 | 5.0 |
| 18.9 | 4.7 |
| 19.8 | 4.5 |
| 20.0 | 4.4 |
| 20.5 | 4.3 |
| 21.2 | 4.2 |
| 22.2 | 4.0 |
| 23.4 | 3.8 |
| 23.9 | 3.7 |
| 24.4 | 3.6 |
| 24.9 | 3.6 |
| 25.4 | 3.5 |
| 26.3 | 3.4 |
| 27.2 | 3.3 |

The X-ray powder diffraction pattern of Form 1 of (trans)-8-({[6-(3,5-difluorophenyl)-3-pyridazinyl]amino}methyl)-3-(3-pyridazinyl)-1-oxa-3-azaspiro[4.5]decan-2-one as shown in FIG. 1 exhibits predominant peaks (expressed in degrees 2θ (+/−0.15 degrees 20 at one or more of the following positions: 4.9, 9.8, 10.2, 14.7, 15.3, 15.9, 16.2, 17.7, 19.8, 20.0, 21.2, 22.2, 23.4, 27.2.

### DESCRIPTION OF FIGURES

FIG. 1 shows X-Ray powder diffraction data obtained for Form 1 of (trans)-8-({[6-(3,5-difluorophenyl)-3-pyridazinyl]amino}methyl)-3-(3-pyridazinyl)-1-oxa-3-azaspiro[4.5]decan-2-one as described before. The hygroscopic form is characterised by having an XRPD pattern with signals substantially as listed in Table 1.

FIG. 2 shows a Differential Scanning Calorimetry (DSC) thermogram of Form 1 of (trans)-8-({[6-(3,5-difluorophenyl)-3-pyridazinyl]amino}methyl)-3-(3-pyridazinyl)-1-oxa-3-azaspiro[4.5]decan-2-one.

FIG. 3 shows the Raman spectrum of Form 1 of (trans)-8-({[6-(3,5-difluorophenyl)-3-pyridazinyl]amino}methyl)-3-(3-pyridazinyl)-1-oxa-3-azaspiro[4.5]decan-2-one.

FIG. 4 shows the FT-IR spectrum of Form 1 of (trans)-8-({[6-(3,5-difluorophenyl)-3-pyridazinyl]amino}methyl)-3-(3-pyridazinyl)-1-oxa-3-azaspiro[4.5]decan-2-one.

### X-Ray Powder Diffraction

X Ray Powder Diffraction (XRPD) analysis was performed on Panalytical X'Pert Pro, using x'celerator/type RTMS detector. The acquisition conditions were: radiation: Cu Kα, generator tension: 40 kV, generator current: 40 mA, start angle: 2.0° 2Φ, end angle: 45.0° 2Φ, step size: 0.0170° 2Φ(depends on actual X'celerator used) time per step: 32.3024 sec (about 10 min) slow scan or 9.5 sec for fast scan. The sample was prepared on a low background sample holder. It will be recognised that spectra and diffraction data will vary slightly according to various factors such as the temperature, concentration and instrumentation used. The skilled person will recognise that XRPD peak positions are affected by differences in sample height. The peak positions quoted herein are thus subject to a variation of +/−0.15 degrees 2-theta.

### Raman Spectroscopy

The Raman spectrum was recorded with the sample on AI sample pan at 4 cm⁻¹ resolution, laser=785 nm, power output 100 mw.

Instrument Configuration Kaiser RXN1 Kaiser Optical System Micro Raman

### Differential Scanning Calorimetry (DSC)

Instrument configuration: PE DSC 7, not ermetic sample pan, run @10K/min to 150° C., sample 1.5-5 mg.

The DSC thermogram was obtained using a TA Instruments Q2000 calorimeter. The sample was weighted into an aluminium pan, a pan lid placed on top and lightly crimped without sealing the pan. The experiment was conducted using a heating rate of 10° C. min⁻¹. A melting endotherm was observed at an onset temperature of ???° C.

It should be recognized that the endotherm peak as measured is dependent under a number of factors including the machine employed, the rate of heating, the calibration standard, humidity and the purity of the sample used. Accordingly, the term “about ??? ° C.” is intended to encompass such instrument variations.

### FT-IR

The infrared spectrum was recorded using a Nexus FTIR (Thermo Nicolet) spectrometer over the range 4000 cm⁻¹ to 400 cm⁻¹ fitted with Attenuated Total Reflectance (ATR) Accessory at 4 cm⁻¹ resolution and equipped with a DTGS KBr detector.

### Example 4-3 B

### Characterisation of Form 2 of (trans)-8-({[6-(3,5-difluorophenyl)-3-pyridazinyl]amino}-methyl)-3-(3-pyridazinyl)-1-oxa-3-azaspiro[4.5]decan-2-one

Form 2 of (trans)-8-({[6-(3,5-difluorophenyl)-3-pyridazinyl]amino}methyl)-3-(3-pyridazinyl)-1-oxa-3-azaspiro[4.5]decan-2-one has been obtained by crystallisation with three different solvents according to the following procedure:

50 mg of (trans)-8-({[6-(3,5-difluorophenyl)-3-pyridazinyl]amino}methyl)-3-(3-pyridazinyl)-1-oxa-3-azaspiro[4.5]decan-2-one obtained according to Example 4.3, procedure b, was slurried in 1 ml of EtOAc and was left to temperature cycle from 0-40° C. over the weekend. The same procedure has been used replacing EtOAc with MIBK (methyl isobutyl ketone) or MeOH.

For all the preparations after temperature cycling for the weekend sub samples were removed for analysis. All samples were concordant with one another both pre- and post-drying.

The following scale up crystallisation was performed:

500 mg of (trans)-8-({[6-(3,5-difluorophenyl)-3-pyridazinyl]amino}methyl)-3-(3-pyridazinyl)-1-oxa-3-azaspiro[4.5]decan-2-one as prepared according to Example 4.3, procedure b was slurried in 15 ml of MeOH and seeded with previous seeds obtained as previously described. The resulting slurry was left to stir overnight. After drying overnight a sample was removed for analysis by IR. The sample was isolated by filtration and was left to dry overnight in vacuo at 40° C. After drying overnight, 436.44 mg was obtained. Samples were removed for analysis by HPLC and NMR (DMSO). The batch was 99% pure and consistent with (trans)-8-({[6-(3,5-difluorophenyl)-3-pyridazinyl]amino}methyl)-3-(3-pyridazinyl)-1-oxa-3-azaspiro[4.5]decan-2-one by NMR.

The product so obtained has been identified as Form 2 of the free base of (trans)-8-({[6-(3,5-difluorophenyl)-3-pyridazinyl]amino}methyl)-3-(3-pyridazinyl)-1-oxa-3-azaspiro[4.5]-decan-2-one and shows the XPRD pattern of FIG. 5.

Table 2 shows the XRPD angles and d spacings for Form 2 of the free base of (trans)-8-({[6-(3,5-difluorophenyl)-3-pyridazinyl]amino}methyl)-3-(3-pyridazinyl)-1-oxa-3-azaspiro[4.5]decan-2-one.

TABLE 2

| Angle | d value |
|---|---|
| 2-Theta ° | Angstrom |
| 5.3 | 16.7 |
| 9.7 | 9.1 |
| 10.6 | 8.4 |
| 12.3 | 7.2 |
| 13.3 | 6.7 |
| 14.3 | 6.2 |
| 15.8 | 5.6 |
| 16.2 | 5.5 |
| 16.5 | 5.4 |
| 16.9 | 5.2 |
| 17.1 | 5.2 |
| 18.0 | 4.9 |
| 18.7 | 4.7 |
| 19.4 | 4.6 |
| 20.6 | 4.3 |
| 20.9 | 4.2 |
| 21.2 | 4.2 |
| 21.5 | 4.1 |
| 23.0 | 3.9 |
| 23.6 | 3.8 |
| 24.6 | 3.6 |
| 25.4 | 3.5 |
| 25.7 | 3.5 |
| 26.6 | 3.3 |
| 27.1 | 3.3 |
| 28.0 | 3.2 |
| 28.4 | 3.1 |
| 28.6 | 3.1 |
| 28.8 | 3.1 |
| 30.0 | 3.0 |
| 30.8 | 2.9 |
| 31.3 | 2.8 |
| 32.7 | 2.7 |
| 33.5 | 2.7 |
| 34.7 | 2.6 |
| 35.3 | 2.5 |
| 36.6 | 2.4 |
| 39.6 | 2.3 |

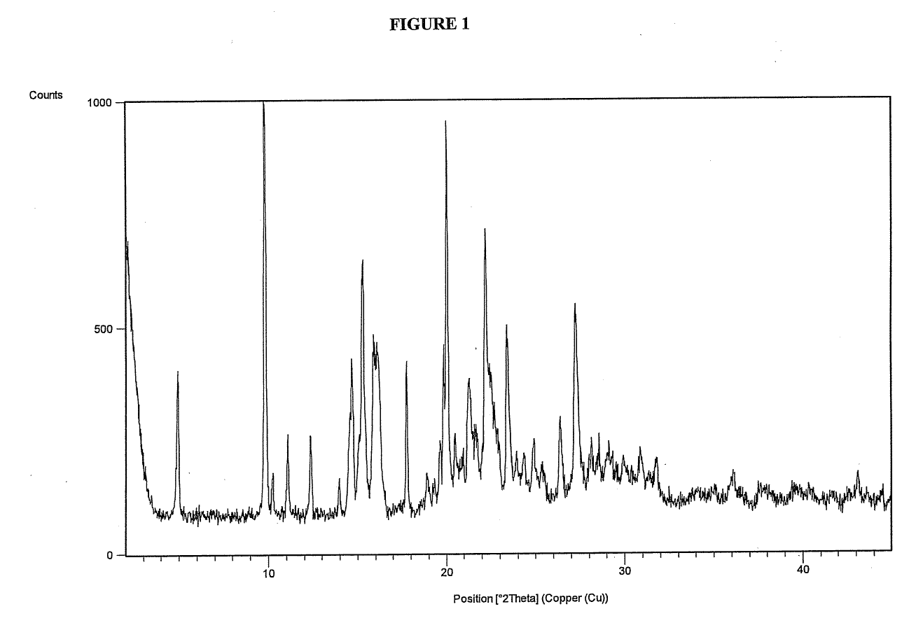

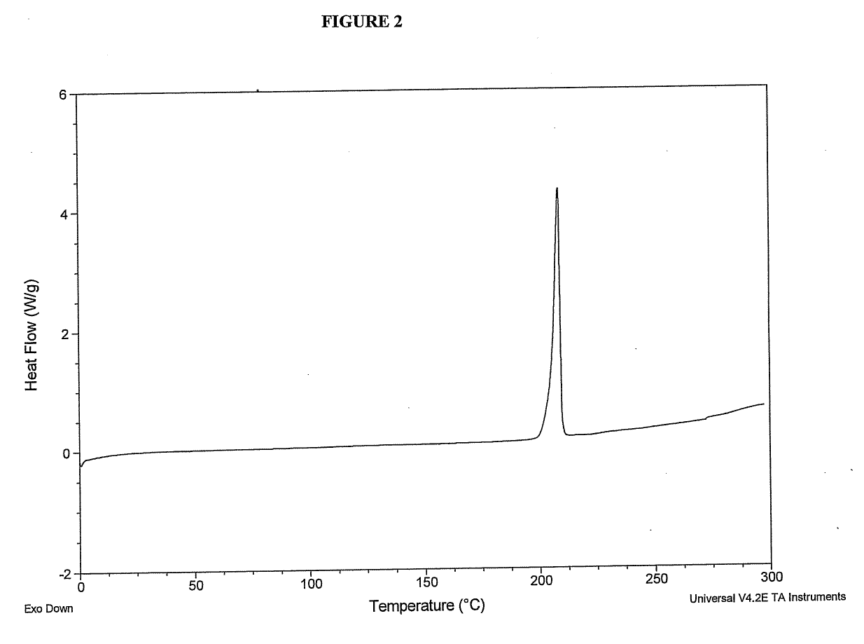

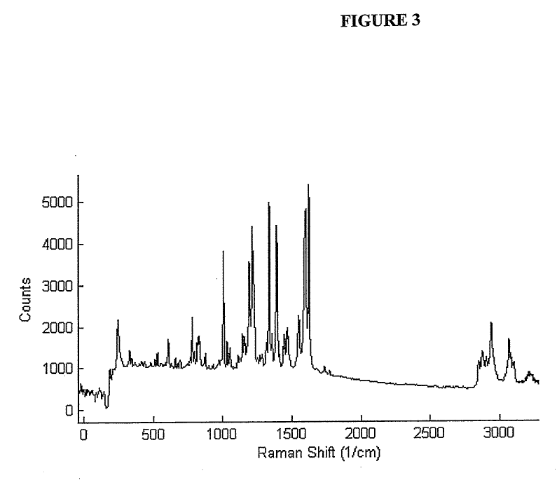

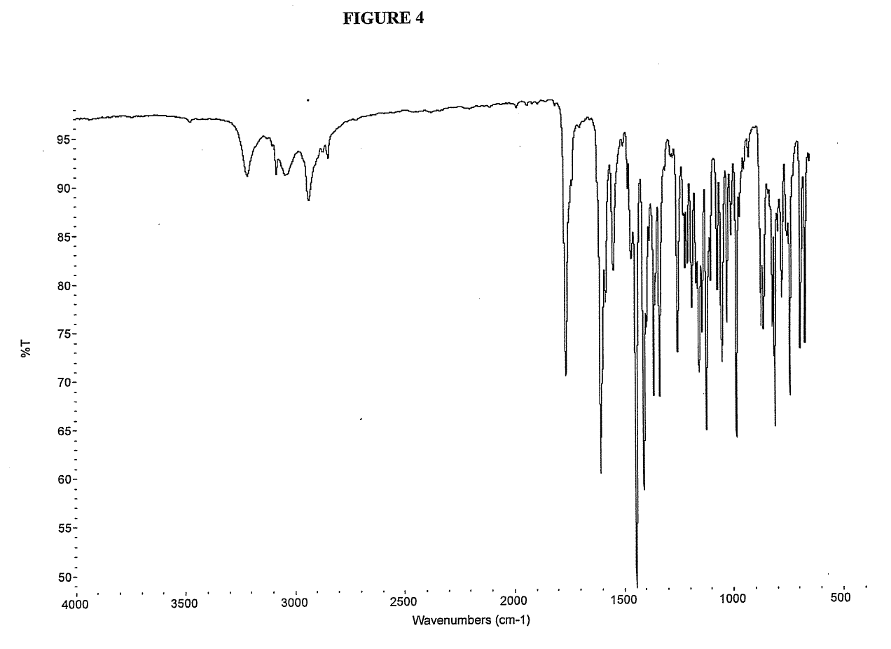

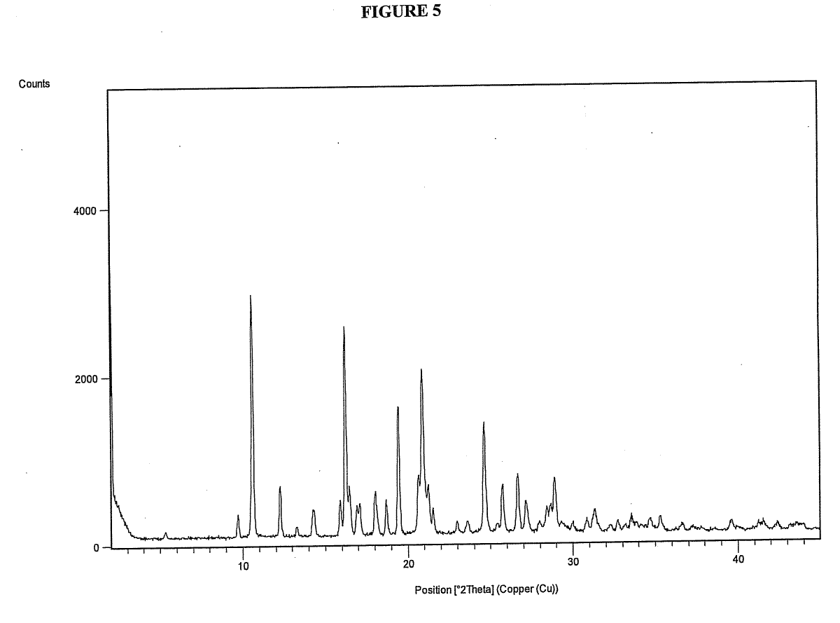

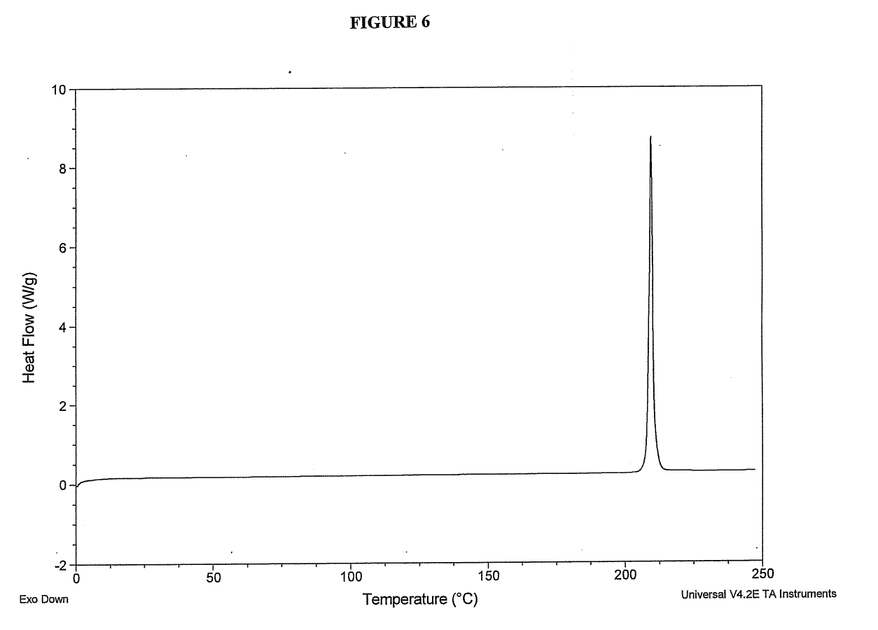

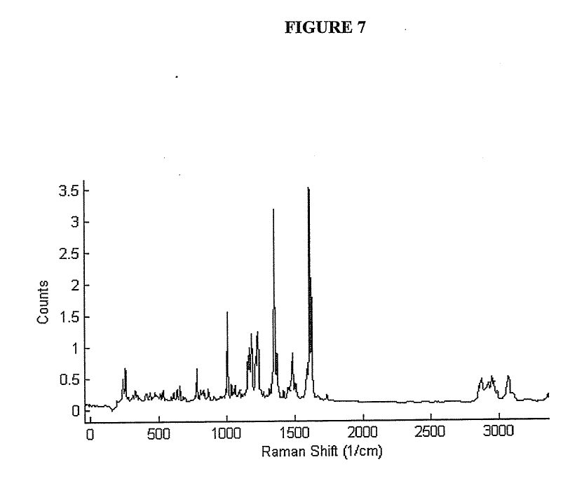

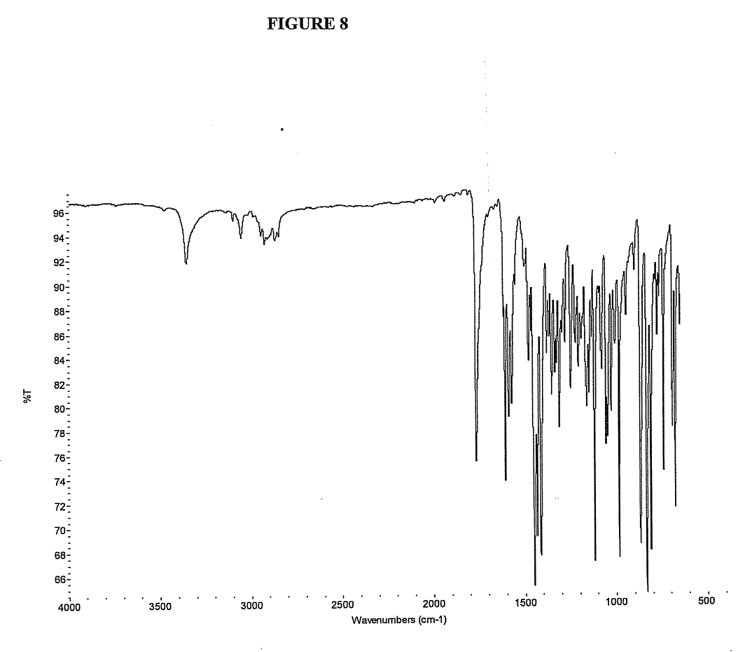

The X-ray powder diffraction pattern of Form 2 of (trans)-8-({[6-(3,5-difluorophenyl)-3-pyridazinyl]amino}methyl)-3-(3-pyridazinyl)-1-oxa-3-azaspiro[4.5]decan-2-one as shown in FIG. 1 exhibits predominant peaks (expressed in degrees 2θ (+/−0.15 degrees 2θ) at one or more of the following positions: 5.3, 9.7, 10.6, 12.3, 16.2, 19.4, 20.9, 24.6.

### DESCRIPTION OF FIGURES

FIG. 5 shows X-Ray powder diffraction data obtained for Form 2 of (trans)-8-({[6-(3,5-difluorophenyl)-3-pyridazinyl]amino}methyl)-3-(3-pyridazinyl)-1-oxa-3-azaspiro[4.5]decan-2-one as described before. Form 2 of (trans)-8-({[6-(3,5-difluorophenyl)-3-pyridazinyl]amino}methyl)-3-(3-pyridazinyl)-1-oxa-3-azaspiro[4.5]decan-2-one is characterised by having an XRPD pattern with signals substantially as listed in Table 2.

FIG. 6 shows a Differential Scanning Calorimetry (DSC) thermogram of Form 2 of (trans)-8-({[6-(3,5-difluorophenyl)-3-pyridazinyl]amino}methyl)-3-(3-pyridazinyl)-1-oxa-3-azaspiro[4.5]decan-2-one.

FIG. 7 shows the Raman spectrum of Form 2 of (trans)-8-({[6-(3,5-difluorophenyl)-3-pyridazinyl]amino}methyl)-3-(3-pyridazinyl)-1-oxa-3-azaspiro[4.5]decan-2-one.

FIG. 8 shows the FT-IR spectrum of Form 2 of (trans)-8-({[6-(3,5-difluorophenyl)-3-pyridazinyl]amino}methyl)-3-(3-pyridazinyl)-1-oxa-3-azaspiro[4.5]decan-2-one.

### X-Ray Powder Diffraction

X Ray Powder Diffraction (XRPD) analysis was performed on Panalytical X'Pert Pro, using x'celerator/type RTMS detector. The acquisition conditions were: radiation: Cu Kα, generator tension: 40 kV, generator current: 40 mA, start angle: 2.0° 2Φ, end angle: 45.0° 2Φ, step size: 0.0170° 2Φ (depends on actual X'celerator used) time per step: 32.3024 sec (about 10 min) slow scan or 9.5 sec for fast scan. The sample was prepared on a low background sample holder. It will be recognised that spectra and diffraction data will vary slightly according to various factors such as the temperature, concentration and instrumentation used. The skilled person will recognise that XRPD peak positions are affected by differences in sample height. The peak positions quoted herein are thus subject to a variation of +/−0.15 degrees 2-theta.

### Raman Spectroscopy

The Raman spectrum was recorded with the sample on AI sample pan at 4 cm⁻¹ resolution, laser=785 nm, power output 100 mw.

Instrument Configuration Kaiser RXN1 Kaiser Optical System Micro Raman

### Differential Scanning Calorimetry (DSC)

The DSC thermogram was obtained using a TA Instruments Q2000 calorimeter. The sample was weighted into an aluminium pan, a pan lid placed on top and lightly crimped without sealing the pan. The experiment was conducted using a heating rate of 10° C. min⁻¹. A melting endotherm was observed at an onset temperature of 208.4° C. It should be recognized that the endotherm peak as measured is dependent under a number of factors including the machine employed, the rate of heating, the calibration standard, humidity and the purity of the sample used. Accordingly, the term “about 208° C.” is intended to encompass such instrument variations.

### FT-IR

The infrared spectrum was recorded using a Nexus FTIR (Thermo Nicolet) spectrometer over the range 4000 cm⁻¹ to 400 cm⁻¹ fitted with Attenuated Total Reflectance (ATR) Accessory at 4 cm⁻¹ resolution and equipped with a DTGS KBr detector.

### Example 4-4

### (Trans)-8-({[6-(2-methylphenyl)-3-pyridazinyl]amino}methyl)-3-(3-pyridazinyl)-1-oxa-3-azaspiro[4.5]decan-2-one dihydrochloride

(Trans)-8-({[6-(2-methylphenyl)-3-pyridazinyl]amino}methyl)-1-oxa-3-azaspiro[4.5]decan-2-one (Intermediate 45, 40.0 mg, 0.114 mmol), 3-chloropyridazine (prepared according to WO/0107416, 13 mg, 0.114 mmol), copper(I) iodide (21.62 mg, 0.114 mmol), trans-1,2-diaminocyclohexane (0.027 ml, 0.227 mmol), K₃PO₄ (72.3 mg, 0.341 mmol) were collected in a closed vial and suspended in 1,4-dioxane (4 ml). The resulting mixture was stirred at 130° C. for 4 hours. Solvent was removed under vacuum. The crude was taken up with DCM (8 ml) and filtered over a separating tube. The resulting solution was purified with Biotage SP1, over a 12M NH₂ Varian cartridge, eluting with a gradient of cyclohexane and ethyl acetate. The title compound was eluted with EtOAc and recovered as colourless oil. Then it was further purified with Biotage SP1, over a 12M NH₂ Varian cartridge, eluting with a gradient of cyclohexane and ethyl acetate to recover the title compound as nearly chemically pure colourless oil (30 mg).

¹H NMR (400 MHz, CDCl₃): δ 8.97 (dd, 1H), 8.58 (dd, 1H), 7.52 (dd, 1H), 7.45-7.41 (m, 1H), 7.36-7.29 (m, 4H), 6.75 (m, 1H), 4.92 (brs, 2H), 3.45 (t, 2H), 2.43 (s, 3H), 2.15-1.11 (m, 9H).

The above compound was dissolved in DCM (2 ml) and reacted with 2.0 equiv. of 1M HCl in Et₂O. The resulting colourless solid was tritured with Et₂O (3×2 ml) and filtered to give the title compound as colourless solid (20 mg, 0.040 mmol);

### Example 4-5

### (Trans)-8-({[6-(3,5-dimethylphenyl)-3-pyridazinyl]amino}methyl)-3-(3-pyridazinyl)-1-oxa-3-azaspiro[4.5]decan-2-one dihydrochloride

(Trans)-8-({[6-(3,5-dimethylphenyl)-3-pyridazinyl]amino}methyl)-1-oxa-3-azaspiro[4.5]decan-2-one (Intermediate 46, 20 mg, 0.055 mmol), trans-1,2-diaminocyclohexane (0.013 ml, 0.109 mmol), 3-chloropyridazine (prepared according to WO/0107416, 12.50 mg, 0.109 mmol), K₃PO₄ (34.8 mg, 0.164 mmol), copper(I) iodide (10.39 mg, 0.055 mmol) were suspended in 1,4-dioxane (4 ml) and shaken at 120° C. in a closed vial overnight. The resulting dark mixture was concentrated under vacuum, taken up with DCM (20 ml) and filtered over a separation tube. The organic solution was concentrated and purified with Biotage SP1 over a 12S NH₂ Varian cartridge, eluting with a gradient of cyclohexane and EtOAc. (Trans)-8-({[6-(3,5-dimethylphenyl)-3-pyridazinyl]amino}methyl)-3-(3-pyridazinyl)-1-oxa-3-azaspiro[4.5]decan-2-one was eluted with ca 70% EtOAc and recovered as a colourless oil (12 mg, 0.027 mmol, 49%).

¹H NMR (400 MHz, CDCl₃): δ 8.97 (dd, 1H), 8.58 (dd, 1H), 7.63-7.61 (m, 3H), 7.50 (dd, 1H), 7.07 (br s, 1H), 6.73 (d, 1H), 4.86 (br t, 1H), 4.23 (s, 2H), 3.46 (t, 2H), 2.41 (s, 6H), 2.12-1.07 (m, 9H); HPLC-MS: 1.69 min, 445 [M+H]+.

The above compound was dissolved in DCM (2 ml), and reacted with 2.0 equiv. of 1M HCl in Et₂O. The resulting solution was concentrated and tritured with Et₂O (2×5 ml) to give the title compound as a colourless solid (12 mg, 0.023 mmol);

### Example 4-6

### (Trans)-8-({[6-(3-fluorophenyl)-3-pyridazinyl]amino}methyl)-3-(2-fluoro-3-pyridinyl)-1-oxa-3-azaspiro[4.5]decan-2-one Hydrochloride

(Trans)-8-({[6-(3-fluorophenyl)-3-pyridazinyl]amino}methyl)-1-oxa-3-azaspiro[4.5]decan-2-one (Intermediate 48, 80 mg, 0.224 mmol), 2-fluoro-3-iodopyridine (50.1 mg, 0.224 mmol), K₃PO₄ (238 mg, 1.122 mmol), CuI (42.8 mg, 0.224 mmol), trans-1,2-diaminocyclohexane (0.027 ml, 0.224 mmol) in 1,4-dioxane (4 ml) in a closed vial were stirred at 120° C. overnight (ca 18 hours). The mixture was dried (in vacuo) and the solid was taken up with DCM filtering on a filter cartridge. The organic phase was dried (vacuo) to afford a crude that was purified by KP-NH chromatography (Biotage SP1 25+M column) eluting in gradient with 0%-50% EtOAc\cyclohexane (in 5 cv), 50% EtOAc\cyclohexane (5 cv), 50%-100% EtOAc\cyclohexane (in 5 cv), then 100% EtOAc (5 cv) to afford (trans)-8-({[6-(3-fluorophenyl)-3-pyridazinyl]amino}methyl)-3-(2-fluoro-3-pyridinyl)-1-oxa-3-azaspiro[4.5]decan-2-one as a pale yellow solid (23 mg, 0.051 mmol).

¹H NMR (400 MHz, CDCl₃): δ 1.09-1.38 (m, 2H), 1.81-1.97 (m, 3H), 1.99-2.14 (m, 4H), 3.48 (t, 2H), 3.91 (d, 2H), 5.12 (br s., 1H), 6.75 (d, 1H), 7.06-7.15 (m, 1H), 7.23-7.31 (m, 1H), 7.44 (td, 1H), 7.61 (d, 1H), 7.70-7.79 (m, 2H), 8.09 (dt, 1H), 8.16 (ddd, 1H); UPLC-MS: 0.56 min, 452 [M+H]+.

The above compound (23 mg, 0.051 mmol) was suspended in Et₂O (0.5 ml) then 1M HCl (0.061 ml, 0.061 mmol) in Et₂O was added and the resulting precipitate was triturated with Et₂O (3×0.3 ml) then dried (vacuo, 40° C. overnight, ca 20 hours) to afford the title compound as a pale yellow solid (22 mg, 20%);

### Example 4-7

### (Trans)-8-({[6-(2-fluorophenyl)-3-pyridazinyl]amino}methyl)-3-(2-fluoro-3-pyridinyl)-1-oxa-3-azaspiro[4.5]decan-2-one Hydrochloride

In a closed vial (trans)-8-({[6-(2-fluorophenyl)-3-pyridazinyl]amino}methyl)-1-oxa-3-azaspiro[4.5]decan-2-one (Intermediate 47, 40 mg, 0.112 mmol), 2-fluoro-3-iodopyridine (25.03 mg, 0.112 mmol), potassium phosphate (119 mg, 0.561 mmol), copper(I) iodide (21.38 mg, 0.112 mmol) and trans-1,2-diaminocyclohexane (0.013 ml, 0.112 mmol) in 1,4-dioxane (2 ml) were stirred at 120° C. overnight (ca 16 hours). The mixture was allowed to cool to r.t., then it was dried (vacuo) and the solid was taken up with DCM filtering on a filter cartridge. The organic phase was dried (vacuo) to afford a crude that was purified by KP-NH chromatography (Biotage SP1 12+M NH column) eluting in gradient with 0%-50% EtOAc\cyclohexane (5 cv), 50% EtOAc\cyclohexane (5 cv); 50%-100% EtOAc\cyclohexane (5 cv), 100% EtOAc (5 cv) to afford a pale yellow solid (20 mg, 0.044 mmol).

¹H NMR (400 MHz, CDCl₃): δ 1.12-1.32 (m, 2H), 1.81-1.98 (m, 3H), 1.99-2.14 (m, 4H), 3.43-3.52 (m, 2H), 3.90 (d, 2H), 4.83-4.98 (m, 1H), 6.72 (d, 1H), 7.11-7.20 (m, 1H), 7.24-7.32 (m, 2H), 7.34-7.45 (m, 1H), 7.70 (dd, 1H), 8.05-8.22 (m, 3H); UPLC-MS: 0.55 min, 452 [M+H]+.

The above compound (20 mg, 0.044 mmol) was suspended in Et₂O (0.5 ml) then 1M HCl (0.053 ml, 0.053 mmol) in Et₂O was added and the resulting precipitate was triturated with Et₂O (3×0.3 ml) then dried (vacuo, 40° C. overnight, ca 18 hours) to afford the title compound as a yellow solid (18.8 mg).

### Example 4-8

### (Trans)-3-(3-pyridazinyl)-8-({[6-(2-pyridinyl)-3-pyridazinyl]amino}methyl)-1-oxa-3-azaspiro[4.5]decan-2-one Dihydrochloride

A mixture of (trans)-8-(aminomethyl)-3-(3-pyridazinyl)-1-oxa-3-azaspiro[4.5]decan-2-one (Intermediate 41 procedure 41a, 25 mg, 0.095 mmol), 3-chloro-6-(2-pyridinyl)pyridazine (Intermediate 49, 27.4 mg, 0.143 mmol), DIPEA (0.033 ml, 0.191 mmol), potassium fluoride (11.07 mg, 0.191 mmol) and 18-CROWN-6 (5.04 mg, 0.019 mmol) was suspended in sulfolane (1 ml) in a microwave reaction vessel and heated to 200° C. for 30 mins irradiating in a microwave oven. This was diluted with methanol (1 ml), loaded onto a 1g SCX cartridge and eluted with MeOH and then a 2.0M solution of NH₃ in MeOH. The basic fractions containing product were evaporated to give a brown gum, which was purified via Biotage (0.5-2% MeOH/CH₂Cl₂; 12M NH column) to give 22 mg of (trans)-3-(3-pyridazinyl)-8-({[6-(2-pyridinyl)-3-pyridazinyl]amino}methyl)-1-oxa-3-azaspiro[4.5]decan-2-one as a brown gum.

¹H NMR (400 MHz, CDCl₃): δ 8.97 (dd, 1H), 8.63-8.66 (m, 1H), 8.58 (dd, 1H), 8.55 (dt, 1H), 8.31 (d, 1H), 7.82 (td, 1H), 7.51 (dd, 1H), 7.26-7.33 (m, 1H), 6.79 (d, 1H), 4.97 (t, 1H), 4.24 (s, 2H), 3.49 (t, 2H), 1.87-2.13 (m, 7H), 1.22-1.35 (m, 2H); UPLC-MS: 0.46 min, 418 [M+H]+.

21 mg of the above compound was dissolved in dichloromethane (1 ml) and 110 μl of 1.0M HCl in diethyl ether was added and a precipitate formed. A few drops of methanol were added until the mixture became homogenous. This was left for 10 minutes. Volatiles were evaporated and the residue was triturated with diethyl ether (2 ml). The tan solid was collected by filtration, washing filter cake with further diethyl ether. This was dried at 50° C. under vacuum for 3 hours to give the title compound as a tan solid (23.4 mg).

### Example 4-9

### (Trans)-3-(3-pyridazinyl)-8-({[6-(tetrahydro-2H-pyran-4-yloxy)-3-pyridazinyl]-amino}methyl)-1-oxa-3-azaspiro[4.5]decan-2-one dihydrochloride

Titanium(IV) isopropoxide (0.135 ml, 0.459 mmol) was added to a stirred solution of (trans)-2-oxo-3-(3-pyridazinyl)-1-oxa-3-azaspiro[4.5]decane-8-carbaldehyde (prepared in a similar fashion to the Intermediate 60, 60 mg, 0.230 mmol) and 6-(tetrahydro-2H-pyran-4-yloxy)-3-pyridazinamine (Intermediate 50, 44.8 mg, 0.230 mmol) in dichloromethane (4 ml) in a screw-topped vial and the resulting mixture is allowed to stir overnight. Sodium borohydride (43.4 mg, 1.148 mmol) and ethanol (1 ml) were added and the resulting mixture stirred for a further 3 hours. This was diluted with dichloromethane (10 ml) and poured into saturated ammonium chloride solution (20 ml). The mixture was stirred vigorously for 20 minutes then filtered through a hydrophobic frit (Phase Separator cartridge). The aqueous phase was extracted with further dichloromethane (3×15 ml). The combined organic phases were evaporated and the residue was purified via Biotage (5%-20% EtOAc/CH₂Cl₂; 12M NH column). Product containing fractions were not completely homogeneous by TLC so they were purified a second time via Biotage (0-10% MeOH-EtOAc; SNAP 10 g SiO₂ column) to give (trans)-3-(3-pyridazinyl)-8-({[6-(tetrahydro-2H-pyran-4-yloxy)-3-pyridazinyl]amino}methyl)-1-oxa-3-azaspiro[4.5]decan-2-one (50 mg) as a colourless gum.

¹H NMR (400 MHz, CDCl₃): δ 8.96 (dd, 1H), 8.57 (dd, 1H), 7.51 (dd, 1H), 6.79 (d, 1H), 6.70 (d, 1H), 5.33 (sept, 1H), 4.39 (t, 1H), 4.21 (s, 2H), 3.96-4.03 (m, 2H), 3.55-3.63 (m, 2H), 3.37 (t, 2H), 2.10-2.20 (m, 2H), 1.96-2.09 (m, 4H), 1.70-1.96 (m, 5H), 1.16-1.30 (m, 2H); UPLC-MS: 0.45 min, 441 [M+H]+.

49 mg (0.112 mmol) of the above compound was dissolved in acetone (2 ml) and treated with a 1.0M solution of HCl in diethyl ether (0.25 ml, 0.25 mmol) giving a white precipitate. The resulting mixture was stirred for 30 min then evaporated under reduced pressure. The residue was triturated with acetone (2 ml) for 30 min then evaporated under reduced pressure. The residue was triturated with acetone (2 ml) then filtered, washing with acetone (1 ml). The white solid recovered was dried at 45° C. under reduced pressure to give the title compound (45 mg) as a white solid.

### Example 4-10

### (Trans)-8-({[6-({2-[(1,1-dimethylethyl)oxy]ethyl}oxy)-3-pyridazinyl]amino}methyl)-3-(3-pyridazinyl)-1-oxa-3-azaspiro[4.5]decan-2-one dihydrochloride

The title compound was made in a similar fashion to the preparation of Example 4-9 replacing 6-(tetrahydro-2H-pyran-4-yloxy)-3-pyridazinamine with 6-({2-[(1,1-dimethylethyl)oxy]ethyl}oxy)-3-pyridazinamine (Intermediate 51, 48.5 mg, 0.230 mmol) to afford the title compound (30 mg).

### Example 4-11

### (Trans)-8-{[(6-{[(3-methyl-3-oxetanyl)methyl]oxy}-3-pyridazinyl)amino]methy}-3-(3-pyridazinyl)-oxa-3-azaspiro[4.5]decan-2-one

The title compound was made in a similar fashion to the preparation of Example 4-9 replacing 6-(tetrahydro-2H-pyran-4-yloxy)-3-pyridazinamine with 6-{[(3-methyl-3-oxetanyl)methyl]oxy}-3-pyridazinamine (Intermediate 52, 44.8 mg, 0.230 mmol) but purifying by reverse phase chromatography on a Biotage (Phase A: water 95%, acetonitrile 4.9%, formic acid 0.1%; Phase B: acetonitrile 99.9%, formic acid 0.1%; elution began with 100% A then a gradient to 5% A/95% B then isocratic at 5% A/95% B then back to 100% A; SNAP 10 g C18 column) to give 14 mg of partially purified product that was then repurified via Biotage (isocratic 20% EtOAcDCM with 0.1% MeOH, 12M NH column) to give the title compound (6 mg) as a colourless gum.

### Example 4-12

### (Trans)-8-{[(6-chloro-3-pyridazinyl)amino]methyl}-3-(2-pyridinyl)-1-oxa-3-azaspiro[4.5]-decan-2-one

(Trans)-8-(aminomethyl)-3-(2-pyridinyl)-1-oxa-3-azaspiro[4.5]decan-2-one (prepared in a similar fashion to Intermediate 19, 100 mg, 0.383 mmol) was dissolved in butanol (1 ml). Triethylamine (0.059 ml, 0.421 mmol) was added followed by 3,6-dichloropyridazine (57.0 mg, 0.383 mmol). The resulting mixture was heated under microwaves irradiation for 15 min (settings: time: 15 min, T°: 150° C., absorption level: high, fixed hold time: on) first and then for 45 min (same settings). 57 mg of 3,6-dichloropyridazine were added and the resulting mixture was first heated at 150° C. under microwaves irradiation for 1 hour and then at 180° C. under microwaves irradiation for 1 hour. Solvents were removed under reduced pressure and the resulting crude compound was purified by flash chromatography (ISCO COMPANION, 2×12g silica gel column) with the following gradient: A: Cyclohexane; B: EtOAc; 0% B for 2.1 min, 0% to 65% B in 21.4 min, 65% B for 4.3 min to give the title compound as a white solid (90 mg, 62%);

### Example 4-13

### (Trans)-8-{[(6-iodo-3-pyridazinyl)amino]methyl}-3-(2-pyridinyl)-1-oxa-3-azaspiro[4.5]decan-2-one

In a sealed tube, (trans)-8-(aminomethyl)-3-(2-pyridinyl)-1-oxa-3-azaspiro[4.5]decan-2-one (prepared in a similar fashion to Intermediate 19, 428 mg, 1.638 mmol) and 3,6-diiodopyridazine (Intermediate 58, 652 mg, 1.965 mmol) were dissolved in isopropanol (8.6 ml). Then TEA (0.274 ml, 1.965 mmol) was added and the mixture was heated at 130° C. for 70 hours 40 minutes. Solvents were removed under reduced pressure and the crude compound was combined with an other crude batch prepared in a similar fashion to be purified by flash chromatography ISCO COMPANION, 40 g silica gel column) with the following gradient: A: cyclohexane, B: EtOAc; 0% B for 1.6 min, 0% to 60% B in 18.8 min, 60% B for 3.1 min to give the title compound as a yellow solid (486 mg, 52%).

### Example 4-14

### (Trans)-3-(2-pyridinyl)-8-({[6-(2-pyridinyl)-3-pyridazinyl]amino}methyl)-1-oxa-3-azaspiro[4.5]decan-2-one Formate

In a microwave flask under nitrogen were dissolved (trans)-8-{[(6-iodo-3-pyridazinyl)amino]methyl}-3-(2-pyridinyl)-1-oxa-3-azaspiro[4.5]decan-2-one (Example 4-13, 50 mg, 0.107 mmol) and 2-(tributylstannanyl)pyridine (41.5 mg, 0.113 mmol) in dimethylsulfoxide (2 ml). Pd(PPh₃)₄ (4.97 mg, 4.30 μmol) was added and the mixture was heated under microwaves irradiation at 120° C. for 20 min and then at 180° C. for 20 min. The mixture was cooled to r.t. and it was filtered on a 2g SCX cartridge. The cartridge was washed with DCM (20 ml), then MeOH (20 ml) and the compound was released with 15 ml of 2M NH₃ in MeOH. Solvents were removed and the resulting crude compound was purified by flash chromatography (ISCO COMPANION, 12g silica gel column) with the following gradient: A: cyclohexane+3% TEA, B: EtOAc+3% TEA; 0% B for 1.4 min, 0% B to 80% B in 14.3 min, 80% B for 2.9 min to give 15.6 mg of a compound which was repurified by MDAP Fraction Lynx. Solvents were removed under reduced pressure and the resulting salt was dried under high vacuum for 2 hours to give the title compound as a white solid (6.7 mg, 12%).

### Example 4-15

### (Trans)-8-({[6-(2-pyrazinyl)-3-pyridazinyl]amino}methyl)-3-(2-pyridinyl)-1-oxa-3-azaspiro[4.5]decan-2-one

In a microwave flask under nitrogen were dissolved (trans)-8-{[(6-iodo-3-pyridazinyl)amino]methyl}-3-(2-pyridinyl)-1-oxa-3-azaspiro[4.5]decan-2-one (Example 4-13, 50 mg, 0.107 mmol) and 2-(tributylstannanyl)pyrazine (39.7 mg, 0.107 mmol) in DMSO (2 ml). Pd(PPh₃)₄ (4.97 mg, 4.30 μmol) was added and the mixture was heated at 180° C. under microwaves irradiation for 20 minutes. The mixture was cooled to r.t. and passed through a 2g SCX cartridge. The cartridge was washed with 20 ml of DCM, 20 ml of MeOH and the compound was released with 15 ml of 2M NH₃ in MeOH. Solvents were removed under reduced pressure and the crude compound was purified by flash chromatography (ISCO COMPANION, 12g silica gel column) with the following eluent: EtOAc+3% TEA for 20 min. The fractions containing the desired compound were evaporated under reduced pressure. The resulting product was dissolved in 10 ml of DCM and the solution was passed through a 2g SCX cartridge. The cartridge was washed with 20 ml of DCM, 20 ml of MeOH and the compound was released with 15 ml of 2M NH₃ in MeOH. Solvents were removed under reduced pressure and the product was dried under high vacuum for 2 hours to give the title compound as a yellow oil (10 mg, 21%).

### Example 4-16

### (Trans)-3-(2-pyridinyl)-8-({[6-(3-pyridinyl)-3-pyridazinyl]amino}methyl)-1-oxa-3-azaspiro[4.5]decan-2-one

(Trans)-8-{[(6-iodo-3-pyridazinyl)amino]methyl}-3-(2-pyridinyl)-1-oxa-3-azaspiro[4.5]decan-2-one (Example 4-13, 50 mg, 0.107 mmol), 3-pyridinylboronic acid (14.53 mg, 0.118 mmol), sodium carbonate (29.0 mg, 0.274 mmol) and Pd(PPh₃)₄ (6.21 mg, 5.37 μmol) were placed in a tube under nitrogen. Toluene (210 μl), water (210 μl) and ethanol (210 μl) were added, the tube was sealed and the mixture was heated at 100° C. for 17 hours. The mixture was cooled to r.t and 15 ml of EtOAc were added followed by 15 ml of a saturated K₂CO₃ solution. The aqueous phase was extracted twice with EtOAc (10 ml). Combined organic layers were washed with brine (15 ml), dried over Na₂SO₄, filtered and evaporated to dryness. The resulting crude compound was then purified by flash chromatography (ISCO COMPANION, 12g silica gel column) with the following gradient: A: EtOAc, B: MeOH; 0% B for 1.6 min, 0% to 5% B in 17.2 min, 5% B for 4.7 min to give the title compound as a white solid (22.1 mg, 48%).

### Example 4-17

### (Trans)-8-({[6-(phenyloxy)-3-pyridazinyl]amino}methyl)-3-(3-pyridazinyl)-1-oxa-3-azaspiro[4.5]decan-2-one Hydrochloride

(Trans)-2-oxo-3-(3-pyridazinyl)-1-oxa-3-azaspiro[4.5]decane-8-carbaldehyde (Intermediate 60, 40 mg, 0.153 mmol) and 6-(phenyloxy)-3-pyridazinamine (can be prepared as described in Australian Journal of Chemistry 1988, 41(11), 1735-42; 28.7 mg, 0.153 mmol) were suspended in dichloromethane (2 ml) in a closed vial. Titanium(IV) isopropoxide (0.090 ml, 0.306 mmol) was added and the resulting solution was stirred at room temperature overnight (ca 18 hours). Additional titanium(IV) isopropoxide (0.030 ml, 0.102 mmol) was added and the mixture was stirred for further 4 hours, then NaBH₄ (29.0 mg, 0.765 mmol) followed by ethanol (1 ml) were added and the mixture was stirred at r.t. overnight. Then the mixture was diluted with DCM (1 ml) and saturated NaHCO₃ solution was added (1 ml) and the mixture was vigorously stirred for 30 min. then extracted with DCM (3×2 ml); the organic extracts were combined and dried (vacuo) to afford a crude that was purified by KP-NH column chromatography (Biotage SP1, 12+M) eluting in gradient with 0%-50% DCM\Et₂O. Product fractions were combined and dried to afford a white solid (19.8 mg).

¹H NMR (400 MHz, CDCl₃): δ 1.11-1.30 (m, 2H), 1.79-1.96 (m, 3H), 1.97-2.07 (m, 4H), 3.36 (t, 2H), 4.21 (s, 2H), 4.59-4.70 (m, 1H), 6.77 (d, 1H), 7.00 (d, 1H), 7.12-7.26 (m, 3H), 7.33-7.45 (m, 2H), 7.50 (dd, 1H), 8.57 (dd, 1H), 8.96 (dd, 1H); UPLC-MS: 0.55 min, 433 [M+H]+

The above compound (19.8 mg, 0.046 mmol) was suspended in Et₂O and DCM (0.5 ml each). 1M HCl in Et₂O (0.055 mmol, 0.055 ml) was added and the resulting slurry was dried (vacuo) then the resulting solid was triturated with Et₂O (3×0.3 ml) and dried (vacuo, 40° C.; 5 hours) to afford the title compound as a whitish solid (15.8 mg, 22%).

### Example 4-18

### (Trans)-8-({[6-(2-fluorophenyl)-3-pyridazinyl]amino}methyl)-3-(5-methyl-1,3,4-thiadiazol-2-yl)-1-oxa-3-azaspiro[4.5]decan-2-one Hydrochloride

(Trans)-8-({[6-(2-fluorophenyl)-3-pyridazinyl]amino}methyl)-1-oxa-3-azaspiro[4.5]decan-2-one (prepared in a similar fashion to Intermediate 47, 22 mg, 0.062 mmol), 2-bromo-5-methyl-1,3,4-thiadiazole (11.05 mg, 0.062 mmol), potassium phosphate (65.5 mg, 0.309 mmol), copper(I) iodide (11.76 mg, 0.062 mmol) and trans-1,2-diaminocyclohexane (7.42 μl, 0.062 mmol) in 1,4-dioxane (2 ml) were stirred in a closed vial at 120° C. for 5 hours then further 2-bromo-5-methyl-1,3,4-thiadiazole (11.05 mg, 0.062 mmol) was added and the mixture was stirred at 120° C. for 3 hours, left standing at r.t. overnight and stirred again at 120° C. for 3 hours. The mixture was dried (vacuo) then the solid was taken up with DCM and filtered over a filter tube. The organic phase was dried (vacuo) to afford a crude that was purified by KP-NH chromatography (Biotage SP1 12+M) eluting in gradient with 0%-100% EtOAc\cyclohexane to afford a white solid (8.3 mg).

UPLC-MS: 0.55 min, 455 [M+H]+

The above compound (8.3 mg, 0.018 mmol) was suspended in Et₂O (0.5 ml), 1M HCl in Et₂O was added (0.021 ml, 0.021 mmol) and the solid was triturated with Et₂O (3×0.3 ml). The solid was dried (vacuo, 40° C. 1 hour) to afford the title compound as a white solid (7 mg, 23%).

### Example 4-19

### (Trans)-8-({[6-(2-fluorophenyl)-3-pyridazinyl]amino}methyl)-3-(4-pyridazinyl)-1-oxa-3-azaspiro[4.5]decan-2-one

(Trans)-8-({[6-(2-fluorophenyl)-3-pyridazinyl]amino}methyl)-1-oxa-3-azaspiro[4.5]decan-2-one (prepared in a similar fashion to Intermediate 47, 22 mg, 0.062 mmol), 4-bromopyridazine (9.81 mg, 0.062 mmol), potassium phosphate (65.5 mg, 0.309 mmol), copper(I) iodide (11.76 mg, 0.062 mmol) and trans-1,2-diaminocyclohexane (7.42 μl, 0.062 mmol) in 1,4-dioxane (2 ml) were stirred in a closed vial at 120° C. for 5 hours. Then further 4-bromopyridazine (9.81 mg, 0.062 mmol) was added and the mixture was stirred at 120° C. for 3 hours, then left standing at r.t overnight and stirred again at 120° C. for 3 hours. The mixture was dried (vacuo) then the solid was taken up with DCM and filtered on a filter tube. The organic was dried (vacuo) to afford a crude that was purified by KP-NH chromatography (Biotage SP1 12+M) eluting in gradient with 0%-100% EtOAc\cyclohexane to afford a mixture that was further purified by silica chromatography (2g) eluting with 0%-5% MeOHDCM to afford the title compound as a yellowish solid (2.9 mg, 11%).

### Example 4-20

### (Trans)-8-({[6-(2-fluorophenyl)-3-pyridazinyl]amino}methyl)-3-(2-fluoro-4-pyridinyl)-1-oxa-3-azaspiro[4.5]decan-2-one Hydrochloride

(Trans)-8-({[6-(2-fluorophenyl)-3-pyridazinyl]amino}methyl)-1-oxa-3-azaspiro[4.5]decan-2-one (prepared in a similar fashion to Intermediate 47, 22 mg, 0.062 mmol), 4-bromo-2-fluoropyridine hydrochloride (13.11 mg, 0.062 mmol), potassium phosphate (65.5 mg, 0.309 mmol), copper(I) iodide (11.76 mg, 0.062 mmol) and trans-1,2-diaminocyclohexane (7.42 μl, 0.062 mmol) in 1,4-dioxane (2 ml) were stirred in a closed vial at 120° C. for 5 hours. The mixture was dried (vacuo) and the solid was taken up with DCM and filtered on a filter cartridge. The organics were combined and dried (vacuo) to afford a crude. The crude was purified by KP-NH chromatography (Biotage SP1 12+M column) eluting in gradient with 0%-100% EtOAc\cyclohexane to afford a mixture that was further purified by silica gel chromatography (2g) eluting in gradient with 0%-5% MeOHDCM to afford a white solid (5.3 mg).

UPLC-MS: 0.58 min, 452 [M+H]+

The above compound (5.3 mg, 0.012 mmol) was suspended in Et₂O (0.5 ml) then 1M HCl in Et₂O (0.014 ml, 0.014 mmol) was added and the resulting precipitate was triturated with Et₂O (3×0.3 ml) then dried (vacuo, 40° C. overnight, ca 18 hours) to afford the title compound as a white solid (5.1 mg, 17%).

### Example 4-21

### (Trans)-8-({[6-(cyclopentyloxy)-3-pyridazinyl]amino}methyl)-3-(3-pyridazinyl)-1-oxa-3-azaspiro[4.5]decan-2-one Hydrochloride

The title compound was made in a similar fashion to the preparation of Example 1-24 replacing 5-(cyclohexyloxy)-2-pyridinamine with 6-(cyclopentyloxy)-3-pyridazinamine (Intermediate 53, 34.3 mg, 0.191 mmol) to afford the title compound as a white solid (33.1 mg, 37%).

### Example 4-22

### (Trans)-8-({[6-(2-fluorophenyl)-3-pyridazinyl]amino}methyl)-3-(6-methyl-3-pyridazinyl)-1-oxa-3-azaspiro[4.5]decan-2-one Hydrochloride

(Trans)-8-({[6-(2-fluorophenyl)-3-pyridazinyl]amino}methyl)-1-oxa-3-azaspiro[4.5]decan-2-one (Intermediate 47, 40 mg, 0.112 mmol), 3-chloro-6-methylpyridazine (14.43 mg, 0.112 mmol), potassium phosphate (119 mg, 0.561 mmol), copper(I) iodide (21.38 mg, 0.112 mmol) and trans-1,2-diaminocyclohexane (0.013 ml, 0.112 mmol) in 1,4-dioxane (2 ml) in a closed vial were stirred at 120° C. overnight (ca 16 hours). 3-Chloro-6-methylpyridazine (5 mg, 0.039 mmol), trans-1,2-diaminocyclohexane (0.005 ml) and copper(I) iodide (5 mg, 0.026 mmol) were further added and the mixture was stirred at 120° C. for additional 5 hours. The mixture was dried (vacuo) and the solid was taken up with DCM filtering on a filter cartridge. The organic was dried (vacuo) to afford a crude which was purified by KP-NH chromatography (Biotage SP1 12+M) eluting in gradient with 0%-50% EtOAc\cyclohexane (in 5 cv) then 50% EtOAc\cyclohexane (5 cv), 50%-100% EtOAc\cyclohexane (in 5 cv) and 100% EtOAc (5 cv) to afford a white solid (17.4 mg).

¹H NMR (400 MHz, CDCl₃): δ 1.17-1.35 (m, 2H), 1.83-2.00 (m, 3H), 2.00-2.13 (m, 4H), 2.69 (s, 3H), 3.46 (t, 2H), 4.19 (s, 2H), 4.91-5.02 (m, 1H), 6.73 (d, 1H), 7.11-7.22 (m, 1H), 7.24-7.31 (m, 1H), 7.36 (d, 2H), 7.71 (dd, 1H), 8.12 (d, 1H), 8.45 (d, 1H); UPLC-MS: 0.54 min, 449 [M+H]+

The above compound (17.4 mg, 0.039 mmol) was suspended in Et₂O (0.5 ml) then 1M HCl in Et₂O (0.047 ml, 0.047 mmol) was added and the resulting precipitate was triturated with Et₂O (3×0.3 ml) then dried (vacuo, 40° C. overnight) to afford the title compound as a white solid (14.8 mg, 27%).

### Example 4-23

### (Trans)-3-(6-chloro-3-pyridazinyl)-8-({[6-(2-fluorophenyl)-3-pyridazinyl]amino}methyl)-1-oxa-3-azaspiro[4.5]decan-2-one Hydrochloride

The title compound was made in a similar fashion to the preparation of Example 4-22 replacing 3-chloro-6-methylpyridazine with 3,6-dichloropyridazine (16.72 mg, 0.112 mmol) to afford the title compound as a white solid (15.4 mg, 27%).

### Example 4-24

### (Trans)-8-({[6-(3,5-difluorophenyl)-3-pyridazinyl]amino}methyl)-3-(2-fluoro-3-pyridinyl)-1-oxa-3-azaspiro[4.5]decan-2-one Hydrochloride

The title compound was made in a similar fashion to the preparation of Example 4-6 replacing (trans)-8-({[6-(3-fluorophenyl)-3-pyridazinyl]amino}methyl)-1-oxa-3-azaspiro-[4.5]decan-2-one with (trans)-8-({[6-(3,5-difluorophenyl)-3-pyridazinyl]amino}methyl)-1-oxa-3-azaspiro[4.5]decan-2-one (Intermediate 63, 50 mg, 0.134 mmol) to afford the title compound as a pale yellow solid (20.7 mg, 30%).

### Example 4-25

### (Trans)-8-({[6-(3,5-difluorophenyl)-3-pyridazinyl]amino}methyl)-3-(6-fluoro-3-pyridinyl)-1-oxa-3-azaspiro[4.5]decan-2-one Hydrochloride

(Trans)-8-({[6-(3,5-difluorophenyl)-3-pyridazinyl]amino}methyl)-1-oxa-3-azaspiro[4.5]-decan-2-one (Intermediate 63, 50 mg, 0.134 mmol), 5-bromo-2-fluoropyridine (23.50 mg, 0.134 mmol), potassium phosphate (142 mg, 0.668 mmol), copper(I) iodide (25.4 mg, 0.134 mmol) and trans-1,2-diaminocyclohexane (0.016 ml, 0.134 mmol) in 1,4-dioxane (2.5 ml) were stirred at 120° C. for ca 6 hours in a closed vial. The mixture was allowed to cool to r.t. and dried (vacuo). Attempts to extract with waterDCM failed (formation of a thick emulsion). Both phases were combined and dried (vacuo) and the solid was taken up with DCMMeOH filtering on a filter cartridge. The organic was dried (vacuo) to afford a crude that was purified by KP-NH chromatography (Biotage SP1 25+M column) eluting in gradient with 0%-50% EtOAc\cyclohexane (in 5 cv); 50% EtOAc (5 cv); 50%-100% EtOAc\cyclohexane (in 5 cv); 100% EtOAc (5 cv) to afford a white solid (11 mg).

¹H NMR (400 MHz, MeOD-d₄) d 1.23-1.43 (m, 2H), 1.77-1.95 (m, 3H), 1.95-2.15 (m, 4H), 3.38-3.48 (m, 2H), 4.00 (s, 2H), 6.96-7.07 (m, 2H), 7.08-7.18 (m, 1H), 7.53-7.66 (m, 2H), 7.76-7.85 (m, 1H), 8.26-8.38 (m, 1H), 8.38-8.45 (m, 1H); UPLC-MS: 0.62 min, 470 [M+H]+.

The above compound (11 mg, 0.023 mmol) was suspended in Et₂O (0.5 ml) then 1.0M HCl (0.028 ml, 0.028 mmol) in Et₂O was added and the resulting precipitate was triturated with Et₂O (3×0.3 ml) then dried (vacuo, 40° C. ca 3 hours) to afford the title compound as a white solid (10.7 mg, 16%).

### Example 4-26

### (Trans)-8-({[6-(3,5-difluorophenyl)-3-pyridazinyl]amino}methyl)-3-(2-methyl-4-pyridinyl)-1-oxa-3-azaspiro[4.5]decan-2-one

(Trans)-8-({[6-(3,5-difluorophenyl)-3-pyridazinyl]amino}methyl)-1-oxa-3-azaspiro[4.5]-decan-2-one (Intermediate 63, 50 mg, 0.134 mmol), 4-bromo-2-methylpyridine (22.97 mg, 0.134 mmol), potassium phosphate (142 mg, 0.668 mmol), copper(I) iodide (25.4 mg, 0.134 mmol) and trans-1,2-diaminocyclohexane (0.016 ml, 0.134 mmol) in 1,4-dioxane (2.5 ml) in a closed vial were stirred at 120° C. for ca 6 hours. The mixture was allowed to cool to r.t. and it was dried (vacuo); the solid was taken up with DCMMeOH filtering on a filter cartridge. The organic phase was dried (vacuo) to afford a crude that was purified by KP-NH chromatography (Biotage SP1, 25+M column) eluting in gradient with 0%-50% EtOAc\cyclohexane (in 5 cv), 50% EtOAc\cyclohexane (5 cv), 50%-100% EtOAc\cyclohexane (in 5 cv), 100% EtOAc\cyclohexane (5 cv) to afford the title compound as a white solid (5.3 mg, 8%).

### Example 4-27

### (Trans)-8-({[6-(3,5-difluorophenyl)-3-pyridazinyl]amino}methyl)-3-[6-(trifluoromethyl)-3-pyridazinyl]-1-oxa-3-azaspiro[4.5]decan-2-one

(Trans)-8-({[6-(3,5-difluorophenyl)-3-pyridazinyl]amino}methyl)-1-oxa-3-azaspiro[4.5]-decan-2-one (Intermediate 63, 20 mg, 0.053 mmol), 3-chloro-6-(trifluoromethyl)pyridazine (9.75 mg, 0.053 mmol), potassium phosphate (56.7 mg, 0.267 mmol), copper(I) iodide (10.17 mg, 0.053 mmol) and trans-1,2-diaminocyclohexane (6.42 μl, 0.053 mmol) in 1,4-dioxane (2.5 ml) were stirred in a closed vial at 120° C. for 5 hours. The mixture was allowed to cool to r.t. and it was dried (vacuo); then the solid was taken up with DCM/MeOH filtering on a filter cartridge. The organic phase was dried (vacuo) to afford a crude that was purified by silica gel chromatography (Biotage SNAP 10 g column) eluting in gradient with 0%-5% MeOHDCM to afford a mixture that was further purified by reverse phase flash-chromatography (Biotage SP1, C18 12+M column), eluting with a gradient of ACN and water made up 0.1% HCOOH. Fractions containing the required compound were collected and passed through an ion exchange SCX cartridge (2g, Varian) eluting with 2M ammonia in MeOH and dried (vacuo) to afford the title compound as a pale yellow solid (4.5 mg, 16%).

### Example 4-28

### (Trans)-8-({[6-(cyclopentyloxy)-3-pyridazinyl]amino}methyl)-3-(2-fluoro-3-pyridinyl)-1-oxa-3-azaspiro[4.5]decan-2-one Hydrochloride

The title compound was made in a similar fashion to the preparation of Example 4-6 replacing (trans)-8-({[6-(3-fluorophenyl)-3-pyridazinyl]amino}methyl)-1-oxa-3-azaspiro-[4.5]decan-2-one with (trans)-8-({[6-(cyclopentyloxy)-3-pyridazinyl]amino}methyl)-1-oxa-3-azaspiro[4.5]decan-2-one (Intermediate 64, 60 mg, 0.173 mmol) to afford the title compound as a white solid (41 mg, 49%).

### Example 4-29

### (Trans)-8-({[6-(cyclopentyloxy)-3-pyridazinyl]amino}methyl)-3-(2-methyl-4-pyridinyl)-1-oxa-3-azaspiro[4.5]decan-2-one Hydrochloride

The title compound was made in a similar fashion to the preparation of Example 4-28 replacing 2-fluoro-3-iodopyridine with 4-bromo-2-methylpyridine (29.8 mg, 0.173 mmol) to afford the title compound as a white solid (62 mg, 76%).

### Example 4-30

### (Trans)-8-({[6-(2-fluorophenyl)-3-pyridazinyl]amino}methyl)-3-(2-methyl-4-pyridinyl)-1-oxa-3-azaspiro[4.5]decan-2-one Hydrochloride

The title compound was made in a similar fashion to the preparation of Example 4-6 replacing (trans)-8-({[6-(3-fluorophenyl)-3-pyridazinyl]amino}methyl)-1-oxa-3-azaspiro-[4.5]decan-2-one with (trans)-8-({[6-(2-fluorophenyl)-3-pyridazinyl]amino}methyl)-1-oxa-3-azaspiro[4.5]decan-2-one (prepared in a similar manner to Intermediate 47 but purifying the crude by SCX cartridge, 50 mg, 0.140 mmol) and 2-fluoro-3-iodopyridine with 4-bromo-2-methylpyridine (24.13 mg, 0.140 mmol) to afford the title compound as a pale yellow solid (25.5 mg, 37%).

### Example 4-31

### (Trans)-8-({[6-(2-fluorophenyl)-3-pyridazinyl]amino}methyl)-3-(2-methyl-3-pyridinyl)-1-oxa-3-azaspiro[4.5]decan-2-one Hydrochloride

The title compound was made in a similar fashion to the preparation of Example 4-30 replacing 4-bromo-2-methylpyridine with 3-bromo-2-methylpyridine (24.13 mg, 0.140 mmol) to afford the title compound as a greyish solid (24 mg, 35%).

### Example 4-32

### (Trans)-8-({[6-(2-fluorophenyl)-3-pyridazinyl]amino}methyl)-3-(6-methyl-3-pyridinyl)-1-oxa-3-azaspiro[4.5]decan-2-one Hydrochloride

The title compound was made in a similar fashion to the preparation of Example 4-30 replacing 4-bromo-2-methylpyridine with 5-bromo-2-methylpyridine (24.13 mg, 0.140 mmol) to afford the title compound as a white solid (23 mg, 34%).

### Example 4-33

### (Trans)-8-({[6-(2-fluorophenyl)-3-pyridazinyl]amino}methyl)-3-(3-pyridazinyl)-1-oxa-3-azaspiro[4.5]decan-2-one Hydrochloride

(Trans)-8-(aminomethyl)-3-(3-pyridazinyl)-1-oxa-3-azaspiro[4.5]decan-2-one (Intermediate 41 procedure 41a, 20.5 mg, 0.078 mmol) and 3-fluoro-6-(2-fluorophenyl)pyridazine (Intermediate 68, 15 mg, 0.078 mmol) were collected and dissolved in N,N-dimethylacetamide (2 ml) in a 2 ml microwave vial and the resulting mixture was irradiated at 150° C. for overall 65 min. The reaction mixture was passed through an ion exchange SCX 2g cartridge (Strata), washing with MeOH and eluting with 2M ammonia in MeOH. The resulting material was purified by flash chromatography (Biotage SP1, 12M Varian NH₂ column) eluting with a gradient of cyclohexane and ethyl acetate. (Trans)-8-({[6-(2-fluorophenyl)-3-pyridazinyl]amino}methyl)-3-(3-pyridazinyl)-1-oxa-3-azaspiro[4.5]decan-2-one was eluted with EtOAc and recovered as a colourless oil (11 mg).

¹H NMR (400 MHz, CDCl₃): δ 8.97 (dd, 1H), 8.58 (dd, 1H), 8.12 (dt, 1H), 7.71 (dd, 1H), 7.50 (ddd, 1H), 7.42-7.35 (m, 1H), 7.30-7.26 (m, 1H), 7.19-7.13 (m, 1H), 6.73 (d, 1H), 4.99 (br s, 1H), 4.22 (s, 2H), 3.46 (m, 2H), 2.14-1.24 (m, 9H). UPLC-MS: 0.56 min, 435 [M+H]+.

The above compound was dissolved in DCM (2 ml) and reacted with 1.0 equiv. of 1M HCl in Et₂O to give the title compound as a colourless solid (10 mg).

### Example 4-34

### (Trans)-8-({[6-(1-piperidinyl)-3-pyridazinyl]amino}methyl)-3-(3-pyridazinyl)-1-oxa-3-azaspiro[4.5]decan-2-one Dihydrochloride

(Trans)-2-oxo-3-(3-pyridazinyl)-1-oxa-3-azaspiro[4.5]decane-8-carbaldehyde (prepared in a similar fashion to Intermediate 60, 51.3 mg, 0.196 mmol), 6-(1-piperidinyl)-3-pyridazinamine (Intermediate 69, 35 mg, 0.196 mmol), titanium(IV) isopropoxide (0.115 ml, 0.393 mmol) were dissolved in dichloromethane (2 ml) and stirred at r.t. overnight. Sodium borohydride (22.29 mg, 0.589 mmol) and ethanol (2.0 ml) were added and the resulting mixture was stirred at r.t. for 5 hours, then it was diluted with DCM (10 ml) and quenched with a saturated NaHCO₃ solution (2 ml) and filtered. The resulting organic phase was concentrated under vacuum and the residue was purified by reverse phase chromatography (Biotage SP1, 12M C18 cartridge), eluting with a gradient of water and ACN (made up with 0.1% HCOOH). The required compound was eluted with ca 30% acetonitrile. (Trans)-8-({[6-(1-piperidinyl)-3-pyridazinyl]amino}methyl)-3-(3-pyridazinyl)-1-oxa-3-azaspiro[4.5]decan-2-one was recovered, after catch and release process on SCX cartridge, as a yellow oil (5 mg). It was also recovered another impure batch of (trans)-8-({[6-(1-piperidinyl)-3-pyridazinyl]amino}methyl)-3-(3-pyridazinyl)-1-oxa-3-azaspiro[4.5]decan-2-one (30 mg) which was further purified by MDAP system to give chemically pure (trans)-8-({[6-(1-piperidinyl)-3-pyridazinyl]amino}methyl)-3-(3-pyridazinyl)-1-oxa-3-azaspiro[4.5]decan-2-one (12 mg).

¹H NMR (400 MHz, CDCl₃): δ 8.95 (dd, 1H), 8.56 (dd, 1H), 7.49 (dd, 1H), 6.95 (d, 1H), 6.63 (d, 1H), 4.49 (br s, 1H), 4.20 (s, 2H), 3.43 (m, 4H), 3.33 (m, 2H), 2.05-1.07 (m, 15H). UPLC-MS: 0.51 min, 424 [M+H]+.

The above two batches (5 mg) and (12 mg) were collected, dissolved in DCM (2 ml) and reacted with 2.0 equiv. 1M HCl in Et₂O to give the title compound as a yellow solid (15 mg).

### Example 4-35

### (Trans)-8-({[6-(2,5-difluorophenyl)-3-pyridazinyl]amino}methyl)-3-(3-pyridazinyl)-1-oxa-3-azaspiro[4.5]decan-2-one Dihydrochloride

The title compound was made in a similar fashion to the preparation of Example 4-3 replacing (3,5-difluorophenyl)boronic acid with (2,5-difluorophenyl)boronic acid (84 mg, 0.534 mmol) and by treating the methanolic solution of (trans)-8-({[6-(2,5-difluorophenyl)-3-pyridazinyl]amino}methyl)-3-(3-pyridazinyl)-1-oxa-3-azaspiro[4.5]decan-2-one with 2 eq of 1M HCl in Et₂O to afford the title compound as a nearly colourless solid (60 mg).

### Example 4-36

### (Trans)-8-({[6-(2,3-difluorophenyl)-3-pyridazinyl]amino}methyl)-3-(3-pyridazinyl)-1-oxa-3-azaspiro[4.5]decan-2-one Dihydrochloride

The title compound was made in a similar fashion to the preparation of Example 4-35 replacing (2,5-difluorophenyl)boronic acid with (2,3-difluorophenyl)boronic acid (84 mg, 0.534 mmol) to afford the title compound as a nearly colourless solid (60 mg).

### Example 4-37

### (Trans)-8-({[6-(6-fluoro-2-pyridinyl)-3-pyridazinyl]amino}methyl)-3-(3-pyridazinyl)-1-oxa-3-azaspiro[4.5]decan-2-one Dihydrochloride

(Trans)-8-{[(6-chloro-3-pyridazinyl)amino]methyl}-3-(3-pyridazinyl)-1-oxa-3-azaspiro[4.5]decan-2-one (Intermediate 42 procedure 42a, 100 mg, 0.267 mmol), 2M K₃PO₄ aq solution (0.267 ml, 0.534 mmol), tetrakis(triphenylphosphine)palladium(0) (15.41 mg, 0.013 mmol), 2-fluoro-6-(4,4,5,5-tetramethyl-1,3,2-dioxaborolan-2-yl)pyridine (Intermediate 70, 130 mg, 0.583 mmol) were collected in a vial, deareated, and then suspended in N,N-dimethylformamide (4 ml). The reaction mixture was then shaken at 80° C. for 5 hours. Further 2-fluoro-6-(4,4,5,5-tetramethyl-1,3,2-dioxaborolan-2-yl)pyridine (120 mg, 0.267 mmol) was added and the reaction mixture was shaken at 80° C. overnight then diluted with DCM (300 ml) and washed with water (3×100 ml). The resulting organic phase was separated and concentrated under vacuum to give a crude. The crude was purified twice by reverse phase flash chromatography (Biotage SP1, C18 60 g SNAP cartridge) eluting with a gradient of ACN and water (made up with 0.1% HCOOH). Fractions containing the required compound were collected and passed through a SCX cartridge (1g, Varian) and eluted with 2M ammonia in MeOH to give (trans)-8-({[6-(6-fluoro-2-pyridinyl)-3-pyridazinyl]amino}methyl)-3-(3-pyridazinyl)-1-oxa-3-azaspiro[4.5]decan-2-one (7.5 mg).

¹H NMR (400 MHz, CDCl₃): δ 8.97 (dd, 1H), 8.57 (dd, 1H), 8.44 (dd, 1H), 8.20 (d, 1H), 7.94 (q, 1H), 7.50 (dd, 1H), 6.94 (ddd, 1H), 6.79 (d, 1H), 5.10 (br s, 1H), 4.24 (s, 2H), 3.52-3.47 (m, 2H), 2.13-1.13 (m, 9H); UPLC-MS: 0.52 min, 436 [M+H]+.

The above compound was dissolved in DCM (2 ml) and reacted with 2.0 equiv. of 1M HCl in Et₂O, Solvent was removed to give the title compound as a colourless solid (7.0 mg).

### Example 4-38

### (Trans)-8-{[(6-phenyl-3-pyridazinyl)amino]methyl}-3-(3-pyridazinyl)-1-oxa-3-azaspiro[4.5]decan-2-one Dihydrochloride

The title compound was made in a similar fashion to the preparation of Example 4-35 replacing (2,5-difluorophenyl)boronic acid with phenylboronic acid (39.0 mg, 0.320 mmol) to afford the title compound as a colourless solid (16 mg).

### Example 4-39

### (Trans)-8-({[6-(2-methylphenyl)-3-pyridazinyl]amino}methyl)-3-(2-methyl-4-pyridinyl)-1-oxa-3-azaspiro[4.5]decan-2-one Dihydrochloride

(Trans)-8-({[6-(2-methylphenyl)-3-pyridazinyl]amino}methyl)-1-oxa-3-azaspiro[4.5]decan-2-one (Intermediate 45, 40.0 mg, 0.113 mmol), 4-bromo-2-methylpyridine (19.5 mg, 0.113 mmol), copper(I) iodide (21.59 mg, 0.113 mmol), trans-1,2-diaminocyclohexane (0.027 ml, 0.227 mmol), K₃PO₄ (72.2 mg, 0.340 mmol) were collected in a closed vial and suspended in 1,4-dioxane (4 ml). The resulting mixture was stirred at 130° C. for 4 hours then solvent was removed under vacuum and the crude was taken up with DCM (8 ml) and filtered over separating tube. The resulting solution was purified with Biotage SP1, over a 12M NH₂ Varian cartridge, eluting with a gradient of cyclohexane and EtOAc. (Trans)-8-({[6-(2-methylphenyl)-3-pyridazinyl]amino}methyl)-3-(2-methyl-4-pyridinyl)-1-oxa-3-azaspiro[4.5]decan-2-one was eluted with EtOAc and recovered as a colourless oil (26 mg) then tritured with Et₂O (3×3 ml) to give the required product as a colourless solid (22 mg).

¹H NMR (400 MHz, CDCl₃): δ 8.44 (d, 1H), 7.44-7.39 (m, 2H), 7.36-7.29 (m, 5H), 6.73 (d, 1H), 4.78 (br s, 1H), 3.79 (s, 2H), 3.57-3.51 (m, 2H), 2.59 (s, 3H), 2.43 (s, 3H), 2.14-1.18 (m, 9H).

The above compound was dissolved in DCM (2 ml) and reacted with 2.0 equiv. of 1M HCl in Et₂O. The resulting colourless solid was tritured with Et₂O (3×2 ml) and filtered to give the title compound as a colourless solid (20 mg).

### Example 4-40

### (Trans)-3-(3-pyridazinyl)-8-[({6-[3-(trifluoromethyl)phenyl]-3-pyridazinyl}amino)methyl]-1-oxa-3-azaspiro[4.5]decan-2-one Hydrochloride

The title compound was made in a similar fashion to the preparation of Example 4-4 replacing (trans)-8-({[6-(2-methylphenyl)-3-pyridazinyl]amino}methyl)-1-oxa-3-azaspiro-[4.5]decan-2-one with (trans)-8-[({6-[3-(trifluoromethyl)phenyl]-3-pyridazinyl}amino)-methyl]-1-oxa-3-azaspiro[4.5]decan-2-one (Intermediate 71, 49.7 mg, 0.122 mmol) to afford the title compound as a colourless solid (30 mg).

### Example 4-41

### (Trans)-3-(2-methyl-4-pyridinyl)-8-[({6-[3-(trifluoromethyl)phenyl]-3-pyridazinyl}amino)-methyl]-1-oxa-3-azaspiro[4.5]decan-2-one Dihydrochloride

The title compound was made in a similar fashion to the preparation of Example 4-39 replacing (trans)-8-({[6-(2-methylphenyl)-3-pyridazinyl]amin3-chloropyridazine with (trans)-8-[({6-[3-(trifluoromethyl)phenyl]-3-pyridazinyl}amino)methyl]-1-oxa-3-azaspiro[4.5]decan-2-one (Intermediate 71, 40.2 mg, 0.099 mmol) to afford the title compound as a colourless solid (30 mg).

### Example 4-42

### (Trans)-8-[({6-[3,5-bis(trifluoromethyl)phenyl]-3-pyridazinyl}amino)methyl]-3-(3-pyridazinyl)-1-oxa-3-azaspiro[4.5]decan-2-one Dihydrochloride

(Trans)-8-[({6-[3,5-bis(trifluoromethyl)phenyl]-3-pyridazinyl}amino)methyl]-1-oxa-3-azaspiro[4.5]decan-2-one (Intermediate 72, 49.7 mg, 0.105 mmol), 3-chloropyridazine (prepared according to WO/0107416, 12 mg, 0.105 mmol), copper(I) iodide (19.95 mg, 0.105 mmol), trans-1,2-diaminocyclohexane (0.025 ml, 0.210 mmol), K₃PO₄ (66.7 mg, 0.314 mmol) were collected in a closed vial and suspended in 1,4-dioxane (4 ml). The resulting mixture was stirred at 130° C. for 4 hours. Solvent was removed under vacuum and the crude was taken up with DCM (8 ml) and filtered. The resulting solution was purified by flash chromatography over a 12M NH₂ Varian cartridge (Biotage SP1), eluting with a gradient of cyclohexane and ethyl acetate. The required product was eluted with EtOAc and recovered as a colourless oil (38 mg). It was further purified by an ion exchange SCX cartridge (0.5 g Varian) washing with MeOH and eluting with 2M ammonia in MeOH and then by KP-NH flash chromatography (Biotage SP1, 12M Biotage cartridge) eluting with cyclohexane and EtOAc to give (trans)-8-[({6-[3,5-bis(trifluoromethyl)phenyl]-3-pyridazinyl}amino)methyl]-3-(3-pyridazinyl)-1-oxa-3-azaspiro[4.5]decan-2-one as a colourless film (33 mg).

¹H NMR (400 MHz, CDCl₃): δ 8.97 (dd, 1H), 8.58 (dd, 1H), 8.49 (s, 2H), 7.91 (s, 1H), 7.71 (dd, 1H), 7.51 (dd, 1H), 6.81 (d, 1H), 5.07 (br s, 1H), 4.24 (s, 2H), 3.51 (t, 2H), 2.17-1.15 (m, 9H).

The above compound was dissolved in DCM (2 ml) and reacted with 2 equiv. of 1M HCl in Et₂O to give the title compound as a colourless solid (28 mg).

### Example 4-43 and Example 4-44

### (Trans)-8-({[6-(3,5-difluorophenyl)-4-methyl-3-pyridazinyl]amino}methyl)-3-(3-Pyridazinyl)-1-oxa-3-azaspiro[4.5]decan-2-one

### Example 4-43 and (Trans)-8-({[6-(3,5-difluorophenyl)-5-methyl-3-pyridazinyl]amino}methyl)-3-(3-pyridazinyl)-1-oxa-3-azaspiro[4,5]decan-2-one

### Example 4-44

A mixture of (trans)-8-{[(6-chloro-4-methyl-3-pyridazinyl)amino]methyl}-3-(3-pyridazinyl)-1-oxa-3-azaspiro[4.5]decan-2-one and (trans)-8-{[(6-chloro-5-methyl-3-pyridazinyl)amino]-methyl}-3-(3-pyridazinyl)-1-oxa-3-azaspiro[4.5]decan-2-one in molar ratio ca 1:1 (Intermediate 73, 50 mg, 0.129 mmol), (3,5-difluorophenyl)boronic acid (40.6 mg, 0.257 mmol), PdCl₂(PPh₃)₂ (4.51 mg, 6.43 μmol), potassium carbonate (35.5 mg, 0.257 mmol) were collected in a closed vial and suspended in water (1 ml) and acetonitrile (2 ml). The resulting mixture was shaken at 100° C. for overall 6 hours. Further (3,5-difluorophenyl)boronic acid (40.6 mg, 0.257 mmol) and PdCl₂(PPh₃)₂ (4.51 mg, 6.43 μmol) were added and the mixture was stirred at 100° C. overnight. Solvent was removed under vacuum and the residue was taken up with DCM (20 ml), filtered and concentrated to obtain a crude which was purified by flash chromatography over a Varian KP-NH₂ 25M cartridge (Biotage SP1), eluting with a gradient of cyclohexane and EtOAc. (Trans)-8-({[6-(3,5-difluorophenyl)-4-methyl-3-pyridazinyl]amino}methyl)-3-(3-pyridazinyl)-1-oxa-3-azaspiro[4.5]decan-2-one was first eluted and recovered as a colourless solid (7 mg, Example 4-43).

(trans)-8-({[6-(3,5-difluorophenyl)-5-methyl-3-pyridazinyl]amino}methyl)-3-(3-pyridazinyl)-1-oxa-3-azaspiro[4.5]decan-2-one was subsequently eluted and recovered as a colourless solid (9 mg, Example 4-44).

### Example 4-45

### Trans-8-({[6-(3,5-difluorophenyl)-3-pyridazinyl]amino}methyl)-3-(2-pyridinyl)-1-oxa-3-azaspiro[4.5]decan-2-one Dihydrochloride

(Trans)-8-{[(6-iodo-3-pyridazinyl)amino]methyl}-3-(2-pyridinyl)-1-oxa-3-azaspiro[4.5]decan-2-one (Example 4-13, 50 mg, 0.107 mmol), (3,5-difluorophenyl) boronic acid (18.67 mg, 0.118 mmol), sodium carbonate (29.0 mg, 0.274 mmol) and Pd(PPh₃)₄ (6.21 mg, 5.37 μmol) were suspended in toluene (210 μl), ethanol (210 μl) and water (210 μl) and the mixture was stirred under nitrogen atmosphere at 90° C. overnight. Then it was cooled to r.t., poured into water (25 ml) and extracted with DCM. The solvent was evaporated (under vacuum) and the resulting crude was purified by KP-NH chromatography (Biotage SP1, 25M NH column) eluting in gradient with 30%-100% EtOAc\cyclohexane to give trans-8-({[6-(3,5-difluorophenyl)-3-pyridazinyl]amino}methyl)-3-(2-pyridinyl)-1-oxa-3-azaspiro[4.5]decan-2-one (22.3 mg, 0.049 mmol, 46%).

¹H NMR (400 MHz, DMSO-d₆): δ 1.22-1.28 (m, 2H), 1.64-1.76 (m, 3H), 1.82-1.91 (m, 2H), 1.95-2.03 (m, 2H), 3.32-3.37 (m, 2H), 4.03 (s, 2H), 6.92 (d, 1H), 7.13-7.18 (m, 1H), 7.26 (d, 1H), 7.52-7.59 (m, 1H), 7.60-7.66 (m, 1H), 7.70-7.76 (m, 1H), 7.83-7.92 (m, 1H), 8.09-8.13 (m, 1H), 8.37 (ddd, 1H); UPLC-MS: 0.65 min, 452 [M+H]+

The above compound (22 mg, 0.049 mmol) was dissolved in DCM (2 ml) and treated with 1.0M HCl in diethyl ether (0.122 ml, 0.122 mmol) and some drops of Et₂O for 30 minutes under stirring. Solvent was evaporated under reduced pressure, the residue was triturated with Et₂O (2.0 ml) and the solid was collected by filtration then dried under vacuum at 60° C. to give the title compound (18 mg, 0.034 mmol, 70%).

### Example 4-46

### (Trans)-8-({[6-(3-fluorophenyl)-3-pyridazinyl]amino}methyl)-3-(3-pyridazinyl)-1-oxa-3-azaspiro[4.5]decan-2-onedihydrochloride

(3-Fluorophenyl)boronic acid (22.40 mg, 0.160 mmol), (trans)-8-{[(6-chloro-3-pyridazinyl)amino]methyl}-3-(3-pyridazinyl)-1-oxa-3-azaspiro[4.5]decan-2-one (Intermediate 42, procedure 42b, 60 mg, 0.160 mmol) PdCl₂(PPh₃) (5.62 mg, 0.008 mmol) and potassium carbonate (44.2 mg, 0.320 mmol) were suspended in acetonitrile (2 ml) and water (1 ml) and the resulting mixture was irradiated in microwave system at 120° C. for 30 min (2 cycles). The reaction mixture was evaporated under vacuum and the residue was taken up with DCM (5 ml) and filtered through a filter tube. The resulting organic phase was concentrated under vacuum and purified by flash chromatography (Biotage SP1, 25M Varian NH₂ cartridge) eluting with a gradient of cyclohexane and ethyl acetate. The required product was eluted with ca 100% EtOAc and recovered as white solid (34.5 mg, 0.079 mmol, 50%).

¹H NMR (400 MHz, MeOD-d₄): δ 1.30-1.43 (m, 2H), 1.83-1.93 (m, 3H), 1.98-2.15 (m, 5H), 3.44 (d, 2H), 4.22-4.26 (m, 2H), 7.00 (d, 1H), 7.13-7.20 (m, 1H), 7.46-7.53 (m, 1H), 7.67-7.82 (m, 4H), 8.58 (dd, 1H), 8.95 (dd, 1H); UPLC-MS: 0.53 min, 435 [M+H]+.

The above compound (34.5 mg, 0.079 mmol) was dissolved in DCM (2 ml) and treated with 1.0M HCl in Et₂O (0.199 ml, 0.199 mmol) and some drops of Et₂O for 30 minutes under stirring. Solvent was evaporated under reduced pressure and the residue was triturated with Et₂O (2.0 ml). The resulting solid was dried under vacuum at 60° C. to afford the title compound (38.6 mg, 0.076 mmol, 96%).

### Example 4-47

### Trans-8-({[6-(cyclohexyloxy)-3-pyridazinyl]amino}methyl)-3-(3-pyridazinyl)-1-oxa-3-azaspiro[4,5]decan-2-one.hydrochloride

6-(Cyclohexyloxy)-3-pyridazinamine (Intermediate 97, 51.8 mg, 0.268 mmol) and (trans)-2-oxo-3-(3-pyridazinyl)-1-oxa-3-azaspiro[4.5]decane-8-carbaldehyde (prepared in a similar fashion to the Intermediate 60, 70 mg, 0.268 mmol) were dissolved in anhydrous dichloromethane (4 ml) and titanium(IV) isopropoxide (0.236 ml, 0.804 mmol) was added. The reaction mixture was stirred overnight. Then sodium borohydride (30.4 mg, 0.804 mmol) and ethanol (2 ml) were added and the mixture was stirred for 1.5 hour, then poured into a saturated NaHCO₃ solution (5 ml) and extracted with DCM (3×50 ml) by a phase separator tube. The organic phase was concentrated under vacuum to give a crude which was purified by KP-NH chromatography (25M NH column) eluting with 0%-50% MeO HEtOAc to afford (trans)-8-({[6-(cyclohexyloxy)-3-pyridazinyl]amino}methyl)-3-(3-pyridazinyl)-1-oxa-3-azaspiro[4.5]decan-2-one (29 mg, 0.066 mmol, 25%).

¹H NMR (400 MHz, CDCl₃): δ 1.34-1.64 (m, 5H), 1.67-1.95 (m, 6H), 1.97-2.05 (m, 5H), 2.06-2.14 (m, 2H), 3.35 (t, 2H), 4.13 (q, 1H), 4.20 (s, 2H), 4.35 (t, 1H), 5.07-5.19 (m, 1H), 6.64-6.69 (m, 1H), 6.71-6.76 (m, 1H), 7.49 (dd, 1H), 8.56 (dd, 1H), 8.95 (dd, 1H); UPLC-MS: 0.57 min, 439 [M+H]+.

The above compound (29 mg, 0.066 mmol) was dissolved in DCM\MeOH (2 ml\0.5 ml) and treated with 1.0M HCl in Et₂O (0.093 ml, 0.093 mmol) and the resulting solution was stirred for 30 minutes. Solvent was evaporated under reduced pressure and the residue was triturated with acetone (3×1 ml) to give a white solid which was dried under vacuum at 60° C. overnight to afford the title compound (31.3 mg, 0.066 mmol, 100%).

### Example 4-48

### Trans-8-({[6-(cyclohexyloxy)-3-pyridazinyl]amino}methyl)-3-(2-pyridinyl)-1-oxa-3-azaspiro [4.5]decan-2-one.hydrochloride

The title compound was made in a similar fashion to the preparation of Example 4-47 replacing (trans)-2-oxo-3-(3-pyridazinyl)-1-oxa-3-azaspiro[4.5]decane-8-carbaldehyde with (trans)-2-oxo-3-(2-pyridinyl)-1-oxa-3-azaspiro[4.5]decane-8-carbaldehyde (may be prepared in a similar fashion to the Intermediate 12, 105 mg, 0.403 mmol) to afford the title compound (41.3 mg, 0.087 mmol).

### Example 4-49

### Trans-8-({[6-(cyclopentyloxy)-3-pyridazinyl]amino}methyl)-3-(2-pyridinyl)-1-oxa-3-azaspiro[4.5]decan-2-one.hydrochloride

The title compound was made in a similar fashion to the preparation of Example 4-48 replacing 6-(cyclohexyloxy)-3-pyridazinamine with 6-(cyclopentyloxy)-3-pyridazinamine

(Intermediate 53, 72.3 mg, 0.403 mmol) to afford the title compound (8.4 mg, 0.018 mmol).

### Example 4-50

### (Trans)-8-({[6-(2-oxo-1(2H)-pyridinyl)-3-pyridazinyl]amino}methyl)-3-(3-pyridazinyl)-1-oxa-3-azaspiro[4.5]decan-2-one.hydrochloride

The title compound was made in a similar fashion to the preparation of Example 4-47 replacing 6-(cyclohexyloxy)-3-pyridazinamine with 1-(6-amino-3-pyridazinyl)-2(1H)-pyridinone (Intermediate 98, 35.9 mg, 0.191 mmol) to afford the title compound (14 mg, 0.030 mmol).

### Example 4-51

### (Trans)-8-[({6-[(2-methylphenyl)oxy]-3-pyridazinyl}amino)methyl]-3-(3-pyridazinyl)-1-oxa-3-azaspiro[4.5]decan-2-one

In a 8 ml vial (trans)-2-oxo-3-(3-pyridazinyl)-1-oxa-3-azaspiro[4.5]decane-8-carbaldehyde (prepared in a similar fashion to Intermediate 60 but purifying the crude by silica gel chromatography on a 50 g SNAP Biotage column eluting in gradient with 60%-100% EtOAC\cyclohexane then 100% EtOAc, 12.46 mg, 0.048 mmol) and titanium(IV) isopropoxide (0.023 ml, 0.080 mmol) were added to a solution of 6-[(2-methylphenyl)oxy]-3-pyridazinamine (Intermediate 112, 8 mg, 0.040 mmol) in dry DCM. The resulting mixture was shaken at room temperature overnight. Ethanol (0.3 ml) and sodium borohydride (4.51 mg, 0.119 mmol) were added to the mixture which was shaken for 3.5 hours then diluted with DCM (1 ml) and treated with a saturated NaHCO₃ solution (1 ml). The organic phase was separated through a phase separator tube, the aq emulsion was back-extracted with DCM; combined organic extracts were concentrated under vacuum to give a crude which was purified by RP-flash chromatography (C18 10 g SNAP column) eluting in gradient with 0% phase B (acetonitrile+0.1% HCO₂H) (2 cv), 0%-90% phase B\phase A (H₂O\5% acetonitrile\0.1% HCO₂H) (in 10 cv), 90% phase B\phase A (2 cv), 90%-0% phase B\phase A (in 2 cv) to recover the title compound as a white solid (5.9 mg).

### Example 4-52

### (Trans)-8-[({6-[(3-fluorophenyl)oxy]-3-pyridazinyl}amino)methyl]-3-(3-pyridazinyl)-1-oxa-3-azaspiro[4.5]decan-2-one

The title compound was made in a similar fashion to the preparation of Example 4-51 replacing 6-[(2-methylphenyl)oxy]-3-pyridazinamine with 6-[(3-fluorophenyl)oxy]-3-pyridazinamine (Intermediate 113, 9.5 mg, 0.046 mmol) to afford the title compound as a white solid (9.6 mg).

### Example 4-53

### (Trans)-3-(3-pyridazinyl)-8-{[(6-{[2-(trifluoromethyl)phenyl]oxy}-3-pyridazinyl)amino]methyl}-1-oxa-3-azaspiro[4.5]decan-2-one

The title compound was made in a similar fashion to the preparation of Example 4-51 replacing 6-[(2-methylphenyl)oxy]-3-pyridazinamine with 6-{[2-(trifluoromethyl)phenyl]oxy}-3-pyridazinamine (Intermediate 114, 10 mg, 0.039 mmol) to afford the title compound as a white solid (3.7 mg).

### Example 4-54

### (Trans)-8-[({6-[(4-fluorophenyl)oxy]-3-pyridazinyl}amino)methyl]-3-(3-pyridazinyl)-1-oxa-3-azaspiro[4.5]decan-2-one Formate

The title compound was made in a similar fashion to the preparation of Example 4-51 replacing 6-[(2-methylphenyl)oxy]-3-pyridazinamine with 6-[(4-fluorophenyl)oxy]-3-pyridazinamine (Intermediate 115, 8.1 mg, 0.039 mmol) to afford the title compound as a white solid (8.9 mg, 0.018 mmol, 45%).

¹H NMR consistent with desired compound assumed to be isolated as formate salt as shown by peak at 8.08 ppm.

### Example 4-55

### (Trans)-8-[({6-[(2,4-dimethylphenyl)oxy]-3-pyridazinyl}amino)methyl]-3-(3-pyridazinyl)-1-oxa-3-azaspiro[4.5]decan-2-one Formate

The title compound was made in a similar fashion to the preparation of Example 4-51 replacing 6-[(2-methylphenyl)oxy]-3-pyridazinamine with 6-[(2,4-dimethylphenyl)oxy]-3-pyridazinamine (Intermediate 116, 11 mg, 0.051 mmol) to afford the title compound as white solid (12.4 mg).

¹H NMR consistent with desired compound assumed to be isolated as formate salt as shown by peak at 8.15 ppm

### Example 4-56

### (Trans)-3-(3-pyridazinyl)-8-({[6-({2-[(trifluoromethyl)oxy]phenyl}oxy)-3-pyridazinyl]amino}methyl)-1-oxa-3-azaspiro[4.5]decan-2-one

The title compound was made in a similar fashion to the preparation of Example 4-51 replacing 6-[(2-methylphenyl)oxy]-3-pyridazinamine with 6-({2-[(trifluoromethyl)oxy]-phenyl}oxy)-3-pyridazinamine (Intermediate 117, 9.1 mg, 0.034 mmol) to afford the title compound as a white solid (5.0 mg, 9.68 μmol, 29%).

All the analytical data are set forth in the following Table 4-1 and in which R, A₄ and B are:

| | |
|---|---|
| | (IID) |
| 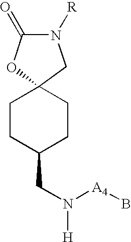 | |

| Cpd. | | | |
|---|---|---|---|
| No. | R | A4-B | Analytical Data |
| 4-1 | 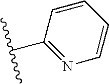 | 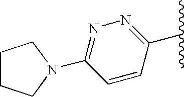 | ¹H NMR (400 MHz, DMSO-d₆): δ 8.38-8.35 (m, 1 H), 8.11 (dt, 1 H), 7.87-7.80 (m, 1 H), 7.55-7.50 (m, 1 H), 7.39-7.26 (m, 1 H), 7.18-7.13 (m, 1 H), 4.02 (s, 2 H), 3.75-3.42 (m, 5 H), 2.04-1.59 (m, 11 H), 1.33-1.15 (m, 2 H); UPLC-MS: 0.58 min, 437 [M + H]+ |
| 4-2 | 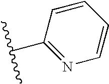 | 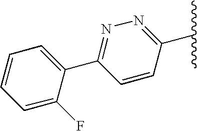 | ¹H NMR (400 MHz, DMSO-d₆): δ 8.32-8.41 (m, 1 H), 8.11 (d, 1 H), 7.79-7.91 (m, 2 H), 7.59 (dd, 1 H), 7.41-7.51 (m, 1 H), 7.27-7.37 (m, 2 H), 7.11-7.20 (m, 2 H), 6.92 (d, 1 H), 4.04 (s, 2 H), 3.34-3.38 (m, 1 H), 2.00 (d, 2 H), 1.88 (dd, 2 H), 1.63-1.81 (m, 3 H), 1.18-1.34 (m, 2 H); Rf = 0.50 (AcOEt); HPLC-MS: 1.83 min, 867.3 [2M + H]+, 434.2 [M + H]+ |
| 4-3 | 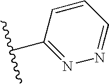 | 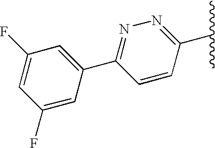 | ¹H NMR (400 MHz, DMSO-d₆): δ 9.94 (br s, 1 H), 9.03 (d, 1 H), 8.41 (d, 2 H), 7.68-7.83 (m, 4 H), 7.51 (t, 1 H), 4.17 (s, 2 H), 3.43 (t, 2 H), 2.02- 2.12 (m, 2 H), 1.87-1.98 (m, 2 H), 1.67-1.85 (m, 3 H), 1.27-1.41 (m, 2 H); UPLC-MS: 0.56 min, 453 [M + H]+ |
| 4-4 | 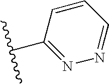 | 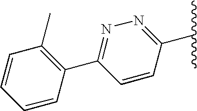 | ¹H NMR (400 MHz, DMSO-d₆): δ 9.02 (dd, 1 H), 8.40 (dd, 1 H), 8.01 d, 1 H), 7.74 (dd, 1 H), 7.66 (m, 1 H), 7.49-7.34 (m, 4 H), 4.18 (s, 2 H), 3.41-3.35 (m, 2 H), 2.37 (s, 3 H), 2.11-1.25 (m, 9 H); UPLC-MS: 0.74 min, 431 [M + H]+. |
| 4-5 | 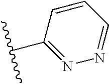 | 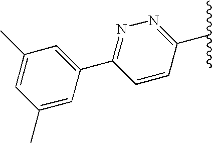 | ¹H NMR (400 MHz, DMSO-d₆): δ 9.02 (dd, 1 H), 8.40 (dd, 1 H), 8.35 (d, 1 H), 7.75 (d, 1 H), 7.65 (br, 1 H), 7.60 (br s, 2 H), 7.22 (s, 1 H), 4.18 (s, 2 H), 3.39 (br t, 2 H), 2.38 (s, 6 H), 2.11-1.25 (m, 9 H); HPLC-MS: 1.69 min, 445 [M + H]+. |
| 4-6 | 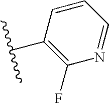 | 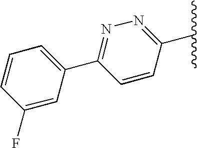 | ¹H NMR (400 MHz, DMSO-d₆) δ 1.13-1.30 (m, 2 H), 1.63-1.83 (m, 3 H), 1.83-1.95 (m, 2 H), 1.98-2.10 (m, 2 H), 3.28-3.36 (m, 2 H), 3.94 (s, 2 H), 7.14-7.37 (m, 2 H), 7.40-7.50 (m, 1 H) 7.52-7.62 (m, 1 H), 7.77- 7.88 (m, 2 H), 8.01-8.12 (m, 1 H), 8.12-8.24 (m, 2 H); UPLC-MS: 0.56 min, 452 [M + H]+. |
| 4-7 | 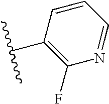 | 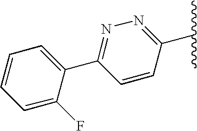 | ¹H NMR (400 MHz, CDCl₃): δ 1.15- 1.34 (m, 2 H), 1.63-1.83 (m, 2 H), 1.83-1.98 (m, 3 H), 1.98-2.14 (m, 2 H), 3.26-3.39 (m, 2 H), 3.93 (s, 2 H), 7.36-7.23 (m, 5 H), 7.77-7.84 (m, 1 H), 7.92-8.05 (m, 1 H), 8.12- 8.22 (m, 2 H); UPLC-MS: 0.55 min, 452 [M + H]+. |
| 4-8 | 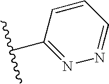 | 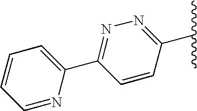 | ¹H NMR (400 MHz, DMSO-d₆): δ 9.03 (dd, 1 H), 8.76-8.79 (m, 1 H), 8.59 (d, 1 H), 8.40 (dd, 1 H), 8.22 (d, 1 H), 8.07 (td, 1 H), 7.73-7.83 (m, 2 H), 7.61 (ddd, 1 H), 4.18 (s, 2 H), 3.43 (t, 2 H), 2.02-2.12 (m, 2 H), 1.89-1.98 (m, 2 H), 1.68-1.88 (m, 3 H), 1.28-1.43 (m, 2 H); UPLC-MS: 0.44 min, 418 [M + H]+ |
| 4-9 | 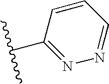 | 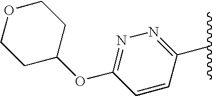 | ¹H NMR (400 MHz, DMSO-d6): δ 13.88 (br s, 1 H), 9.60 (br s, 1 H), 9.03 (dd, 1 H), 8.40 (dd, 1 H), 7.76 (dd, 1 H), 7.65 (d, 1 H), 7.52 (d, 1 H), 5.02 (sept, 1 H), 4.16 (s, 2 H), 3.83- 3.92 (m, 2 H), 3.45-3.54 (m, 2 H), 3.29 (t, 2 H), 2.00-2.12 (m, 4 H), 1.82-1.95 (m, 2 H), 1.62-1.80 (m, 5 H), 1.23-1.38 (m, 2 H); UPLC-MS: 0.45 min, 441 [M + H]+ |
| 4-10 | 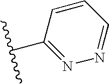 | 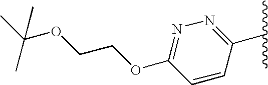 | ¹H NMR (400 MHz, DMSO-d6): δ 13.89 (br s, 1 H), 9.64 (br s, 1 H), 9.02 (dd, 1 H), 8.40 (dd, 1 H), 7.76 (dd, 1 H), 7.47-7.70 (m, 2 H), 4.29 (t, 2 H), 4.16 (s, 2 H), 3.67 (t, 2 H), 3.30 (t, 2 H), 2.01-2.10 (m, 2 H), 1.84- 1.94 (m, 2 H), 1.66-1.80 (m, 3 H), 1.22-1.38 (m, 2 H), 1.17 (s, 3 H); UPLC-MS: 0.51 min, 457 [M + H]+ |
| 4-11 | 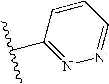 | 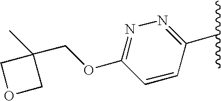 | ¹H NMR (400 MHz, CDCl₃): δ 8.97 (dd, 1 H), 8.58 (dd, 1 H), 7.50 (dd, 1 H), 6.86 (d, 1 H), 6.72 (d, 1 H), 4.47 (d, 2 H), 4.44-4.48 (m, 4 H), 4.41 (t, 1 H), 4.22 (s, 2 H), 3.37 (t, 2 H), 2.00- 2.10 (m, 4 H), 1.84-1.98 (m, 3 H), 1.44 (s, 3 H), 1.15-1.31 (m, 2 H); UPLC-MS: 0.44 min, 441 [M + H]+ |
| 4-12 | 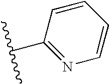 | 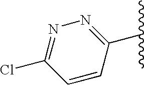 | ¹H-NMR (400 MHz, DMSO-d6): δ 8.37 (dt, 1 H), 8.05-8.15 (m, 1 H), 7.84 (ddd, 1 H), 7.35 (d, 1 H), 7.10- 7.22 (m, 2 H), 6.92 (d, 1 H), 3.97- 4.05 (m, 2 H), 3.25 (t, 2 H), 1.93- 2.04 (m, 2 H), 1.78-1.89 (m, 2 H), 1.62-1.75 (m, 3 H), 1.14-1.31 (m, 2 H); UPLC-MS: 0.59 min, 374 [M + H]⁺ |
| 4-13 | 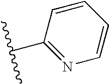 | 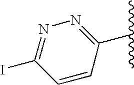 | ¹H-NMR (400 MHz, DMSO-d6): δ 8.37 (dt, 1 H), 8.07-8.13 (m, 1 H), 7.84 (ddd, 1 H), 7.53 (d, 1 H), 7.15 (ddd, 1 H), 7.09 (t, 1 H), 6.63 (d, 1 H), 4.01 (s, 2 H), 3.22 (t, 2 H), 1.98 (d, 2 H), 1.82 (dd, 2 H), 1.67 (td, 3 H), 1.13-1.31 (m, 2 H); UPLC-MS: 0.60 min, 466 [M + H]+ |
| 4-14 | 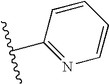 | 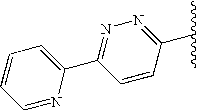 | ¹H-NMR (400 MHz, MeOD-d4): δ 8.64 (br s., 1 H), 8.36 (ddd, 3 H), 8.17 (d, 2 H), 7.93 (t, 1 H), 7.80 (ddd, 1 H), 7.43 (br s., 1 H), 7.10- 7.17 (m, 1 H), 6.97-7.08 (m, 1 H), 4.09-4.17 (m, 2 H), 3.45 (d, 2 H), 1.97-2.13 (m, 4 H), 1.76-1.95 (m, 3 H), 1.23-1.43 (m, 2 H); UPLC-MS: 0.54 min, 417 [M + H]+ |
| 4-15 | 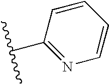 | 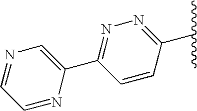 | ¹H-NMR (400 MHz, CDCl₃): δ 1.22- 1.39 (m, 2 H), 1.78-1.97 (m, 3 H), 1.98-2.11 (m, 4 H), 3.46-3.55 (m, 2 H), 4.00-4.12 (m, 2 H), 5.11 (br s., 1 H), 6.81 (d, 1 H), 7.06 (ddd, 1 H), 7.73 (ddd, 1 H), 8.19-8.30 (m, 2 H), 8.35 (ddd, 1 H), 8.53-8.62 (m, 2 H), 9.78 (s, 1 H); UPLC-MS 0.53 min, 418 [M + H]+, 209 [M + H/2]+ |
| 4-16 | 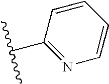 | 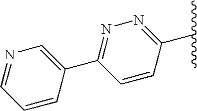 | ¹H NMR (400 MHz, CDCl₃): δ 1.21- 1.35 (m, 2 H), 1.81-1.98 (m, 3 H), 1.99-2.10 (m, 4 H), 3.44-3.53 (m, 2 H), 4.08 (s, 2 H), 4.83-4.92 (m, 1 H), 6.78 (d, 1 H), 7.06 (ddd, 1 H), 7.40-7.46 (m, 1 H), 7.66 (d, 1 H), 7.73 (ddd, 1 H), 8.25-8.31 (m, 1 H), 8.35 (ddd, 1 H), 8.37-8.42 (m, 1 H), 8.66 (dd, 1 H), 9.15 (d, 1 H); UPLC-MS: 0.48 min, 417 [M + H]+, 209 [M + H/2]+ |
| 4-17 | 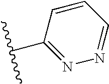 | 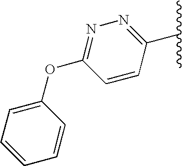 | ¹H NMR (400 MHz, DMSO-d₆): δ 1.16-1.39 (m, 2 H), 1.60-1.78 (m, 3 H), 1.78-1.93 (m, 2 H), 1.94-2.16 (m, 2 H), 3.16-3.29 (m, 2 H), 4.15 (s, 2 H), 7.14-7.37 (m, 3 H), 7.39-7.54 (m, 2 H), 7.54-7.81 (m, 3 H), 8.27- 8.49 (m, 1 H), 8.91-9.15 (m, 1 H); UPLC-MS: 0.53 min, 433 [M + H]+ |
| 4-18 | 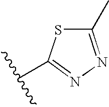 | 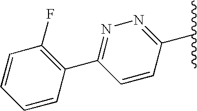 | ¹H NMR (400 MHz, DMSO-d₆): δ 1.19-1.38 (m, 2 H), 1.65-1.82 (m, 3 H), 1.82-1.97 (m, 2 H), 1.99-2.15 (m, 2 H), 2.64 (s, 3 H), 3.34 (br s, 2 H), 4.08 (s, 2 H), 7.25-7.43 (m, 3 H), 7.47-7.61 (m, 1 H), 7.75-7.87 (m, 2 H); UPLC-MS: 0.55 min, 455 [M + H]+ |
| 4-19 | 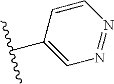 | 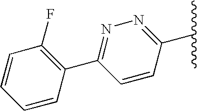 | ¹H NMR (400 MHz, CDCl₃): δ 1.22- 1.46 (m, 2 H), 1.81-2.00 (m, 3 H), 2.00-2.16 (s, 4 H), 3.49-3.59 (m, 2 H), 3.74 (m, 1 H), 3.87 (s, 2 H), 6.73-6.86 (m, 1 H), 7.12-7.22 (m, 1 H), 7.25-7.35 (m, 1 H), 7.36-7.48 (m, 1 H), 7.69-7.82 (m, 1 H), 7.82- 7.93 (m, 1 H), 8.04-8.16 (m, 1 H), 9.04-9.16 (m, 1 H), 9.40-9.50 (m, 1 H); UPLC-MS: 0.49 min, 435 [M + H]+ |
| 4-20 | 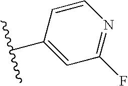 | 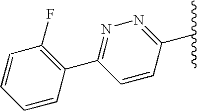 | ¹H NMR (400 MHz, CDCl₃): δ 1.22- 1.46 (m, 2 H), 1.63-1.82 (m, 3 H), 1.83-1.94 (m, 2 H), 1.94-2.08 (m, 2 H), 3.29-3.48 (m, 2 H), 3.97 (s, 2 H), 7.12-7.31 (m, 1 H), 7.32-7.45 (m, 2 H), 7.45-7.58 (m, 2 H), 7.58- 7.67 (m, 1 H), 7.67-7.93 (m, 2 H), 8.13-8.24 (m, 1 H); UPLC-MS: 0.58 min, 452, [M + H]+ |
| 4-21 | 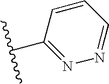 | 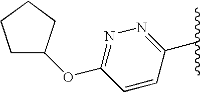 | ¹H NMR (400 MHz, DMSO-d₆): δ 1.19-1.39 (m, 2 H), 1.50-2.15 (m, 15 H), 3.21-3.30 (m, 2 H), 4.16 (s, 2 H), 5.15-5.27 (m, 1 H), 7.34-7.59 (m, 2 H), 7.67-7.80 (m, 1 H), 8.34- 8.43 (m, 1 H), 8.97-9.06 (m, 1 H), 13.65-14.05 (br s, 1 H); UPLC-MS: 0.54 min, 425 [M + H]+ |
| 4-22 | 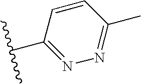 | 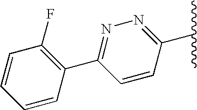 | ¹H NMR (400 MHz, DMSO-d₆): δ 1.24-1.40 (m, 2 H), 1.67-1.84 (m, 3 H), 1.85-1.96 (m, 2 H), 1.99-2.11 (m, 2 H), 2.58 (s, 3 H), 3.32-3.42 (m, 2 H), 4.14 (s, 2 H), 7.35-7.55 (m, 3 H), 7.55-7.67 (m, 2 H), 7.77-7.86 (m, 1 H), 7.95-8.05 (m, 1 H), 8.26- 8.33 (m, 1 H); UPLC-MS: 0.54 min, 449 [M + H]+ |
| 4-23 | 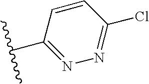 | 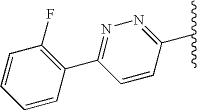 | ¹H NMR (400 MHz, DMSO-d₆): δ 1.22-1.41 (m, 2 H), 1.65-1.84 (m, 3 H), 1.84-1.96 (m, 2 H), 2.00-2.13 (m, 2 H), 3.32-3.40 (m, 2 H), 4.13 (s, 2 H), 7.33-7.47 (m, 3 H), 7.50-7.63 (m, 1 H), 7.77-7.86 (m, 1 H), 7.93 (d, 2 H), 8.46 (d, 1 H); UPLC-MS: 0.59 min, 469 [M + H]+. |
| 4-24 | 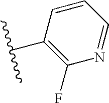 | 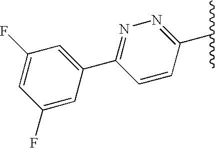 | ¹H NMR (400 MHz, DMSO-d₆): δ 1.13-1.32 (m, 2 H), 1.65-1.83 (m, 3 H), 1.84-1.96 (m, 2 H), 1.98-2.11 (m, 2 H), 3.31-3.39 (m, 2 H), 3.94 (s, 2 H), 7.36-7.55 (m, 3 H), 7.67-7.80 (m, 2 H), 8.10-8.34 (m, 3 H); UPLC-MS: 0.59 min, 470 [M + H]+. |
| 4-25 | 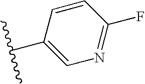 | 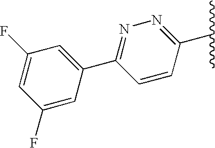 | ¹H NMR (400 MHz, DMSO-d₆): δ 1.14-1.34 (m, 2 H), 1.62-1.82 (m, 3 H), 1.82-1.95 (m, 2 H), 1.95-2.06 (m, 2 H), 3.20-3.46 (m, 2 H, under water signal), 3.98 (s, 2 H), 7.11- 7.23 (m, 1 H), 7.23-7.30 (m, 1 H), 7.30-7.42 (m, 1 H), 7.67-7.80 (m, 2 H), 7.99-8.17 (m, 1 H), 8.25-8.33 (m, 1 H), 8.33-8.41 (m, 1 H); UPLC-MS: 0.62 min, 470 [M + H]+ |
| 4-26 | 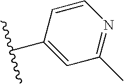 | 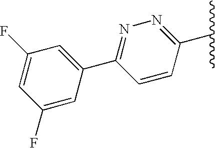 | ¹H NMR (400 MHz, CDCl₃): δ 1.21- 1.36 (m, 2 H), 1.82-1.96 (m, 3 H), 1.96-2.14 (m, 4 H), 2.59 (s, 3 H), 3.53-3.61 (m, 2 H), 3.79 (s, 2 H), 4.80-4.89 (m, 1 H), 6.75 (d, 1 H), 6.81-6.90 (m, 1 H), 7.32-7.36 (m, 1 H), 7.39-7.42 (m, 1 H), 7.50-7.60 (m, 3 H), 8.42-8.49 (m, 1 H); UPLC-MS: 0.53 min, 466 [M + H]+ |
| 4-27 | 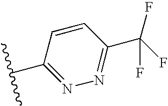 | 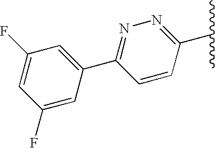 | ¹H NMR (400 MHz, CDCl₃): δ 1.22- 1.38 (m, 2 H), 1.86-2.01 (m, 3 H), 2.02-2.18 (m, 4 H), 3.45-3.55 (m, 2 H), 4.27 (s, 2 H), 5.14-5.30 (m, 1 H), 6.76-6.82 (m, 1 H), 6.82-6.92 (m, 1 H), 7.49-7.58 (m, 2 H), 7.60 (d, 1 H), 7.83 (d, 1 H), 8.73-8.82 (m, 1 H); UPLC-MS: 0.68 min, 521 [M + H]+ |
| 4-28 | 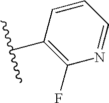 | 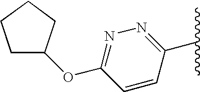 | ¹H NMR (400 MHz, DMSO-d₆): δ 1.12-1.30 (m, 2 H), 1.50-2.14 (m, 15 H), 3.18-3.30 (m, 2 H), 3.93 (s, 2 H), 5.14-5.28 (m, 1 H), 7.35-7.50 (m, 2 H), 7.50-7.66 (m, 1 H), 8.10- 8.25 (m, 2 H), 9.10-9.60 (m, 1 H), 13.6-14.1 (br s, 1 H); UPLC-MS: 0.56 min, 442 [M + H]+ |
| 4-29 | 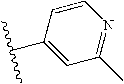 | 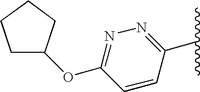 | ¹H NMR (400 MHz, DMSO-d₆): δ 1.17-1.36 (m, 2 H), 1.52-1.85 (m, 9 H), 1.85-2.11 (m, 6 H), 2.65 (s, 3 H), 3.16-3.28 (m, 2 H), 4.06 (s, 2 H), 5.16-5.28 (m, 1 H), 7.34-7.49 (m, 1 H), 7.50-7.73 (m, 1 H), 7.73- 7.86 (m, 1 H), 7.89-8.03 (m, 1 H), 8.56-8.68 (m, 1 H); UPLC-MS: 0.51 min, 438 [M + H]+ |
| 4-30 | 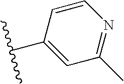 | 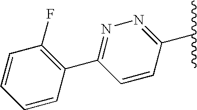 | ¹H NMR (400 MHz, DMSO-d₆): δ 1.15-1.35 (m, 2 H), 1.65-1.83 (m, 3 H), 1.84-1.98 (m, 2 H), 1.98-2.08 (m, 2 H), 2.64 (s, 3 H), 3.20-3.50 (m, 2 H, under water signal), 4.05 (s, 2 H), 7.00-7.28 (m, 1 H), 7.28-7.41 (m, 2 H), 7.45-7.56 (m, 1 H), 7.65- 7.75 (m, 1 H), 7.75-7.81 (m, 1 H), 7.81-7.92 (m, 1 H), 7.92-8.04 (m, 1 H), 8.57-8.67 (m, 1 H); UPLC-MS: 0.50 min, 448 [M + H]+ |
| 4-31 | 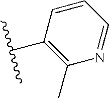 | 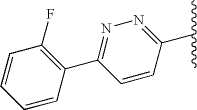 | ¹H NMR (400 MHz, DMSO-d₆): δ 1.15-1.38 (m, 2 H), 1.67-1.86 (m, 3 H), 1.86-1.99 (m, 2 H), 2.04-2.17 (m, 2 H), 2.53 (br s., 3 H), 3.28-3.42 (m, 2 H), 3.87-3.99 (m, 2 H), 7.36- 7.52 (m, 2 H), 7.53-7.69 (m, 2 H), 7.69-7.88 (m, 2 H), 8.02-8.24 (m, 2 H), 8.47-8.64 (m, 1 H); UPLC-MS: 0.72 min, 448 [M + H]+ |
| 4-32 | 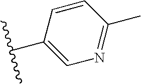 | 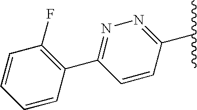 | ¹H NMR (400 MHz, DMSO-d₆): δ 1.18-1.38 (m, 2 H), 1.65-1.87 (m, 3 H), 1.87-1.98 (m, 2 H), 1.98-2.09 (m, 2 H), 2.61 (s, 3 H), 3.32-3.39 (m, 2 H), 3.97-4.08 (m, 2 H), 7.37-7.51 (m, 2 H), 7.56-7.76 (m, 3 H), 7.76- 7.85 (m, 1 H), 8.02-8.14 (m, 1 H), 8.30-8.43 (m, 1 H), 8.72-8.85 (m, 1 H); UPLC-MS: 0.76 min, 448 [M + H]+ |
| 4-33 | 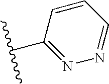 | 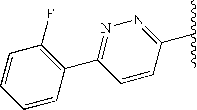 | ¹H NMR (400 MHz, DMSO-d₆): δ 9.02 (dd, 1 H), 8.38 (dd, 1 H), 8.10- 8.02 (m, 1 H), 7.82-7.73 (m, 2 H), 7.65-7.57 (m, 2 H), 7.47-7.39 (m, 2 H), 4.2 (s, 2 H), 3.39 (m, 2 H), 2.11- 1.20 (m, 9 H); UPLC-MS: 0.56 min, 435 [M + H]+. |
| 4-34 | 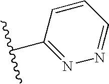 | 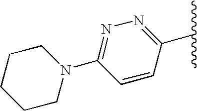 | ¹H NMR (400 MHz, DMSO-d₆): δ 9.02 (dd, 1 H), 8.40 (dd, 1 H), 7.86 (d, 1 H), 7.75 (dd, 1 H), 7.43 (br s, 1 H), 4.15 (s, 2 H), 3.44 (br s, 4 H), 3.22 (br s, 2 H), 2.05-1.17 (m, 15 H); UPLC-MS: 0.51 min, 424 [M + H]+ |
| 4-35 | 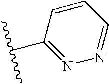 | 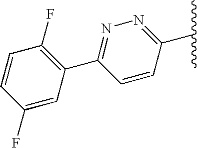 | ¹H NMR (400 MHz, DMSO-d₆): δ 9.02 (dd, 1 H), 8.40 (dd, 1 H), 8.08 (d, 1 H), 7.75 (dd, 1 H), 7.69-7.61 (m, 2 H), 7.55-7.45 (m, 2 H), 4.17 (s, 2 H), 3.40 (br s, 2 H), 2.13-1.10 (m, 9 H); HPLC-MS: 1.75 min, 453 [M + H]+ |
| 4-36 | 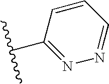 | 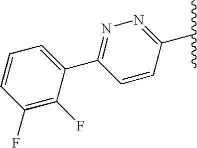 | ¹H NMR (400 MHz, DMSO-d₆): δ 9.01 (dd, 1 H), 8.40 (dd, 1 H), 8.07 (d, 1 H), 7.75 (dd, 1 H), 7.68-7.58 (m, 2 H), 7.55-7.44 (m, 2 H), 4.18 (s, 2 H), 3.40 (t, 2 H), 2.11-1.10 (m, 9 H); HPLC-MS: 1.75 min, 453 [M + H]+ |
| 4-37 | 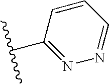 | 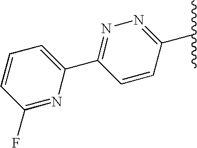 | ¹H NMR (400 MHz, DMSO-d₆): δ 9.02 (dd, 1 H), 8.42-8.34 (m, 2 H), 8.26-8.45 (m, 2 H), 7.75 (dd, 1 H), 7.60 (br s, 1 H), 7.36 (br s, 1 H), 4.18 (s, 2 H), 3.41 (br s, 2 H), 2.12- 1.10 (m, 9 H); HPLC-MS: 1.63 min, 436 [M + H]+ |
| 4-38 | 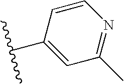 | 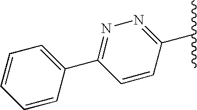 | ¹H NMR (400 MHz, DMSO-d₆): δ 9.02 (dd, 1 H), 8.40 (dd, 2 H), 8.02- 7.98 (m, 2 H), 7.75 (dd, 2 H), 7.61- 7.57 (m, 3 H), 4.16 (s, 2 H), 3.40 (br s, 2 H), 2.11-1.24 (m, 9 H); HPLC-MS: 1.50 min, 417 [M + H]+ |
| 4-39 | 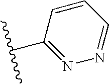 | 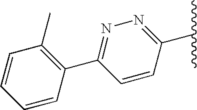 | ¹H NMR (400 MHz, DMSO-d₆): δ 8.67 (d, 1 H), 8.04 (d, 1 H), 8.01- 7.92 (m, 1 H), 7.86 (s, 1 H), 7.73 (br s, 1 H), 7.49-7.33 (m, 4 H), 4.10 (s, 2 H), 3.34 (br s, 2 H), 2.68 (s, 3 H), 2.36 (s, 3 H), 2.10-1.17 (m, 9 H); HPLC-MS: 1.34 min, 444 [M + H]+. |
| 4-40 | 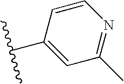 | 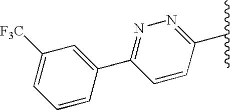 | ¹H NMR (400 MHz, DMSO-d₆): δ 9.02 (dd, 1 H), 8.47 (d, 1 H), 8.40 (dd, 1 H), 8.33-8.29 (m, 2 H), 7.95 (d, 1 H), 7.84 (t, 1 H), 7.75 (dd, 1 H), 7.73-7.63 (m, 1 H), 4.18 (s, 2 H), 3.41 (m, 2 H), 2.12-1.20 (m, 9 H); HPLC-MS: 1.99 min, 485 [M + H]+. |
| 4-41 | 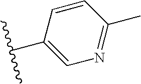 | 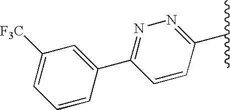 | ¹H NMR (400 MHz, DMSO-d₆): δ 8.68 (d, 1 H), 8.34-8.28 (m, 2 H), 8.06 (m, 1 H), 7.94-7.76 (m, 3 H), 7.48 (br s, 2 H), 4.09 (s, 2 H), 3.37 (m, 2 H), 2.69 (s, 3 H), 2.10-1.16 (m, 9 H); HPLC-MS: 1.62 min, 498 [M + H]+. |
| 4-42 | 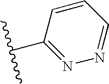 | 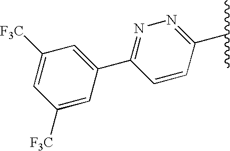 | ¹H NMR (400 MHz, DMSO-d₆): δ 9.02 (dd, 1 H), 8.64 (s, 2 H), 8.51 (d, 1 H), 8.40 (dd, 1 H), 8.31 (br s, 1 H), 7.75 (dd, 1 H), 7.60 (br s, 1 H), 4.18 (s, 2 H), 3.43 (m, 2 H), 2.12-1.22 (m, 9 H); UPLC-MS: 0.94 min, 553 [M + H]+. |
| 4-43 | 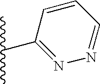 | 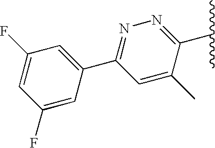 | ¹H NMR (400 MHz, CDCl₃): δ 8.97 (dd, 1 H), 8.57 (dd, 1 H), 7.72-7.64 (m, 2 H), 7.60-7.45 (m, 2 H), 7.41 (s, 1 H), 6.87-6.82 (m, 1 H), 4.59 (br t, 1 H), 4.23 (s, 2 H), 3.63 (t, 2 H), 2.24 (s, 3 H), 2.12-1.06 (m, 9 H); UPLC-MS: 0.57 min, 467 [M + H]+. |
| 4-44 | 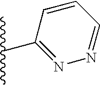 | 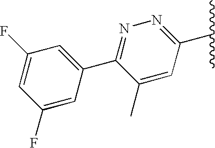 | ¹H NMR (400 MHz, acetone-d₆): δ 8.84 (d, 1 H), 8.33 (d, 1 H), 7.54 (m, 1 H), 7.04-7.15 (m, 2 H), 6.93 (t, 1 H), 6.66 (s, 1 H), 6.22 (t, 1 H), 4.06 (s, 2 H), 3.37 (t, 2 H), 2.14 (s, 3 H), 1.92-1.29 (m, 9 H); UPLC-MS: 0.56 min, 467 [M + H]+. |
| 4-45 | 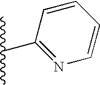 | 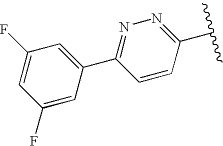 | ¹H NMR (400 MHz, MeOD-d₄): δ 1.34-1.49 (m, 2 H) 1.87-1.98 (m, 3 H) 2.11 (s, 4 H) 3.40-3.49 (m, 2 H) 4.18 (s, 2 H) 7.22 (s, 1 H) 7.32-7.39 (m, 1 H) 7.59-7.78 (m, 3 H) 8.01- 8.06 (m, 1 H) 8.11 (s, 1 H) 8.30-8.37 (m, 1 H) 8.40-8.47 (m, 1 H); UPLC-MS: 0.64 min, 452 [M + H]+ |
| 4-46 | 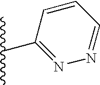 | 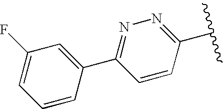 | ¹H NMR (400 MHz, DMSO-d₆): δ 1.34 (d, 2 H), 1.68-1.85 (m, 4 H), 1.91 (br s, 2 H), 2.06 (d, 2 H), 3.32- 3.47 (m, 2 H), 4.17 (s, 2 H), 7.40- 7.50 (m, 1 H), 7.56-7.90 (m, 5 H), 8.39 (dd, 2 H), 9.02 (dd, 1 H); UPLC-MS: 0.54 min, 435 [M + H]+. |
| 4-47 | 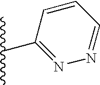 | 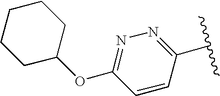 | ¹H NMR (400 MHz, CDCl₃): δ 1.10- 1.25 (m, 2 H), 1.51-1.64 (m, 1 H), 1.75-1.86 (m, 2 H), 1.87-2.00 (m, 4 H), 2.29 (br s, 1 H), 3.47 (d, 2 H), 4.00 (s, 2 H), 6.97-7.05 (m, 1 H), 7.64-7.72 (m, 1 H), 8.21 (dt, 1 H), 8.25-8.32 (m, 1 H); UPLC-MS: 0.57 min, 439 [M + H]+. |
| 4-48 | 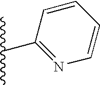 | 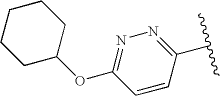 | ¹H NMR (400 MHz, MeOD-d₄): δ 1.30-1.54 (m, 6 H), 1.63 (br s, 3 H), 1.79-1.98 (m, 4 H), 2.00-2.19 (m, 4 H), 3.31 (s, 1 H), 3.33 (dt, 4 H), 4.18 (s, 2 H), 7.38-7.45 (m, 2 H), 7.50 (s, 1 H) 7.94-8.00 (m, 1 H), 8.22 (d, 1 H), 8.42-8.47 (m, 1 H); UPLC-MS: 0.61 min, 438 [M + H]+. |
| 4-49 | 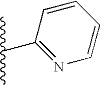 | 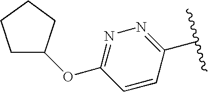 | ¹H NMR (400 MHz, CDCl₃): δ 1.59- 1.90 (m, 5 H), 1.96-2.07 (m, 4 H), 2.96-3.07 (m, 2 H), 3.29-3.43 (m, 5 H), 3.82-3.88 (m, 4 H), 4.02-4.11 (m, 2 H), 6.56-6.65 (m, 1 H), 6.94- 6.99 (m, 1 H), 7.04-7.09 (m, 1 H), 7.69-7.78 (m, 2 H), 8.27-8.31 (m, 1 H), 8.31-8.38 (m, 1 H); UPLC-MS: 0.59 min, 424 [M + H]+. |
| 4-50 | 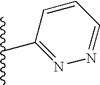 | 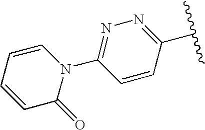 | ¹H NMR (400 MHz, DMSO-d₆): δ 1.26-1.38 (m, 2 H), 1.68-1.83 (m, 4 H), 1.87-1.96 (m, 2 H), 2.02-2.07 (m, 2 H), 3.31-3.41 (m, 2 H), 4.16 (s, 2 H), 6.43 (s, 1 H), 6.55 (dd, 1 H), 7.45-7.51 (m, 1 H), 7.57-7.63 (m, 1 H), 7.74-7.82 (m, 2 H), 7.83-7.92 (m, 1 H), 8.39 (dd, 1 H), 9.02 (dd, 1 H); UPLC-MS: 0.46 min, 434 [M + H]+. |
| 4-51 | 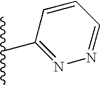 | 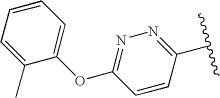 | ¹H NMR (400 MHz, CDCl₃): δ 1.14- 1.30 (m, 2 H), 1.81-1.95 (m, 3 H), 1.96-2.09 (m, 4 H), 2.23 (s, 3 H), 3.30-3.38 (m, 2 H), 4.20 (s, 2 H), 4.62-4.73 (m, 1 H), 6.78 (d, 1 H), 6.98-7.04 (m, 1 H), 7.05-7.15 (m, 2 H), 7.17-7.27 (m, 2 H), 7.46-7.56 (m, 1 H), 8.54-8.61 (m, 1 H), 8.92- 8.99 (m, 1 H); UPLC\MS: 0.78 min, 447 [M + H]+. |
| 4-52 | 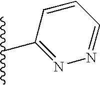 | 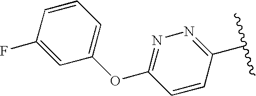 | ¹H NMR (400 MHz, CDCl₃): δ 1.15- 1.33 (m, 2 H), 1.81-1.96 (m, 3 H), 1.96-2.10 (m, 4 H), 3.32-3.43 (m, 2 H), 4.21 (s, 2 H), 4.71-4.82 (m, 1 H), 6.79 (d, 1 H), 6.84-7.00 (m, 3 H), 7.02 (d, 1 H), 7.28-7.38 (m, 1 H), 7.45-7.56 (m, 1 H), 8.53-8.61 (m, 1 H), 8.93-9.01 (m, 1 H); UPLC\MS: 0.76 min, 451 [M + H]+. |
| 4-53 | 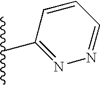 | 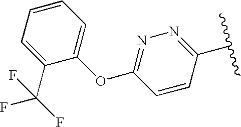 | ¹H NMR (400 MHz, CDCl₃) δ 1.12- 1.30 (m, 2 H), 1.81-2.08 (m, 7 H), 3.36 (t, 2 H), 4.20 (s, 2 H), 4.52 (t, 1 H), 6.79 (d, 1 H), 7.08 (d, 1 H), 7.22-7.29 (m, 1 H), 7.37-7.42 (m, 1 H), 7.46-7.61 (m, 2 H), 7.66-7.72 (m, 1 H), 8.54-8.60 (m, 1 H), 8.94- 8.98 (m, 1 H); UPLC\MS: 0.82 min, 501 [M + H]+. |
| 4-54 | 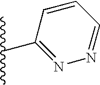 | 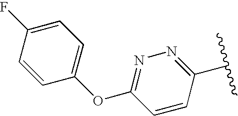 | ¹H NMR (400 MHz, CDCl₃) δ 1.15- 1.31 (m, 2 H), 1.67-2.09 (m, 7 H), 3.31-3.39 (m, 2 H), 4.22 (s, 2 H), 6.99-7.11 (m, 3 H), 7.13-7.20 (m, 2 H), 7.45-7.56 (m, 1 H), 8.04-8.10 (m, 0.6 H), 8.58 (dd, 1 H), 8.95-9.00 (m, 1 H); UPLC-MS: 0.75 min, 451 [M + H]+. |
| 4-55 | 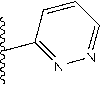 | 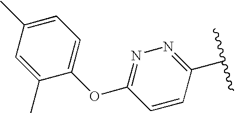 | ¹H NMR (400 MHz, CDCl₃): δ 1.11- 1.32 (m, 2 H), 1.81-1.96 (m, 3 H), 1.98-2.08 (m, 4 H), 2.19 (s, 3 H), 2.33 (s, 3 H), 3.32 (d, 2 H), 4.22 (s, 2 H), 6.82 (d, 1 H), 6.92-7.10 (m, 4 H), 7.47-7.56 (m, 1 H), 8.15 (br s, 0.6 H), 8.55-8.63 (m, 1 H), 8.94-9.01 (m, 1 H); UPLC\MS: 0.83 min, 461 [M + H]+. |
| 4-56 | 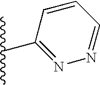 | 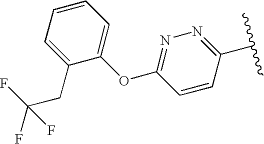 | ¹H NMR (400 MHz, CDCl₃): δ 1.14- 1.33 (m, 2 H), 1.83-1.97 (m, 3 H), 1.97-2.10 (m, 4 H), 3.36 (t, 2 H), 4.22 (s, 2 H), 6.81-6.91 (m, 1 H), 7.10-7.20 (m, 1 H), 7.20-7.46 (m, 4 H), 7.47-7.56 (m, 1 H), 8.55-8.61 (m, 1 H), 8.94-9.00 (m, 1 H); UPLC\MS: 0.84 min, 517 [M + H]+. |

### Example 5

### Preparation of Compounds of Formula (IIE)

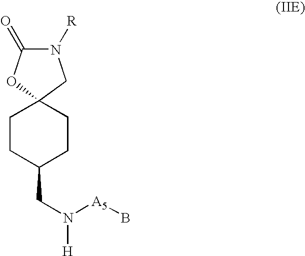

### Example 5-1

### (Trans)-8-[(1,3-benzothiazol-2-ylamino)methyl]-3-(2-pyridinyl)-1-oxa-3-azaspiro[4.5]decan-2-one

(Trans)-2-oxo-3-(2-pyridinyl)-1-oxa-3-azaspiro[4.5]decane-8-carbaldehyde (Intermediate 12 procedure 12b, 21 mg, 0.081 mmol) and 1,3-benzothiazol-2-amine (12.12 mg, 0.081 mmol) were combined in a closed vial in dichloromethane (2 ml). Chlorotitanium triisopropoxide (0.039 ml, 0.161 mmol) was added and the mixture was stirred at r.t. overnight (ca 15 hrs). Then acetic acid (0.092 ml, 1.614 mmol) and sodium triacetoxyborohydride (85 mg, 0.403 mmol) were added and the mixture was stirred at r.t. for 6 hours. The mixture was diluted with DCM (2 ml), NaOH 20% aqueous was added (2 ml) and the mixture was extracted with DCM (3×3 ml). The organic phases were combined, filtered through a filter tube and evaporated under reduced pressure to afford a crude that was purified by MDAP (Preparative Column: AXIA 50×21 mm; Focus 01: 30% (B) to 35% (B) in 1 min; 35% (B) to 65% (B) in 7 min; 65% (B) to 100% (B) in 1 min; 100% (B) for 1.5 min; where Mobile phase are A: NH₄HCO₃ so1.10 mM, pH10; B: ACN). Product fractions were combined and evaporated under reduced pressure to afford the title compound as a yellowish solid (6.0 mg, 19%);

### Example 5-2

### (Trans)-8-{[(5,6-dichloro-1,3-benzothiazol-2-yl)amino]methyl}-3-(2-pyridinyl)-1-oxa-3-azaspiro[4.5]decan-2-one Hydrochloride

(Trans)-2-oxo-3-(2-pyridinyl)-1-oxa-3-azaspiro[4.5]decane-8-carbaldehyde (Intermediate 12, procedure 12b, 30 mg, 0.115 mmol) and 5,6-dichloro-1,3-benzothiazol-2-amine (25.3 mg, 0.115 mmol) were combined in a closed vial in dichloromethane (2 ml). Chlorotitanium triisopropoxide (0.055 ml, 0.231 mmol) was added and the mixture was stirred at r.t. overnight (ca 15 hours). Then acetic acid (0.132 ml, 2.305 mmol) and sodium triacetoxyborohydride (122 mg, 0.576 mmol) were added and the mixture was stirred at r.t. for 6 hours. The mixture was diluted with DCM (2 ml), 20% aqueous NaOH was added (2 ml) and the mixture was extracted with DCM (3×3 ml). The organic were combined, filtered through a filtering tube and dried (vacuo) to afford a crude that was purified by Fraction Lynx (Preparative Column: AXIA 50×21 mm; Method: Focus 03:40% (B) to 45% (B) in 1 min; 45% (B) to 100% (B) in 8 min; 100% (B) for 1.5 min; where mobile phase are A: NH₄HCO₃ so1.10 mM, pH10; B: ACN). Product fractions were combined and dried to afford (trans)-8-{[(5,6-dichloro-1,3-benzothiazol-2-yl)amino]methyl}-3-(2-pyridinyl)-1-oxa-3-azaspiro[4.5]decan-2-one as a white solid (8 mg, 15%).

HPLC-MS: 2.999 min, 463 [M+H]+

The above compound (8 mg, 0.017 mmol) was suspended in Et₂O (0.5 ml) and DCM (0.2 ml) and 1M HCl in Et₂O (0.02 ml, 0.02 mmol) were added. The resulting solid was triturated with Et₂O (3×0.3 ml) and dried (vacuo at 40° C. for 6 hours) to afford the title compound as a white solid (6.3 mg, 11%).

### Example 5-3

### (Trans)-3-(3-pyridazinyl)-8-[({6-[(trifluoromethyl)oxy]-1,3-benzothiazol-2-yl}amino)methyl]-1-oxa-3-azaspiro[4.5]decan-2-one Hydrochloride

(Trans)-2-oxo-3-(3-pyridazinyl)-1-oxa-3-azaspiro[4.5]decane-8-carbaldehyde

(Intermediate 60, 50 mg, 0.191 mmol), 6-[(trifluoromethyl)oxy]-1,3-benzothiazol-2-amine (44.8 mg, 0.191 mmol), titanium(IV) isopropoxide (0.112 ml, 0.383 mmol) were collected in dichloromethane (2 ml) and shaken at r.t. overnight The reaction was stirred at rt for further 24 hours. A small amount of Na₂SO₄ (100 mg) was added and shaken for further 24 hours then sodium borohydride (21.72 mg, 0.574 mmol) and ethanol (2.0 ml) were added and the resulting mixture was shaken overnight. The reaction mixture was diluted with DCM (20 ml), quenched with NaHCO₃ (5 ml) and filtered by a separation tube. The resulting organic phase was concentrated under vacuum and the residue (87 mg) was purified by flash chromatography (Biotage SP1) over a Varian Metaflash NH₂ 12S cartridge, using cyclohexane and EtOAc as eluent. (Trans)-3-(3-pyridazinyl)-8-[({6-[(trifluoromethyl)oxy]-1,3-benzothiazol-2-yl}amino)methyl]-1-oxa-3-azaspiro[4.5]decan-2-one was recovered as a colourless oil (30 mg) contaminated by ca 50% of starting amine. It was further purified by silica gel chromatography (Biotage SP1) over a Varian Metaflash 12S cartridge using DCM and MeOH as eluent and finally by reverse phase chromatography (KP-C18 12M cartridge), using a gradient of ACN and water (made up with 0.1% HCOOH). The required pure compound was recovered as a colourless solid (20 mg).

¹H NMR (400 MHz, CDCl₃): δ 8.97 (dd, 1H), 8.57 (dd, 1H), 7.55-7.45 (m, 3H), 7.19 (d, 1H), 4.23 (s, 2H), 3.39 (d, 2H), 2.13-1.82 (m, 7H), 1.36-1.15 (m, 2H); UPLC-MS: 0.74 min 480 [M+H]+.

The above compound was dissolved in DCM (2 ml) and reacted with 1.0 equiv. of 1M HCl in Et₂O, Solvent was removed in vacuo to give the title compound as a colourless solid (14.8 mg).

### Example 5-4

### (Trans)-3-(3-pyridazinyl)-8-({[6-(trifluoromethyl)-1,3-benzothiazol-2-yl]amino}methyl)-1-oxa-3-azaspiro[4.5]decan-2-one Hydrochloride

(Trans)-2-oxo-3-(3-pyridazinyl)-1-oxa-3-azaspiro[4.5]decane-8-carbaldehyde (Intermediate 60, 50 mg, 0.191 mmol), 6-(trifluoromethyl)-1,3-benzothiazol-2-amine (41.8 mg, 0.191 mmol), titanium(IV) isopropoxide (0.112 ml, 0.383 mmol) were collected in DCM (2 ml) and stirred at rt overnight. The reaction was stirred at rt for further 24h. A small amount of Na₂SO₄ (100 mg) was added and shaken for further 24 hours at r.t. then sodium borohydride (21.72 mg, 0.574 mmol) and ethanol (2.000 ml) were added. After 5 hours stirring a further amount of solvent DCM/EtOH (2/1 ml) and NaBH₄ (20 mg) were added and the reaction mixture was shaken overnight. It was rinsed with DCM (20 ml) and quenched with NaHCO₃ aq (5 ml), filtering over a separation tube. The resulting organic phase was concentrated under vacuum and the crude was purified by flash chromatography (Biotage SP1) over a Varian Metaflash NH₂ 12S cartridge, using cyclohexane and EtOAc as eluent. The required compound was recovered as colourless oil (25 mg) contaminated by ca 50% of starting amine. The residue was purified by KP-RP flash chromatography (Biotage SP1) over a 12M C18 cartridge eluting with a gradient of ACN and water (made up with 0.1% HCOOH) to give (trans)-3-(3-pyridazinyl)-8-({[6-(trifluoromethyl)-1,3-benzothiazol-2-yl]amino}methyl)-1-oxa-3-azaspiro[4.5]decan-2-one as a colourless solid (22 mg).

¹H NMR (400 MHz, CDCl₃): δ 8.99 (dd, 1H), 8.58 (dd, 1H), 7.86 (s, 1H), 7.61-7.49 (m, 3H), 6.07 (br s, 1H), 4.23 (s, 2H), 3.43 (d, 2H), 2.13-1.82 (m, 7H), 1.38-1.18 (m, 2H). UPLC-MS: 0.73 min, 464 [M+H]+.

The above compound was dissolved in DCM (2 ml) and reacted with 1.0 equiv. of 1M HCl in Et₂O, Solvent was removed to give the title compound as a colourless solid (14.4 mg).

### Example 5-5

### (Trans)-8-[(1,2-benzisothiazol-3-ylamino)methyl]-3-(3-pyridazinyl)-1-oxa-3-azaspiro[4.5]-decan-2-one Hydrochloride

(Trans)-2-oxo-3-(3-pyridazinyl)-1-oxa-3-azaspiro[4.5]decane-8-carbaldehyde (Intermediate 60, 50 mg, 0.191 mmol), 1,2-benzoisothiazol-3-amine (28.7 mg, 0.191 mmol), titanium(IV) isopropoxide (0.112 ml, 0.383 mmol) were collected in dichloromethane (2 ml) and shaken at r.t. overnight. The reaction was stirred at rt for further 24 h. Na₂SO₄ (100 mg) was added and the mixture was shaken for further 24 hours then sodium borohydride (21.72 mg, 0.574 mmol) and ethanol (2.0 ml) were added and shaking 5 hours. The reaction mixture was diluted with DCM (20 ml) and quenched with NaHCO₃ (5 ml), filtering over a separation tube. The resulting organic phase was concentrated under vacuum and the crude (100 mg) was purified by flash chromatography with Biotage SP1, over a Varian Metaflash NH₂ 12S cartridge, eluting with cyclohexane and EtOAc. The residue was further purified by KP-RP flash chromatography (Biotage SP1) over a 12M C18 cartridge, eluting with a gradient of ACN and water (made up with 0.1% HCOOH) to afford (trans)-8-[(1,2-benzoisothiazol-3-ylamino)methyl]-3-(3-pyridazinyl)-1-oxa-3-azaspiro[4.5]decan-2-one as a colourless solid (25 mg).

¹H NMR (400 MHz, CDCl₃): δ 8.97 (dd, 1H), 8.57 (dd, 1H), 7.80 (d, 1H), 7.70 (d, 1H), 7.53-7.47 (m, 2H), 7.37 (m, 1H), 5.20 (br s, 1H), 4.22 (s, 2H), 3.53 (br s, 2H), 2.13-1.82 (m, 7H), 1.35-1.18 (m, 2H). UPLC-MS: 0.69 min, 396 [M+H]+.

The above compound was dissolved in DCM (2 ml) and reacted with 1.0 equiv. of 1M HCl in Et₂O, Solvent was removed to give the title compound as a yellow solid (16.6 mg).

All the analytical data are set forth in the following Table 5-1 and in which R, A₅ and B are:

| | |
|---|---|
| | (IIE) |
| 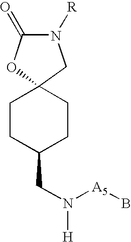 | |

| Cpd. | | | |
|---|---|---|---|
| No. | R | A5-B | Analytical Data |
| 5-1 | 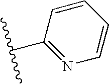 | 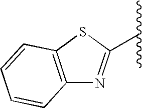 | ¹H NMR (400MHz, CDCl₃): δ 1.18- 1.37 (m, 2 H), 1.76-1.94 (m, 3 H), 1.96- 2.10 (m, 4 H), 3.39 (d, 2 H), 4.06 (s, 2 H), 5.51-5.65 (br s, 1 H), 7.00-7.17 (m, 2 H), 7.29-7.37 (m, 1 H), 7.51-7.64 (m, 2 H), 7.69-7.76 (m, 1 H), 8.28 (dt, 1 H), 8.35 (ddd, 1 H); HPLC-MS: 2.096 min, 395 [M + H]+ |
| 5-2 | 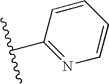 | 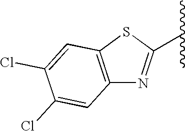 | ¹H NMR (400 MHz, DMSO-d₆): δ 1.19-1.40 (m, 2 H), 1.61-1.78 (m, 3 H), 1.78-1.89 (m, 2 H), 1.91-2.07 (m, 2 H), 3.27-3.36 (m, 2 H), 4.02 (s, 2 H), 7.01- 7.22 (m, 1 H), 7.32-7.44 (m, 1 H), 7.75- 7.90 (m, 2 H), 8.05-8.16 (m, 1 H), 8.31-8.41 (m, 1 H), 8.42-8.57 (m, 1 H); MS: 463, 465 [M + H]+. |
| 5-3 | 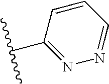 | 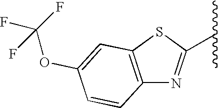 | ¹H NMR (400 MHz, DMSO-d₆): δ 9.03 (dd, 1 H), 8.40 (dd, 1 H), 7.87 (br s, 1 H), 7.76 (dd, 1 H), 7.50 (d, 1 H), 7.28 (d, 1 H), 4.15 (s, 2 H), 3.40 (br s, 2 H), 2.12-0.77 (m, 9 H); UPLC-MS: 0.74 min, 480 [M + H]+. |
| 5-4 | 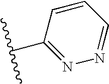 | 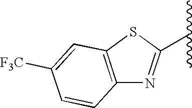 | ¹H NMR (400 MHz, DMSO-d₆): δ 9.02 (dd, 1 H), 8.72 (br s, 1 H), 8.40 (dd, 1 H), 8.15 (s, 1 H), 7.75 (dd, 1 H), 7.56 (m, 1 H), 4.16 (s, 2 H), 3.38 (br s, 2 H), 2.12-0.82 (m, 9 H); UPLC-MS: 0.73 min, 464 [M + H]+. |
| 5-5 | 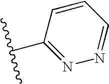 | 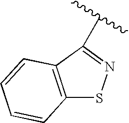 | ¹H NMR (400 MHz, DMSO-d₆): δ 9.02 (dd, 1 H), 8.40 (dd, 1 H), 8.19 (dt, 1 H), 7.93 (dt, 1 H), 7.76 (dd, 1 H), 7.55-750 (m, 1 H), 7.42-7.35 (m, 1 H), 4.16 (s, 2 H), 3.36 (br s, 1 H), 2.10-0.80 (m, 9 H); UPLC-MS: 0.69 min, 396 [M + H]+. |

### Example 6

### Preparation of Compounds of Formula (IIF)

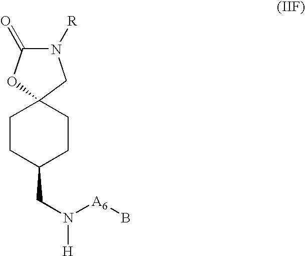

### Example 6-1

### (Trans)-8-{[(5,6-dichloro-1H-benzimidazol-2-yl)amino]methyl}-3-(2-pyridinyl)-1-oxa-3-azaspiro[4.5]decan-2-one

(Trans)-2-oxo-3-(2-pyridinyl)-1-oxa-3-azaspiro[4.5]decane-8-carbaldehyde (Intermediate 12 procedure 12b, 30 mg, 0.115 mmol) and 5,6-dichloro-1H-benzimidazol-2-amine (23.29 mg, 0.115 mmol) were dissolved in dichloromethane (2 ml) in a closed vial. Titanium(IV) isopropoxide (0.068 ml, 0.231 mmol) was added and the mixture was stirred at r.t. overnight (ca 15 hours) then it was stirred at 60° C. for 1 hour. The mixture was allowed to cool to r.t. and NaBH₄ (17.44 mg, 0.461 mmol) followed by ethanol (0.5 ml) were added and the mixture was stirred at r.t. for 6 hours. The mixture was diluted with DCM (2 ml), a saturated aqueous solution of K₂CO₃ (2 ml) was added and the mixture was stirred at r.t. for 30 minutes and then extracted with DCM (3×3 ml). The organic phases were combined, filtered through a filter tube and evaporated under reduced pressure to afford a crude that was purified by MDAP (Preparative Column: AXIA 50×21 mm; Focus 01: 30% (B) to 35% (B) in 1 min; 35% (B) to 65% (B) in 7 min; 65% (B) to 100% (B) in 1 min; 100% (B) for 1.5 min; where Mobile phase are A: NH₄HCO₃ so1.10 mM, pH10; B: ACN). Product fractions were combined and evaporated under reduced pressure to afford the title compound as a white solid (6.7 mg, 13%);

All the analytical data are set forth in the following Table 6-1 and in which R, A₆ and B are:

| | |
|---|---|
| | (IIF) |
| 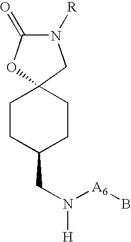 | |

| Cpd. | | | |
|---|---|---|---|
| No. | R | A6-B | Analytical Data |
| 6-1 | 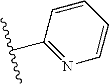 | 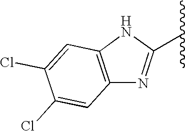 | ¹H NMR (400 MHz, CDCl₃): δ 1.06-1.23 (m, 2 H), 1.63-1.83 (m, 3 H), 1.83-2.06 (m, 4 H), 3.26-3.39 (m, 2 H), 4.01 (s, 2 H), 5.17-5.34 (br s, 1 H), 7.08 (ddd, 1 H), 7.30- 7.38 (m, 2 H), 7.74 (ddd, 1 H), 8.20 (dt, 1 H), 8.35 (ddd, 1 H); HPLC-MS: 1.785 min, 446 and 448 [M + H]+ |

### Example 7

### Preparation of Compounds of Formula (IIG)

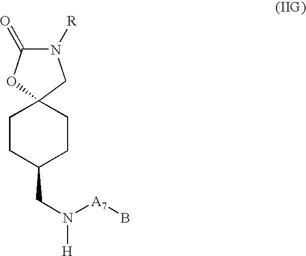

### Example 7-1

### (Trans)-3-(3-pyridazinyl)-8-({[2-(trifluoromethyl)imidazo[1,2-b]pyridazin-6-yl]amino}methyl)-1-oxa-3-azaspiro[4.5]decan-2-one

6-chloro-2-(trifluoromethyl)imidazo[1,2-b]pyridazine (Intermediate 74, 100 mg, 0.451 mmol), 18-crown-6 (11.93 mg, 0.045 mmol), potassium fluoride (39.3 mg, 0.677 mmol) and sulfolane (4 ml) were collected in a microwave vial and irradiated at 200° C. for overall 60 min. The reaction mixture was diluted with DCM (200 ml) and washed with water (4×40 ml) filtering over a separation tube. The resulting organic phase was concentrated to remove DCM and the resulting yellow oil (4 ml) was placed in a vial. (Trans)-8-(aminomethyl)-3-(3-pyridazinyl)-1-oxa-3-azaspiro[4.5]decan-2-one (Intermediate 41 procedure 41c, 70 mg, 0.267 mmol) was added, followed by DIPEA (0.394 ml, 2.257 mmol). The resulting mixture was shaken at 140° C. for 3 hours. The reaction mixture was partially evaporated under vacuum, then it was purified over an ion exchange SCX cartridge (Varian, 2g) washing with MeOH and eluting with 2M ammonia in MeOH to give 130 mg of yellow oil. The crude was purified with Biotage SP1, over a KP-NH cartridge, eluting with a gradient of cyclohexane and ethyl acetate. The title compound was eluted with EtOAc and recovered as a colourless oil (84 mg).

¹H NMR showed the presence of an impurity (singlet at 4.22 ppm) which was assumed to be the corresponding cis isomer (˜10%)

### Example 7-2

### (Trans)-8-{[(2-phenylimidazo[1,2-b]pyridazin-6-yl)amino]methyl}-3-(3-pyridazinyl)-1-oxa-3-azaspiro[4.5]decan-2-one Dihydrochloride

6-Chloro-2-phenylimidazo[1,2-b]pyridazine (65.8 mg, 0.287 mmol), potassium fluoride (24.97 mg, 0.430 mmol), 18-crown-6 (15.15 mg, 0.057 mmol) and sulfolane (4 ml) were collected in a microwave vial and the reaction mixture was irradiated at 200° C. for overall 90 min. Then, it was taken up with DCM (200 ml) and washed with water (4×40 ml) filtering over a separation tube. The resulting organic phase was concentrated to give a brown oil (2 ml) which was placed in a closed vial followed by (trans)-8-(aminomethyl)-3-(3-pyridazinyl)-1-oxa-3-azaspiro[4.5]decan-2-one (Intermediate 41 procedure 41c, 50.1 mg, 0.191 mmol) and DIPEA (0.167 ml, 0.955 mmol) and the resulting mixture was shaken at 140° C. overnight then it was partially evaporated under vacuum and the resulting residue was passed through a SCX cartridge, washed with MeOH and eluted with 2M ammonia in MeOH. The resulting crude was further purified by flash chromatography (Biotage SP1) over a Varian NH₂ 25M cartridge eluting with a gradient of cyclohexane and ethyl acetate. (Trans)-8-{[(2-phenylimidazo[1,2-b]pyridazin-6-yl)amino]methyl}-3-(3-pyridazinyl)-1-oxa-3-azaspiro[4.5]decan-2-one was eluted with 100% EtOAc and recovered as a yellow solid (45 mg).

¹H NMR (400 MHz, CDCl₃): δ 8.97 (dd, 1H), 8.59 (dd, 1H), 8.00 (s, 1H), 7.94-7.90 (m, 2H), 7.67 (dd, 1H), 7.51 (dd, 1H), 7.47-7.42 (m, 2H), 7.35-7.30 (m, 1H), 6.44 (d, 1H), 4.48 (brs, 1H), 4.25 (s, 2H), 3.34 (t, 2H), 2.11-1.10 (m, 9H); UPLC-MS: 0.57 min, 456 [M+H]+.

The above compound was dissolved in DCM (2 ml) and reacted with 2 equiv. of 1M HCl in Et₂O. Solvent was removed and the resulting solid was tritured with Et₂O and filtered to give the title compound as colourless solid (40 mg).

### Example 7-3

### (Trans)-3-(3-pyridazinyl)-8-({[2-(2-pyridinyl)imidazo[1,2-b]pyridazin-6-yl]amino}methyl)-1-oxa-3-azaspiro[4.5]decan-2-one Dihydrochloride

The title compound was made in a similar fashion to the preparation of Example 7-2 replacing 6-chloro-2-phenylimidazo[1,2-b]pyridazine with 6-chloro-2-(2-pyridinyl)imidazo[1,2-b]pyridazine (Intermediate 75, 44.0 mg, 0.191 mmol) to afford the title compound (24 mg, 0.045 mmol) as a colourless solid.

### Example 7-4

### (Trans)-8-({[2-(3-fluorophenyl)imidazo[1,2-b]pyridazin-6-yl]amino}methyl)-3-(3-pyridazinyl)-1-oxa-3-azaspiro[4.5]decan-2-one Dihydrochloride

The title compound was made in a similar fashion to the preparation of Example 7-2 replacing 6-chloro-2-phenylimidazo[1,2-b]pyridazine with 6-chloro-2-(3-fluorophenyl)imidazo[1,2-b]pyridazine (Intermediate 76, 47.2 mg, 0.191 mmol) to afford the title compound as a colourless solid (39 mg, 0.071 mmol).

### Example 7-5

### (Trans)-8-({[2-(2-fluorophenyl)imidazo[1,2-b]pyridazin-6-yl]amino}methyl)-3-(3-pyridazinyl)-1-oxa-3-azaspiro[4.5]decan-2-one Dihydrochloride

The title compound was made in a similar fashion to the preparation of Example 7-2 replacing 6-chloro-2-phenylimidazo[1,2-b]pyridazine with 6-chloro-2-(2-fluorophenyl)-imidazo[1,2-b]pyridazine (Intermediate 78, 47.2 mg, 0.191 mmol) to afford the title compound as a colourless solid (35 mg, 0.064 mmol).

### Example 7-6

### (Trans)-8-({[2-(3,5-difluorophenyl)imidazo[1,2-b]rpyridazin-6-yl]amino}methyl)-3-(3-pyridazinyl)-1-oxa-3-azaspiro[4.5]decan-2-one Dihydrochloride

The title compound was made in a similar fashion to the preparation of Example 7-2 replacing 6-chloro-2-phenylimidazo[1,2-b]pyridazine with 6-chloro-2-(3,5-difluorophenyl)imidazo[1,2-b]pyridazine (Intermediate 80, 50.6 mg, 0.191 mmol) to afford the title compound as a colourless solid (48 mg, 0.085 mmol). All the analytical data are set forth in the following Table 7-1 and in which R, A₇ and B are:

| | |
|---|---|
| | (IIG) |
| 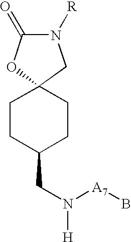 | |

| Cpd. | | | |
|---|---|---|---|
| No. | R | A7-B | Analytical Data |
| 7-1 | 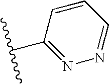 | 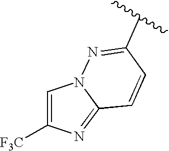 | ¹H NMR (400 MHz, CDCl₃): δ 8.98 (dd, 1 H), 8.59 (dd, 1 H), 7.95 (s, 1 H), 7.67 (d, 1 H), 7.52 (dd, 1 H), 6.55 (d, 1 H), 4.57 (br s), 1 H), 4.25 (s, 2 H), 3.34 (t, 2 H), 2.11- 1.44 (m, 9 H); UPLC-MS: 0.77 min, 448 [M + H]+ |
| 7-2 | 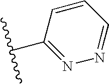 | 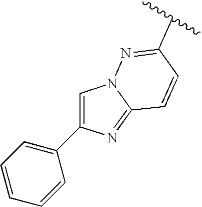 | ¹H NMR (400 MHz, DMSO-d₆): δ 9.02 (dd, 1 H), 8.83 (s, 1 H), 8.40 (dd, 1 H), 8.00-7.93 (m, 3 H), 7.84-7.71 (m, 2 H), 7.60-7.46 (m, 3 H), 7.20 (d, 1 H), 4.18 (s, 2 H), 3.23 (m, 2 H), 2.12-1.20 (m, 9 H); HPLC-MS: 1.78 min, 456 [M + H]+ |
| 7-3 | 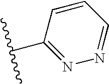 | 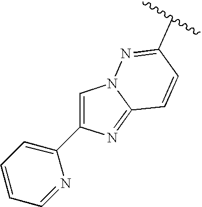 | ¹H NMR (400 MHz, DMSO-d₆): δ 9.01 (m, 2 H), 8.73 (m, 1 H), 8.39 (dd, 1 H), 8.25-8.16 (m, 2 H), 7.89 (d, 1 H), 7.89 (d, 1 H), 7.74 (dd, 2 H), 7.61 (m, 1 H), 7.16 (d, 1 H), 4.17 (s, 2 H), 3.22 (br t, 2 H), 2.11-1.17 (m, 9 H). UPLC-MS: 0.66 min, 457 [M + H]+; UPLC-MS: 0.66 min, 457 [M + H]+. |
| 7-4 | 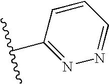 | 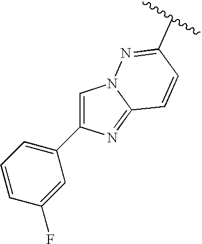 | ¹H NMR (400 MHz, DMSO-d₆): δ 9.01 (dd, 1 H), 8.76 (br s, 1 H), 8.40 (dd, 1 H), 7.90 (d, 1 H), 7.82-7.77 (m, 3 H), 7.64- 7.52 (m, 1 H), 7.75 (dd, 1 H), 7.29-7.02 (m, 2 H), 4.17 (s, 2 H), 3.21 (br s, 1 H), 2.14-1.18 (m, 9 H); UPLC-MS: 0.82 min, 474 [M + H]+. |
| 7-5 | 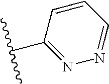 | 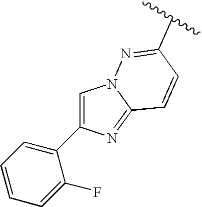 | ¹H NMR (400 MHz, DMSO-d₆): δ 9.01 (dd, 1 H), 8.45 (br s), 8.40 (dd, 1 H), 8.10- 8.04 (m, 1 H), 7.93 (d, 1 H), 7.76 (dd, 1 H), 7.71-7.35 (m, 4 H), 7.12 (d, 1 H), 4.17 (s, 2 H), 3.22 (br s, 1 H), 2.09-1.15 (m, 9 H); UPLC-MS: 0.83 min, 474 [M + H]+. |
| 7-6 | 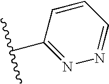 | 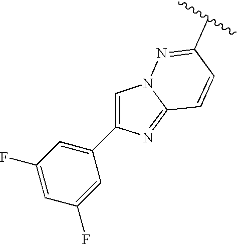 | ¹H NMR (400 MHz, DMSO): δ 9.02 (dd, 1 H), 8.72 (s, 1 H), 8.40 (dd, 1 H), 7.84 (d, 1 H), 7.75 (dd, 1 H), 7.71-7.63 (m, 1 H), 7.47 (br s, 1 H), 7.29 (m, 1 H), 6.99 (m, 1 H), 4.17 (s, 2 H), 3.20 (br s, 1 H), 2.14- 1.12 (m, 9 H); UPLC-MS: 0.69 min, 492 [M + H]+. |

### Example 8

### In Vitro Profile

The in vitro assessment of the NPY Y5 antagonist compounds used different assay systems to determine the potency and affinities against the NPY Y5 receptor.

The affinities of the compounds of the invention for the NPY Y5 receptor may be determined by the binding assays described below. Such affinity is typically calculated from the IC₅₀ obtained in competition experiments as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22: 3099, 1973). In the context of the present invention pKi values (corresponding to the antilogarithm of Ki) are used instead of Ki; pKi results are only estimated to be accurate to about 0.3-0.5.

### Binding Affinities at Human and Rat NPY Y5 Receptors

The assays used to measure compound affinity to human and rat NPY Y5 receptors were binding assays using Scintillation Proximity Assay (SPA) technology. The SPA involves the coupling of cell membrane fragments, via their glycosylated residues, to the wheat germ agglutinin (WGA) present on the surface of SPA beads. This coupling mechanism immobilises receptors in close proximity to the scintillant within the SPA beads and binding to the receptors of a radiolabelled ligand can thus be measured directly without the need to separate bound from free ligand.

Binding experiments are carried out in 384-well plates. The assay buffer contains 50 mM HEPES/NaOH pH 7.4, 1 mM MgCl2, 2.5 mM CaCl₂ and 0.05% pluronic acid. Specific binding is defined as the portion of [125I]-porcinePYY that is displaceable by 1 μM human PYY. A non-linear, 4 parameter logistic curve-fit of the data generated pIC₅₀ and pKi values.

### 125I-PYY Binding on Human NPY Y5 BacMam Membranes

Competition experiments are carried out in 384-well white with clear bottom plates in a final volume of 50 μL. PVT-WGA beads and membranes (prepared from HEK293F G0 cells) are diluted in assay buffer to have 2.5 mg/ml and 50 μg/ml, respectively and precoupled at 4° C. for 60 min. [125I]-PYY is added to the membrane-beads mix to achieve a concentration of 20 μM. 50 μL of the SPA mix is added to each well containing 0.5 μL compound solution. Compound solutions are prepared by serially diluting compounds in neat DMSO. The incubation lasted 3 hours at room temperature under gentle shaking. Then plates are left overnight at room temperature to allow the beads to settle and bound radioactivity is measured using Trilux MicroBeta.

### 125I-PYY Binding on Rat NPY Y5 BacMam Membranes

Competition experiments are carried out in 384-well white plates in a final volume of 30 μl. WGA-Polystyrene LEADseeker imaging beads and membranes (prepared from HEK293F GO cells), are diluted in assay buffer to have 2.5 mg/ml and 30 μg/ml, respectively and precoupled at 4° C. for 60 min. [125I]-PYY is added to the membrane-beads mix to achieve a concentration of 75 pM. 30 μl of the SPA mix is added to each well containing 0.3 μl compounds solution. Compound solutions are prepared by serially diluting compounds in neat DMSO. The incubation lasted 3 hours at room temperature under gentle shaking. Then plates are left overnight at room temperature and bound radioactivity is measured using ViewLux.

The functional activity of the compounds of the invention for the NPY Y5 receptor may be determined by the FLIPR/Ca²⁺ assay as described below. Such potency is typically calculated from the IC₅₀ obtained in FLIPR experiments as the concentration of a compound necessary to decrease 50% of the calcium release following cells exposure to a concentration of PYY eliciting 80% response (i.e. EC80), and is reported as a “fKᵢ” value calculated by the following equation:

$${fK}_{i} = \frac{{IC}_{50}}{1 + EC 80 / EC 50}$$

where EC80 and EC50 corresponding to the agonist (PYY) concentrations that eliciting 80% and 50% response, respectively (corresponding to the Cheng and Prusoff equation). In the context of the present invention pfKi values (corresponding to the antilogarithm of fKi) are used instead of fKi; pfKi results are only estimated to be accurate to about 0.3-0.5.

### Functional Activity at Recombinant Human NPY Y5 Receptor

The functional activity at the human NPY Y5 receptor stably expressed in HEK293 cells was assessed using FLIPR/Ca²⁺ methodology (cell line name: HEK 293 signal-hNPY Y5/G16z49). The assay is configured to re-direct receptor-mediated signalling to the calcium release from intracellular stores via the promiscuous Gal 6z49 protein. PYY (peptide YY) is an endogenous agonist and can activate the receptor, thereupon causing an increase in the level of calcium in the cells sensed by Fluo4-AM and measured by FLIPR. Antagonist effects are monitored by the blockade or decrease in calcium release once cells co-expressing hNPY Y5 receptor and Ga16z49 are exposed to a concentration of PYY eliciting 80% response (i.e. EC80). A non-linear, 4 parameter logistic curve-fit of the data generated pIC₅₀ value. Applying the Cheng-Prusoff equation to antagonist concentration-response for inhibition of fixed PYY concentration yielded the fpKi values.

Cells are cultured in DMEM/F12 supplemented with 10% FBS, 2 mM Glutamine, 200 μg/ml hygromycin B and 500 μg/ml G418. The day before a FLIPR experiment, cells are plated out into 384-well Poly-D-Lysine coated FLIPR plates at a density of 200'000 cells/ml corrects to give 10'000 cells per 50 μL per well using medium without antibiotics. On the day of experiment, cells are washed with an assay buffer containing 20 mM HEPES/NaOH, 145 mM NaCl, 5 mM KCl, 1 mM MgCl₂, 2 mM CaCl₂, 1 g/L D-glucose and 2.5 mM probenecid, pH 7.3 and loaded with 2 μM Fluo-4 AM for 60 min at 37° C. and 5% CO₂. The excess of dye solution is removed by washing cells with buffer. Compound solutions, prepared by serially diluting compounds in neat DMSO and then a final 1:50 dilution step in assay buffer added with 0.05% pluronic acid, are added and incubated with the loaded cells for 30 min at 37° C. and 5% CO₂.

Cells are then put in the FLIPR for the stimulus addition corresponding to a concentration of PYY eliciting 80% of the response. The response of cells to the agonist is fast and measured for 2 min after PYY addition.

All compounds of formula (I) are believed to bind the NPY Y5 receptor.

Preferred compounds show pKi comprised between 6 and 10 and fpKi comprised between 6 and 11 towards NPY Y5 receptor.

Compounds disclosed in Examples: 1-39, 1-55, 1-57, 3-6, 3-7, 3-9, 3-11, 3-12, 4-10, 4-12, 5-5 don't show activity at the highest concentration set by the experiment.

Compounds disclosed in Examples: 1-1, 1-2, 1-3, 1-5, 1-8, 1-9, 1-10, 1-11, 1-15, 1-16, 1-17, 1-20, 1-22, 1-23, 1-24, 1-29, 1-33, 1-34, 1-35, 1-36, 1-37, 1-40, 1-42, 1-43, 1-45, 1-46, 1-47, 1-49, 1-50, 1-51, 1-52, 1-54, 2-1, 3-1, 3-3, 4-1, 4-2, 4-3, 4-4, 4-5, 4-6, 4-7, 4-14, 4-15, 4-17, 4-20, 4-21, 4-22, 4-23, 4-24, 4-25, 4-26, 4-27, 4-28, 4-29, 4-30, 4-32, 4-33, 4-35, 4-36, 4-37, 4-38, 4-39, 4-40, 4-41, 4-42, 4-44, 4-45, 4-46, 4-47, 4-48, 4-49, 4-51, 4-52, 4-53, 4-55, 4-56, 5-1, 5-3, 5-4, 6-1, 7-2, 7-3, 7-5 show fpKi≧9.

Compounds disclosed in Examples: 1-9, 1-10, 1-11, 1-15, 1-17, 1-22, 1-24, 1-33, 1-34, 1-36, 1-37, 1-42, 1-43, 1-46, 1-49, 1-51, 1-52, 1-54, 2-1, 3-1, 4-2, 4-3, 4-4, 4-5, 4-6, 4-7, 4-14, 4-20, 4-23, 4-24, 4-25, 4-26, 4-30, 4-33, 4-35, 4-36, 4-37, 4-39, 4-40, 4-41, 4-42, 4-44, 4-45, 4-46, 4-47, 4-48, 4-49, 4-51, 4-53, 4-56, 6-1, 7-2, 7-5 show fpKi≧9.5.

Compounds disclosed in Examples: 1-10, 1-15, 1-24, 1-42, 1-52, 1-54, 2-1, 4-2, 4-3, 4-4, 4-5, 4-25, 4-39, 4-40, 4-45, 4-53, 4-56 show fpKi≧10.

1. A compound of formula (IID)′, or a pharmaceutically acceptable salt thereof, 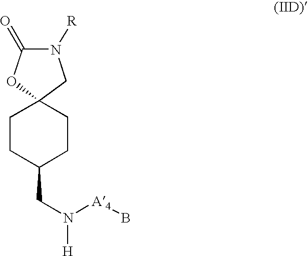
R is 2-pyridinyl, 3-pyridinyl, 4-pyridinyl, 4-pyridazinyl, 3-pyridazinyl or 1,3,4-thiadiazol-2-yl; which may be substituted by one or more: fluorine, bromine, chlorine, C₁-C₄ alkyl, C₁-C₄ alkoxy, C₁-C₄ haloalkyl, C₁-C₄ haloalkoxy, cyano;
A₄′ is 3,6-pyridazinyl, which may be substituted by one or more: halogen, C₁-C₄ alkyl, C₁-C₄ alkoxy, C₁-C₄ haloalkyl, C₁-C₄ haloalkoxy, cyano;
B is hydrogen, phenyl, pyridinyl, pyrazinyl, pyrrolidinyl or piperidinyl, which may be substituted by one or more: fluorine, bromine, chlorine, C₁-C₄ alkyl, C₁-C₄ alkoxy, C₁-C₄ haloalkyl, C₁-C₄ haloalkoxy, hydroxyl, cyano; A₄′ and B being linked via any atom.

2. A compound, according to claim 1, or a pharmaceutically acceptable salt thereof, wherein
R is 2-pyridinyl, 3-pyridinyl, 4-pyridinyl or 3-pyridazinyl, which may be substituted by one or more: fluorine, C₁-C₄ alkyl, C₁-C₄ alkoxy, C₁-C₄ haloalkyl, C₁-C₄ haloalkoxy, cyano;
A₄′ is 3,6-pyridazinyl, which may be substituted by one or more: fluorine, C₁-C₄ alkyl;
B is phenyl or 2-pyridyl, which may be substituted by one or more: fluorine. CF₃, C₁-C₄ alkyl.

3. A compound selected among: (trans)-8-({[6-(3,5-difluorophenyl)-3-pyrida-zinyl]amino}methyl)-3-(3-pyridazinyl)-1-oxa-3-azaspiro[4.5]decan-2-one of formula; 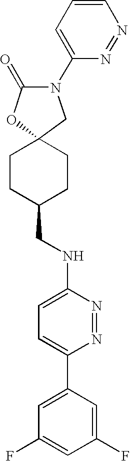
or a pharmaceutically acceptable salt thereof; and dihydrochloride salt of (trans)-8-({[6-(3,5-difluorophenyl)-3-pyridazinyl]amino}methyl)-3-(3-pyridazinyl)-1-oxa-3-azaspiro[4.5]decan-2-one of formula

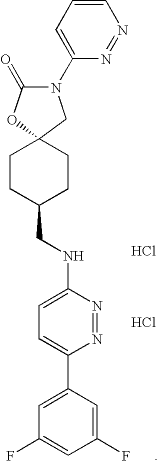

4. A compound selected among: (trans)-8-({[6-(2-fluorophenyl)-3-pyridazinyl]amino}methyl)-3-(2-fluoro-3-pyridinyl)-1-oxa-3-azaspiro[4.5]decan-2-one of formula; 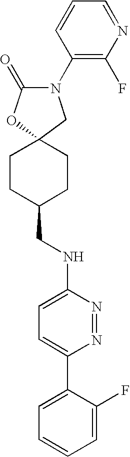
or a pharmaceutically acceptable salt thereof; and hydrochloride salt of (trans)-8-({[6-(2-fluorophenyl)-3-pyridazinyl]amino}methyl)-3-(2-fluoro-3-pyridinyl)-1-oxa-3-azaspiro[4.5]decan-2-one of formula

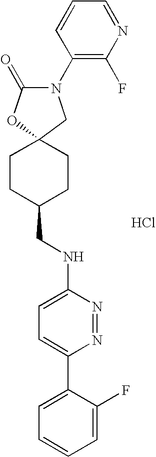

5. A method of treating a condition for which modulation of NPY Y5 receptors is beneficial, which comprises administering to a mammal in need thereof a therapeutically effective amount of a compound of claim 1.

6. A method as claimed in claim 5, wherein the mammal is human.

7. A method as claimed in claim 5, wherein the condition is selected among: eating disorders, binge eating, obesity or depression.

8. A pharmaceutical composition comprising a compound as claimed in claim 1 and a pharmaceutically acceptable carrier.
